(19) United States
(12) Patent Application Publication
(10) Pub. No.: US 2004/0018514 A1
(43) Pub. Date: Jan. 29, 2004

(54) Listeria inocua, genome and applications

(76) Inventors: Frederik Kunst, Ivry Sur Seine (FR); Philippe Glaser, Paris (FR)

Correspondence Address: FINNEGAN, HENDERSON, FARABOW, GARRETT & DUNNER, LLP, 1300 I STREET, NW, WASHINGTON, DC, 20005, US

(21) Appl. No.: 10/398,221

(22) PCT Filed: Oct. 4, 2001
(86) PCT No.: PCT/FR01/03061

(30) Foreign Application Priority Data
Oct. 4, 2000 (FR) 00/12697

(51) Int. Cl. C12Q 1/68; A61K 31/00; G06F 19/00; G01N 33/48; G01N 33/50; C07H 21/04; C12N 1/21; C12N 15/74

(52) U.S. Cl. 435/6; 536/23.7; 702/20; 435/252.3; 435/320.1; 514/1

(57) ABSTRACT

The invention concerns a nucleotide sequence derived from Listeria inocua corresponding to a sequence selected among SEQ ID NO: 1 to SEQ ID NO: 11 and the comparative analysis of said genome with that of Listeria monocytogenes.

The invention relates to a method for demonstrating the nucleotide sequences specific for the genome of a strain of bacterium of the genus Listeria, in particular of a strain of L. innocua or L. monocytogenes. The present invention also relates to the genomic sequence and to nucleotide sequences encoding polypeptides of Listeria innocua, such as cell envelope polypeptides secreted polypeptides or specific polypeptides or polypeptides involved in metabolism and in the replication process, and also to vectors including said sequences and to cells or animals transformed with these vectors. The invention also relates to the comparison of these nucleotide sequences with those encoding the polypeptides of Listeria monocytogenes, strain EGDe or L. monocytogenes 4b, and also the nucleotide sequences specific to these strains of Listeria. The invention also relates to methods for detecting these nucleic acids or polypeptides and kits for diagnosing contamination with bacteria of the genus Listeria and kits for typing contaminating strains. The invention is also directed toward a method for selecting compounds capable of modulating the bacterial infection engendered by other Listeria, and a method of biosynthesis or biodegradation of molecules of interest using said nucleotide sequences or said polypeptides. Finally, the invention comprises pharmaceutical, in particular vaccine, compositions for preventing and/or treating bacterial infections, in particular infections with Listeria, in particular monocytogenes, and compositions containing antibodies directed against polypeptides specific for L. innocua or for L. monocytogenes, strain EGDe or L. monocytogenes 4b.

In Listeria infections, Listeria monocytogenes is the most common and the most dangerous. Listeria monocytogenes is a facultative intracellular pathogen. It is the etiological agent of listeriosis, a food-related infection which poses increasingly great public health problems, with a considerable economic impact for the food industry. Listeriosis is the most lethal food-related infection (approximately 30% mortality). Listeria monocytogenes has the unusual property of being able to cross three barriers: the intestinal barrier, the blood-brain barrier and the placental barrier. Clinical manifestations of listeriosis include meningitis, meningoencephalitis, abortions and septicemia. This infection is opportunistic and affects mainly pregnant women, babies, elderly individuals and immunodepressed individuals, in particular individuals suffering from AIDS. This disease also affects healthy individuals and is responsible for a considerable amount of epidemics due to contaminated food products. Listeria monocytogenes is also important in veterinary terms, with a main risk for members of the sheep family and bovines. Listeria monocytogenes is particularly resistant to stress or to extreme conditions and it is important to search for its presence with care, not only for food safety problems but also for environmental safety problems.

Following the discovery of a contamination, it is necessary to type the strain(s) isolated in order to identify the origin of the contamination. Moreover, when the same installation is contaminated by two successive events, it is important to show with certainty whether these are two independent contaminations or whether the same strain is responsible for these two events. The most effective method currently used, the pulsed field gel electrophoresis (PFGE) profile after digestion of the chromosomal DNA, is a very laborious method which cannot be carried out systematically. An alternative method, less effective but automated, ribotyping, is very expensive per analysis, which limits its use.

It should also be underlined that the risk of listeriosis is very variable depending on the contaminating strain of Listeria. In extreme terms, some strains might be considered to be dangerous and others innocuous (such as Listeria innocua). Thus, although contaminations with Listeria are very common, the number of cases described is small. In this perspective, the availability of a tool for identifying the risk associated with a contamination (as a function of the genomic type of strain and of the number of bacteria per gram of food) would allow industrial companies to react as a function of this risk.

The complete sequence of the Listeria monocytogenes genome has been established for the strain EGDe deposited with the CNCM under the No. I-2440 on Apr. 11, 2000, and described in French patent application No. 00 04629 filed on Apr. 11, 2000. The genome of this bacterium is circular and comprises approximately 3 000 kilobases. Its GC content is approximately 38%. Virulence factor studies have made it possible to identify a 15 kb locus which can be considered to be a pathogenicity island insofar as it contains most of the genes whose function in virulence has been clearly identified.

A subject of the present invention is thus a method for demonstrating nucleotide sequences specific for the genome of a strain of bacterium of the genus Listeria, in particular specific for a strain of L. innocua or L. monocytogenes, such as the strain L. monocytogenes EGDe or L. monocytogenes 4b.

Such a method according to the invention in particular makes it possible to identify sequences specific for:

L. innocua compared to L. monocytogenes, in particular compared to L. monocytogenes EGDe and/or L. monocytogenes 4b;

L. monocytogenes, in particular L. monocytogenes EGDe or L. monocytogenes 4b, compared to L. innocua;

L. monocytogenes EGDe compared to L. innocua and/or L. monocytogenes 4b; and

L. monocytogenes 4b compared to L. innocua and/or L. monocytogenes EGDe.

Said method according to the invention is preferably characterized in that it comprises at least the following steps:

a) aligning the nucleotide sequences of L. monocytogenes, in particular those of L. monocytogenes EGDe and/or L. monocytogenes 4b, and those of L. innocua according to the invention; and

b) processing the data obtained with this alignment in order to isolate said specific sequences.

In a preferred embodiment, the method according to the invention is characterized in that the nucleotide sequences of L. monocytogenes, in particular those of L. monocytogenes EGDe and/or L. monocytogenes 4b, are chosen from the genomic nucleotide sequences:

as described in French patent application No. 00 04629 filed on Apr. 11, 2000, or in international patent application PCT/FR 01/01118 filed on Apr. 11, 2001 for L. monocytogenes EGDe, in particular the sequence SEQ ID No. 1 of the complete genome of L. monocytogenes EGDe; and

the sequences SEQ ID Nos. 1068 to 2041 or Nos. 2872 to 3891 for L. monocytogenes 4b.

In an embodiment which is also preferred, the method according to the invention is characterized in that the nucleotide sequences specific for L. innocua or L. monocytogenes, in particular those for L. monocytogenes EGDe and/or L. monocytogenes 4b, hybridize, under high stringency conditions, with respectively the nucleotide sequences, or the sequence complementary thereto, of L. innocua or L. monocytogenes, in particular those of L. monocytogenes EGDe and/or L. monocytogenes 4b.

The present invention relates to the nucleotide sequences and polypeptides of Listeria innocua and the comparison of the corresponding sequences with those of Listeria monocytogenes strain EGDe and/or 4b.

The invention relates in particular to:

the nucleic acid sequences SEQ ID Nos. 12 to 689 (cf. table V) and SEQ ID Nos. 2059 to 2601 (cf. table VI), in particular SEQ ID Nos. 2059 to 2601, specific for Listeria innocua compared to Listeria monocytogenes strain EGDe;

the nucleic acid sequences SEQ ID Nos. 690 to 1067 (cf. table V) and SEQ ID Nos. 2602 to 2871 (cf. table VII), in particular SEQ ID Nos. 2602 to 2871, specific for Listeria monocytogenes strain EGDe compared to Listeria innocua;

the nucleic acid sequences SEQ ID Nos. 3892 to 4025 (cf. table IX) specific for Listeria monocytogenes 4b compared to Listeria innocua and Listeria monocytogenes strain EGDe,

their fragments of sufficient length to conserve their abovementioned specificity, the sequence complementary thereto, their specific primers or probes, the peptides encoded by these nucleic acid sequences or antibodies directed against these peptides, and also in particular their uses for identifying a strain of Listeria or for distinguishing between a pathogenic or nonpathogenic strain of Listeria in a biological sample, in particular using methods or a diagnostic kit as presented below or known to those skilled in the art.

Using these specific sequences according to the invention, those skilled in the art will be able to design the primers or probes, or to produce the specific peptides or the antibodies directed against these peptides, required to carry out these diagnostic methods or the development of a diagnostic kit, as presented below, or which are standard.

Thus, an object of the present invention is to disclose the complete sequence of the genome of Listeria innocua, in particular CLIP 11262 contained in the genomic library prepared from the genome of this strain and deposited with the CNCM on Oct. 2, 2000, under the number I-2565, and also of all the genes and noncoding regulatory sequences contained in said genome.

The strain CLIP 11262 was isolated from a dairy product. This strain is conserved at the Centre National de Référence des Listeria [National Listeria Reference Center] at the Pasteur Institute (WHO collaborating center).

Comparison of the complete sequences of the genomes of L. monocytogenes strain EGDe and Listeria innocua, strain CLIP 11262, shows that approximately 86% of these genomes is very highly conserved (80 to 95% DNA identity). On the other hand, the remaining 14% is specific for each strain. In practical terms, a chip representing all of the genes of each species would give a positive signal for the DNA of the two strains for 86% of the probes and for 14%, would give a signal only with the DNA of one of the two strains.

These results are in agreement with the data from the literature on the diversity of Listeria strains. Moreover, recent laboratory data on the sequencing of an epidemic strain of L. monocytogenes (serotype 4b (CLIP 80459)) confirm this diversity but especially show that the strains of serotype 4b are without doubt as close to L. innocua as to the strain of L. monocytogenes serotype 1/2a, the genome of which has been sequenced. The strain CLIP 80459 is an epidemic strain. It is conserved at the Centre National de Référence des Listeria [National Listeria Reference Center] of the Pasteur Institute (WHO collaborating center). It should also be underlined that the innocua strain is not pathogenic and, consequently, that the genes specific for L. monocytogenes are potentially involved in the pathogenicity. Moreover, analysis of the genome of the EGDe strain has made it possible to identify the main competence genes, i.e. the genes which promote horizontal gene transfers. Certain strains of Listeria should, consequently, have the ability to be transformed. Horizontal transfers between strains should thus be common and explain the great diversity observed between the isolates.

The strain Listeria monocytogenes serotype 4b is also interchangeably identified in the present application by Listeria monocytogenes 4b.

All of these observations indicate that the genes identified as being variable between L. monocytogenes strain EGDe and L. innocua should be representative of the genomic diversity of Listeria.

The invention also relates to novel tools for typing Listeria strains. These tools might be of the DNA “chip” type or of another type. The novel characteristics of these typing tools will be as follows:

rapidity and simplicity of use;

high capacity for discrimination between strains;

possibility of providing information on the genomic content of the strain analyzed and of making it possible optionally to predict the risk associated with a Listeria contamination.

The present invention therefore relates to a nucleotide sequence of Listeria innocua, characterized in that it corresponds to a sequence chosen from SEQ ID No. 1 to SEQ ID No. 11, SEQ ID No. 2057 and SEQ ID No. 2058, in particular from SEQ ID No. 2057 and SEQ ID No. 2058.

The present invention also relates to a nucleotide sequence derived from Listeria innocua, characterized in that it is chosen from:

a) a nucleotide sequence comprising at least 75%, 80%, 85%, 90%, 95% or 98% identity with a sequence chosen from SEQ ID No. 1 to SEQ ID No. 11, SEQ ID No. 2057 and SEQ ID No. 2058, in particular from SEQ ID No. 2057 and SEQ ID No. 2058;

b) a nucleotide sequence which hybridizes, under high stringency conditions, with a sequence chosen from SEQ ID No. 1 to SEQ ID No. 11, SEQ ID No. 2057 and SEQ ID No. 2058;

c) a nucleotide sequence complementary to a sequence chosen from SEQ ID No. 1 to SEQ ID No. 11, SEQ ID No. 2057 and SEQ ID No. 2058, or complementary to a nucleotide sequence as defined in a) or b), or a nucleotide sequence of the RNA corresponding to either of the sequences a) and b);

d) a nucleotide sequence of a representative fragment of a sequence chosen from SEQ ID No. 1 to SEQ ID No. 11, SEQ ID No. 2057 and SEQ ID No. 2058, or of a representative fragment of a nucleotide sequence as defined in a), b) or c);

e) a nucleotide sequence comprising a sequence as defined in a), b), c) or d); and

f) a nucleotide sequence as defined in a), b), c), d) or e) which has been modified.

More particularly, a subject of the present invention is also the nucleotide sequences characterized in that they are derived from SEQ ID No. 1 to SEQ ID No. 11, SEQ ID No. 2057 and SEQ ID No. 2058 and in that they encode a polypeptide, chosen from the sequences SEQ ID No. 12 to SEQ ID No. 689, SEQ ID No. 2053 to SEQ ID No. 2056 and SEQ ID No. 2059 to SEQ ID No. 2601, in particular from SEQ ID No. 2059 to SEQ ID No. 2601.

The present invention also relates, more generally, to the nucleotide sequences derived from SEQ ID No. 1 to SEQ ID No. 11, SEQ ID No. 2057 and SEQ ID No. 2058 and encoding a polypeptide of L. innocua, such as they may be isolated from SEQ ID No. 1 to SEQ ID No. 11, SEQ ID No. 2057 and SEQ ID No. 2058, in particular from SEQ ID No. 2057 and SEQ ID No. 2058.

In addition, the nucleotide sequences, characterized in that they comprise a nucleotide sequence chosen from:

a) a nucleotide sequence encoding a polypeptide, chosen from the sequences SEQ ID No. 12 to SEQ ID No. 689, SEQ ID No. 2053 to SEQ ID No. 2056 and SEQ ID No. 2059 to SEQ ID No. 2601, in particular from SEQ ID No. 2059 to SEQ ID No. 2601;

b) a nucleotide sequence comprising at least 75%, 80%, 85%, 90%, 95% or 98% identity with a nucleotide sequence encoding a polypeptide, chosen from the sequences SEQ ID No. 12 to SEQ ID No. 689, SEQ ID No. 2053 to SEQ ID No. 2056 and SEQ ID No. 2059 to SEQ ID No. 2601, in particular from SEQ ID No. 2059 to SEQ ID No. 2601;

c) a nucleotide sequence which hybridizes, under high stringency conditions, with a nucleotide sequence encoding a polypeptide, chosen from the sequences SEQ ID No. 12 to SEQ ID No. 689, SEQ ID No. 2053 to SEQ ID No. 2056 and SEQ ID No. 2059 to SEQ ID No. 2601, in particular from SEQ ID No. 2059 to SEQ ID No. 2601;

d) a complementary or RNA sequence corresponding to a sequence as defined in a), b) or c);

e) a nucleotide sequence of a representative fragment of a sequence as defined in a), b), c) or d); and

f) a sequence as defined in a), b), c), d) or e) which has been modified, are also subjects of the invention.

The present invention also relates to a nucleotide sequence of Listeria monocytogenes serotype 4b of sequence SEQ ID No. 1068 to SEQ ID No. 2041 and SEQ ID No. 2872 to SEQ ID No. 3891, in particular SEQ ID No. 2872 to SEQ ID No. 3891.

The present invention also relates to a nucleotide sequence of Listeria monocytogenes serotype 4b, characterized in that it is chosen from:

a) a nucleotide sequence comprising at least 75%, 80%, 85%, 90%, 95% or 98% identity with SEQ ID No. 1068 to SEQ ID No. 2041, SEQ ID No. 2872 to SEQ ID No. 3891, in particular with SEQ ID No. 2872 to SEQ ID No. 3891;

b) a nucleotide sequence which hybridizes, under high stringency conditions, with SEQ ID No. 1068 to SEQ ID No. 2041, SEQ ID No. 2872 to SEQ ID No. 3891, in particular with SEQ ID No. 2872 to SEQ ID No. 3891;

c) a nucleotide sequence complementary to SEQ ID No. 1068 to SEQ ID No. 2041, SEQ ID No. 2872 to SEQ ID No. 3891, in particular to SEQ ID No. 2872 to SEQ ID No. 3891, or complementary to a nucleotide sequence as defined in a) or b), or a nucleotide sequence of the RNA corresponding to either of the sequences a) and b);

d) a nucleotide sequence of a representative fragment of SEQ ID No. 1068 to SEQ ID No. 2041, SEQ ID No. 2872 to SEQ ID No. 3891, in particular of SEQ ID No. 2872 to SEQ ID No. 3891, or of a representative fragment of a nucleotide sequence as defined in a), b) or c);

e) a nucleotide sequence comprising a sequence as defined in a), b), c) or d); and

f) a nucleotide sequence as defined in a), b), c), d) or e) which has been modified.

More particularly, the subject of the present invention is also the nucleotide sequences characterized in that they are derived from SEQ ID No. 1068 to SEQ ID No. 2041, SEQ ID No. 2872 to SEQ ID No. 3891, in particular from SEQ ID No. 2872 to SEQ ID No. 3891, and in that they encode a polypeptide, chosen from the sequences SEQ ID No. 690 to SEQ ID No. 1067, SEQ ID No. 2049 to SEQ ID No. 2052 and SEQ ID No. 2602 to SEQ ID No. 2871, in particular from SEQ ID No. 2602 to SEQ ID No. 2871.

The present invention also relates, more generally, to the nucleotide sequences derived from SEQ ID No. 1068 to 2041, SEQ ID No. 2872 to SEQ ID No. 3891, in particular from SEQ ID No. 2872 to SEQ ID No. 3891, and encoding a polypeptide of L. monocytogenes, such as they may be isolated from SEQ ID No. 690 to 1067, SEQ ID No. 2049 to SEQ ID No. 2052 and SEQ ID No. 2602 to SEQ ID No. 2871, in particular from SEQ ID No. 2602 to SEQ ID No. 2871.

In addition, the nucleotide sequences characterized in that they comprise a nucleotide sequence chosen from:

a) a nucleotide sequence encoding a polypeptide, chosen from the sequences SEQ ID No. 690 to SEQ ID No. 1067, SEQ ID No. 2602 to SEQ ID No. 2871, in particular from SEQ ID No. 2602 to SEQ ID No. 2871;

b) a nucleotide sequence comprising at least 75%, 80%, 85%, 90%, 95% or 98% identity with a nucleotide sequence encoding a polypeptide, chosen from the sequences SEQ ID No. 690 to SEQ ID No. 1067, SEQ ID No. 2602 to SEQ ID No. 2871, in particular from SEQ ID No. 2602 to SEQ ID No. 2871;

c) a nucleotide sequence which hybridizes, under high stringency conditions, with a nucleotide sequence encoding a polypeptide, chosen from the sequences SEQ ID No. 690 to SEQ ID No. 1067, SEQ ID No. 2602 to SEQ ID No. 2871, in particular from SEQ ID No. 2602 to SEQ ID No. 2871;

d) a complementary or RNA nucleotide sequence corresponding to a sequence as defined in a), b) or c);

e) a nucleotide sequence of a representative fragment of a sequence as defined in a), b), c) or d); and

f) a sequence as defined in a), b), c), d) or e) which has been modified, are also subjects of the invention.

The terms “nucleic acid”, “nucleic acid sequence”, “polynucleotide”, “oligonucleotide”, “polynucleotide sequence” and “nucleotide sequence”, terms which will be used indifferently in the present description, are intended to denote a precise chain of nucleotides, which may or may not be modified, which makes it possible to define a fragment or region of a nucleic acid, which may or may not comprise unnatural nucleotides, and which may correspond equally to a double-stranded DNA, a single-stranded DNA and transcription products of said DNAs. Thus, the nucleic acid sequences according to the invention also encompass PNAs (Peptide Nucleic Acids).

It should be understood that the present invention does not concern the nucleotide sequences in their natural chromosomal environment, i.e. in the natural state. They are sequences which have been isolated and/or purified, i.e. they have been removed directly or indirectly, for example by copying, their environment having been at least partially modified. Nucleic acids obtained by chemical synthesis are thus also intended to be denoted.

For the purpose of the present invention, the term “percentage identity” between two nucleic acid or amino acid sequences is intended to denote a percentage of nucleotides or amino acid residues which are identical between the two sequences to be compared, obtained after the best alignment, this percentage being purely statistical and the differences between the two sequences being distributed randomly and throughout their length. The term “best alignment” or “optimal alignment” is intended to denote the alignment for which the percentage identity determined as below is the highest. Sequence comparisons between two nucleic acid or amino acid sequences are conventionally carried out by comparing these sequences after having optimally aligned them, said comparison being carried out by segment or by “window of comparison” in order to identify and compare local regions of sequence similarity. The optimal alignment of the sequences for the comparison may be carried out, besides manually, by means of the local homology algorithm of Smith and Waterman, (1981, Ad. App. Math. 2:482), by means of the local homology algorithm of Neddleman and Wunsch (1970, J. Mol. Biol. 48:443), by means of the similarity search method of Pearson and Lipman (1988, Proc. Natl. Acad. Sci. USA 85:2444), by means of computer software using these algorithms (GAP, BESTFIT, BLAST P, BLAST N, FASTA and TFASTA in the Wisconsin Genetics Software Package, Genetics Computer Group, 575 Science Drive, Madison, Wis.). In order to obtain the optimal alignment, the BLAST program is preferably used, with the BLOSUM 62 matrix. The PAM or PAM250 matrices can also be used.

The percentage identity between two nucleic acid or amino acid sequences is determined by comparing these two sequences, which have been optimally aligned, the nucleic acid or amino acid sequence to be compared possibly comprising additions or deletions with respect to the reference sequence for optimal alignment between these two sequences. The percentage identity is calculated by determining the number of identical positions for which the nucleotide or the amino acid residue is identical in the two sequences, dividing this number of identical positions by the total number of positions compared and multiplying the result obtained by 100 so as to obtain the percentage identity between these two sequences.

The expression “nucleic acid sequences exhibiting a percentage identity of at least 75%, preferably 80%, 85% or 90%, more preferably 95%, or even 98%, after optimal alignment with a reference sequence” is intended to denote the nucleic acid sequences exhibiting, with respect to the reference nucleic acid sequence, certain modifications, such as in particular a deletion, a truncation, an extension, a chimeric fusion and/or a substitution, in particular a point substitution, and the nucleic acid sequence of which exhibits at least 75%, preferably 80%, 85%, 90%, 95% or 98%, identity, after optimal alignment, with the reference nucleic acid sequence. They are preferably sequences whose complementary sequences are capable of hybridizing specifically with the reference sequences. Preferably, the specific or high stringency hybridization conditions will be such that they ensure at least 75%, preferably 80%, 85%, 90%, 95% or 98%, identity, after optimal alignment, between one of the two sequences and the sequence complementary thereto.

Hybridization under high stringency conditions means that the conditions of temperature and of ionic strength are chosen such that they allow the hybridization between two complementary DNA fragments to be maintained. By way of illustration, high stringency conditions in the hybridization step for the purposes of defining the polynucleotide fragments described above are advantageously as follows.

The DNA-DNA or DNA-RNA hybridization is carried out in two steps: (1) prehybridization at 42° C. for 3 hours in phosphate buffer (20 mM, pH 7.5) containing 5×SSC (1×SSC corresponds to a solution of 0.15 M NaCl+0.015 M sodium citrate), 50% of formamide, 7% of sodium dodecyl sulfate (SDS), 10× Denhardt's, 5% of dextran sulfate and 1% of salmon sperm DNA; (2) actual hybridization for 20 hours at a temperature which depends on the length of the probe (i.e.: 42° C. for a probe >100 nucleotides in length), followed by 2 washes for 20 minutes at 20° C. in 2×SSC+2% SDS, 1 wash for 20 minutes at 20° C. in 0.1×SSC+0.1% SDS. The final wash is carried out in 0.1×SSC+0.1% SDS for 30 minutes at 60° C. for a probe >100 nucleotides in length. The high stringency hybridization conditions described above for a polynucleotide of defined length can be adjusted by those skilled in the art for longer or shorter oligonucleotides, according to the teaching of Sambrook et al. (1989, Molecular cloning: a laboratory manual, 2^{nd} Ed. Cold Spring Harbor).

In addition, the expression “representative fragment of sequences according to the invention” is intended to denote any nucleotide fragment having at least 15 consecutive nucleotides, preferably at least 30, 75, 150, 300 and 450 consecutive nucleotides, of the sequence from which it is derived.

The term “representative fragment” is intended to mean in particular a nucleic acid sequence encoding a biologically active fragment of a polypeptide, as defined later.

The term “representative fragment” is also intended to mean the intergenic sequences, and in particular the nucleotide sequences carrying the regulatory signals (promoters, terminators, or even enhancers, etc.).

Among said representative fragments, preference is given to those having nucleotide sequences corresponding to open reading frames, called ORF sequences, generally included between an initiation codon and a stop codon, or between two stop codons, and encoding polypeptides, preferably of at least 100 amino acids, such as, for example, without being limited thereto, the ORF sequences which will be described subsequently.

The numbering of the ORF nucleotide sequences which will be used subsequently in the present description corresponds to the numbering of the amino acid sequences of the proteins encoded by said ORFs.

The representative fragments according to the invention can be obtained, for example, by specific amplification such as PCR or after digestion with suitable restriction enzymes of nucleotide sequences according to the invention, this method being described in particular in the work by Sambrook et al.. Said representative fragments can also be obtained by chemical synthesis when they are not too great in length, according to methods well known to those skilled in the art.

The sequences which are naturally framed by sequences which exhibit at least 75%, 80%, 85%, 90%, 95% or 98% identity with the sequences according to the invention are also intended to be included among the sequences containing sequences of the invention, or representative fragments.

The expression “nucleotide sequence which has been modified” is intended to mean any nucleotide sequence obtained by mutagenesis according to techniques well known to those skilled in the art, and comprising modifications compared to the normal sequences, for example mutations in the sequences regulating and/or promoting the expression of the polypeptide, in particular leading to a modification of the level of expression or of the activity of said polypeptide.

The expression “nucleotide sequence which has been modified” is also intended to mean any nucleotide sequence encoding a modified polypeptide as defined below.

The representative fragments according to the invention may also be probes or primers, which can be used in methods for detecting, identifying, assaying or amplifying nucleic acid sequences.

For the purpose of the invention, a probe or primer is defined as being a single-stranded fragment of nucleic acid or a denatured double-stranded fragment comprising, for example, from 12 bases to a few kb, in particular from 15 to a few hundred bases, preferably from 15 to 50 or 100 bases, and having a specificity of hybridization under given conditions so as to form a hybridization complex with a target nucleic acid.

The probes and primers according to the invention can be labeled directly or indirectly with a radioactive or nonradioactive compound using methods well known to those skilled in the art, in order to obtain a detectable and/or quantifiable signal (patent FR 78 10975 and bDNA of Chiron EP 225 807 and EP 510 085).

The unlabeled sequences of polynucleotides according to the invention can be used directly as a probe or primer.

The sequences are generally labeled so as to obtain sequences which can be used for many applications. The labeling of the primers or of the probes according to the invention is carried out with radioactive elements or with nonradioactive molecules.

Among the radioactive isotopes used, mention may be made of ³²P, ³³P, ³⁵S, ³H and 125I. The nonradioactive entities are selected from ligands such as biotin, avidin or streptavidin, digoxigenin, haptens, dyes and luminescent agents, such as radioluminescent, chemiluminescent, bioluminescent, fluorescent or phosphorescent agents.

The polynucleotides according to the invention can thus be used as a primer and/or probe in methods using in particular the PCR (polymerase chain reaction) technique (Rolfs et al., 1991, Berlin: Springer-Verlag). This technique requires the choice of pairs of oligonucleotide primers framing the fragment which must be amplified. Reference may, for example, be made to the technique described in U.S. Pat. No. 4,683,202. The amplified fragments can be identified, for example after agarose or polyacrylamide gel electrophoresis or after a chromatographic technique such as gel filtration or ion exchange chromatography, and then sequenced. The specificity of the amplification can be controlled using the nucleotide sequences of polynucleotides of the invention as matrix, plasmids containing these sequences or else the derived amplification products. The amplified nucleotide fragments can be used as reagents in hybridization reactions in order to demonstrate the presence, in a biological sample, of a target nucleic acid of sequence complementary to that of said amplified nucleotide fragments.

The invention is also directed toward the nucleic acids which can be obtained by amplification using primers according to the invention.

Other techniques for amplifying the target nucleic acid can advantageously be used as an alternative to a PCR (PCR-like) using a pair of primers of nucleotide sequences according to the invention. The term “PCR-like” is intended to denote all the methods using direct or indirect reproductions of nucleic acid sequences, or else in which the labeling systems have been amplified; these techniques are, of course, known. In general, this involves amplification of the DNA with a polymerase; when the sample of origin is an RNA, a reverse transcription should be carried out beforehand. A very large number of methods currently exist for this amplification, such as, for example, the SDA (Strand Displacement Amplification) technique (Walker et al., 1992, Nucleic Acids Res. 20:1691), the TAS (Transcription-based Amplification System) technique described by Kwoh et al. (1989, Proc. Natl. Acad. Sci. USA, 86, 1173), the 3SR (Self-Sustained Sequence Replication) technique described by Guatelli et al. (1990, Proc. Natl. Acad. Sci. USA, 87:1874), the NASBA (Nucleic Acid Sequence Based Amplification) technique described by Kievitis et al. (1991, J. Virol. Methods, 35, 273), the TMA (Transcription Mediated Amplification) technique, the LCR (Ligase Chain Reaction) technique described by Landegren et al. (1988, Science 241, 1077), the RCR (Repair Chain Reaction) technique described by Segev (1992, C. Kessler, Springer Verlag, Berlin, New-York, 197-205), the CPR (Cycling Probe Reaction) technique described by Duck et al. (1990, Biotechniques, 9, 142), and the Q-beta-replicase amplification technique described by Miele et al. (1983, J. Mol. Biol., 171, 281). Some of these techniques have since been improved.

When the target polynucleotide to be detected is an mRNA, an enzyme of the reverse transcriptase type is advantageously used, prior to carrying out an amplification reaction using the primers according to the invention or to carrying out a method of detection using the probes of the invention, in order to obtain a cDNA from the mRNA contained in the biological sample. The cDNA obtained will then be used as a target for the primers or the probes used in the method of amplification or of detection according to the invention.

The probe hybridization technique can be carried out in various ways (Matthews et al., 1988, Anal. Biochem., 169, 1-25). The most general method consists in immobilizing the nucleic acid extracted from the cells of various tissues or from cells in culture on a support (such as nitrocellulose, nylon or polystyrene) and in incubating, under well-defined conditions, the immobilized target nucleic acid with the probe. After hybridization, the excess probe is removed and the hybrid molecules formed are detected using the appropriate method (measuring the radioactivity, the fluorescence or the enzyme activity associated with the probe).

According to another embodiment of the nucleic acid probes according to the invention, the latter may be used as capture probes. In this case, a probe, termed “capture probe”, is immobilized on a support and is used to capture, by specific hybridization, the target nucleic acid obtained from the biological sample to be tested, and the target nucleic acid is then detected using a second probe, termed “detection probe”, labeled with a readily detectable element.

Among the advantageous nucleic acid fragments, mention should thus be made in particular of antisense oligonucleotides, i.e. oligonucleotides whose structure provides, by hybridization with the target sequence, inhibition of the expression of the corresponding product. Mention should also be made of sense oligonucleotides which, by interaction with proteins involved in regulating the expression of the corresponding product, will induce either an inhibition or an activation of this expression.

Preferably, the probes or primers according to the invention are immobilized on a support, covalently or noncovalently. In particular, the support may be a DNA chip or a high- or medium-density filter, which are also subjects of the present invention (patents WO 97/29212, WO 98/27317, WO 97/10365 and WO 92/10588).

The term “DNA chip” or “high density filter” is intended to mean a support to which are attached DNA sequences, each one of them being able to be pinpointed by its geographical location. These chips or filters differ mainly by their size, the material of the support and, optionally, the number of DNA sequences which are attached thereto.

The probes or primers according to the present invention can be attached to the solid supports, in particular DNA chips, by various methods of production. In particular, in situ synthesis can be carried out by photo-chemical addressing or by inkjet. Other techniques consist in carrying out an ex situ synthesis and attaching the probes to the support of the DNA chip by mechanical or electronic addressing or by inkjet. These various methods are well known to those skilled in the art.

A nucleotide sequence (probe or primer) according to the invention therefore makes it possible to detect and/or amplify specific nucleic acid sequences. In particular, the detection of these said sequences is facilitated when the probe is attached to a DNA chip or to a high density filter.

The use of DNA chips or high density filters in fact makes it possible to determine the gene expression in an organism having a genomic sequence close to L. monocytogenes or innocua, and to type the strain in question.

The genomic sequence of L. innocua and the partial sequences of L. monocytogenes 4b, completed by the identification of the genes of these organisms, as presented in the present invention, serve as a basis to construct these DNA chips or filters.

The preparation of these filters or chips consists in synthesizing oligonucleotides, corresponding to the 5′ and 3′ ends of the genes or to more internal fragments, in order to amplify fragments of an appropriate length, for example between approximately 300 and 800 bases. These oligonucleotides are chosen using the genomic sequence and its annotations disclosed by the present invention. The temperature of pairing of these oligonucleotides at the corresponding places on the DNA should be approximately the same for each oligonucleotide. This makes it possible to prepare DNA fragments corresponding to each gene using appropriate PCR conditions in a highly automated environment. The amplified fragments are then immobilized on filters or supports made of glass, silicon or synthetic polymers and these media are used for the hybridization.

The availability of such filters and/or chips and of the corresponding annotated genomic sequence makes it possible to study the expression of large sets, or even of all, of the genes in the microorganisms associated with Listeria innocua and L. monocytogenes 4b, by preparing the complementary DNAs and hybridizing them to the DNA or to the oligonucleotides immobilized on the filters or chips. Similarly, the filters and/or the chips make it possible to study the strain or species variability by preparing the DNA of these organisms and hybridizing it to the DNA or to the oligonucleotides immobilized on the filters or the chips.

The differences between the genomic sequences of the various strains or species can greatly affect the intensity of the hybridization and, consequently, disturb the interpretation of the results. It may therefore be necessary to have the precise sequence of the genes of the strain intended to be studied. The method for detecting the genes described later in detail, involving determining the sequence of random fragments of a genome and organizing them according to the sequence of the complete genome of L. innocua and L. monocytogenes 4b disclosed in the present invention will be very useful.

The nucleotide sequences according to the invention can be used in DNA chips to carry out mutation analysis. This analysis is based on constituting chips capable of analyzing each base of a nucleotide sequence according to the invention. For this purpose, use may in particular be made of the techniques of microsequencing on a DNA chip. The mutations are detected by extension of immobilized primers which hybridize to the matrix of the sequences analyzed, just at a position adjacent to that of the mutated nucleotide sought. A single-stranded, RNA or DNA matrix of the sequences to be analyzed will be advantageously prepared according to conventional methods, using products amplified according to PCR-type techniques. The single-stranded DNA or RNA matrices thus obtained are then deposited onto the DNA chip, under conditions which allow their specific hybridization to the immobilized primers. A thermostable polymerase, for example Tth or Taq DNA polymerase, specifically extends the 3′ end of the immobilized primer with a labeled nucleotide analog complementary to the nucleotide at the position of the variable site; for example, thermal cycling is carried out in the presence of fluorescent dideoxyribonucleotides. The experimental conditions will be adjusted in particular to the chips used, to the immobilized primers, to the polymerases used and to the labeling system chosen. An advantage of the microsequencing, compared to techniques based on probe hybridization, is that it makes it possible to identify all the variable nucleotides with optimal discrimination under homogeneous reaction conditions; when used on DNA chips, it allows optimal resolution and specificity for the routine and industrial detection of mutations in a multiplex.

The use of the high density filters and/or the chips thus makes it possible to obtain new knowledge regarding gene regulation in organisms of industrial importance, and in particular the listeria propagated under diverse conditions. It also allows rapid identification of the differences between the genomes of the strains used in many industrial applications.

In addition, a DNA chip or a filter can be an extremely advantageous tool for determining, detecting and/or identifying a microorganism. Thus, the DNA chips according to the invention which also contain at least one nucleotide sequence of the microorganism other than Listeria monocytogenes 4b or Listeria innocua, immobilized on the support of said chip, are also preferred. The microorganism chosen is preferably done so from the bacteria of the genus Listeria (hereinafter referred to as L. monocytogenes-related bacteria), or the variants of Listeria monocytogenes EGD-e.

A DNA chip or a filter according to the invention is a very useful element of certain kits or sets for detecting and/or identifying microorganisms, in particular the bacteria belonging to the species Listeria monocytogenes or the related microorganisms, which are also subjects of the invention.

Moreover, the DNA chips or the filters according to the invention, containing probes or primers specific for Listeria innocua or monocytogenes, are very advantageous elements of kits or sets for detecting and/or quantifying the expression of genes of Listeria innocua or monocytogenes (or related microorganisms).

Specifically, the control of gene expression is a critical point for optimizing the growth and yield of a strain, either by allowing the expression of one or more new genes, or by modifying the expression of genes already present in the cell. The present invention provides all of the sequences naturally active in L. innocua which allow gene expression. It thus makes it possible to determine all the sequences expressed in L. innocua. It also provides a tool for pinpointing the genes whose expression follows a given scheme. To do this, the DNA of all or some of the genes of L. innocua and monocytogenes can be amplified using the primers according to the invention, and then attached to a support such as, for example, glass or nylon or a DNA chip, in order to construct a tool for following the expression profile of these genes. This tool, consisting of this support containing the coding sequences, serves as a matrix of hybridization to a mixture of labeled molecules reflecting the messenger RNAs expressed in the cell (in particular the labeled probes according to the invention). By repeating this experiment at various moments and combining all of the data using suitable processing, the expression profiles of all these genes are then obtained. The knowledge of the sequences which follow a given regulatory scheme can also be taken advantage of to search for, in a directed manner, for example by homology, other sequences following overall, but in a slightly different way, the same regulatory scheme. In addition, it is possible to isolate each control sequence present upstream of the segments acting as probes and to follow the activity thereof using suitable means such as a reporter gene (luciferase, β-galactosidase, GFP). These isolated sequences can then be modified and assembled by metabolic engineering with sequences of interest with a view to the optimal expression thereof.

The invention also relates to the polypeptides encoded by a nucleotide sequence according to the invention, preferably by a representative fragment of the preceding sequences, corresponding to an ORF sequence. In particular, the polypeptides of Listeria innocua encoded by the sequences SEQ ID No. 12 to SEQ ID No. 689, SEQ ID Nos. 2042 and 2043, SEQ ID Nos. 2047 and 2048, SEQ ID Nos. 2053 to 2056 and SEQ ID Nos. 2059 to 2601, in particular by SEQ ID Nos. 2059 to 2601, or those of Listeria monocytogenes EGDe, characterized in that they are chosen from the polypeptides encoded by the sequences SEQ ID No. 690 to SEQ ID No. 1067, SEQ ID No. 2049 to SEQ ID No. 2052 and SEQ ID Nos. 2602 to 2871, in particular SEQ ID Nos. 2602 to 2871, or those of Listeria monocytogenes 4b, characterized in that they are chosen from polypeptides encoded by the sequences SEQ ID No. 3892 to SEQ ID No. 4025, are subjects of the invention.

The invention also comprises the polypeptides characterized in that they comprise a polypeptide chosen from:

a) a polypeptide according to the invention;

b) a polypeptide exhibiting at least 80%, preferably 85%, 90%, 95% and 98%, identity with a polypeptide according to the invention;

c) a fragment of at least 5 amino acids of a polypeptide according to the invention, or as defined in b);

d) a biologically active fragment of a polypeptide according to the invention, or as defined in b) or c); and

e) a polypeptide according to the invention or as defined in b), c) or d) which has been modified.

The nucleotide sequences encoding the polypeptides described above are also subjects of the invention.

In the present description, the terms “polypeptides”, “polypeptide sequences”, “peptides” and “proteins” are interchangeable. The term “polypeptide” comprises any amino acid sequence making it possible to generate an antibody response.

It should be understood that the invention does not concern the polypeptides in natural form, i.e. they are not taken in their natural environment. On the other hand, it concerns those which it has been possible to isolate or obtain by purification from natural sources, or as those obtained by genetic recombination or by chemical synthesis, and they can then comprise unnatural amino acids as already described below.

The expression “polypeptide exhibiting a certain percentage identity with another”, which will also be referred to as “homologous polypeptide”, is intended to denote the polypeptides exhibiting, compared to the natural polypeptides, certain modifications, in particular deletion, addition or substitution of at least one amino acid, a truncation, an extension, a chimeric solution and/or mutation, or the polypeptides exhibiting post-translation modifications. Among the homologous polypeptides, preference is given to those in which the amino acid sequence exhibits at least 80%, preferably 85%, 90%, 95% and 98%, homology with the amino acid sequences of the polypeptides according to the invention. In the case of a substitution, one or more consecutive or nonconsecutive amino acid(s) is (are) replaced with “equivalent” amino acids. The expression “equivalent amino acids” is here aimed at denoting any amino acid capable of being substituted for one of the amino acids of the basic structure without, however, essentially modifying the biological activities of the corresponding peptides as they will be defined subsequently.

These equivalent amino acids can be determined either based on their structural homology with the amino acids for which they substitute, or on results of comparative biological activity assays between the various polypeptides liable to be produced.

By way of example, mention is made of the substitution possibilities which can be made without it resulting in a profound modification of the biological activity of the corresponding modified polypeptide. It is thus possible to replace leucine with valine or isoleucine, aspartic acid with glutamic acid, glutamine with asparagine, arginine with lysine, etc., it naturally being possible to envision the reverse substitutions under the same conditions.

The homologous polypeptides also correspond to the polypeptides encoded by the homologous or identical nucleotide sequences as defined previously and thus comprise, in the present definition, mutated polypeptides or polypeptides corresponding to inter- or intraspecies variations which can exist in Listeria and which correspond in particular to truncations, substitutions, deletions and/or additions of at least one amino acid residue.

It is understood that the percentage identity between two polypeptides is calculated in the same way as between two nucleic acid sequences. Thus, the percentage identity between two polypeptides is calculated after optimal alignment of these two sequences, on a window of maximum homology. To define said window of maximum homology, the same algorithms as for the nucleic acid sequences can be used.

The expression “biologically active fragment of a polypeptide according to the invention” is intended to denote in particular a polypeptide fragment, as defined below, exhibiting at least one of the biological characteristics of the polypeptides according to the invention, particularly in that it is capable of exercising in general even a partial activity, such as, for example:

an enzymatic (metabolic) activity or an activity which may be involved in the biosynthesis or the biodegradation of organic or inorganic compounds;

a structural activity (cell envelope, chaperone molecule, ribosome);

a transport activity (energy transport, ion transport); or an activity in protein secretion;

an activity in the process of replication, amplification, preparation, transcription, translation or maturation, in particular of DNA, of RNA or of proteins.

The expression “polypeptide fragment according to the invention” is intended to denote a polypeptide comprising at least 5 amino acids, preferably 10, 15, 25, 50, 100 and 150 amino acids.

The polypeptide fragments can correspond to isolated or purified fragments naturally present in the strains of Listeria, or to fragments which can be obtained by cleavage of said polypeptide with a proteolytic enzyme, such as trypsin or chymotrypsin or collagenase, or with a chemical reagent (cyanogen bromide, CNBr) or by placing said polypeptide in a very acidic environment (for example at pH=2.5). Polypeptide fragments can also be prepared by chemical synthesis, or from hosts transformed with an expression vector according to the invention which contain a nucleic acid which allows expression of said fragment and is placed under the control of the appropriate regulatory and/or expression element.

The term “modified polypeptide” of a polypeptide according to the invention is intended to denote a polypeptide obtained by genetic recombination or by chemical synthesis as described later, which exhibits at least one modification compared to the normal sequence. These modifications may in particular be made on amino acids necessary for the specificity or the effectiveness of the activity, or responsible for the structural conformation, for the charge or for the hydrophobicity of the polypeptide according to the invention. It is thus possible to create polypeptides with equivalent, increased or decreased activity, or with equivalent, stricter or broader specificity. Among the modified polypeptides, mention should be made of the polypeptides in which up to five amino acids can be modified, truncated at the N- or C-terminal end, or else deleted, or added.

As is indicated, the aim of the modifications to a polypeptide is in particular:

to allow its use in methods of biosynthesis or of biodegradation of organic or inorganic compounds,

to allow its use in methods of replication, of amplification, of repair and of regulation of transcription, translation or maturation in particular of DNA, RNA or proteins,

to allow its improved secretion,

to modify its solubility, or the effectiveness or specificity of its activity, or else to facilitate its purification.

Chemical synthesis also has the advantage of being able to use unnatural amino acids or nonpeptide bonds. Thus, it may be advantageous to use unnatural amino acids, for example in the D form, or amino acid analogs, in particular the sulfur-containing forms.

The present invention provides the nucleotide sequence of the Listeria innocua genome and the partial sequence of Listeria monocytogenes serotype 4b, and also some polypeptide sequences.

Preferably, the invention relates to a nucleotide sequence according to the invention, characterized in that it encodes a polypeptide of Listeria innocua or monocytogenes 4b or one of its fragments, involved in amino acid biosynthesis.

Preferably, the invention relates to a nucleotide sequence according to the invention, characterized in that it encodes a polypeptide of Listeria innocua or monocytogenes 4b, or one of its fragments, involved in the biosynthesis of cofactors, prosthetic groups and transporters.

Preferably, the invention relates to a nucleotide sequence according to the invention, characterized in that it encodes a cell envelope polypeptide or a polypeptide present at the surface of Listeria innocua or monocytogenes 4b, or one of its fragments.

Preferably, the invention relates to a nucleotide sequence according to the invention, characterized in that it encodes a polypeptide of Listeria innocua or monocytogenes 4b, or one of its fragments, involved in the cellular machinery.

Preferably, the invention relates to a nucleotide sequence according to the invention, characterized in that it encodes a polypeptide of Listeria innocua or monocytogenes 4b, or one of its fragments, involved in central intermediate metabolism.

Preferably, the invention relates to a nucleotide sequence according to the invention, characterized in that it encodes a polypeptide of Listeria innocua or monocytogenes 4b, or one of its fragments, involved in energy metabolism.

Preferably, the invention relates to a nucleotide sequence according to the invention, characterized in that it encodes a polypeptide of Listeria innocua or monocytogenes 4b, or one of its fragments, involved in fatty acid and phospholipid metabolism.

Preferably, the invention relates to a nucleotide sequence according to the invention, characterized in that it encodes a polypeptide of Listeria innocua or monocytogenes 4b, or one of its fragments, involved in the metabolism of nucleotides, purines, pyrimidines or nucleosides.

Preferably, the invention relates to a nucleotide sequence according to the invention, characterized in that it encodes a polypeptide of Listeria innocua or monocytogenes 4b, or one of its fragments, involved in regulatory functions.

Preferably, the invention relates to a nucleotide sequence according to the invention, characterized in that it encodes a polypeptide of Listeria innocua or monocytogenes 4b, or one of its fragments, involved in the replication process.

Preferably, the invention relates to a nucleotide sequence according to the invention, characterized in that it encodes a polypeptide of Listeria innocua or monocytogenes 4b, or one of its fragments, involved in the transcription process.

Preferably, the invention relates to a nucleotide sequence according to the invention, characterized in that it encodes a polypeptide of Listeria innocua or monocytogenes 4b, or one of its fragments, involved in the translation process.

Preferably, the invention relates to a nucleotide sequence according to the invention, characterized in that it encodes a polypeptide of Listeria innocua or monocytogenes 4b, or one of its fragments, involved in the process of protein transport and binding.

Preferably, the invention relates to a nucleotide sequence according to the invention, characterized in that it encodes a polypeptide of Listeria innocua or monocytogenes 4b, or one of its fragments, involved in adaptation to atypical conditions.

Preferably, the invention relates to a nucleotide sequence according to the invention, characterized in that it encodes a polypeptide of Listeria innocua or monocytogenes 4b, or one of its fragments, involved in sensitivity to medicinal products and analogs.

Preferably, the invention relates to a nucleotide sequence according to the invention, characterized in that it encodes a polypeptide of Listeria innocua or monocytogenes 4b, or one of its fragments, involved in functions relating to transposons.

Preferably, the invention relates to a nucleotide sequence according to the invention, characterized in that it encodes a polypeptide specific for Listeria innocua or monocytogenes 4b, or one of its fragments.

In another aspect, a subject of the invention is preferably a polypeptide according to the invention, characterized in that it is a polypeptide of Listeria innocua or monocytogenes 4b, or one of its fragments, involved in amino acid biosynthesis.

In another aspect, a subject of the invention is preferably a polypeptide according to the invention, characterized in that it is a polypeptide of Listeria innocua or monocytogenes 4b, or one of its fragments, involved in the biosynthesis of cofactors, prosthetic groups and transporters.

According to another aspect, a subject of the invention is preferably a polypeptide according to the invention, characterized in that it is a cell envelope polypeptide or a surface polypeptide of Listeria innocua or monocytogenes 4b, or one of its fragments.

In another aspect, a subject of the invention is preferably a polypeptide according to the invention, characterized in that it is a polypeptide of Listeria innocua or monocytogenes 4b, or one of its fragments, involved in the cellular machinery.

In another aspect, a subject of the invention is preferably a polypeptide according to the invention, characterized in that it is a polypeptide of Listeria innocua or monocytogenes 4b, or one of its fragments, involved in central intermediate metabolism.

In another aspect, a subject of the invention is preferably a polypeptide according to the invention, characterized in that it is a polypeptide of Listeria innocua or monocytogenes 4b, or one of its fragments, involved in energy metabolism.

In another aspect, a subject of the invention is preferably a polypeptide according to the invention, characterized in that it is a polypeptide of Listeria innocua or monocytogenes 4b, or one of its fragments, involved in fatty acid and phospholipid metabolism.

In another aspect, a subject of the invention is preferably a polypeptide according to the invention, characterized in that it is a polypeptide of Listeria innocua or monocytogenes 4b, or one of its fragments, involved in the metabolism of nucleotides, purines, pyrimidines or nucleosides.

In another aspect, a subject of the invention is preferably a polypeptide according to the invention, characterized in that it is a polypeptide of Listeria innocua or monocytogenes 4b, or one of its fragments, involved in regulatory functions.

In another aspect, a subject of the invention is preferably a polypeptide according to the invention, characterized in that it is a polypeptide of Listeria innocua or monocytogenes 4b, or one of its fragments, involved in the replication process.

In another aspect, a subject of the invention is preferably a polypeptide according to the invention, characterized in that it is a polypeptide of Listeria innocua or monocytogenes 4b, or one of its fragments, involved in the transcription process.

In another aspect, a subject of the invention is preferably a polypeptide according to the invention, characterized in that it is a polypeptide of Listeria innocua or monocytogenes 4b, or one of its fragments, involved in the translation process.

In another aspect, a subject of the invention is preferably a polypeptide according to the invention, characterized in that it is a polypeptide of Listeria innocua or monocytogenes 4b, or one of its fragments, involved in the process of protein transport and binding.

In another aspect, a subject of the invention is preferably a polypeptide according to the invention, characterized in that it is a polypeptide of Listeria innocua or monocytogenes 4b, or one of its fragments, involved in adaptation to atypical conditions.

In another aspect, a subject of the invention is preferably a polypeptide according to the invention, characterized in that it is a polypeptide of Listeria innocua or monocytogenes 4b, or one of its fragments, involved in sensitivity to medicinal products and analogs.

In another aspect, a subject of the invention is preferably a polypeptide according to the invention, characterized in that it is a polypeptide of Listeria innocua or monocytogenes 4b, or one of its fragments, involved in functions relating to transposons.

In another aspect, a subject of the invention is preferably a polypeptide according to the invention, characterized in that it is a polypeptide specific for Listeria innocua or monocytogenes 4b, or one of its fragments.

It is important to note, however, that a living organism is a whole entity and should be taken as such. Thus, in order to be able to develop and exhibit its properties, any organism needs interactions between the various metabolic pathways. Thus, the classification stated above should not be considered to be limiting, it being possible for a gene to be involved in two distinct metabolic pathways.

A subject of the present invention is also the nucleotide and/or polypeptide sequences according to the invention, characterized in that said sequences are recorded on a recording medium, the form and nature of which facilitate the reading, analysis and/or exploitation of said sequence(s). These media may also contain other information extracted from the present invention, in particular the analogies with already known sequences, and/or information concerning the nucleotide and/or polypeptide sequences of other microorganisms, in order to facilitate the comparative analysis and exploitation of the results obtained.

Among these recording media, preference is given in particular to computer-readable media, such as magnetic, optical, electrical or hybrid media, in particular computer disks, CD-ROMs and computer servers. Such recording media are also a subject of the invention.

The recording media according to the invention, with the information provided, are very useful for choosing nucleotide primers or probes for determining genes in Listeria innocua or monocytogenes 4b or strains close to this organism. Similarly, the use of these media for studying the genetic polymorphism of strains close to Listeria innocua or monocytogenes 4b, in particular by determining the regions of colinearity, is very useful insofar as these media provide not only the nucleotide sequence of the genome of Listeria innocua or monocytogenes 4b, but also the genomic organization in said sequence. Thus, the uses of recording media according to the invention are also subjects of the invention.

The analysis of homology between various sequences is in fact advantageously performed using sequence comparison programs, such as the Blast program, or the programs of the GCG package, described above.

The invention is also directed toward the cloning and/or expression vectors which contain a nucleotide sequence according to the invention.

The vectors according to the invention preferably comprise elements which allow the expression and/or the secretion of the nucleotide sequences in a given host cell.

The vector should then comprise a promoter, translation initiation and termination signals, and also regions suitable for regulating transcription. It must be possible for it to be maintained stably in the host cell and it may optionally contain particular signals which specify secretion of the translated protein. These various elements are chosen and optimized by those skilled in the art, depending on the cellular host used. To this effect, the nucleotide sequences according to the invention can be inserted into vectors which replicate autonomously in the host chosen, or may be vectors which integrate in the host chosen.

Such vectors are prepared by methods commonly used by those skilled in the art, and the resulting clones can be introduced into a suitable host using standard methods, such as lipofection, electroporation, heat shock or chemical methods.

The vectors according to the invention are, for example, vectors of plasmid or viral origin. They are of use in transforming host cells in order to clone or express the nucleotide sequences according to the invention.

The invention also comprises the host cells transformed with a vector according to the invention.

The cellular host may be chosen from prokaryotic or eukaryotic systems, for example bacterial cells but also yeast cells or animal cells, in particular mammalian cells. Insect cells or plant cells may also be used. The preferred host cells according to the invention are in particular prokaryotic cells, preferably bacteria belonging to the genus Listeria, to the species Listeria innocua or monocytogenes 4b, or the microorganisms related to the species Listeria innocua or monocytogenes 4b. The invention also relates to the plants and the animals, except humans, which comprise a transformed cell according to the invention. The transformed cells according to the invention can be used in methods for preparing recombinant polypeptides according to the invention. The methods for preparing a polypeptide according to the invention in recombinant form, characterized in that they use a vector and/or a cell transformed with a vector according to the invention are themselves included in the present invention. Preferably, a cell transformed with a vector according to the invention is cultured under conditions which allow the expression of said polypeptide, and said recombinant polypeptide is recovered.

As has been mentioned, the cellular host can be chosen from prokaryotic or eukaryotic systems. In particular, it is possible to identify nucleotide sequences according to the invention which facilitate secretion in such a prokaryotic or eukaryotic system. A vector according to the invention carrying such a sequence can therefore be advantageously used for producing recombinant proteins intended to be secreted. As a result, the purification of these recombinant proteins of interest will be facilitated by the fact that they are present in the cell culture supernatant rather than inside the host cells.

The polypeptides according to the invention may also be prepared by chemical synthesis. Such a method of preparation is also a subject of the invention. Those skilled in the art are aware of the methods of chemical synthesis, for example the techniques using solid phases (see in particular Steward et al., 1984, Solid phase peptides synthesis, Pierce Chem. Company, Rockford, 111, 2nd Ed., (1984)) or techniques using partial solid phases, by fragment condensation or by conventional synthesis in solution. The polypeptides obtained by chemical synthesis, and possibly comprising corresponding unnatural amino acids, are also included in the invention.

The invention also relates to hybrid polypeptides having at least one polypeptide or one of its fragments according to the invention, and a sequence of a polypeptide capable of inducing an immune response in humans or animals.

Advantageously, the antigenic determinant is such that it is capable of inducing a humoral and/or cellular response.

Such a determinant may comprise a polypeptide or one of its fragments according to the invention in a glycosylated form used for the purpose of obtaining immunogenic compositions capable of inducing the synthesis of antibodies directed against multiple epitopes. Said polypeptides or their glycosylated fragments are also part of the invention.

These hybrid molecules may consist partly of a molecule bearing polypeptides or their fragments according to the invention, combined with an optionally immunogenic component, in particular an epitope of the diphtheria toxin, the tetanus toxin, a surface antigen of the hepatitis B virus (patent FR 79 21811), the VP1 antigen of the poliomyelitis virus or any other viral or bacterial antigen or toxin.

The methods for synthesizing the hybrid molecules encompass the methods used in gene therapy to construct hybrid nucleotide sequences encoding desired polypeptide sequences. Reference may, for example, advantageously be made to the technique for obtaining genes encoding fusion proteins, described by Minton in 1984.

Said hybrid nucleotide sequences encoding a hybrid polypeptide and also the hybrid polypeptides according to the invention, characterized in that they are recombinant polypeptides obtained by the expression of said hybrid nucleotide sequences, are also part of the invention.

The invention also comprises the vectors characterized in that they contain one of said hybrid nucleotide sequences. The host cells transformed by said vectors, the transgenic animals comprising one of said transformed cells and also the methods for preparing recombinant polypeptides using said vectors, said transformed cells and/or said transgenic animals are also part of the invention.

The coupling between a polypeptide according to the invention and an immunogenic polypeptide may be carried out chemically or biologically. Thus, according to the invention, it is possible to introduce one or more attachment element(s), in particular amino acids, so as to facilitate the reactions for coupling between the polypeptide according to the invention and the immunostimulatory polypeptide, the covalent coupling of the immunostimulatory antigen possibly being carried out at the N- or C-terminal end of the polypeptide according to the invention. Bifunctional reagents for this coupling are determined as a function of the end chosen for carrying out this coupling, and the coupling techniques are well known to those skilled in the art.

The conjugates derived from a peptide coupling may be prepared by genetic recombination. The hybrid peptide (conjugate) may in fact be produced by recombinant DNA techniques, by insertion or addition of a sequence encoding the antigenic, immunogenic or hapten peptide (s), into the DNA sequence encoding the polypeptide according to the invention. These techniques for preparing hybrid peptides by genetic recombination are well known to those skilled in the art (see, for example, Makrides, 1996, Microbiological Reviews 60, 512-538).

Preferably, said immuno polypeptide is chosen from the group of peptides containing toxoids, in particular diphtheria toxoid or tetanus toxoid, Streptococcus-derived proteins (such as human serum albumin-binding protein), OMPA membrane proteins and outer membrane protein complexes, outer membrane vesicles or heat shock proteins.

The hybrid polypeptides according to the invention are very useful for obtaining monoclonal or polyclonal antibodies capable of specifically recognizing the polypeptides according to the invention. In fact, a hybrid polypeptide according to the invention allows potentiation of the immune response, against the polypeptide according to the invention coupled to the immunogenic molecule. Such monoclonal or polyclonal antibodies, fragments thereof or the chimeric antibodies, which recognize the polypeptides according to the invention, are also subjects of the invention.

The specific monoclonal antibodies may be obtained according to the conventional method of hybridoma culturing described by Köhler and Milstein (1975, Nature 256, 495).

The antibodies according to the invention are, for example, chimeric antibodies, humanized antibodies, or Fab or F(ab′)² fragments. They may also be in the form of immunoconjugates or of antibodies which are labeled in order to obtain a detectable and/or quantifiable signal.

Thus, the antibodies according to the invention can be used in a method for detecting and/or identifying bacteria belonging to the species Listeria innocua or monocytogenes 4b, or to a related microorganism, in a biological sample, characterized in that it comprises the following steps:

a) bringing the biological sample into contact with an antibody according to the invention;

b) demonstrating the antigen-antibody complex possibly formed.

The antibodies according to the present invention can also be used in order to detect expression of a gene of Listeria innocua or monocytogenes 4b or related microorganisms. Specifically, the presence of the expression product of a gene recognized by an antibody specific for said expression product can be detected by the presence of an antigen-antibody complex formed after the strain of Listeria innocua or monocytogenes 4b or of the related microorganism has been brought into contact with an antibody according to the invention. The bacterial strain used may have been “prepared”, i.e. centrifuged, lyzed, placed in an appropriate reagent for constituting the medium suitable for the immunoreaction. In particular, preference is given to a method for detecting the expression in the gene, corresponding to Western blotting, which may be performed after polyacrylamide gel electrophoresis of a lysate of the bacterial strain, in the presence or absence of reducing conditions (SDS-PAGE). After migration and separation of the proteins on the polyacrylamide gel, said proteins are transferred onto a suitable membrane (for example made of nylon) and the presence of the protein or of the polypeptide of interest is detected by bringing said membrane into contact with an antibody according to the invention.

Thus, the present invention also comprises the kits or sets necessary for carrying out a method as described (for detecting the expression of a gene of Listeria innocua or monocytogenes 4b, or their related microorganism, or for detecting and/or identifying bacteria belonging to the species Listeria innocua or monocytogenes 4b, or a related microorganism), comprising the following elements:

a) a polyclonal or monoclonal antibody according to the invention;

b) optionally, the reagents for constituting the medium suitable for the immunoreaction;

c) optionally, the reagents for demonstrating the antigen-antibody complexes produced by the immunoreaction.

The polypeptides and the antibodies according to the invention can advantageously be immobilized on a support, in particular a protein chip. Such a protein chip is a subject of the invention and may also contain at least one polypeptide of a microorganism other than Listeria innocua or monocytogenes 4b, or an antibody directed against a compound of a microorganism other than Listeria innocua or monocytogenes 4b.

The protein chips or high density filters containing proteins according to the invention can be constructed in the same way as the DNA chips according to the invention. In practice, it is possible to carry out the synthesis of the polypeptides directly attached to the protein chip, or to carry out an ex situ synthesis followed by a step of attaching the synthesized polypeptide to said chip. The latter method is preferable when the intention is to attach proteins of considerable size to the support, these proteins being advantageously prepared by genetic engineering. However, if the intention is to attach only peptides to the support of said chip, it may be more advantageous to synthesize said peptides directly in situ.

The protein chips according to the invention may advantageously be used in kits or sets for detecting and/or identifying bacteria related to the species Listeria innocua or monocytogenes 4b, or to a microorganism, or more generally in kits or sets for detecting and/or identifying microorganisms. When the polypeptides according to the invention are attached to DNA chips, the presence of antibodies in the samples tested is sought, the attachment of an antibody according to the invention to the support of the protein chip allowing identification of the protein for which said antibody is specific.

Preferably, an antibody according to the invention is attached to the support of the protein chip and the presence of the corresponding antigen, specific for Listeria innocua or monocytogenes 4b, or for a related microorganism, is detected.

A protein chip described above can be used to detect gene products, in order to establish an expression profile for said genes, in addition to a DNA chip according to the invention.

The protein chips according to the invention are also extremely useful for proteomic experiments, which study interactions between the various proteins of a given microorganism. In a simplified manner, representative peptides of the various proteins of an organism are attached to a support. Said support is then brought into contact with labeled proteins and, after an optional rinsing step, interactions between said labeled proteins and the peptides attached to the protein chip are detected.

Thus, the protein chips comprising a polypeptide sequence according to the invention or an antibody according to the invention are a subject of the invention, as are the kits or sets containing them.

The present invention also covers a method for detecting and/or identifying bacteria belonging to the species Listeria innocua or monocytogenes 4b, or to a related microorganism, in a biological sample, which uses a nucleotide sequence according to the invention.

It should be understood that, in the present invention, the term “biological sample” concerns samples taken from a living organism (in particular blood, tissues, organs or others taken from a mammal) or a sample containing biological material, i.e. DNA or RNA. Such a biological sample also comprises food compositions containing bacteria (for example cheeses, dairy products), but also food compositions containing yeast (beers, breads) or others. The term “biological sample” also concerns bacteria isolated from these samples or food compositions.

The method of detection and/or identification using the nucleotide sequences according to the invention may be diverse in nature.

A method comprising the following steps is preferred:

a) optionally isolating the DNA from the biological sample to be analyzed, or obtaining a cDNA from the RNA of the biological sample;

b) specifically amplifying the DNA of bacteria belonging to the species Listeria innocua or monocytogenes 4b, or to a related microorganism, using at least one primer according to the invention;

c) demonstrating the amplification products.

This method is based on the specific amplification of the DNA, in particular via a polymerase chain reaction.

A method comprising the following steps is also preferred:

a) bringing a nucleotide probe according to the invention into contact with a biological sample, the nucleic acid contained in the biological sample having, where appropriate, previously been made accessible to hybridization, under conditions which allow hybridization of the probe to the nucleic acid of a bacterium belonging to the species Listeria innocua or monocytogenes 4b, or to a related microroganism;

b) demonstrating the hybrid possibly formed between the nucleotide probe and the DNA of the biological sample.

Such a method should not be limited to detecting the presence of the DNA contained in the biological sample to be tested, it can also be used to detect the RNA contained in said sample. This method in particular encompasses Southern and Northern blotting.

Another preferred method according to the invention comprises the following steps:

a) bringing a nucleotide probe immobilized on a support according to the invention into contact with a biological sample, the nucleic acid of the sample having, where appropriate, previously been made accessible to hybridization, under conditions which allow hybridization of the probe to the nucleic acid of a bacterium belonging to the species Listeria innocua or monocytogenes 4b, or to a related microorganism;

b) bringing the hybrid formed between the nucleotide probe immobilized on a support and the nucleic acid contained in the biological sample, where appropriate after removing the DNA of the biological sample which has not hybridized with the probe, into contact with a labeled nucleotide probe according to the invention;

c) demonstrating the new hybrid formed in step b).

This method is advantageously used with a DNA chip according to the invention, the nucleic acid being sought hybridizing with a probe present at the surface of said chip, and being detected using a labeled probe. This method is advantageously carried out by combining a prior step of amplifying the DNA or the complementary DNA optionally obtained by reverse transcription, using primers according to the invention.

Thus, the present invention also encompasses the kits or sets for detecting and/or identifying bacteria belonging to the species Listeria innocua or monocytogenes 4b, or to a related microorganism, characterized in that they comprise the following elements:

a) a nucleotide probe according to the invention;

b) optionally, the reagents required for carrying out a hybridization reaction;

c) optionally, at least one primer according to the invention and also the reagents required for a DNA amplification reaction.

Similarly, the present invention also encompasses the kits or sets for detecting and/or identifying bacteria belonging to the species Listeria innocua or monocytogenes 4b, or to a related microorganism, characterized in that they comprise the following elements:

a) a nucleotide probe, termed capture probe, according to the invention;

b) an oligonucleotide probe, termed detection probe, according to the invention;

c) optionally, at least one primer according to the invention and also the reagents required for a DNA amplification reaction.

Finally, the kits or sets for detecting and/or identifying bacteria belonging to the species Listeria innocua or monocytogenes 4b, or to a related microorganism, characterized in that they comprise the following elements:

a) at least one primer according to the invention;

b) optionally, the reagents required to carry out a DNA amplification reaction;

c) optionally, a component for verifying the sequence of the amplified fragment, more particularly an oligonucleotide probe according to the invention, are also subjects of the present invention.

Preferably, said primers and/or probes and/or polypeptides and/or antibodies according to the present invention, used in the methods and/or kits or sets according to the present invention, are chosen from the primers and/or probes and/or polypeptides and/or antibodies specific for the species Listeria innocua or monocytogenes 4b. Preferably, these elements are chosen from the nucleotide sequences encoding a secreted protein, from the secreted polypeptides, or from the antibodies directed against secreted polypeptides of Listeria innocua or monocytogenes 4b.

A subject of the present invention is also the strains of Listeria innocua or monocytogenes 4b, and/or of related microorganisms, containing one or more mutation(s) in a nucleotide sequence according to the invention, in particular an ORF sequence, or regulatory elements thereof (in particular promoters).

According to the invention, preference is given to the strains of Listeria innocua or monocytogenes 4b exhibiting one or more mutation(s) in the nucleotide sequences encoding polypeptides involved in the cellular machinery, in particular secretion, central intermediate metabolism, energy metabolism, and processes of amino acid synthesis, of transcription and translation, and of polypeptide synthesis.

Said mutations may lead to inactivation of the gene or, in particular when they are located in the regulatory elements of said gene, to overexpression of this gene.

The invention also relates to the use of a nucleotide sequence according to the invention, of a polypeptide according to the invention, of an antibody according to the invention, of a cell according to the invention, and/or of a transformed animal according to the invention, for selecting an organic or inorganic compound capable of modulating, regulating, inducing or inhibiting gene expression, and/or of modifying cell replication in eukaryotic or prokaryotic cells, or capable of inducing, inhibiting or worsening pathological conditions associated with an infection with Listeria innocua or monocytogenes 4b or one of its related microorganisms.

The invention also comprises a method for selecting compounds capable of binding to a polypeptide, or one of its fragments, according to the invention, capable of binding to a nucleotide sequence according to the invention, or capable of recognizing an antibody according to the claim, and/or capable of modulating, regulating, inducing or inhibiting gene expression, and/or of modifying cell growth or replication in eukaryotic or prokaryotic cells, or capable of inducing, inhibiting or worsening, in an animal or human organism, pathological conditions associated with an infection with Listeria, for example with L. monocytogenes 4b, or one of its related microorganisms, characterized in that it comprises the following steps:

a) bringing said compound into contact with said polypeptide or said nucleotide sequence or with a transformed cell according to the invention and/or administering said compound to a transformed animal according to the invention;

b) determining the ability of said compound to bind to said polypeptide or said nucleotide sequence, or to modulate, regulate, induce or inhibit gene expression, or to modulate cell growth or replication, or to induce, inhibit or worsen, in the said transformed animal, pathological conditions associated with an infection with Listeria, for example L. monocytogenes 4b or one of its related microorganisms.

The transformed cells and/or animals according to the invention may advantageously serve as a model and be used in methods for studying, identifying and/or selecting compounds which may be responsible for pathological conditions induced or worsened by Listeria monocytogenes, or which may prevent and/or treat these pathological conditions. In particular, the transformed host cells, in particular the bacteria of the Listeria family, the transformation of which with a vector according to the invention may, for example, increase or inhibit its infectious capacity, or modulate the pathological conditions usually induced or worsened by the infection, may be used to infect animals in which the appearance of the pathological conditions will be followed. These nontransformed animals, infected for example with transformed Listeria bacteria, may serve as a study model. In the same way, the transformed animals according to the invention may be used in methods for selecting compounds capable of preventing and/or treating diseases due to Listeria. Said methods using said transformed cells and/or transformed animals are part of the invention.

The compounds liable to be selected may be organic compounds such as polypeptides or carbohydrates or any other organic or inorganic compounds which are already known, or new organic compounds developed using molecular modeling techniques and obtained by chemical or biochemical synthesis, these techniques being known to those skilled in the art.

Said selected compounds may be used to model the cell growth and/or replication of Listeria innocua or monocytogenes 4b, or any other related microorganism, and thus to control infection with these microorganisms. Said compounds according to the invention may also be used to model cell growth and/or replication of any eukaryotic or prokaryotic cells, in particular tumor cells and infectious microorganisms, for which said compounds will prove to be active, the methods for determining said modulations being well known to those skilled in the art.

The expression “compound capable of modulating the growth of a microorganism” is intended to denote any compound making it possible to intervene in, modify, limit and/or reduce the development, growth, proliferation rate and/or viability of said microorganism.

This modulation may be carried out, for example, using an agent capable of binding to a protein and thus of inhibiting or potentiating its biological activity, or capable of binding to an outer surface membrane protein of a microorganism and blocking the penetration of said microorganism into the host cell or promoting the action of the immune system of the infected organism, directed against said microorganism. This modulation may also be carried out using an agent capable of binding to a nucleotide sequence of a DNA or RNA of a microorganism and blocking, for example, the expression of a polypeptide, the biological or structural activity of which is necessary for the growth or for the reproduction of said microorganism.

In the present invention, the term “related microorganism” is intended to denote any microorganism in which the gene expression may be modulated, regulated, induced or inhibited, or in which cell growth or replication may also be modulated, by a compound of the invention. In the present invention, the term “related microorganism” is also intended to denote any microorganism comprising nucleotide sequences or polypeptides according to the invention. These microorganisms may, in certain cases, comprise polypeptides or nucleotide sequences identical or homologous to those of the invention and may also be detected and/or identified using the methods or kit for detection and/or identification according to the invention and may also serve as a target for the compounds of the invention. The term “microorganism” is also intended to denote any Listeria monocytogenes microorganism of any serotype.

The invention relates to the compounds which can be selected using a selection method according to the invention.

The invention also relates to a pharmaceutical composition comprising a compound chosen from the following compounds:

a) a nucleotide sequence according to the invention;

b) a polypeptide according to the invention;

c) a vector according to the invention;

d) an antibody according to the invention; and

e) a compound which can be selected using a selection method according to the invention, optionally in combination with a pharmaceutically acceptable vehicle.

The term “effective amount” is intended to denote a sufficient amount of said compound or antibody, or polypeptide according to the invention, for modulating the growth of Listeria innocua or monocytogenes 4b or of a related microorganism.

The invention also relates to a pharmaceutical composition according to the invention, for preventing or treating an infection with a bacterium belonging to the genus Listeria, or with a related microorganism.

The invention is also directed toward an immunogenic and/or vaccine composition, characterized in that it comprises one or more polypeptides according to the invention and/or one or more hybrid polypeptides according to the invention.

The invention also comprises the use of a transformed cell according to the invention, for preparing a vaccine composition.

The invention is also directed toward a vaccine composition, characterized in that it contains a nucleotide sequence according to the invention, a vector according to the invention and/or a transformed cell according to the invention.

The invention also relates to the vaccine compositions according to the invention, for preventing or treating an infection with a bacterium belonging to the genus Listeria, or with a related microorganism.

Preferably, the immunogenic and/or vaccine compositions according to the invention intended for the prevention and/or treatment of infection with Listeria, or with a related microorganism, will be chosen from the immunogenic and/or vaccine compositions comprising a polypeptide, or one of its fragments, corresponding to a protein, or one of its fragments, of the cell envelope of Listeria. The vaccine compositions comprising nucleotide sequences will preferably also comprise nucleotide sequences encoding a polypeptide, or one of its fragments, corresponding to a protein, or one of its fragments, of the cell envelope of Listeria.

The polypeptides of the invention, or their fragments, which are part of the immunogenic compositions according to the invention can be selected using techniques known to those skilled in the art, such as, for example, the ability of said polypeptides to stimulate T cells, which causes, for example, the proliferation thereof or the secretion of interleukins, and which results in the production of antibodies directed against said polypeptides.

In mice, to which a weight dose of the vaccine composition comparable to the dose used in humans is administered, the antibody reaction is tested by taking a serum sample and then studying the formation of a complex between the antibodies present in the serum and the antigen of the vaccine composition, according to usual techniques.

According to the invention, said vaccine compositions will preferably be in combination with a pharmaceutically acceptable vehicle and, where appropriate, with one or more suitable adjuvants of immunity.

Today, various types of vaccine are available for protecting humans against infectious diseases: attenuated live microorganisms (M. bovis—BCG for tuberculosis), inactivated microorganisms (flu virus), acellular extracts (Bordetella pertussis for whooping cough), recombined proteins (hepatitis B virus surface antigen) and polysaccharides (pneumococci). Vaccines prepared from synthetic peptides or from genetically modified microorganisms expressing heterologous antigens are undergoing experimentation. Even more recently, recombined plasmid DNAs carrying genes encoding protective antigens have been proposed as an alternative vaccine strategy. This type of immunization is carried out with a particular plasmid derived from an E. coli plasmid which does not replicate in vivo and which encodes only the immunizing protein. Animals have been immunized by simply injecting the naked plasmid DNA into muscle. This technique leads to the expression of the vaccine protein in situ and to an immune response of the cellular type (CTL) and of the humoral type (antibodies). This double induction of the immune response is one of the main advantages of the technique of immunization with naked DNA.

The vaccine compositions comprising nucleotide sequences or vectors into which said sequences are inserted are in particular described in international application No. WO 90/11092 and also in international application No. WO 95/11307.

The nucleotide sequence constituting the vaccine composition according to the invention can be injected into the host after having been coupled to compounds which promote the penetration of this polynucleotide into the cell or its transport to the cell nucleus. The resulting conjugates can be encapsulated in polymeric microparticles, as described in international application No. WO 94/27238 (Medisorb Technologies International).

According to another embodiment of the vaccine composition according to the invention, the nucleotide sequence, preferably a DNA, is complexed with DEAE-dextran, with nuclear proteins or lipids, or encapsulated in liposomes or else introduced in the form of a gel which facilitates its transfection into cells. The polynucleotide or the vector according to the invention may also be in suspension in a buffer solution or may be associated with liposomes.

Advantageously, such a vaccine will be prepared in accordance with the technique described by Tacson et al. or Huygen et al. in 1996 or else in accordance with the technique described by Davis et al. in international application No. WO 95/11307.

Such a vaccine may also be prepared in the form of a composition containing a vector according to the invention, placed under the control of regulatory elements for its expression in humans or animals. As a vector for in vivo expression of the polypeptide antigen of interest, use may, for example, be made of the plasmid pcDNA3 or the plasmid pcDNA1/neo, both marketed by Invitrogen (R & D Systems, Abingdom, United Kingdom). Such a vaccine will advantageously comprise, besides the recombinant vector, a saline solution, for example a sodium chloride solution.

The expression “pharmaceutically acceptable vehicle” is intended to denote a compound, or a combination of compounds, included in a pharmaceutical or vaccine composition, which does not cause any side reactions and which makes it possible, for example, to facilitate administration of the active compound, to increase the lifetime thereof and/or the effectiveness thereof in the organism, to increase the solubility thereof in solution or else to improve the conservation thereof. These pharmaceutically acceptable vehicles are well known and will be adjusted by those skilled in the art as a function of the nature and of the method of administration of the active compound chosen.

With regard to the vaccine formulations, they may comprise suitable adjuvants of immunity which are known to those skilled in the art, such as, for example, aluminum hydroxide, a representative of the muramyl peptide family, such as one of the peptide derivatives of N-acetylmuramyl, a bacterial lysate, or else incomplete Freund's adjuvant.

Preferably, these compounds will be administered systemically, in particular intravenously, intramuscularly, intradermally or subcutaneously, or orally. More preferably, the vaccine composition comprising the polypeptides according to the invention will be administered several times, spread out over time, intradermally or subcutaneously.

The optimal methods of administration, doses and pharmaceutical forms of these compounds can be determined according to the criteria generally taken into account in establishing a suitable treatment for a patient, such as, for example, the age or body weight of the patient, the seriousness of his or her general condition, the tolerance to the treatment and the side effects noted.

Finally, the invention comprises the use of a composition according to the invention, for treating or preventing diseases induced or worsened by the presence of Listeria.

Moreover, a subject of the present invention is also a genomic DNA library of a bacterium of the genus Listeria, preferably Listeria innocua or monocytogenes, preferably the 4b strain.

The genomic DNA libraries described in the present invention, in particular the Li-shotgun library deposited with the CNCM [National Collection of Cultures and Microorganisms] on Oct. 2, 2000, under the accession No. I-2565 and the Lmb4b-shotgun library deposited with the CNCM on Oct. 2, 2000, under the accession No. I-2566, in fact cover the genome of Listeria innocua and Listeria monocytogenes 4b, respectively. However, although certain regions could not be cloned into said library, due to problems of lethality in Escherichia coli, these regions can easily be amplified and identified by those skilled in the art, using oligonucleotides specific for the sequences of the ends of the various clones which form the contigs.

The present invention also relates to the methods for isolating a polynucleotide of interest present in a strain of Listeria and absent from another strain, which use at least one DNA library based, for example, on a plasmid pcDNA2.1 containing the Listeria genome. The method according to the invention for isolating a polynculeotide of interest may comprise the following steps:

a) isolating at least one polynucleotide contained in a clone of the library of DNA from Listeria;

b) isolating:

at least one genomic polynucleotide or cDNA of a listeria, said listeria belonging to a strain which is different from the strain used to construct the DNA library of step a) or, alternatively,

at least one polynucleotide contained in a clone from a DNA library prepared from the genome of a Listeria which is different from the Listeria used to construct the DNA library of step a);

c) hybridizing the polynucleotide of step a) to the polynucleotide of step b);

d) selecting the polynucleotides of step a) which have not formed a hybridization complex with the polynucleotides of step b);

e) characterizing the polynucleotide selected.

The polynucleotide of step a) can be prepared by digesting at least one recombinant clone with a suitable restriction enzyme and, optionally, amplifying the polynucleotide insert which results therefrom.

Thus, the method of the invention allows those skilled in the art to perform comparative genomic studies between the various strains or species of the genus Listeria, for example between the pathogenic strains and their nonpathogenic equivalents.

In particular, it is possible to study and determine the regions of polymorphism between said strains.

### EXAMPLES

### 1. Construction of the Libraries

The chromosomal DNA of the strains studied was prepared using a conventional method including treatment with proteinase K and extraction with phenol (C. Jacquet et al., Zentralbl Bakteriol., 276:356-365, 1992). Approximately 10 μg of DNA were broken up by nebulization (1 minute under a pressure of 1 bar) (C. Buchrieser et al., Infect. Immun., 67:4851-4861, 1999). The ends of the DNA fragments were blunt-ended by the action of T4 bacteriophage DNA polymerase for 15 minutes at 37° C. in the presence of the 4 nucleotide triphosphates. The enzyme was inactivated by incubation at 75° C. for 15 min. Linkers (invitrogen Cat. No. 408-18) were then ligated to these ends. After ligation, the chromosomal DNA fragments of between 1 000 and 3 000 base pairs in size were purified after agarose gel electrophoresis. The vector used to construct the library, pcDNA2.1 (Invitrogen), was digested with the BstX1 enzyme and purified with geneclean (BIO-101) after agarose gel electrophoresis. The chromosomal DNA and the purified vector were ligated by the action of T4 bacteriophage ligase. The ligation mixture was introduced into the Escherichia coli strain XL2-blue (Stratagene) by transformation. Approximately 4 000 colonies were obtained per μl of the ligation mixture.

This method is used to construct the Li-shotgun library deposited with the CNCM on Oct. 2, 2000, under the No. I-2565 for the strain Listeria innocua (CLIP 11262) and the Lm4b-shotgun library deposited with the CNCM on Oct. 2, 2000, under the No. I-2566 for the strain Listeria monocytogenes serotype 4b (CLIP 80459).

### 2. Preparation of the Plasmids and Sequencing

Plasmids were prepared by a semi-automatic method of preparation developed in the GMP laboratory (Génomique des Micro-organismes Pathogènes de l'Institut Pasteur [Genomics of Pathogenic Microorganisms of the Pasteur Institute]) based on the alkaline lysis method (H. C. Bimboim, Methods Enzymol., 100:243-255, 1983). The chromosomal inserts were sequenced from both of their ends using the T7 and universal primers, according to the supplier's recommendations (PE-biosystems). The sequences were determined using automatic sequencers of the 377 and 3700 type (PE-Biosystem).

### 3. Sequence Assembly

The sequences were assembled using the software package developed at the University of Washington, Phred, Phrap and Consed (B. Ewing et al., Genome Res., 8:186-194, 1998; D. Gordon et al., Genome Res., 8:195-202, 1998). The sequence was finished using the software package GMPTB (L. Frangeul et al., Microbiology, 145:2625-2634, 1999). The finishing step corresponds to the resequencing of the regions in which the sequence is relatively unsure and the sequencing of the regions located between the contigs. It was carried out either by sequencing PCR products or by walking on the clones from the library. The sequences of the oligonucleotides were defined using the consed and Primo software (D. Gordon et al., 1998; P. Li et al., Genomics, 40:476-485, 1997).

### 4. Sequence Annotation

The coding sequences (CDS) were identified using the GMPTB software package. This program combines the results of various methods: (i) identification of open reading frames and sorting thereof as a function of their size, (ii) analysis of the probability of being coding using the Genemark software (A. V. Lukashin et al., Nucleic Acids Res., 15:1107-1115, 1998), (iii) identification of a start of translation (initiation codon and ribosome-binding sequence), (iv) similarity of the deduced protein sequence with the protein sequences contained in the sequence banks using the BLASTP software.

The functions of the proteins encoded by the coding sequences identified were predicted by analyzing the results from searching for similarities in the banks using the BLASTP software (S. F. Altschul et al., Nucleic Acids Research, 25:3389-402, 1997).

### 5. Comparison of Genomes

a) Identification of the CDS Specific for the Strain of L. monocytogenes EGDe and for the Strain of L. innocua

All of the protein sequences deduced from the predicted coding sequences of each genome were compared to all of the protein sequences possibly encoded by the other genome using the BLASTP software. A threshold of 75% identity over the entire length of the protein was selected in order to identify the proteins specific for an isolate. This very high value was selected since it is the best for discriminating between orthologous genes and paralogous genes (W. S. Fitch, Syst. Zool., 19:99-113, 1970). For the protein sequences for which the sequence conservation is high (>70%), conservation of the nucleotide sequences of the genes will itself also be high and might give a signal under relatively nonstringent hybridization conditions. It will be necessary to take this eventuality into account in analyzing the result of the test.

b) Identification of the CDS Specific for the L. monocytogenes Serotype 4b Strain Compared to the L. monocytogenes EGDe Strain and the L. innocua Strain

The chromosomal regions of the L. monocytogenes serotype 4b strain which are absent from the L. monocytogenes EGDe and L. innocua strains were identified using the Crossmatch/Phrap package (Phil Green, University of Washington, Seattle, unpublished). This software makes it possible to assemble nucleotide sequences (Phrap) by masking all the sequences or parts of sequences similar to one or more reference sequences (Crossmatch). The reference sequences used were: the complete sequence of the genome of L. monocytogenes EGD, the complete sequence of the genome of L. innocua and the sequence of its plasmid. The identification by Crossmatch of the regions which will be masked is based on the search for words of 11 identical letters between the sequence analyzed and the reference sequences, and the extension of these words using the default parameters of the software. More information on the Crossmatch and Phrap software is available on the website: http://bozeman.mbt.washington.edu/.

### 6. Examples of Annotations

6.1. Genes specific for L. monocytogenes. There is no significant similarity between the nucleotide sequences of the gene of L. monocytogenes and the genome of L innocua.

TABLE I

| | ID of the gene of | % identity of the | % identity of |
|---|---|---|---|
| ID of the gene of | L. innocua | protein sequences | the nucleotide |
| L. monocytogenes | (best score) | (% of the sequence) | sequences |
| 1814.1 SEQ ID No. 779 | — | — | — |
| 2601.1 SEQ ID No. 851 | — | — | — |
| 615.1 SEQ ID No. 1034 | — | — | — |
| 1713.1 SEQ ID No. 772 | 5385.1 SEQ ID No. 2042 | 25% (100%) | ≦40% |
| 1656.1 SEQ ID No. 768 | 526.1 SEQ ID No. 684 | 36% (50%) | ≦40% |
| 3477.1 SEQ ID No. 908 | 1614.1 SEQ ID No. 632 | 33% (60%) | ≦40% |
| 3418.2 SEQ ID No. 904 | 1235.1 SEQ ID No. 2043 | 30% (70%) | ≦40% |

6.2. Genes specific for L. innocua. There is no significant similarity between the nucleotide sequence of the gene of L. innocua and the genome of L. monocytogenes.

TABLE II

| | ID of the gene of | % identity of the | % identity of the |
|---|---|---|---|
| ID of the gene of | L. monocytogenes | protein | nucleotide |
| L. innocua | (best score) | sequences | sequences |
| 1259.1 SEQ ID No. 689 | — | — | — |
| 3320.1 SEQ ID No. 527 | — | — | — |
| 1348.1 SEQ ID No. 596 | 1545.1 SEQ ID No. 2044 | 26% (70%) | ≦40% |
| 4232.1 SEQ ID No. 681 | 894.1 SEQ ID No. 2045 | 30% (90%) | ≦40% |
| 5550.1 SEQ ID No. 519 | 312.1 SEQ ID No. 2046 | 31% (60%) | ≦40% |
| 3320.1 SEQ ID No. 527 | 558.1 SEQ ID No. 1025 | 25% (50%) | ≦40% |

6.3. Genes common to the two strains for which the similarity (identity) of the deduced protein sequences is less than 75% and value of the similarity at the nucleotide level.

TABLE III

| | ID of the gene of | % identity of the | % identity of |
|---|---|---|---|
| ID of the gene of | L. innocua | protein sequences | the nucleotide |
| L. monocytogenes | (best score) | (% of the sequence) | sequences |
| 1343 SEQ ID No. 727 | 36.1 SEQ ID No. 528 | 57% (100%) | 67% |
| 725.1 SEQ ID No. 1046 | 1402.1 SEQ ID No. 2047 | 52% (100%) | 60% |
| 1002.1 SEQ ID No. 690 | 1021.1 SEQ ID No. 666 | 50% (100%) | 65% |
| 1974.3 SEQ ID No. 789 | 897.1 SEQ ID No. 2048 | 49% (100%) | 58% |

6.4. Genes common to L. monocytogenes and L. innocua

TABLE IV

| | ID of the gene of | % identity of the | % identity of |
|---|---|---|---|
| ID of the gene of | L. innocua | protein sequences | the nucleotide |
| L. monocytogenes | (best score) | (% of the sequence) | sequences |
| 1976.1 SEQ ID No. 2049 | 5481.1 SEQ ID No. 2053 | 88% (100%) | 82% |
| 1979.1 SEQ ID No. 2050 | 5476.1 SEQ ID No. 2054 | 98% (100%) | 86% |
| 1980.1 SEQ ID No. 2051 | 5474.1 SEQ ID No. 2055 | 84% (100%) | 80% |
| 1983.1 SEQ ID No. 2052 | 5471.1 SEQ ID No. 2056 | 87% (100%) | 79% |

Table V: Legends

SEQ ID Nos. 1-11: nucleotide sequences of 10 Contigs and 1 plasmid originating from the assembly of Listeria innocua

SEQ ID Nos. 12-689: nucleotide sequences of the proteins specific for L. innocua (absent from L. monocytogenes-EGD).

SEQ ID Nos. 690-1067: nucleotide sequences of proteins specific for L. monocytogenes-EGD (absent from L. innocua).

SEQ ID Nos. 1068-2041: nucleotide sequences of 974 contigs originating from the assembly of Listeria monocytogenes-4b (1 231 537 bases).

SEQ ID Nos. 2042-2056: additional sequences for annotation examples.

TABLE V

| | | Blastp result on non-redondant protein bank. | | |
|---|---|---|---|---|
| SEQ ID | Prot N^{o} | homology/ | Comments/ | % |
| SEQ ID N^{o} 12 | LI-1779.1 | | No Hits found | |
| SEQ ID N^{o} 13 | LI-4603.1 | | No Hits found | |
| SEQ ID N^{o} 14 | LI-4611.1 | | No Hits found | |
| SEQ ID N^{o} 15 | LI-6102.1 | | No Hits found | |
| SEQ ID N^{o} 16 | LI-738.2 | | | |
| SEQ ID N^{o} 17 | LI-1983.1 | | No Hits found | |
| SEQ ID N^{o} 18 | LI-2952.1 | | No Hits found | |
| SEQ ID N^{o} 19 | LI-3070.1 | | No Hits found | |
| SEQ ID N^{o} 20 | LI-1237.1 | | No Hits found | |
| SEQ ID N^{o} 21 | LI-1718.1 | 56 | emb\|CAB83919.1\| (AL162753) hypothetical protein NMA0630 [Neisseria | Length = 304 |
| | | | meningitidis] | |
| SEQ ID N^{o} 22 | LI-1869.1 | | No Hits found | |
| SEQ ID N^{o} 23 | LI-1910.1 | | No Hits found | |
| SEQ ID N^{o} 24 | LI-3373.1 | 78 | emb\|CAB53845.1\| (AJ242593) gp55 [Bacteriophage A118] | Length = 69 |
| SEQ ID N^{o} 25 | LI-372.1 | | No Hits found | |
| SEQ ID N^{o} 26 | LI-3727.1 | | No Hits found | |
| SEQ ID N^{o} 27 | LI-3807.1 | | No Hits found | |
| SEQ ID N^{o} 28 | LI-438.1 | | No Hits found | |
| SEQ ID N^{o} 29 | LI-4981.1 | | No Hits found | |
| SEQ ID N^{o} 30 | LI-4992.1 | | No Hits found | |
| SEQ ID N^{o} 31 | LI-6200.1 | | | |
| SEQ ID N^{o} 32 | LI-1487.1 | | No Hits found | |
| SEQ ID N^{o} 33 | LI-1603.1 | | No Hits found | |
| SEQ ID N^{o} 34 | LI-2986.1 | | No Hits found | |
| SEQ ID N^{o} 35 | LI-3365.1 | 41 | emb\|CAB53854.1\| (AJ242593) gp64 [Bacteriophage A118] | Length = 41 |
| SEQ ID N^{o} 36 | LI-5554.1 | | No Hits found | |
| SEQ ID N^{o} 37 | LI-5726.1 | | No Hits found | |
| SEQ ID N^{o} 38 | LI-1495.1 | | No Hits found | |
| SEQ ID N^{o} 39 | LI-2951.1 | | No Hits found | |
| SEQ ID N^{o} 40 | LI-2959.1 | | No Hits found | |
| SEQ ID N^{o} 41 | LI-5588.1 | | No Hits found | |
| SEQ ID N^{o} 42 | LI-5922.1 | | No Hits found | |
| SEQ ID N^{o} 43 | LI-6071.1 | | No Hits found | |
| SEQ ID N^{o} 44 | LI-6072.1 | | No Hits found | |
| SEQ ID N^{o} 45 | LI-2374.1 | | No Hits found | |
| SEQ ID N^{o} 46 | LI-2937.1 | 51 | pir\|\|F70357 lipoprotein - Aquifex aeolicus gb\|AAC06844.1\| (AE000700) | Length = 349 |
| | | | lipoprotein [Aquifex aeolicus] | |
| SEQ ID N^{o} 47 | LI-4114.1 | | No Hits found | |
| SEQ ID N^{o} 48 | LI-5780.1 | | No Hits found | |
| SEQ ID N^{o} 49 | LI-5706.1 | 65 | emb\|CAB53820.1\| (AJ242593) gp34 [Bacteriophage A118] | Length = 72 |
| SEQ ID N^{o} 50 | LI-3139.1 | | No Hits found | |
| SEQ ID N^{o} 51 | LI-3800.1 | | No Hits found | |
| SEQ ID N^{o} 52 | LI-385.1 | | No Hits found | |
| SEQ ID N^{o} 53 | LI-5458.2 | | | |
| SEQ ID N^{o} 54 | LI-5803.1 | | No Hits found | |
| SEQ ID N^{o} 55 | LI-128.1 | | No Hits found | |
| SEQ ID N^{o} 56 | LI-2428.1 | | No Hits found | |
| SEQ ID N^{o} 57 | LI-3087.1 | | No Hits found | |
| SEQ ID N^{o} 58 | LI-5915.1 | | No Hits found | |
| SEQ ID N^{o} 59 | LI-6036.1 | | No Hits found | |
| SEQ ID N^{o} 60 | LI-175.1 | | No Hits found | |
| SEQ ID N^{o} 61 | LI-1816.1 | | No Hits found | |
| SEQ ID N^{o} 62 | LI-2777.1 | 46 | gb\|AAC36979.1\| (L15633) [Conjugative transposon Tn916 (from Enterococcus faecalis, | |
| | | | DS16), 3′ end.], gene products [Transposon Tn916] gb\|AAB60027.1\| (U09422) ORF8 | |
| | | | [Enterococcus faecalis] prf\|\|2114402V ORF 8 [Enterococcus faecalis] | |
| SEQ ID N^{o} 63 | LI-2932.1 | | No Hits found | |
| SEQ ID N^{o} 64 | LI-1611.1 | | No Hits found | |
| SEQ ID N^{o} 65 | LI-2702.1 | | No Hits found | |
| SEQ ID N^{o} 66 | LI-2989.1 | | No Hits found | |
| SEQ ID N^{o} 67 | LI-4125.1 | | No Hits found | |
| SEQ ID N^{o} 68 | LI-4628.1 | | No Hits found | |
| SEQ ID N^{o} 69 | LI-5606.1 | | No Hits found | |
| SEQ ID N^{o} 70 | LI-6148.2 | | | |
| SEQ ID N^{o} 71 | LI-2237.1 | | No Hits found | |
| SEQ ID N^{o} 72 | LI-6190.1 | | | |
| SEQ ID N^{o} 73 | LI-4167.1 | | No Hits found | |
| SEQ ID N^{o} 74 | LI-5459.2 | | | |
| SEQ ID N^{o} 75 | LI-568.1 | | No Hits found | |
| SEQ ID N^{o} 76 | LI-6191.1 | | | |
| SEQ ID N^{o} 77 | LI-1368.1 | | No Hits found | |
| SEQ ID N^{o} 78 | LI-1538.1 | | No Hits found | |
| SEQ ID N^{o} 79 | LI-2694.1 | | No Hits found | |
| SEQ ID N^{o} 80 | LI-678.1 | | No Hits found | |
| SEQ ID N^{o} 81 | LI-2181.1 | | No Hits found | |
| SEQ ID N^{o} 82 | LI-3662.1 | | No Hits found | |
| SEQ ID N^{o} 83 | LI-6121.1 | | No Hits found | |
| SEQ ID N^{o} 84 | LI-6163.1 | | No Hits found | |
| SEQ ID N^{o} 85 | LI-1240.1 | | No Hits found | |
| SEQ ID N^{o} 86 | LI-2030.1 | | No Hits found | |
| SEQ ID N^{o} 87 | LI-3363.1 | | No Hits found | |
| SEQ ID N^{o} 88 | LI-4294.1 | | No Hits found | |
| SEQ ID N^{o} 89 | LI-5611.2 | | | |
| SEQ ID N^{o} 90 | LI-6041.1 | | No Hits found | |
| SEQ ID N^{o} 91 | LI-6162.1 | | No Hits found | |
| SEQ ID N^{o} 92 | LI-1082.1 | | No Hits found | |
| SEQ ID N^{o} 93 | LI-1928.1 | | No Hits found | |
| SEQ ID N^{o} 94 | LI-2878.1 | 36 | pir\|\|C72489 hypothetical protein APE2554 - Aeropyrum pernix (strain K1) | Length = 105 |
| | | | dbj\|BAA81571.1\| (AP000064) 105aa long hypothetical protein [Aeropyrum pernix] | |
| SEQ ID N^{o} 95 | LI-3676.1 | | No Hits found | |
| SEQ ID N^{o} 96 | LI-4023.1 | | No Hits found | |
| SEQ ID N^{o} 97 | LI-4475.1 | | No Hits found | |
| SEQ ID N^{o} 98 | LI-5540.1 | | No Hits found | |
| SEQ ID N^{o} 99 | LI-2946.1 | | No Hits found | |
| SEQ ID N^{o} 100 | LI-2988.1 | | No Hits found | |
| SEQ ID N^{o} 101 | LI-2990.1 | | No Hits found | |
| SEQ ID N^{o} 102 | LI-439.1 | | No Hits found | |
| SEQ ID N^{o} 103 | LI-5607.1 | | No Hits found | |
| SEQ ID N^{o} 104 | LI-4005.1 | | No Hits found | |
| SEQ ID N^{o} 105 | LI-4022.1 | | No Hits found | |
| SEQ ID N^{o} 106 | LI-4683.1 | 31 | gb\|AAA72562.1\| (M15619) ORF16-lacZ fusion protein [synthetic construct] | Length = 68 |
| SEQ ID N^{o} 107 | LI-4931.1 | | No Hits found | |
| SEQ ID N^{o} 108 | LI-1139.1 | | No Hits found | |
| SEQ ID N^{o} 109 | LI-3703.1 | 43 | gb\|AAC97745.1\| (AF063866) ORF MSV233 hypothetical protein [Melanoplus | Length = 92 |
| | | | sanguinipes entomopoxvirus] | |
| SEQ ID N^{o} 110 | LI-6145.1 | | No Hits found | |
| SEQ ID N^{o} 111 | LI-2179.1 | | No Hits found | |
| SEQ ID N^{o} 112 | LI-3431.1 | | No Hits found | |
| SEQ ID N^{o} 113 | LI-478.1 | | No Hits found | |
| SEQ ID N^{o} 114 | LI-711.1 | | No Hits found | |
| SEQ ID N^{o} 115 | LI-1092.1 | | No Hits found | |
| SEQ ID N^{o} 116 | LI-2061.1 | | No Hits found | |
| SEQ ID N^{o} 117 | LI-3990.1 | | No Hits found | |
| SEQ ID N^{o} 118 | LI-4006.1 | | No Hits found | |
| SEQ ID N^{o} 119 | LI-5373.1 | | No Hits found | |
| SEQ ID N^{o} 120 | LI-5551.1 | 52 | gb\|AAA32614.1\| (L31364) holin [Bacteriophage Tuc2009] | Length = 88 |
| SEQ ID N^{o} 121 | LI-3362.1 | | No Hits found | |
| SEQ ID N^{o} 122 | LI-4107.1 | | No Hits found | |
| SEQ ID N^{o} 123 | LI-4121.1 | | No Hits found | |
| SEQ ID N^{o} 124 | LI-5546.1 | | No Hits found | |
| SEQ ID N^{o} 125 | LI-5548.1 | | No Hits found | |
| SEQ ID N^{o} 126 | LI-5710.1 | 72 | emb\|CAB53829.1\| (AJ242593) gp40 [Bacteriophage A118] | Length = 78 |
| SEQ ID N^{o} 127 | LI-5957.1 | | No Hits found | |
| SEQ ID N^{o} 128 | LI-6149.2 | | | |
| SEQ ID N^{o} 129 | LI-1615.1 | | No Hits found | |
| SEQ ID N^{o} 130 | LI-2926.1 | | No Hits found | |
| SEQ ID N^{o} 131 | LI-3068.1 | | No Hits found | |
| SEQ ID N^{o} 132 | LI-5560.1 | 59 | sp\|P45937\|YQCB_BACSU HYPOTHETICAL 10.4 KD PROTEIN IN SPOIIIC- | |
| | | | CWLA INTERGENIC REGION pir\|\|A69949 hypothetical protein yqcB - Bacillus subtilis | |
| | | | dbj\|BAA06954.1\| (D32216) ORF130 [Bacillus subtilis] dbj\|BAA12418.1\| (D84432) YqcB | |
| | | | [Bacillus subtilis] emb\|CAB | |
| SEQ ID N^{o} 133 | LI-6026.1 | | No Hits found | |
| SEQ ID N^{o} 134 | LI-6098.1 | | No Hits found | |
| SEQ ID N^{o} 135 | LI-2884.1 | | No Hits found | |
| SEQ ID N^{o} 136 | LI-5567.1 | 57 | sp\|P45933\|YQBR_BACSU HYPOTHETICAL 9.5 KD PROTEIN IN SPOIIIC- | |
| | | | CWLA INTERGENIC REGION pir\|\|E69948 phage-related protein homolog yqbR - | |
| | | | Bacillus subtilis dbj\|BAA06950.1\| (D32216) ORF87 [Bacillus subtilis] | |
| | | | dbj\|BAA12414.1\| (D84432) YqbR [Bacillus subtilis] | |
| SEQ ID N^{o} 137 | LI-5709.1 | | No Hits found | |
| SEQ ID N^{o} 138 | LI-4219.1 | | No Hits found | |
| SEQ ID N^{o} 139 | LI-5874.1 | | No Hits found | |
| SEQ ID N^{o} 140 | LI-6101.1 | | No Hits found | |
| SEQ ID N^{o} 141 | LI-943.1 | | No Hits found | |
| SEQ ID N^{o} 142 | LI-3279.1 | | No Hits found | |
| SEQ ID N^{o} 143 | LI-3551.1 | | No Hits found | |
| SEQ ID N^{o} 144 | LI-4065.1 | 47 | gb\|AAB23085.1\|S43512_2 (S43512) orf2 immediately 5′ to ejl [Bacteriophage | Length = 85 |
| | | | EJ-1] | |
| SEQ ID N^{o} 145 | LI-4113.1 | 57 | pir\|\|T09011 probable transposase TnpA - Streptococcus pyogenes (fragment) | Length = 364 |
| | | | gb\|AAB92607.1\| (AF026542) TnpA [Streptococcus pyogenes] | |
| SEQ ID N^{o} 146 | LI-5362.1 | | No Hits found | |
| SEQ ID N^{o} 147 | LI-1694.1 | 63 | pir\|\|F75297 hypothetical protein - Deinococcus radiodurans (strain R1) | Length = 133 |
| | | | gb\|AAF11800.1\|AE002057_8 (AE002057) hypothetical protein [Deinococcus | |
| | | | radiodurans] | |
| SEQ ID N^{o} 148 | LI-4272.1 | | No Hits found | |
| SEQ ID N^{o} 149 | LI-4674.1 | 63 | pir\|\|F75297 hypothetical protein - Deinococcus radiodurans (strain R1) | Length = 133 |
| | | | gb\|AAF11800.1\|AE002057_8 (AE002057) hypothetical protein [Deinococcus | |
| | | | radiodurans] | |
| SEQ ID N^{o} 150 | LI-5637.1 | | No Hits found | |
| SEQ ID N^{o} 151 | LI-6188.1 | | No Hits found | |
| SEQ ID N^{o} 152 | LI-1431.1 | | No Hits found | |
| SEQ ID N^{o} 153 | LI-3567.1 | | No Hits found | |
| SEQ ID N^{o} 154 | LI-3993.1 | | No Hits found | |
| SEQ ID N^{o} 155 | LI-4194.1 | 71 | gb\|AAD40364.1\| (AF036485) putative transposase [Plasmid pNZ4000] | Length = 226 |
| SEQ ID N^{o} 156 | LI-5299.1 | | No Hits found | |
| SEQ ID N^{o} 157 | LI-5570.1 | | No Hits found | |
| SEQ ID N^{o} 158 | LI-1493.1 | | No Hits found | |
| SEQ ID N^{o} 159 | LI-3374.1 | | No Hits found | |
| SEQ ID N^{o} 160 | LI-3654.1 | | No Hits found | |
| SEQ ID N^{o} 161 | LI-4040.1 | | No Hits found | |
| SEQ ID N^{o} 162 | LI-4069.2 | | | |
| SEQ ID N^{o} 163 | LI-4088.1 | | No Hits found | |
| SEQ ID N^{o} 164 | LI-6132.1 | | No Hits found | |
| SEQ ID N^{o} 165 | LI-1533.1 | | No Hits found | |
| SEQ ID N^{o} 166 | LI-5750.1 | | No Hits found | |
| SEQ ID N^{o} 167 | LI-6127.1 | 55 | pir\|\|A41902 arsenical resistance operon repressor - Staphylococcus xylosus | Length = 104 |
| | | | plasmid pSX267 | |
| SEQ ID N^{o} 168 | LI-708.1 | | No Hits found | |
| SEQ ID N^{o} 169 | LI-2517.1 | | No Hits found | |
| SEQ ID N^{o} 170 | LI-1438.1 | | No Hits found | |
| SEQ ID N^{o} 171 | LI-3307.1 | | No Hits found | |
| SEQ ID N^{o} 172 | LI-4151.1 | | No Hits found | |
| SEQ ID N^{o} 173 | LI-550.1 | | No Hits found | |
| SEQ ID N^{o} 174 | LI-5573.1 | | No Hits found | |
| SEQ ID N^{o} 175 | LI-2618.1 | | No Hits found | |
| SEQ ID N^{o} 176 | LI-298.1 | | No Hits found | |
| SEQ ID N^{o} 177 | LI-3652.1 | | No Hits found | |
| SEQ ID N^{o} 178 | LI-5585.1 | | No Hits found | |
| SEQ ID N^{o} 179 | LI-5707.1 | 44 | gb\|AAC38975.1\| (AF041330) NADH dehydrogenase subunit 5 [Bodo saltans] | Length = 212 |
| SEQ ID N^{o} 180 | LI-31.1 | | No Hits found | |
| SEQ ID N^{o} 181 | LI-4041.1 | | No Hits found | |
| SEQ ID N^{o} 182 | LI-4828.1 | | No Hits found | |
| SEQ ID N^{o} 183 | LI-2188.1 | | No Hits found | |
| SEQ ID N^{o} 184 | LI-5616.1 | | No Hits found | |
| SEQ ID N^{o} 185 | LI-2350.1 | | No Hits found | |
| SEQ ID N^{o} 186 | LI-269.1 | | No Hits found | |
| SEQ ID N^{o} 187 | LI-329.1 | | No Hits found | |
| SEQ ID N^{o} 188 | LI-3992.1 | | No Hits found | |
| SEQ ID N^{o} 189 | LI-5409.1 | | No Hits found | |
| SEQ ID N^{o} 190 | LI-6056.1 | | No Hits found | |
| SEQ ID N^{o} 191 | LI-1183.1 | 49 | sp\|P10023\|YGI2_BACTU HYPOTHETICAL 30.3 KD PROTEIN (ORF 2) | Length = 270 |
| | | | emb\|CAA31837.1\| (X13481) ORF 2 [Bacillus thuringiensis] | |
| SEQ ID N^{o} 192 | LI-1282.1 | | No Hits found | |
| SEQ ID N^{o} 193 | LI-2924.1 | | No Hits found | |
| SEQ ID N^{o} 194 | LI-4020.1 | 57 | gb\|AAD21914.1\| (AF085222) unknown [Streptococcus thermophilus | Length = 107 |
| | | | bacteriophage DT1] | |
| SEQ ID N^{o} 195 | LI-5642.1 | 54 | sp\|P02909\|PTLA_STAAU PTS SYSTEM, LACTOSE-SPECIFIC IIA | |
| | | | COMPONENT (EIIA-LAC) (LACTOSE-PERMEASE IIA COMPONENT) | |
| | | | (PHOSPHOTRANSFERASE ENZYME II, A COMPONENT) (EIII-LAC) | |
| | | | gb\|AAA26648.1\| (J03479) enzyme III-lac (lacF) [Staphylococcus aureus] | |
| SEQ ID N^{o} 196 | LI-577.1 | | No Hits found | |
| SEQ ID N^{o} 197 | LI-6012.1 | | No Hits found | |
| SEQ ID N^{o} 198 | LI-3900.1 | | No Hits found | |
| SEQ ID N^{o} 199 | LI-4195.1 | 91 | pir\|\|S49318 transposase - Enterococcus hirae insertion sequence IS1216 | Length = 226 |
| | | | emb\|CAA57312.1\| (X81654) transposase [Enterococcus hirae] | |
| | | | emb\|CAA48844.1\| (X69092) transposase [Enterococcus hirae] | |
| SEQ ID N^{o} 200 | LI-443.1 | | No Hits found | |
| SEQ ID N^{o} 201 | LI-4484.1 | | No Hits found | |
| SEQ ID N^{o} 202 | LI-4869.1 | | No Hits found | |
| SEQ ID N^{o} 203 | LI-5183.1 | 45 | gb\|AAC38975.1\| (AF041330) NADH dehydrogenase subunit 5 [Bodo saltans] | Length = 212 |
| SEQ ID N^{o} 204 | LI-2365.1 | 55 | pir\|\|G72548 hypothetical protein APE1675 - Aeropyrum pernix (strain K1) | Length = 155 |
| | | | dbj\|BAA80676.1\| (AP000062) 155aa long hypothetical protein [Aeropyrum | |
| | | | pernix] | |
| SEQ ID N^{o} 205 | LI-3400.1 | | No Hits found | |
| SEQ ID N^{o} 206 | LI-3989.1 | 56 | emb\|CAB53822.1\| (AJ242593) putative repressor protein [Bacteriophage A118] | Length = 101 |
| SEQ ID N^{o} 207 | LI-4136.1 | | No Hits found | |
| SEQ ID N^{o} 208 | LI-480.1 | | No Hits found | |
| SEQ ID N^{o} 209 | LI-6104.1 | | No Hits found | |
| SEQ ID N^{o} 210 | LI-1935.1 | | No Hits found | |
| SEQ ID N^{o} 211 | LI-2169.1 | | No Hits found | |
| SEQ ID N^{o} 212 | LI-4179.1 | 54 | pir\|\|S38640 replication protein B - Pediococcus halophilus (ATCC 33315) | Length = 168 |
| | | | (cryptic plasmid) emb\|CAA53279.1\| (X75607) RepB [Tetragenococcus | |
| | | | halophilus] prf\|\|2207193B repB gene [Tetragenococcus halophilus] | |
| SEQ ID N^{o} 213 | LI-4533.1 | | No Hits found | |
| SEQ ID N^{o} 214 | LI-5677.1 | 44 | pir\|\|G72510 hypothetical protein APE2061 - Aeropyrum pernix (strain K1) | Length = 114 |
| | | | dbj\|BAA81071.1\| (AP000063) 114aa long hypothetical protein [Aeropyrum | |
| | | | pernix] | |
| SEQ ID N^{o} 215 | LI-623.1 | | No Hits found | |
| SEQ ID N^{o} 216 | LI-807.1 | | No Hits found | |
| SEQ ID N^{o} 217 | LI-2866.1 | 40 | ref\|NP_010291.1\| Ydr008cp pir\|\|S70313 hypothetical protein YDR008c - yeast | Length = 114 |
| | | | (Saccharomyces cerevisiae) | |
| SEQ ID N^{o} 218 | LI-3022.1 | 42 | dbj\|BAA87194.1\| (AB027890) Hypothetical protein [Schizosaccharomyces | Length = 210 |
| | | | pombe] | |
| SEQ ID N^{o} 219 | LI-508.1 | | No Hits found | |
| SEQ ID N^{o} 220 | LI-552.1 | | No Hits found | |
| SEQ ID N^{o} 221 | LI-4086.1 | | No Hits found | |
| SEQ ID N^{o} 222 | LI-4935.1 | | No Hits found | |
| SEQ ID N^{o} 223 | LI-1312.1 | 30 | pir\|\|H72754 hypothetical protein APE0029 - Aeropyrum pernix (strain K1) | Length = 138 |
| | | | dbj\|BAA78938.1\| (AP000058) 138aa long hypothetical protein [Aeropyrum | |
| | | | pernix] | |
| SEQ ID N^{o} 224 | LI-2970.1 | | No Hits found | |
| SEQ ID N^{o} 225 | LI-234.1 | 49 | pir\|\|T31613 hypothetical protein Y50E8A.i - Caenorhabditis elegans | Length = 836 |
| SEQ ID N^{o} 226 | LI-1553.1 | | No Hits found | |
| SEQ ID N^{o} 227 | LI-2935.1 | | No Hits found | |
| SEQ ID N^{o} 228 | LI-2941.1 | | No Hits found | |
| SEQ ID N^{o} 229 | Ll-3325.1 | | No Hits found | |
| SEQ ID N^{o} 230 | LI-4061 .1 | | No Hits found | |
| SEQ ID N^{o} 231 | LI-6086.1 | | No Hits found | |
| SEQ ID N^{o} 232 | LI-2646.1 | 49 | pir\|\|S72740 B1177_F1_12 protein - Mycobacterium leprae gb\|AAA17104.1\| | Length = 219″ |
| | | | (U00011) p1177c; B1177_F1_12 [Mycobacterium leprae] | |
| SEQ ID N^{o} 233 | LI-3823.1 | 55 | pir\|\|C72455 hypothetical protein APE2287 - Aeropyrum pernix (strain K1) | Length = 191 |
| | | | dbj\|BAA81299.1\| (AP000064) 191aa long hypothetical protein [Aeropyrum | |
| | | | pernix] | |
| SEQ ID N^{o} 234 | LI-5622.1 | | No Hits found | |
| SEQ ID N^{o} 235 | LI-1499.1 | 45 | pir\|\|H72469 hypothetical protein APE2401 - Aeropyrum pernix (strain K1) | Length = 252 |
| | | | dbj\|BAA81416.1\| (AP000064) 252aa long hypothetical protein [Aeropyrum | |
| | | | pernix] | |
| SEQ ID N^{o} 236 | LI-4044.1 | | No Hits found | |
| SEQ ID N^{o} 237 | LI-4799.1 | 34 | emb\|CAB57660.1\| (Y18930) hypothetical protein [Sulfolobus solfataricus] | Length = 108 |
| SEQ ID N^{o} 238 | LI-1044.1 | | No Hits found | |
| SEQ ID N^{o} 239 | LI-1247.1 | | No Hits found | |
| SEQ ID N^{o} 240 | LI-5276.1 | | No Hits found | |
| SEQ ID N^{o} 241 | LI-4038.1 | 53 | pir\|\|T39903 serine-rich protein - fission yeast (Schizosaccharomyces pombe) | Length = 534 |
| | | | emb\|CAA22127.1\| (AL033534) hypothetical serine-rich secreted protein | |
| | | | [Schizosaccharomyces pombe] | |
| SEQ ID N^{o} 242 | LI-543.1 | | No Hits found | |
| SEQ ID N^{o} 243 | LI-5937.1 | 55 | gb\|AAC27928.1\| (AF062070) thermosensitive mutant immunity repressor | Length = 147 |
| | | | [bacteriophage phi-105] | |
| SEQ ID N^{o} 244 | LI-2068.1 | 32 | pir\|\|F71456 hypothetical protein PH0308 - Pyrococcus horikoshii | Length = 215 |
| | | | dbj\|BAA29381.1\| (AP000001) 215aa long hypothetical protein [Pyrococcus | |
| | | | horikoshii] | |
| SEQ ID N^{o} 245 | LI-3142.1 | | No Hits found | |
| SEQ ID N^{o} 246 | LI-4047.1 | | No Hits found | |
| SEQ ID N^{o} 247 | LI-4073.1 | | No Hits found | |
| SEQ ID N^{o} 248 | LI-4483.1 | | No Hits found | |
| SEQ ID N^{o} 249 | LI-547.1 | 35 | pir\|\|A70002 protein kinase homolog ytvA - Bacillus subtilis gb\|AAC00382.1\| | Length = 261 |
| | | | (AF008220) putative protein kinase [Bacillus subtilis] emb\|CAB15012.1\| | |
| | | | (Z99119) similar to protein kinase [Bacillus subtilis] | |
| SEQ ID N^{o} 250 | LI-5640.1 | 44 | sp\|Q45399\|PTCB_BACST PTS SYSTEM, CELLOBIOSE-SPECIFIC IIB | |
| | | | COMPONENT (EIIB-CEL) (CELLOBIOSE-PERMEASE IIB | |
| | | | COMPONENT) (PHOSPHOTRANSFERASE ENZYME II, B | |
| | | | COMPONENT) pir\|\|B49898 cellobiose phosphotransferase system celA - | |
| | | | Bacillus | |
| SEQ ID N^{o} 251 | LI-689.1 | 56 | gi\|6321628 Cell wall protein; Crh1p sp\|P53301\|YG46_YEAST HYPOTHETICAL | |
| | | | 52.8 KD PROTEIN IN BUB1-HIP1 INTERGENIC REGION pir\|\|S64507 probable | |
| | | | membrane protein YGR189c - yeast (Saccharomyces cerevisiae) | |
| | | | emb\|CAA97215.1\| (Z72974) ORF YGR189c [Sacch | |
| SEQ ID N^{o} 252 | LI-2066.1 | | No Hits found | |
| SEQ ID N^{o} 253 | LI-4008.1 | | No Hits found | |
| SEQ ID N^{o} 254 | LI-4042.1 | | No Hits found | |
| SEQ ID N^{o} 255 | LI-4145.1 | 95 | gb\|AAB37344.1\| (U78967) cadmium resistance regulatory protein [Lactococcus | Length = 119 |
| | | | lactis] | |
| SEQ ID N^{o} 256 | LI-5586.1 | 51 | sp\|P45924\|YQBH_BACSU HYPOTHETICAL 14.3 KD PROTEIN IN SPOIIIC- | |
| | | | CWLA INTERGENIC REGION pir\|\|C69947 phage-related protein homolog yqbH - | |
| | | | Bacillus subtilis dbj\|BAA12403.1\| (D84432) YqbH [Bacillus subtilis] | |
| | | | emb\|CAB14552.1\| (Z99117) similar to phage-related p | |
| SEQ ID N^{o} 257 | LI-4152.1 | | No Hits found | |
| SEQ ID N^{o} 258 | LI-4154.1 | | No Hits found | |
| SEQ ID N^{o} 259 | LI-4181.1 | 54 | pir\|\|T09011 probable transposase TnpA - Streptococcus pyogenes (fragment) | Length = 364 |
| | | | gb\|AAB92607.1\| (AF026542) TnpA [Streptococcus pyogenes] | |
| SEQ ID N^{o} 260 | LI-4220.1 | | No Hits found | |
| SEQ ID N^{o} 261 | LI-4477.1 | | No Hits found | |
| SEQ ID N^{o} 262 | LI-2014.1 | | No Hits found | |
| SEQ ID N^{o} 263 | LI-5182.1 | 50 | dbj\|BAA97098.1\| (AP002460) gene_id: F1D9.26˜unknown protein [Arabidopsis | Length = 260 |
| | | | thaliana] | |
| SEQ ID N^{o} 264 | LI-5545.1 | | No Hits found | |
| SEQ ID N^{o} 265 | LI-5549.1 | | No Hits found | |
| SEQ ID N^{o} 266 | LI-5556.1 | | No Hits found | |
| SEQ ID N^{o} 267 | LI-6015.1 | | No Hits found | |
| SEQ ID N^{o} 268 | LI-6125.1 | 54 | sp\|Q01256\|ARSR_STAXY ARSENICAL RESISTANCE OPERON REPRESSOR | Length = 104 |
| | | | gb\|AAA27587.1\| (M80565) ars operon regulatory protein (repressor) [Plasmid | |
| | | | pSX267] | |
| SEQ ID N^{o} 269 | LI-6186.1 | | No Hits found | |
| SEQ ID N^{o} 270 | LI-2659.1 | 39 | pir\|\|C69982 hypothetical protein yrzD - Bacillus subtilis emb\|CAB14726.1\| | Length = 98 |
| | | | (Z99118) yrzD [Bacillus subtilis] | |
| SEQ ID N^{o} 271 | LI-2949.1 | | No Hits found | |
| SEQ ID N^{o} 272 | LI-2961.1 | | No Hits found | |
| SEQ ID N^{o} 273 | LI-4478.1 | | No Hits found | |
| SEQ ID N^{o} 274 | LI-283.1 | 29 | pir\|\|D72568 hypothetical protein APE1830 - Aeropyrum pernix (strain K1) | Length = 100 |
| | | | dbj\|BAA80833.1\| (AP000062) 100aa long hypothetical protein [Aeropyrum | |
| | | | pernix] | |
| SEQ ID N^{o} 275 | LI-4899.1 | | No Hits found | |
| SEQ ID N^{o} 276 | LI-521.1 | 58 | gb\|AAF41678.1\| (AE002479) transcriptional regulator, MerR family [Neisseria | Length = 135 |
| | | | meningitidis MC58] emb\|CAB84745.1\| (AL162756) putative transcriptional | |
| | | | regulator [Neisseria meningitidis] | |
| SEQ ID N^{o} 277 | LI-6126.1 | 51 | gb\|AAD51848.1\|AF178758_4 (AF178758) ArsD [Sinorhizobium sp. As4] | Length = 119 |
| SEQ ID N^{o} 278 | LI-1390.1 | | No Hits found | |
| SEQ ID N^{o} 279 | LI-2968.1 | | No Hits found | |
| SEQ ID N^{o} 280 | LI-4132.1 | 46 | gb\|AAF13645.1\|AF188935_43 (AF188935) pXO2-40 [Bacillus anthracis] | Length = 128 |
| SEQ ID N^{o} 281 | LI-6032.1 | | No Hits found | |
| SEQ ID N^{o} 282 | LI-4028.1 | | No Hits found | |
| SEQ ID N^{o} 283 | LI-773.1 | | No Hits found | |
| SEQ ID N^{o} 284 | LI-2038.1 | 46 | sp\|P75869\|YCCR_ECOLI HYPOTHETICAL 24.1 KDA PROTEIN IN SULA-HELD | |
| | | | INTERGENIC REGION pir\|\|F64836 probable membrane protein b0959 - | |
| | | | Escherichia coli gb\|AAC74045.1\| (AE000198) orf, hypothetical protein | |
| | | | [Escherichia coli] dbj\|BAA35717.1\| (D90733) ORF_ID: o223 | |
| SEQ ID N^{o} 285 | LI-2973.1 | | No Hits found | |
| SEQ ID N^{o} 286 | LI-2019.1 | | No Hits found | |
| SEQ ID N^{o} 287 | LI-2052.1 | 50 | gb\|AAD01956.1\| (AF033016) unknown [Listeria monocytogenes] | Length = 75 |
| SEQ ID N^{o} 288 | LI-2945.1 | | No Hits found | |
| SEQ ID N^{o} 289 | LI-4030.1 | | No Hits found | |
| SEQ ID N^{o} 290 | LI-4522.1 | | No Hits found | |
| SEQ ID N^{o} 291 | LI-6030.1 | | No Hits found | |
| SEQ ID N^{o} 292 | LI-2456.1 | | No Hits found | |
| SEQ ID N^{o} 293 | LI-4215.1 | | No Hits found | |
| SEQ ID N^{o} 294 | LI-5926.1 | 61 | emb\|CAA70358.1\| (Y09161) antigen C [Listeria monocytogenes] | Length = 138 |
| SEQ ID N^{o} 295 | LI-2316.1 | 38 | sp\|P25958\|CMG6_BACSU COMG OPERON PROTEIN 6 pir\|\|G69603 DNA | Length = 127 |
| | | | transport machinery comGF - Bacillus subtilis emb\|CAB14399.1\| (Z99116) | |
| | | | probably part of the DNA transport machinery [Bacillus subtilis] | |
| SEQ ID N^{o} 296 | LI-394.1 | | No Hits found | |
| SEQ ID N^{o} 297 | LI-5039.1 | | No Hits found | |
| SEQ ID N^{o} 298 | LI-6068.1 | | No Hits found | |
| SEQ ID N^{o} 299 | LI-6070.1 | | No Hits found | |
| SEQ ID N^{o} 300 | LI-825.1 | 32 | gb\|AAC69727.1\| (AF061128) merozoite surface protein 1 [Plasmodium | Length = 402 |
| | | | falciparum] | |
| SEQ ID N^{o} 301 | LI-2972.1 | | No Hits found | |
| SEQ ID N^{o} 302 | LI-3339.1 | | No Hits found | |
| SEQ ID N^{o} 303 | LI-3371.1 | 80 | emb\|CAB53849.1\| (AJ242593) gp59 [Bacteriophage A118] | Length = 133 |
| SEQ ID N^{o} 304 | LI-3529.1 | | No Hits found | |
| SEQ ID N^{o} 305 | LI-4063.1 | | No Hits found | |
| SEQ ID N^{o} 306 | LI-4808.1 | 38 | sp\|Q09788\|YA9A_SCHPO HYPOTHETICAL 54.2 KD SERINE-RICH PROTEIN | |
| | | | C13G6.10C IN CHROMOSOME I PRECURSOR pir\|\|S62439 hypothetical | |
| | | | protein SPAC13G6.10c - fission yeast (Schizosaccharomyces pombe) | |
| | | | pir\|\|T37645 hypothetical serine rich protei | |
| SEQ ID N^{o} 307 | LI-4099.1 | | No Hits found | |
| SEQ ID N^{o} 308 | LI-5587.1 | 55 | sp\|P45923\|YQBG_BACSU HYPOTHETICAL 14.7 KD PROTEIN IN SPOIIIC- | |
| | | | CWLA INTERGENIC REGION pir\|\|B69947 hypothetical protein yqbG - Bacillus | |
| | | | subtilis dbj\|BAA06940.1\| (D32216) ORF76 [Bacillus subtilis] dbj\|BAA12402.1\| | |
| | | | (D84432) YqbG [Bacillus subtilis] emb\|CAB1 | |
| SEQ ID N^{o} 309 | LI-4963.1 | 35 | pir\|\|E69801 hypothetical protein yfhL - Bacillus subtilis emb\|CAB12686.1\| | Length = 110 |
| | | | (Z99108) yfhL [Bacillus subtilis] dbj\|BAA24478.1\| (D85082) YfhL [Bacillus | |
| | | | subtilis] | |
| SEQ ID N^{o} 310 | LI-3199.1 | | No Hits found | |
| SEQ ID N^{o} 311 | LI-6057.1 | 26 | emb\|CAB01605.1\| (Z78205) UL36 [Bovine herpesvirus 1] emb\|CAA06097.1\| | Length = 3247 |
| | | | (AJ004801) very large virion protein (tegument) [Bovine herpesvirus type | |
| | | | 1.1] | |
| SEQ ID N^{o} 312 | LI-776.1 | 34 | sp\|P42622\|YHAI_ECOLI HYPOTHETICAL 13.5 KD PROTEIN IN EXUR-TDCC | |
| | | | INTERGENIC REGION pir\|\|E65099 hypothetical 13.5 kD protein in exuR-tdcC | |
| | | | intergenic region - Escherichia coli (strain K-12) gb\|AAA57908.1\| | |
| | | | (U18997) ORF_o118 [Escherichia coli] gb\| | |
| SEQ ID N^{o} 313 | LI-3375.1 | | No Hits found | |
| SEQ ID N^{o} 314 | LI-3377.1 | | No Hits found | |
| SEQ ID N^{o} 315 | LI-5565.1 | 68 | sp\|P54338\|XKDS_BACSU PHAGE-LIKE ELEMENT PBSX PROTEIN XKDS | |
| | | | pir\|\|B69733 PBSX prophage ORF xkdS - Bacillus subtilis emb\|CAA94040.1\| | |
| | | | (Z70177) homologous to yqbS of the skin element [Bacillus subtilis] | |
| | | | emb\|CAB13129.1\| (Z99110) PBSX prophage [Bacillus subtil | |
| SEQ ID N^{o} 316 | LI-5935.1 | 52 | dbj\|BAA36659.1\| (AB016282) ORF2 [bacteriophage phi-105] | Length = 148 |
| SEQ ID N^{o} 317 | LI-3998.1 | | No Hits found | |
| SEQ ID N^{o} 318 | LI-6005.1 | 57 | dbj\|BAA36659.1\| (AB016282) ORF2 [bacteriophage phi-105] | Length = 148 |
| SEQ ID N^{o} 319 | LI-1341.1 | | No Hits found | |
| SEQ ID N^{o} 320 | LI-2487.1 | 30 | sp\|Q45460\|OPBA_BACSU CHOLINE TRANSPORT ATP-BINDING PROTEIN | |
| | | | OPUBA pir\|\|G69669 choline ABC transporter (ATP-binding protein) opuBA - | |
| | | | Bacillus subtilis gb\|AAC14356.1\| (AF008930) ATPase [Bacillus subtilis] | |
| | | | emb\|CAB15378.1\| (Z99121) choline ABC tr | |
| SEQ ID N^{o} 321 | LI-3180.1 | | No Hits found | |
| SEQ ID N^{o} 322 | LI-2997.1 | 44 | emb\|CAB53856.1\| (AJ242593) gp66 [Bacteriophage A118] | Length = 144 |
| SEQ ID N^{o} 323 | LI-6045.1 | 44 | emb\|CAB53856.1\| (AJ242593) gp66 [Bacteriophage A118] | Length = 144 |
| SEQ ID N^{o} 324 | LI-1517.1 | 40 | pir\|\|A28298 myosin heavy chain beta, cardiac muscle - golden hamster | Length = 974″ |
| | | | (fragment) emb\|CAA30256.1\| (X07273) beta-myosin heavy chain (974 AA); S2 | |
| | | | fragment and LMM region [Mesocricetus auratus] | |
| SEQ ID N^{o} 325 | LI-6006.2 | | | |
| SEQ ID N^{o} 326 | LI-3335.1 | 34 | gb\|AAF66771.1\|AF147806_35 (AF147806) major tegument protein [Gallid | Length = 3325 |
| | | | herpesvirus 2] | |
| SEQ ID N^{o} 327 | LI-51.1 | | No Hits found | |
| SEQ ID N^{o} 328 | LI-1931.1 | | No Hits found | |
| SEQ ID N^{o} 329 | LI-4521.1 | | No Hits found | |
| SEQ ID N^{o} 330 | LI-6028.1 | 51 | sp\|P45911\|YQAN_BACSU HYPOTHETICAL 16.1 KD PROTEIN IN SPOIIIC- | |
| | | | CWLA INTERGENIC REGION pir\|\|E69945 hypothetical protein yqaN - Bacillus | |
| | | | subtilis dbj\|BAA06928.1\| (D32216) ORF39 [Bacillus subtilis] dbj\|BAA12389.1\| | |
| | | | (D84432) YqaN [Bacillus subtilis] emb\|CAB1 | |
| SEQ ID N^{o} 331 | LI-3319.1 | | No Hits found | |
| SEQ ID N^{o} 332 | LI-4250.1 | | No Hits found | |
| SEQ ID N^{o} 333 | LI-108.1 | | No Hits found | |
| SEQ ID N^{o} 334 | LI-4959.1 | 41 | sp\|P54940\|YXEA_BACSU HYPOTHETICAL 13.0 KD PROTEIN IN IDH-DEOR | |
| | | | INTERGENIC REGION PRECURSOR pir\|\|C70074 hypothetical protein | |
| | | | yxeA - Bacillus subtilis dbj\|BAA08317.1\| (D45912) yxeA [Bacillus subtilis] | |
| | | | emb\|CAB15998.1\| (Z99124) yxeA [Bacillus sub | |
| SEQ ID N^{o} 335 | LI-846.1 | | No Hits found | |
| SEQ ID N^{o} 336 | LI-4170.1 | | No Hits found | |
| SEQ ID N^{o} 337 | LI-5577.1 | 83 | sp\|P45929\|YQBM_BACSU HYPOTHETICAL 16.3 KD PROTEIN IN SPOIIIC- | |
| | | | CWLA INTERGENIC REGION pir\|\|H69947 phage-related protein homolog yqbM - | |
| | | | Bacillus subtilis dbj\|BAA06945.1\| (D32216) ORF71 [Bacillus subtilis] | |
| | | | dbj\|BAA12408.1\| (D84432) YqbM [Bacillus subtilis] | |
| SEQ ID N^{o} 338 | LI-779.1 | 40 | pir\|\|F69168 hypothetical protein MTH520 - Methanobacterium | Length = 104 |
| | | | thermoautotrophicum (strain Delta H) gb\|AAB85026.1\| (AE000835) | |
| | | | unknown [Methanobacterium thermoautotrophicum] | |
| SEQ ID N^{o} 339 | LI-2822.1 | | No Hits found | |
| SEQ ID N^{o} 340 | LI-2931.1 | 83 | emb\|CAB53814.1\| (AJ242593) gp28 [Bacteriophage A118] | Length = 149 |
| SEQ ID N^{o} 341 | LI-2976.1 | | No Hits found | |
| SEQ ID N^{o} 342 | LI-3835.1 | 36 | emb\|CAB57639.1\| (Y18930) hypothetical protein [Sulfolobus solfataricus] | Length = 106 |
| SEQ ID N^{o} 343 | LI-3613.1 | | No Hits found | |
| SEQ ID N^{o} 344 | LI-3988.1 | 51 | emb\|CAB53821.1\| (AJ242593) gp35 [Bacteriophage A118] | Length = 163 |
| SEQ ID N^{o} 345 | LI-2287.1 | | No Hits found | |
| SEQ ID N^{o} 346 | LI-338.1 | | No Hits found | |
| SEQ ID N^{o} 347 | LI-4098.1 | 25 | pir\|\|I40868 hypothetical protein 3 nanH region - Clostridium perfringens | Length = 265 |
| | | | emb\|CAA60798.1\| (X87369) ORF3 [Clostridium perfringens] | |
| SEQ ID N^{o} 348 | LI-1716.1 | | No Hits found | |
| SEQ ID N^{o} 349 | LI-6063.1 | | No Hits found | |
| SEQ ID N^{o} 350 | LI-1117.1 | | No Hits found | |
| SEQ ID N^{o} 351 | LI-4137.1 | | No Hits found | |
| SEQ ID N^{o} 352 | LI-2778.1 | 33 | gb\|AAB60026.1\| (U09422) ORF7 [Enterococcus faecalis] prf\|\|2114402U ORF 7 | Length = 157 |
| | | | [Enterococcus faecalis] | |
| SEQ ID N^{o} 353 | LI-2783.1 | 47 | gb\|AAF41678.1\| (AE002479) transcriptional regulator, MerR family [Neisseria | Length = 135 |
| | | | meningitidis MC58] emb\|CAB84745.1\| (AL162756) putative transcriptional | |
| | | | regulator [Neisseria meningitidis] | |
| SEQ ID N^{o} 354 | LI-3846.1 | | No Hits found | |
| SEQ ID N^{o} 355 | LI-929.1 | | No Hits found | |
| SEQ ID N^{o} 356 | LI-664.1 | | No Hits found | |
| SEQ ID N^{o} 357 | LI-3625.1 | 51 | pir\|\|B70030 conserved hypothetical protein yvbK - Bacillus subtilis | Length = 155 |
| | | | emb\|CAB15394.1\| (Z99121) similar to hypothetical proteins from B. subtilis | |
| | | | [Bacillus subtilis] | |
| SEQ ID N^{o} 358 | LI-4119.1 | | No Hits found | |
| SEQ ID N^{o} 359 | LI-4479.1 | | No Hits found | |
| SEQ ID N^{o} 360 | LI-2939.1 | | No Hits found | |
| SEQ ID N^{o} 361 | LI-4012.1 | 52 | dbj\|BAA97823.1\| (AB044554) orf 16 [Staphylococcus aureus prophage phiPV83] | Length = 159 |
| SEQ ID N^{o} 362 | LI-5553.1 | | No Hits found | |
| SEQ ID N^{o} 363 | LI-5583.1 | 50 | sp\|P54330\|XKDJ_BACSU PHAGE-LIKE ELEMENT PBSX PROTEIN XKDJ | |
| | | | pir\|\|B69732 PBSX prophage ORF xkdJ - Bacillus subtilis emb\|CAA94065.1\| | |
| | | | (Z70177) homologous to yqbJ of the skin element [Bacillus subtilis] | |
| | | | emb\|CAB13121.1\| (Z99110) PBSX prophage [Bacillus subtil | |
| SEQ ID N^{o} 364 | LI-5612.2 | | | |
| SEQ ID N^{o} 365 | LI-4016.1 | 49 | gb\|AAC97922.1\| (AF077306) gp151 [Streptococcus thermophilus bacteriophage | Length = 151 |
| | | | Sfi19] gb\|AAD44072.1\|AF115102_31 (AF115102) orf151 gp [Streptococcus | |
| | | | thermophilus bacteriophage Sfi19] | |
| SEQ ID N^{o} 366 | LI-4193.1 | 78 | emb\|CAA63529.1\| (X92946) transposase [Lactococcus lactis] | Length = 228 |
| SEQ ID N^{o} 367 | LI-2609.1 | 26 | pir\|\|F72598 hypothetical protein APE1254 - Aeropyrum pernix (strain K1) | Length = 109 |
| | | | dbj\|BAA80244.1\| (AP000061) 109aa long hypothetical protein [Aeropyrum | |
| | | | pernix] | |
| SEQ ID N^{o} 368 | LI-2940.1 | | No Hits found | |
| SEQ ID N^{o} 369 | LI-4459.1 | | No Hits found | |
| SEQ ID N^{o} 370 | LI-5315.1 | 69 | sp\|O66911\|UVRA_AQUAE EXCINUCLEASE ABC SUBUNIT A pir\|\|C70360 | Length = 926 |
| | | | repair excision nuclease subunit A - Aquifex aeolicus gb\|AAC06874.1\| | |
| | | | (AE000702) repair excision nuclease subunit A [Aquifex aeolicus] | |
| SEQ ID N^{o} 371 | LI-5576.1 | 65 | sp\|P45930\|YQBN_BACSU HYPOTHETICAL 17.1 KD PROTEIN IN SPOIIIC- | |
| | | | CWLA INTERGENIC REGION pir\|\|A69948 phage-related protein homolog yqbN - | |
| | | | Bacillus subtilis dbj\|BAA0946.1\| (D32216) ORF55 [Bacillus subtilis] | |
| | | | dbj\|BAA12410.1\| (D84432) YqbN [Bacillus subtilis] | |
| SEQ ID N^{o} 372 | LI-5578.1 | | No Hits found | |
| SEQ ID N^{o} 373 | LI-2967.1 | | No Hits found | |
| SEQ ID N^{o} 374 | LI-2836.1 | 38 | pir\|\|H72608 hypothetical protein APE1334 - Aeropyrum pernix (strain K1) | Length = 211 |
| | | | dbj\|BAA80326.1\| (AP000061) 211aa long hypothetical protein [Aeropyrum | |
| | | | pernix] | |
| SEQ ID N^{o} 375 | LI-3370.1 | | No Hits found | |
| SEQ ID N^{o} 376 | LI-5584.1 | 70 | sp\|P45925\|YQBI_BACSU HYPOTHETICAL 19.9 KD PROTEIN IN SPOIIIC- | |
| | | | CWLA INTERGENIC REGION pir\|\|D69947 phage-related protein homolog yqbl - | |
| | | | Bacillus subtilis dbj\|BAA06941.1\| (D32216) ORF67 [Bacillus subtilis] | |
| | | | dbj\|BAA12404.1\| (D84432) Yqbl [Bacillus subtilis] | |
| SEQ ID N^{o} 377 | LI-5801.1 | 67 | gb\|AAF04740.1\| (AF102169) multidrug resistance-like protein [Listeria | Length = 228 |
| | | | monocytogenes] | |
| SEQ ID N^{o} 378 | LI-3338.1 | 42 | pir\|\|T33369 hypothetical protein H02F09.3 - Caenorhabditis elegans | Length = 1275 |
| | | | gb\|AAC64622.1\| (AF077538) unknown [Caenorhabditis elegans] | |
| SEQ ID N^{o} 379 | LI-4106.1 | 65 | sp\|Q99338\|ISTB_BACTB INSERTION SEQUENCE IS232 PUTATIVE ATP- | Length = 250 |
| | | | BINDING PROTEIN pir\|\|B37801 IstB protein homolog - Bacillus thuringiensis | |
| | | | gb\|AAA98141.1\| (M38370) ORF2 [Insertion sequence IS232] | |
| SEQ ID N^{o} 380 | LI-4155.1 | 21 | pir\|\|F72491 hypothetical protein APE2573 - Aeropyrum pernix (strain K1) | Length = 238 |
| | | | dbj\|BAA81590.1\| (AP000064) 238aa long hypothetical protein [Aeropyrum | |
| | | | pernix] | |
| SEQ ID N^{o} 381 | LI-2451.1 | | No Hits found | |
| SEQ ID N^{o} 382 | LI-3987.1 | | No Hits found | |
| SEQ ID N^{o} 383 | LI-4474.1 | | No Hits found | |
| SEQ ID N^{o} 384 | LI-494.1 | 42 | gb\|AAF41678.1\| (AE002479) transcriptional regulator, MerR family [Neisseria | Length = 135 |
| | | | meningitidis MC58] emb\|CAB84745.1\| (AL162756) putative transcriptional | |
| | | | regulator [Neisseria meningitidis] | |
| SEQ ID N^{o} 385 | LI-2964.1 | | No Hits found | |
| SEQ ID N^{o} 386 | LI-6202.1 | | | |
| SEQ ID N^{o} 387 | LI-2102.1 | | No Hits found | |
| SEQ ID N^{o} 388 | LI-1260.1 | | No Hits found | |
| SEQ ID N^{o} 389 | LI-2820.1 | 94 | gb\|AAF04738.1\| (AF102167) stress protein-like protein [Listeria monocytogenes] | Length = 174 |
| SEQ ID N^{o} 390 | LI-3283.1 | | No Hits found | |
| SEQ ID N^{o} 391 | LI-3378.1 | | No Hits found | |
| SEQ ID N^{o} 392 | LI-6009.2 | | | |
| SEQ ID N^{o} 393 | LI-624.1 | | No Hits found | |
| SEQ ID N^{o} 394 | LI-2969.1 | | No Hits found | |
| SEQ ID N^{o} 395 | LI-2999.1 | | No Hits found | |
| SEQ ID N^{o} 396 | LI-4003.1 | | No Hits found | |
| SEQ ID N^{o} 397 | LI-6109.1 | | No Hits found | |
| SEQ ID N^{o} 398 | LI-4081.1 | 61 | emb\|CAB83893.1\| (AL162753) hypothetical protein NMA0603 [Neisseria | Length = 182 |
| | | | meningitidis] | |
| SEQ ID N^{o} 399 | LI-4696.1 | 67 | sp\|P96674\|YDEQ_BACSU PUTATIVE NAD(P)H OXIDOREDUCTASE YDEQ | |
| | | | pir\|\|C69779 NAD(P)H oxidoreductase homolog ydeQ - Bacillus subtilis | |
| | | | dbj\|BAA19364.1\| (AB001488) PROBABLE NAD(P)H OXIDOREDUCTASE. | |
| | | | [Bacillus subtilis] emb\|CAB12337.1\| (Z99106) similar to NAD(P)H ox | |
| SEQ ID N^{o} 400 | LI-3361.1 | 87 | emb\|CAB53787.1\| (AJ242593) putative terminase small subunit [Bacteriophage | Length = 180 |
| | | | A118] | |
| SEQ ID N^{o} 401 | LI-2383.1 | 25 | gb\|AAC45309.1\| (U81957) putative DNA binding protein [Streptococcus gordonii] | Length = 122 |
| SEQ ID N^{o} 402 | LI-2787.1 | | No Hits found | |
| SEQ ID N^{o} 403 | LI-4184.1 | 72 | gb\|AAB91419.1\| (AF037091) RepB homolog [Lactobacillus rhamnosus] | Length = 172 |
| SEQ ID N^{o} 404 | LI-4150.1 | 89 | sp\|P19241\|BINR_STAAU DNA-INVERTASE BINR (TRANSPOSON TN552) | Length = 192 |
| | | | pir\|\|S16509 DNA-invertase - Staphylococcus aureus transposon Tn552 | |
| | | | gb\|AAA26640.1\| (M36694) DNA invertase (ttg start codon) [Staphylococcus | |
| | | | aureus] | |
| SEQ ID N^{o} 405 | LI-1323.1 | | No Hits found | |
| SEQ ID N^{o} 406 | LI-3996.1 | | No Hits found | |
| SEQ ID N^{o} 407 | LI-5572.1 | 41 | pir\|\|S49915 extensin-like protein - maize emb\|CAA84230.1\| (Z34465) extensin- | Length = 1188 |
| | | | like protein [Zea mays] prf\|\|2111476A extensin-like domain [Zea mays] | |
| SEQ ID N^{o} 408 | LI-4485.1 | | No Hits found | |
| SEQ ID N^{o} 409 | LI-6029.1 | | No Hits found | |
| SEQ ID N^{o} 410 | LI-6076.1 | 35 | pir\|\|S00250 myosin heavy chain - slime mold (Dictyostelium discoideum) | Length = 160 |
| | | | (fragment) | |
| SEQ ID N^{o} 411 | LI-4309.1 | | No Hits found | |
| SEQ ID N^{o} 412 | LI-4140.1 | 83 | gb\|AAF86683.1\| (AF179848) unknown [Lactococcus lactis subsp. lactis] | Length = 196 |
| SEQ ID N^{o} 413 | LI-6189.2 | | | |
| SEQ ID N^{o} 414 | LI-1239.1 | | No Hits found | |
| SEQ ID N^{o} 415 | LI-4079.1 | 57 | emb\|CAB83894.1\| (AL162753) hypothetical protein NMA0604 [Neisseria | Length = 187 |
| | | | meningitidis] | |
| SEQ ID N^{o} 416 | LI-4046.1 | 38 | pir\|\|T13523 hypothetical protein 34 - Bacillus phage phi-105 dbj\|BAA36640.1\| | Length = 200 |
| | | | (AB016282) ORF34 [bacteriophage phi-105] | |
| SEQ ID N^{o} 417 | LI-5934.1 | | No Hits found | |
| SEQ ID N^{o} 418 | LI-2998.1 | 35 | pir\|\|T13190 hypothetical protein R242 - Lactobacillus phage phi-gle | Length = 242 |
| | | | emb\|CAA66766.1\| (X98106) Rorf242 [Bacteriophage phig1e] | |
| SEQ ID N^{o} 419 | LI-304.1 | 20 | emb\|CAA49825.1\| (X70360) ORF2 [Azospirillum brasilense] | Length = 173 |
| SEQ ID N^{o} 420 | LI-4945.1 | | No Hits found | |
| SEQ ID N^{o} 421 | LI-1552.1 | 29 | dbj\|BAA82251.1\| (AB014440) orf3 [Staphylococcus aureus] | Length = 151 |
| SEQ ID N^{o} 422 | LI-4037.1 | 51 | dbj\|BAA97848.1\| (AB044554) orf 41 ˜phi PVL orf 5 and 6 [Staphylococcus | Length = 194 |
| | | | aureus prophage phiPV83] | |
| SEQ ID N^{o} 423 | LI-5108.1 | 38 | sp\|P54396\|YPMB_BACSU HYPOTHETICAL 17.9 KD PROTEIN IN DING-ASPB | |
| | | | INTERGENIC REGION pir\|\|F69938 hypothetical protein ypmB - Bacillus subtilis | |
| | | | gb\|AAB38453.1\| (L47709) putative [Bacillus subtilis] emb\|CAB14154.1\| (Z99115) | |
| | | | ypmB [Bacillus subtilis] | |
| SEQ ID N^{o} 424 | LI-5561.1 | 53 | sp\|P54340\|XKDU_BACSU PHAGE-LIKE ELEMENT PBSX PROTEIN XKDT | |
| | | | pir\|\|D69733 PBSX prophage ORF xkdU - Bacillus subtilis emb\|CAA94042.1\| | |
| | | | (Z70177) homologous to yqcA of the skin element [Bacillus subtilis] | |
| | | | emb\|CAB13131.1\| (Z99110) PBSX prophage [Bacillus subtil | |
| SEQ ID N^{o} 425 | LI-1717.1 | | No Hits found | |
| SEQ ID N^{o} 426 | LI-6043.1 | 34 | pir\|\|T13190 hypothetical protein R242 - Lactobacillus phage phi-gle | Length = 242 |
| | | | emb\|CAA66766.1\| (X98106) Rorf242 [Bacteriophage phig 1e] | |
| SEQ ID N^{o} 427 | LI-3740.1 | 44 | sp\|P46330\|YXBF_BACSU HYPOTHETICAL 44.3 KDA PROTEIN IN HTPG- | |
| | | | ALDX INTERGENIC REGION pir\|\|A70073 hypothetical protein yxbF - Bacillus | |
| | | | subtilis dbj\|BAA21600.1\| (AB005554) yxbF [Bacillus subtilis] emb\|CAB16021.1\| | |
| | | | (Z99124) alternate gene name: yxaT [Bacillus | |
| SEQ ID N^{o} 428 | LI-6087.1 | 49 | sp\|P39784\|PCF_BACSU POSITIVE CONTROL FACTOR pir\|\|I40413 positive | |
| | | | control factor (xre region) xpf - Bacillus subtilis emb\|CAA84046.1\| (Z34287) | |
| | | | ORF7; homology to regions 4.1 and 4.2 of sigma factors [Bacillus subtilis] | |
| | | | emb\|CAA94057.1\| (Z70177) | |
| SEQ ID N^{o} 429 | LI-2151.1 | | No Hits found | |
| SEQ ID N^{o} 430 | LI-4097.1 | | No Hits found | |
| SEQ ID N^{o} 431 | LI-4146.1 | 78 | gb\|AAB08926.1\| (U39859) invertase-enterococcal [Enterococcus faecalis] | Length = 202 |
| SEQ ID N^{o} 432 | LI-1236.1 | | No Hits found | |
| SEQ ID N^{o} 433 | LI-3291.1 | | No Hits found | |
| SEQ ID N^{o} 434 | LI-4512.1 | 30 | pir\|\|F71011 hypothetical protein PH1388 - Pyrococcus horikoshii | Length = 119 |
| | | | dbj\|BAA30494.1\| (AP000006) 119aa long hypothetical protein [Pyrococcus | |
| | | | horikoshii] | |
| SEQ ID N^{o} 435 | LI-4120.1 | 32 | gb\|AAC17959.1\| (AF022806) unknown [Pantoea citrea] | Length = 208 |
| SEQ ID N^{o} 436 | LI-4223.1 | 35 | emb\|CAB88960.1\| (AL353864) hypothetical protein SC8F11.09. [Streptomyces | Length = 137 |
| | | | coelicolor A3(2)] | |
| SEQ ID N^{o} 437 | LI-6183.2 | | | |
| SEQ ID N^{o} 438 | LI-1345.1 | 43 | emb\|CAB75242.1\| (AL139075) putative penplasmic protein [Campylobacter | Length = 390 |
| | | | jejuni] | |
| SEQ ID N^{o} 439 | LI-3379.1 | 67 | emb\|CAB53841.1\| (AJ242593) gp51 [Bacteriophage A118] | Length = 186 |
| SEQ ID N^{o} 440 | LI-5543.1 | | No Hits found | |
| SEQ ID N^{o} 441 | LI-6018.1 | 47 | gb\|AAF43118.1\|AF208055_5 (AF208055) Orf245 [bacteriophage phi31.1] | |
| | | | gb\|AAF74061.1\| (AF212844) ORF245 [Lactococcus lactis] gb\|AAF74095.1\| | |
| | | | (AF212846) ORF245 [Lactococcus lactis bacteriophage ul36.1] gb\|AAF74110.1\| | |
| | | | (AF212847) ORF245 [Lactococcus lactis bac | |
| SEQ ID N^{o} 442 | LI-1951.1 | | No Hits found | |
| SEQ ID N^{o} 443 | LI-4535.1 | | No Hits found | |
| SEQ ID N^{o} 444 | LI-1171.1 | 37 | gb\|AAF36806.1\| (AF155139) VanZF [Paenibacillus popilliae] | Length = 206 |
| SEQ ID N^{o} 445 | LI-1215.1 | 59 | pir\|\|A69898 conserved hypothetical protein yoaZ - Bacillus subtilis | Length = 210 |
| | | | gb\|AAB84456.1\| (AF027868) YoaZ [Bacillus subtilis] emb\|CAB13771.1\| | |
| | | | (Z99114) similar to hypothetical proteins from B. subtilis [Bacillus subtilis] | |
| SEQ ID N^{o} 446 | LI-4153.1 | 64 | sp\|P94606\|ATKC_CLOAB POTASSIUM-TRANSPORTING ATPASE C CHAIN | Length = 204 |
| | | | (ATP PHOSPHOHYDROLASE [POTASSIUM-TRANSPORTING] C | |
| | | | CHAIN) gb\|AAC45479.1\| (U44892) KdpC [Clostridium acetobutylicum] | |
| SEQ ID N^{o} 447 | LI-2943.1 | | No Hits found | |
| SEQ ID N^{o} 448 | LI-2942.1 | | No Hits found | |
| SEQ ID N^{o} 449 | LI-1120.1 | 35 | sp\|P31078\|PETP_RHOCA PETP PROTEIN pir\|\|S22631 petP protein - | Length = 166 |
| | | | Rhodobacter capsulatus emb\|CAA78097.1\| (Z12113) protein of unknown | |
| | | | function [Rhodobacter capsulatus] | |
| SEQ ID N^{o} 450 | LI-4532.1 | | No Hits found | |
| SEQ ID N^{o} 451 | LI-3387.1 | 99 | emb\|CAB53838.1\| (AJ242593) putative recombinase [Bacteriophage A118] | Length = 177 |
| SEQ ID N^{o} 452 | LI-1125.1 | 34 | gb\|AAC978451\| (AF063866) ORF MSV027 tryptophan repeat gene family | Length = 297 |
| | | | protein [Melanoplus sanguinipes entomopoxvirus] | |
| SEQ ID N^{o} 453 | LI-4217.1 | | No Hits found | |
| SEQ ID N^{o} 454 | LI-5704.1 | | No Hits found | |
| SEQ ID N^{o} 455 | LI-2923.1 | 41 | gb\|AAC440021.1\| (U40830) Orf14.9 protein [Streptococcus thermophilus] | Length = 191 |
| | | | prf\|\|2209356Q ORF 14.9 [Streptococcus thermophilus] | |
| SEQ ID N^{o} 456 | LI-1393.1 | | No Hits found | |
| SEQ ID N^{o} 457 | LI-2461.1 | 39 | gb\|AAC44021.1\| (U40830) Orf14.9 protein [Streptococcus thermophilus] | Length = 191 |
| | | | prf\|\|2209356Q ORE 14.9 [Streptococcus thermophilus] | |
| SEQ ID N^{o} 458 | LI-5483.1 | 14 | sp\|P05790\|FBOH_BOMMO FIBROIN HEAVY CHAIN PRECURSOR (FIB-H) (H- | Length = 5263 |
| | | | FIBROIN) gb\|AAF76983.1\|AF226688_1 (AF226688) fibroin heavy chain Fib-H | |
| | | | [Bombyx mori] | |
| SEQ ID N^{o} 459 | LI-392.1 | 49 | emb\|CAB73650.1\| (AL139079) putative ABC transport system ATP-binding | Length = 217 |
| | | | protein [Campylobacter jejuni] | |
| SEQ ID N^{o} 460 | LI-2950.1 | | No Hits found | |
| SEQ ID N^{o} 461 | LI-6003.1 | 48 | pir\|\|B69784 conserved hypothetical protein ydhF - Bacillus subtilis | Length = 236 |
| | | | emb\|CAB12392.1\| (Z99107) similar to hypothetical proteins from B. subtilis | |
| | | | [Bacillus subtilis] | |
| SEQ ID N^{o} 462 | LI-6065.1 | | No Hits found | |
| SEQ ID N^{o} 463 | LI-1578.1 | 49 | sp\|P46903\|NATA_BACSU ATP-BINDING TRANSPORT PROTEIN NATA (NA+ | |
| | | | ABC TRANSPORTER) pir\|\|A69666 Na+ ABC transporter (extrusion) (ATP- | |
| | | | binding protein) natA - Bacillus subtilis gb\|AAB53022.1\| (U30873) NatA | |
| | | | [Bacillus subtilis] dbj\|BAA22236.1\| (AB0006 | |
| SEQ ID N^{o} 464 | LI-4124.1 | 82 | gb\|AAC64335.1\| (AF036485) putative transposase [Plasmid pNZ4000] | Length = 226 |
| SEQ ID N^{o} 465 | LI-4139.1 | 82 | gb\|AAC64335.1\| (AF036485) putative transposase [Plasmid pNZ4000] | Length = 226 |
| SEQ ID N^{o} 466 | LI-4166.1 | 94 | emb\|CAA64387.1\| (X94761) transposase [Streptococcus thermophilus] | Length = 226 |
| | | | emb\|CAB52228.1\| (Z98171) transposase [Streptococcus thermophilus] | |
| SEQ ID N^{o} 467 | LI-4182.1 | 82 | gb\|AAC64335.1\| (AF036485) putative transposase [Plasmid pNZ4000] | Length = 226 |
| SEQ ID N^{o} 468 | LI-6038.1 | | No Hits found | |
| SEQ ID N^{o} 469 | LI-4021.1 | | No Hits found | |
| SEQ ID N^{o} 470 | LI-5569.1 | 66 | sp\|P45932\|YQBP_BACSU HYPOTHETICAL 25.3 KD PROTEIN IN SPOIIIC- | |
| | | | CWLA INTERGENIC REGION pir\|\|C69948 phage-related protein homolog yqbP - | |
| | | | Bacillus subtilis dbj\|BAA06948.1\| (D32216) ORF85 [Bacillus subtilis] | |
| | | | dbj\|BAA12412.1\| (D84432) YqbP [Bacillus subtilis] | |
| SEQ ID N^{o} 471 | LI-4084.1 | 96 | emb\|CAA63529.1\| (X92946) transposase [Lactococcus lactis] | Length = 228 |
| SEQ ID N^{o} 472 | LI-4177.1 | 97 | emb\|CAA63529.1\| (X92946) transposase [Lactococcus lactis] | Length = 228 |
| SEQ ID N^{o} 473 | LI-6066.1 | 23 | emb\|CAB53799.1\| (AJ242593) gp13 [Bacteriophage A118] | Length = 110 |
| SEQ ID N^{o} 474 | LI-6106.1 | 97 | emb\|CAA63529.1\| (X92946) transposase [Lactococcus lactis] | Length = 228 |
| SEQ ID N^{o} 475 | LI-6113.1 | 97 | emb\|CAA63529.1\| (X92946) transposase [Lactococcus lactis] | Length = 228 |
| SEQ ID N^{o} 476 | LI-4013.1 | 64 | gb\|AAC48871.1\| (U51128) ORF245 [Lactococcus bacteriophage phi31] | Length = 245 |
| SEQ ID N^{o} 477 | LI-5978.1 | 61 | emb\|CAB40581.1\| (AJ010128) DNA alkylation repair enzyme [Bacillus cereus] | Length = 237 |
| SEQ ID N^{o} 478 | LI-6085.1 | 53 | sp\|P45915\|YQAS_BACSU HYPOTHETICAL 27.7 KD PROTEIN IN SPOIIIC- | |
| | | | CWLA INTERGENIC REGION pir\|\|B69946 phage-related terminase small | |
| | | | subunit homolog yqaS - Bacillus subtilis dbj\|BAA06932.1\| (D32216) | |
| | | | ORF43 [Bacillus subtilis] dbj\|BAA12394.1\| (D84432 | |
| SEQ ID N^{o} 479 | LI-1346.1 | 15 | pir\|\|E75471 transcription regulator, GntR family - Deinococcus radiodurans | Length = 267 |
| | | | (strain R1) gb\|AAF10394.1\|AE001936_3 (AE001936) transcriptional regulator, | |
| | | | GntR family [Deinococcus radiodurans] | |
| SEQ ID N^{o} 480 | LI-4506.1 | | No Hits found | |
| SEQ ID N^{o} 481 | LI-3994.1 | 54 | gb\|AAB18697.1\| (U38906) ORF22 [Bacteriophage r1t] | Length = 228 |
| SEQ ID N^{o} 482 | LI-6131.1 | 54 | gb\|AAB18697.1\| (U38906) ORF22 [Bacteriophage r1t] | Length = 228 |
| SEQ ID N^{o} 483 | LI-4050.1 | | No Hits found | |
| SEQ ID N^{o} 484 | LI-284.1 | 57 | pir\|\|F69375 ABC transporter, ATP-binding protein homolog - Archaeoglobus | Length = 285 |
| | | | fulgidus gb\|AAB90232.1\| (AE001034) ABC transporter, ATP-binding protein | |
| | | | [Archaeoglobus fulgidus] | |
| SEQ ID N^{o} 485 | LI-4160.1 | 65 | gb\|AAB39096.1\| (U39673) KdpE [Clostridium acetobutylicum] | Length = 232 |
| SEQ ID N^{o} 486 | LI-529.1 | 47 | dbj\|BAA03592.1\| (D14877) positive regulator for virulence factors [Clostridium | Length = 236 |
| | | | perfringens] | |
| SEQ ID N^{o} 487 | LI-3559.1 | | No Hits found | |
| SEQ ID N^{o} 488 | LI-5643.1 | 49 | pir\|\|D49898 cellobiose phosphotransferase system celC - Bacillus | Length = 245 |
| | | | stearothermophilus gb\|AAA17391.1\| (U07818) putative phospho-beta- | |
| | | | glucosidase [Bacillus stearothermophilus] | |
| SEQ ID N^{o} 489 | LI-5720.1 | 90 | emb\|CAB53837.1\| (AJ242593) gp47 [Bacteriophage A118] | Length = 319 |
| SEQ ID N^{o} 490 | LI-2788.1 | 46 | gbj\|AAF35174.1\| (AF193610) TndX [Clostridium difficile] | Length = 533 |
| SEQ ID N^{o} 491 | LI-4054.1 | 28 | emb\|CAB52531.1\| (AJ131519) hypothetical protein [Lactobacillus bacteriophage | Length = 247 |
| | | | phi adh] | |
| SEQ ID N^{o} 492 | LI-4095.1 | 47 | pir\|\|E71905 hypothetical protein jhp0651 - Helicobacter pylori (strain J99) | Length = 234 |
| | | | gb\|AAD06227.1\| (AE001497) putative [Helicobacter pylori J99] | |
| SEQ ID N^{o} 493 | LI-5200.1 | 34 | prf\|\|2119294A YFW1 gene [Saccharomyces cerevisiae] | Length = 605 |
| SEQ ID N^{o} 494 | LI-2995.1 | 52 | pir\|\|T12864 probable antirepressor - Bacillus subtilis phage SPBc2 | |
| | | | emb\|CAB13959.1\| (Z99114) similar to phage-related DNA-binding protein | |
| | | | anti-repressor [Bacillus subtilis] emb\|CAB13985.1\| (Z99115) similar to phage- | |
| | | | related DNA-binding protein | |
| SEQ ID N^{o} 495 | LI-5715.1 | 58 | dbj\|BAA97816.1\| (AB044554) antirepressor [Staphylococcus aureus prophage | Length = 265 |
| | | | phiPV83] | |
| SEQ ID N^{o} 496 | LI-6019.1 | 38 | pir\|\|T00178 hypothetical protein 44 - Staphylococcus aureus phage phi PVL | Length = 161 |
| | | | dbj\|BAA31918.1\| (AB009866) orf 44 [bacteriophage phi PVL] | |
| SEQ ID N^{o} 497 | LI-2779.1 | 27 | pir\|\|G69486 probable iron-sulfur flavoprotein isf-3 - Archaeoglobus fulgidus | Length = 201 |
| | | | gb\|AAB89371.1\| (AE000972) iron-sulfur flavoprotein (isf-3) [Archaeoglobus | |
| | | | fulgidus] | |
| SEQ ID N^{o} 498 | LI-6144.1 | 69 | dbj\|BAA97816.1\| (AB044554) antirepressor [Staphylococcus aureus prophage | Length = 265 |
| | | | phiPV83] | |
| SEQ ID N^{o} 499 | LI-1926.1 | | No Hits found | |
| SEQ ID N^{o} 500 | LI-4026.1 | | No Hits found | |
| SEQ ID N^{o} 501 | LI-1388.1 | | No Hits found | |
| SEQ ID N^{o} 502 | LI-2781.1 | 47 | pir\|\|E70761 probable ketoacyl reductase - Mycobacterium tuberculosis (strain | Length = 267 |
| | | | H37RV) emb\|CAA98318.1\| (Z74020) hypothetical protein Rv1544 | |
| | | | [Mycobacterium tuberculosis] | |
| SEQ ID N^{o} 503 | LI-3157.1 | 38 | pir\|\|G75518 probable beta-lactamase - Deinococcus radiodurans (strain R1) | Length = 277 |
| | | | gb\|AAF10013.1\|AE001903_1 (AE001903) beta-lactamase, putative | |
| | | | [Deinococcus radiodurans] | |
| SEQ ID N^{o} 504 | LI-485.1 | 46 | pir\|\|H71283 conserved hypothetical integral membrane protein TP0771 - syphilis | Length = 593 |
| | | | spirochete gb\|AAC65739.1\| (AE001248) conserved hypothetical integral | |
| | | | membrane protein [Treponema pallidum] | |
| SEQ ID N^{o} 505 | LI-5604.1 | 31 | sp\|P45915\|YQAS_BACSU HYPOTHETICAL 27.7 KD PROTEIN IN SPOIIIC- | |
| | | | CWLA INTERGENIC REGION pir\|\|B69946 phage-related terminase small | |
| | | | subunit homolog yqaS - Bacillus subtilis dbj\|BAA06932.1\| (D32216) | |
| | | | ORF43 [Bacillus subtilis] dbj\|BAA12394.1\| (D84432 | |
| SEQ ID N^{o} 506 | LI-4169.1 | | No Hits found | |
| SEQ ID N^{o} 507 | LI-522.1 | 40 | ref\|NP_013953.1\| Ymr226cp sp\|Q05016\|YM71_YEAST HYPOTHETICAL | |
| | | | OXIDOREDUCTASE IN MRPL44-MTF1 INTERGENIC REGION pir\|\|S57593 | |
| | | | hypothetical protein YMR226c - yeast (Saccharomyces cerevisiae) | |
| | | | emb\|CAA90197.1\| (Z49939) unknown [Saccharomyces cerevisiae] | |
| SEQ ID N^{o} 508 | LI-1513.1 | | No Hits found | |
| SEQ ID N^{o} 509 | LI-6136.1 | 98 | emb\|CAB53838.1\| (AJ242593) putative recombinase [Bacteriophage A118] | Length = 271 |
| SEQ ID N^{o} 510 | LI-3148.1 | 75 | pir\|\|S16647 sporulation dipeptide ABC transporter dppA - Bacillus subtilis | Length = 274 |
| SEQ ID N^{o} 511 | LI-5558.1 | | No Hits found | |
| SEQ ID N^{o} 512 | LI-4123.1 | 68 | gb\|AAB01067.1\| (U23813) transposase [Lactococcus lactis] | Length = 385 |
| SEQ ID N^{o} 513 | LI-6052.1 | 33 | emb\|CAB53803.1\| (AJ242593) gp17 [Bacteriophage A118] | Length = 272 |
| SEQ ID N^{o} 514 | LI-1577.1 | 42 | pir\|\|B71197 hypothetical protein PH1848 - Pyrococcus horikoshii | Length = 255 |
| | | | dbj\|BAA30969.1\| (AP000007) 255aa long hypothetical protein [Pyrococcus | |
| | | | horikoshii] | |
| SEQ ID N^{o} 515 | LI-3333.1 | 61 | emb\|CAB53803.1\| (AJ242593) gp17 [Bacteriophage A118] | Length = 272 |
| SEQ ID N^{o} 516 | LI-523.1 | 37 | pir\|\|B59099 hypothetical protein pXO1-66 - Bacillus anthracis virulence plasmid | Length = 361 |
| | | | pXO1 gb\|AAD32370.1\|AAD32370 (AF065404) pXO1-66 [Bacillus anthracis] | |
| SEQ ID N^{o} 517 | LI-1025.1 | | No Hits found | |
| SEQ ID N^{o} 518 | LI-2947.1 | | No Hits found | |
| SEQ ID N^{o} 519 | LI-5550.1 | 30 | pir\|\|T12790 N-acetylmuramoyl-L-alanine amidase homolog - Bacillus subtilis | |
| | | | phage SPBc2 emb\|CAB14059.1\| (Z99115) similar to N-acetylmuramoyl-L- | |
| | | | alanine amidase [Bacillus subtilis] gb\|AAC38300.1\| (AF021803) N- | |
| | | | acetylmuramoyl-L-alanine | |
| SEQ ID N^{o} 520 | LI-733.1 | 20 | pir\|\|A71161 hypothetical protein PH0486 - Pyrococcus horikoshii | Length = 170 |
| | | | dbj\|BAA29574.1\| (AP000002) 170aa long hypothetical protein [Pyrococcus | |
| | | | horikoshii] | |
| SEQ ID N^{o} 521 | LI-4067.1 | 39 | gb\|AAA63619.1\| (U10992) abiD [Lactococcus lactis] | Length = 366 |
| SEQ ID N^{o} 522 | LI-4670.1 | 50 | emb\|CAA78595.1\| (Z14225) SpoIIIJ [Bacillus subtilis] | Length = 259 |
| SEQ ID N^{o} 523 | LI-4460.1 | | No Hits found | |
| SEQ ID N^{o} 524 | LI-4174.1 | 61 | gb\|AAF37879.1\|AF234619_2 (AF234619) OpuABC [Lactococcus lactis] | Length = 573 |
| SEQ ID N^{o} 525 | LI-4148.1 | 66 | sp\|P18179\|ATBP_STAAU POTENTIAL ATP-BINDING PROTEIN (ORF 271) | |
| | | | pir\|\|S11779 probable ATP-binding protein - Staphylococcus aureus transposon | |
| | | | Tn552 emb\|CAA36948.1\| (X52734) ORF271 (pot. ATP-binding protein) (AA 1-271) | |
| | | | [Staphylococcus au | |
| SEQ ID N^{o} 526 | LI-454.1 | | No Hits found | |
| SEQ ID N^{o} 527 | LI-3320.1 | 97 | sp\|Q37979\|AEPE_BPA50 L-ALANOYL-D-GLUTAMATE PEPTIDASE | Length = 289 |
| | | | pir\|\|S69801 L-alanoyl-D-glutamate peptidase, 33.4 K - phage A500 | |
| | | | emb\|CAA59365.1\| (X85009) L-alanoyl-D-glutamate peptidase [Bacteriophage | |
| | | | A500] | |
| SEQ ID N^{o} 528 | LI-36.1 | 26 | gi\|6324372 anchorage subunit of a-agglutinin; Aga1p sp\|P32323\|AGA1_YEAST | |
| | | | A-AGGLUTININ ATTACHMENT SUBUNIT PRECURSOR pir\|\|A41258 a- | |
| | | | agglutinin core protein AGA1 - yeast (Saccharomyces cerevisiae) | |
| | | | gb\|AAA34382.1\| (M60590) a-agglutinin core subunit [Saccharo | |
| SEQ ID N^{o} 529 | LI-6075.1 | 32 | pir\|\|H45691 main capsid protein Gp34 - Lactobacillus delbrueckii subsp. | Length = 286 |
| | | | bulgaricus phage mv4 | |
| SEQ ID N^{o} 530 | LI-781.1 | 49 | gb\|AAC56014.1\| (AE001272) conserved hypothetical protein [Lactococcus lactis] | Length = 286 |
| SEQ ID N^{o} 531 | LI-4053.1 | | No Hits found | |
| SEQ ID N^{o} 532 | LI-6025.1 | 42 | dbj\|BAA97828.1\| (AB044554) orf 21 [Staphylococcus aureus prophage phiPV83] | Length = 257 |
| SEQ ID N^{o} 533 | LI-2966.1 | 32 | gb\|AAC24147.1\| (AF071201) unknown [bacteriophage Felix 01] | Length = 262 |
| SEQ ID N^{o} 534 | LI-4130.1 | 59 | pir\|\|B47092 copy control protein repB - Enterococcus faecalis plasmid pAD1 | Length = 281 |
| | | | gb\|AAB00504.1\| (L01794) replication-associated protein [Enterococcus faecalis] | |
| SEQ ID N^{o} 535 | LI-3354.1 | 96 | emb\|CAB53792.1\| (AJ242593) major capsid protein [Bacteriophage A118] | Length = 299 |
| SEQ ID N^{o} 536 | LI-2957.1 | 19 | gb\|AAC99858.1\| (U31159) CR16 [Rattus norvegicus] gb\|AAC99859.1\| (U31169) | Length = 485 |
| | | | SH3 domain binding protein [Rattus norvegicus] | |
| SEQ ID N^{o} 537 | LI-2975.1 | 40 | gb\|AAF84386.1\|AE003986_16 (AE003986) hypothetical protein [Xylella | Length = 327 |
| | | | fastidiosa] gb\|AAF84491.1\|AE003993_10 (AE003993) hypothetical protein | |
| | | | [Xylella fastidiosa] | |
| SEQ ID N^{o} 538 | LI-4138.1 | 48 | gb\|AAC56014.1\| (AE001272) conserved hypothetical protein [Lactococcus lactis] | Length = 286 |
| SEQ ID N^{o} 539 | LI-5052.1 | 48 | pir\|\|B70067 hypothetical protein ywqG - Bacillus subtilis emb\|CAB07445.1\| | Length = 261 |
| | | | (Z92952) unknown [Bacillus subtilis] emb\|CAB15639.1\| (Z99122) ywqG [Bacillus | |
| | | | subtilis] | |
| SEQ ID N^{o} 540 | LI-3386.1 | 67 | emb\|CAB53839.1\| (AJ242593) gp49 [Bacteriophage A118] | Length = 310 |
| SEQ ID N^{o} 541 | LI-2987.1 | 34 | sp\|P45915\|YQAS_BACSU HYPOTHETICAL 27.7 KD PROTEIN IN SPOIIIC- | |
| | | | CWLA INTERGENIC REGION pir\|\|B69946 phage-related terminase small | |
| | | | subunit homolog yqaS - Bacillus subtilis dbj\|BAA06932.1\| (D32216) | |
| | | | ORF43 [Bacillus subtilis] dbj\|BAA12394.1\| (D84432) | |
| SEQ ID N^{o} 542 | LI-395.1 | 29 | gb\|AAB49627.1\| (U63134) the 5′ end of the open reading frame shows similarity | Length = 252 |
| | | | to the rgg protein of Streptococcus gordonii, Swiss-Prot Accession | |
| | | | Number P49330 [Streptococcus pyogenes] | |
| SEQ ID N^{o} 543 | LI-3159.1 | 47 | pir\|\|A69856 polysugar degrading enzyme homolog ykfC - Bacillus subtilis | Length = 296 |
| | | | emb\|CAA05579.1\| (AJ002571) YkfC [Bacillus subtilis] emb\|CAB13156.1\| | |
| | | | (Z99110) similar to polysugar degrading enzyme [Bacillus subtilis] | |
| SEQ ID N^{o} 544 | LI-5590.1 | 81 | sp\|P45921\|YQBE_BACSU HYPOTHETICAL 34.5 KD PROTEIN IN SPOIIIC- | |
| | | | CWLA INTERGENIC REGION pir\|\|H69946 phage-related protein homolog yqbE - | |
| | | | Bacillus subtilis dbj\|BAA06938.1\| (D32216) ORF53 [Bacillus subtilis] | |
| | | | dbj\|BAA12400.1\| (D84432) YqbE [Bacillus subtilis] | |
| SEQ ID N^{o} 545 | LI-1216.1 | 51 | pir\|\|D69900 conserved hypothetical protein yobV - Bacillus subtilis | Length = 313 |
| | | | gb\|AAB84472.1\| (AF027868) transcription regulator [Bacillus subtilis] | |
| | | | emb\|CAB13802.1\| (Z99114) similar to hypothetical proteins [Bacillus subtilis] | |
| SEQ ID N^{o} 546 | LI-6135.1 | 97 | emb\|CAB53839.1\| (AJ242593) gp49 [Bacteriophage A118] | Length = 310 |
| SEQ ID N^{o} 547 | LI-6037.1 | 57 | pir\|\|T13262 integrase - Lactococcus lactis phage BK5-T gb\|AAA98585.1\| | Length = 374 |
| | | | (L44593) integrase [Lactococcus lactis phage BK5-T] | |
| SEQ ID N^{o} 548 | LI-2934.1 | 39 | emb\|CAA61519.1\| (X89234) phagelysin [Listeria innocua] | Length = 287 |
| SEQ ID N^{o} 549 | LI-4066.1 | 42 | emb\|CAA61519.1\| (X89234) phagelysin [Listeria innocua] | Length = 287 |
| SEQ ID N^{o} 550 | LI-1087.1 | 55 | pir\|\|T13262 integrase - Lactococcus lactis phage BK5-T gb\|AAA98585.1\| | Length = 374 |
| | | | (L44593) integrase [Lactococcus lactis phage BK5-T] | |
| SEQ ID N^{o} 551 | LI-6138.1 | 94 | emb\|CAB53837.1\| (AJ242593) gp47 [Bacteriophage A118] | Length = 319 |
| SEQ ID N^{o} 552 | LI-2142.1 | 47 | emb\|CAA66252.1\| (X97651) abortive phage resistance mechanism [Lactococcus | Length = 346 |
| | | | lactis] | |
| SEQ ID N^{o} 553 | LI-3995.1 | 54 | pir\|\|T13262 integrase - Lactococcus lactis phage BK5-T gb\|AAA98585.1\| | Length = 374 |
| | | | (L44593) integrase [Lactococcus lactis phage BK5-T] | |
| SEQ ID N^{o} 554 | LI-6130.1 | 53 | pir\|\|T13262 integrase - Lactococcus lactis phage BK5-T gb\|AAA98585.1\| | Length = 374 |
| | | | (L44593) integrase [Lactococcus lactis phage BK5-T] | |
| SEQ ID N^{o} 555 | LI-5313.1 | 41 | gb\|AAD46982.1\| (AF070520) unknown [Sinorhizobium meliloti] | Length = 332 |
| SEQ ID N^{o} 556 | LI-6021.1 | | No Hits found | |
| SEQ ID N^{o} 557 | LI-5319.1 | 19 | sp\|Q00753\|MSMR_STRMU MSM OPERON REGULATORY PROTEIN | Length = 278 |
| | | | gb\|AAA26932.1\| (M77351) regulatory protein [Streptococcus mutans] | |
| SEQ ID N^{o} 558 | LI-5568.1 | 69 | sp\|P45950\|YQBQ_BACSU HYPOTHETICAL 37.0 KD PROTEIN IN SPOIIIC- | |
| | | | CWLA INTERGENIC REGION pir\|\|D69948 phage-related protein homolog yqbQ - | |
| | | | Bacillus subtilis dbj\|BAA06949.1\| (D32216) ORF86 [Bacillus subtilis] | |
| | | | dbj\|BAA12413.1\| (D84432) YqbQ [Bacillus subtilis] | |
| SEQ ID N^{o} 559 | LI-2785.1 | 59 | pir\|\|S78538 site-specific recombinase tnpX - Clostridium perfringens transposon | Length = 707 |
| | | | Tn4451 gb\|AAB51419.1\| (U15027) TnpX [Clostridium perfringens] | |
| SEQ ID N^{o} 560 | LI-4060.1 | 33 | emb\|CAB53806.1\| (AJ242593) gp20 [Bacteriophage A118] | Length = 357 |
| SEQ ID N^{o} 561 | LI-14.1 | 71 | sp\|O34755\|YKOT_BACSU HYPOTHETICAL 38.5 KDA PROTEIN IN TNRA- | |
| | | | SSPD INTERGENIC REGION pir\|\|F69860 dolichol phosphate mannose | |
| | | | synthase homolog ykoT - Bacillus subtilis emb\|CAB13196.1\| (Z99110) | |
| | | | similar to dolichol phosphate mannose synthase [Bacill | |
| SEQ ID N^{o} 562 | LI-4122.1 | | No Hits found | |
| SEQ ID N^{o} 563 | LI-4955.1 | 43 | gb\|AAB67968.1\| (U77367) internalin [Listeria monocytogenes] | Length = 821 |
| SEQ ID N^{o} 564 | LI-4077.1 | 70 | pir\|\|A24455 repB protein - Bacillus sp. plasmids emb\|CAA33714.1\| (X15670) | |
| | | | repB protein (AA 1-334) [Bacillus sp.] gb\|AAA88362.1\| (M19465) alpha protein | |
| | | | [Plasmid pUB110] gb\|AAA84919.1\| (U32369) ORF alpha; RepU is encoded | |
| | | | within ORF alpha [Plasmid | |
| SEQ ID N^{o} 565 | LI-4116.1 | 67 | gb\|AAB01067.1\| (U23813) transposase [Lactococcus lactis] | Length = 385 |
| SEQ ID N^{o} 566 | LI-6051.1 | 54 | pir\|\|T13217 hypothetical protein R372 - Lactobacillus phage phi-gle | Length = 372 |
| | | | emb\|CAA66746.1\| (X98106) Rorf372 [Bacteriophage phig1e] | |
| SEQ ID N^{o} 567 | LI-3329.1 | 52 | emb\|CAB53805.1\| (AJ242593) gp19 [Bacteriophage A118] | Length = 342 |
| SEQ ID N^{o} 568 | LI-9.1 | | No Hits found | |
| SEQ ID N^{o} 569 | LI-6050.1 | 47 | emb\|CAB53805.1\| (AJ242593) gp19 [Bacteriophage A118] | Length = 342 |
| SEQ ID N^{o} 570 | LI-6078.1 | 37 | emb\|CAB53790.1\| (AJ242593) gp4 [Bacteriophage A118] | Length = 379 |
| SEQ ID N^{o} 571 | LI-1308.1 | 26 | sp\|P19934\|TOLA_ECOLI TOLA PROTEIN pir\|\|JV0057 toIA protein - Escherichia | |
| | | | coli gb\|AAA24683.1\| (M28232) toIA [Escherichia coli] dbj\|BAA35405.1\| | |
| | | | (D90713) TolA protein. [Escherichia coli] gb\|AAC73833.1\| (AE000177) | |
| | | | membrane spanning protein, required for o | |
| SEQ ID N^{o} 572 | LI-5609.1 | 23 | pir\|\|C71907 probable type II DNA modification enzyme (methyltransferase) - | |
| | | | Helicobacter pylori (strain J99) gb\|AAD06206.1\| (AE001495) putative TYPE II | |
| | | | DNA MODIFICATION ENZYME (METHYLTRANSFERASE) [Helicobacter | |
| | | | pylori J99] L | |
| SEQ ID N^{o} 573 | LI-6120.1 | 83 | gb\|AAD51846.1\|AF178758_2 (AF178758) ArsB [Sinorhizobium sp. As4] | Length = 351 |
| SEQ ID N^{o} 574 | LI-6185.1 | 50 | pir\|\|T00175 hypothetical protein 41 - Staphylococcus aureus phage phi PVL | Length = 332 |
| | | | dbj\|BAA31915.1\| (AB009866) orf 41 [bacteriophage phi PVL] | |
| SEQ ID N^{o} 575 | LI-6046.1 | 56 | emb\|CAB53806.1\| (AJ242593) gp20 [Bacteriophage A118] | Length = 357 |
| SEQ ID N^{o} 576 | LI-6122.1 | 76 | emb\|CAB85311.1\| (AL162758) putative integral membrane efflux protein | Length = 350 |
| | | | [Neisseria meningitidis] | |
| SEQ ID N^{o} 577 | LI-3326.1 | 55 | emb\|CAB53806.1\| (AJ242593) gp20 [Bacteriophage A118] | Length = 357 |
| SEQ ID N^{o} 578 | LI-1191.1 | 35 | emb\|CAB39003.1\| (AL034558) predicted using hexExon; MAL3P2.16 | Length = 1213″ |
| | | | (PF0235w), Hypothetical protein, len: 1214 aa [Plasmodium | |
| | | | falciparum] | |
| SEQ ID N^{o} 579 | LI-3160.1 | 61 | pir\|\|H69855 chloromuconate cycloisomerase homolog ykfB - Bacillus subtilis | Length = 366 |
| | | | emb\|CAA05578.1\| (AJ002571) YkfB [Bacillus subtilis] emb\|CAB13155.1\| | |
| | | | (Z99110) similar to chloromuconate cycloisomerase [Bacillus subtilis] | |
| SEQ ID N^{o} 580 | LI-5564.1 | 66 | sp\|P45935\|YQBT_BACSU HYPOTHETICAL 35.0 KD PROTEIN IN SPOIIIC- | |
| | | | CWLA INTERGENIC REGION pir\|\|G69948 phage-related protein homolog yqbT - | |
| | | | Bacillus subtilis dbj\|BAA06952.1\| (D32216) ORF89 [Bacillus subtilis] | |
| | | | dbj\|BAA12416.1\| (D84432) YqbT [Bacillus subtilis] | |
| SEQ ID N^{o} 581 | LI-1601.1 | 48 | gb\|AAB67970.1\| (U77368) inID [Listeria monocytogenes] | Length = 567 |
| SEQ ID N^{o} 582 | LI-5593.1 | 63 | sp\|P45920\|YQBD_BACSU HYPOTHETICAL 36.2 KD PROTEIN IN SPOIIIC- | |
| | | | CWLA INTERGENIC REGION pir\|\|G69946 phage-related protein homolog yqbD - | |
| | | | Bacillus subtilis dbj\|BAA06937.1\| (D32216) ORF52 [Bacillus subtilis] | |
| | | | dbj\|BAA12399.1\| (D84432) YqbD [Bacillus subtilis] | |
| SEQ ID N^{o} 583 | LI-2944.1 | 43 | gb\|AAF84513.1\|AE003994_12 (AE003994) hypothetical protein [Xylella | Length = 387 |
| | | | fastidiosa] | |
| SEQ ID N^{o} 584 | LI-2978.1 | 34 | gb\|AAF84384.1\|AE003986_14 (AE003986) hypothetical protein [Xylella | Length = 397 |
| | | | fastidiosa] gb\|AAF84489.1\|AE003993_8 (AE003993) hypothetical protein | |
| | | | [Xylella fastidiosa] | |
| SEQ ID N^{o} 585 | LI-455.1 | 34 | pir\|\|A71237 hypothetical protein PH0155 - Pyrococcus horikoshii | Length = 317 |
| | | | dbj\|BAA29224.1\| (AP000001) 317aa long hypothetical protein [Pyrococcus | |
| | | | horikoshii] | |
| SEQ ID N^{o} 586 | LI-6111.1 | 35 | gb\|AAD20952.1\| (AF060119) methyltransferase [Pasteurella haemolytica] | Length = 706 |
| SEQ ID N^{o} 587 | LI-5933.1 | 38 | emb\|CAB96616.1\| (AJ400629) integrase [Streptococcus pneumoniae | Length = 375 |
| | | | bacteriophage MM1] | |
| SEQ ID N^{o} 588 | LI-4502.2 | | | |
| SEQ ID N^{o} 589 | LI-5275.1 | 32 | pir\|\|B69981 N-acetylmuramoyl-L-alanine amidase homolog yrvJ - Bacillus subtilis | Length = 518 |
| | | | emb\|CAB14717.1\| (Z99118) similar to N-acetylmuramoyl-L-alanine amidase | |
| | | | [Bacillus subtilis] | |
| SEQ ID N^{o} 590 | LI-4111.1 | 55 | emb\|CAA51756.1\| (X73329) Na/H antiporter homolog [Lactococcus lactis] | Length = 379 |
| SEQ ID N^{o} 591 | LI-4183.1 | 87 | gb\|AAB52513.1\| (U44843) replication protein [Lactococcus lactis] | Length = 383 |
| SEQ ID N^{o} 592 | LI-2.2 | | | |
| SEQ ID N^{o} 593 | LI-3985.1 | 40 | pir\|\|T13182 integrase - Lactobacillus phage phi-gle emb\|CAA66758.1\| (X98106) | Length = 391 |
| | | | integrase [Bacteriophage phig1e] emb\|CAA62092.1\| (X90510) integrase | |
| | | | [Bacteriophage phig1e] | |
| SEQ ID N^{o} 594 | LI-4486.1 | | No Hits found | |
| SEQ ID N^{o} 595 | LI-1342.1 | 61 | pir\|\|S76946 hypothetical protein - Synechocystis sp. (strain PCC 6803) | Length = 407 |
| | | | dbj\|BAA18858.1\| (D90917) hypothetical protein [Synechocystis sp.] | |
| SEQ ID N^{o} 596 | LI-1348.1 | 26 | dbj\|BAA34922.1\| (AB012764) Chitinase A [Clostridium paraputrificum] | Length = 832 |
| SEQ ID N^{o} 597 | LI-390.1 | | No Hits found | |
| SEQ ID N^{o} 598 | LI-4039.1 | 59 | emb\|CAB63685.1\| (AJ251790) hypothetical protein [Lactobacillus casei | Length = 400 |
| | | | bacteriophage A2] | |
| SEQ ID N^{o} 599 | LI-1088.1 | 32 | gb\|AAF17614.1\|AF207855_3 (AF207855) specificity subunit LIa33I [Lactococcus | Length = 414 |
| | | | lactis] | |
| SEQ ID N^{o} 600 | LI-3255.1 | 84 | sp\|P44099\|YA38_HAEIN HYPOTHETICAL PROTEIN HI1038 pir\|\|I64018 | Length = 400 |
| | | | hypothetical protein HI1038 - Haemophilus influenzae (strain Rd KW20) | |
| | | | gb\|AAC22698.1\| (U32784) conserved hypothetical protein [Haemophilus | |
| | | | influenzae Rd] | |
| SEQ ID N^{o} 601 | LI-4036.1 | | No Hits found | |
| SEQ ID N^{o} 602 | LI-1086.1 | 35 | pir\|\|T30324 type I site-specific deoxyribonuclease (EC 3.1.21.3) LIdl chain | Length = 405 |
| | | | hsdS - Lactococcus lactis plasmid pIL2614 gb\|AAC15898.1\| (U90222) type IC | |
| | | | specificity subunit [Lactococcus lactis] | |
| SEQ ID N^{o} 603 | LI-4103.1 | 41 | gb\|AAC23674.1\| (AF064765) putative transposase [Lactococcus lactis] | Length = 216 |
| SEQ ID N^{o} 604 | LI-4112.1 | 79 | sp\|O52191\|ENO_STRTR ENOLASE (2-PHOSPHOGLYCERATE | Length = 422 |
| | | | DEHYDRATASE) (2-PHOSPHO-D-GLYCERATE HYDRO-LYASE) | |
| | | | gb\|AAC64907.1\| (AF027167) enolase [Streptococcus thermophilus] | |
| SEQ ID N^{o} 605 | LI-1186.1 | | No Hits found | |
| SEQ ID N^{o} 606 | LI-4508.1 | 41 | pir\|\|A69774 integrase homolog ydcL - Bacillus subtilis dbj\|BAA19318.1\| | |
| | | | (AB001488) PROBABLE INTEGRASE. [Bacillus subtilis] emb\|CAB12287.1\| | Length = 368 |
| | | | (Z99106) similar to integrase [Bacillus subtilis] | |
| SEQ ID N^{o} 607 | LI-4015.1 | 64 | emb\|CAB63670.1\| (AJ251789) hypothetical protein [Lactobacillus casei | Length = 455 |
| | | | bacteriophage A2] | |
| SEQ ID N^{o} 608 | LI-589.1 | 38 | pir\|\|T28677 rhoptry protein - Plasmodium yoelii gb\|AAA21304.1\| (L27838) | Length = 2269 |
| | | | rhoptry protein [Plasmodium yoelii] | |
| SEQ ID N^{o} 609 | LI-391.1 | 31 | sp\|Q58207\|Y797_METJA HYPOTHETICAL PROTEIN MJ0797 pir\|\|E64399 | Length = 367 |
| | | | hypothetical protein MJ0797 - Methanococcus jannaschii gb\|AAB98792.1\| | |
| | | | (U67524) hypothetical protein [Methanococcus jannaschii] | |
| SEQ ID N^{o} 610 | LI-4715.1 | 41 | ref\|NP_013898.1\|Ymr173w-ap pir\|\|S69870 hypothetical protein YMR173w-a - | Length = 394 |
| | | | yeast (Saccharomyces cerevisiae) | |
| SEQ ID N^{o} 611 | LI-5638.1 | 51 | pir\|\|H70216 PTS system, cellobiose-specific IIC component (celB) homolog - | Length |
| | | | Lyme disease spirochete plasmid B/cp26 gb\|AAC66324.1\| (AE000792) | |
| | | | PTS system, cellobiose-specific IIC component (celB) [Borrelia | |
| | | | burgdorferi] | |
| SEQ ID N^{o} 612 | LI-5645.1 | 65 | sp\|O06901\|MALH_FUSMR MALTOSE-6′-PHOSPHATE GLUCOSIDASE (6- | Length = 441 |
| | | | PHOSPHO-ALPHA-D-GLUCOSIDASE) gb\|AAB63015.1\| (U81185) MalH | |
| | | | [Fusobacterium mortiferum] | |
| SEQ ID N^{o} 613 | LI-4714.1 | 26 | sp\|P04929\|HRPX_PLALO HISTIDINE-RICH GLYCOPROTEIN PRECURSOR | |
| | | | pir\|\|KGZQHL histidine-rich glycoprotein precursor - Plasmodium lophurae | |
| | | | emb\|CAA25698.1\| (X01469) histidine-rich protein [Plasmodium lophurae] | |
| | | | prf\|\|1101401A protein, His rich [Plasmodium sp.] | |
| SEQ ID N^{o} 614 | LI-5602.1 | 72 | sp\|P45916\|YQAT_BACSU HYPOTHETICAL 50.9 KD PROTEIN IN SPOIIIC- | |
| | | | CWLA INTERGENIC REGION (ORF50) pir\|\|C69946 phage-related | |
| | | | terminase large subunit homolog yqaT - Bacillus subtilis dbj\|BAA06933.1\| | |
| | | | (D32216) ORF50 [Bacillus subtilis] dbj\| | |
| SEQ ID N^{o} 615 | LI-2993.1 | 17 | pir\|\|C71907 probable type II DNA modification enzyme (methyltransferase) - | |
| | | | Helicobacter pylori (strain J99) gb\|AAD06206.1\| (AE001495) putative TYPE II | |
| | | | DNA MODIFICATION ENZYME (METHYLTRANSFERASE) [Helicobacter | |
| | | | pylori J99] L | |
| SEQ ID N^{o} 616 | LI-6084.1 | | No Hits found | |
| SEQ ID N^{o} 617 | LI-4092.1 | 46 | gb\|AAF13688.1\|AF188935_86 (AF188935) pXO2-84 [Bacillus anthracis] | Length = 490 |
| SEQ ID N^{o} 618 | LI-5579.1 | 65 | sp\|P54331\|XKDK_BACSU PHAGE-LIKE ELEMENT PBSX PROTEIN XKDK | Length = 464 |
| | | | pir\|\|C69732 PBSX prophage ORF xkdK - Bacillus subtilis emb\|CAA94066.1\| | |
| | | | (Z70177) xkdK [Bacillus subtilis] emb\|CAB13122.1\| (Z99110) PBSX prophage | |
| | | | [Bacillus subtilis] | |
| SEQ ID N^{o} 619 | LI-1526.1 | 40 | pir\|\|H72265 hypothetical protein TM1336 - Thermotoga maritima (strain MSB8) | Length = 390 |
| | | | gb\|AAD36408.1\|AE001788_3 (AE001788) permease, putative [Thermotoga | |
| | | | maritima] | |
| SEQ ID N^{o} 620 | LI-528.1 | 45 | pir\|\|C55521 virS protein - Clostridium perfringens gb\|AAA58950.1\| (U04966) | Length = 440 |
| | | | VirS [Clostridium perfringens] | |
| SEQ ID N^{o} 621 | LI-4175.1 | 60 | sp\|P37062\|NAPE_ENTFA NADH PEROXIDASE (NPXASE) pir\|\|S18332 NADH | |
| | | | peroxidase (EC 1.11.1.1) - Enterococcus faecalis pdb\|1NPX\| Nadh Peroxidase | |
| | | | (E.C.1.11.1.1) Non-Active Form With Cys 42 Oxidized To A Sulfonic Acid | |
| | | | (Cys42-So3h) pdb\|2NPX\| Nadh | |
| SEQ ID N^{o} 622 | LI-6035.1 | | No Hits found | |
| SEQ ID N^{o} 623 | LI-2455.1 | | No Hits found | |
| SEQ ID N^{o} 624 | LI-4482.1 | | No Hits found | |
| SEQ ID N^{o} 625 | LI-2982.1 | 40 | gb\|AAF84380.1\|AE003986_10 (AE003986) conserved hypothetical protein | Length = 467 |
| | | | [Xylella fastidiosa] gb\|AAF84485.1\|AE003993_4 (AE003993) conserved | |
| | | | hypothetical protein [Xylella fastidiosa] | |
| SEQ ID N^{o} 626 | LI-4147.1 | 67 | sp\|P18416\|TRA3_STAAU TRANSPOSASE FOR TRANSPOSON TN552 (ORF480) | Length = |
| | | | pir\|\|S11780 probable transposase - Staphylococcus aureus transposon | |
| | | | Tn552 emb\|CAA36949.1\| (X52734) ORF480 (pot. transposase) (AA 1-480) | |
| | | | [Staphylococcus aureus] | |
| SEQ ID N^{o} 627 | LI-2981.1 | 28 | sp\|P71385\|YE07_HAEIN HYPOTHETICAL PROTEIN HI1407 pir\|\|B64122 | Length = 447 |
| | | | hypothetical protein HI1407 - Haemophilus influenzae (strain Rd KW20) | |
| | | | gb\|AAC23048.1\| (U32820) traN-related protein [Haemophilus influenzae Rd] | |
| SEQ ID N^{o} 628 | LI-2985.1 | 45 | emb\|CAA72650.1\| (Y11901) hypothetical protein [Lactococcus lactis] | Length = 462 |
| SEQ ID N^{o} 629 | LI-3.1 | 41 | sp\|P46917\|GGAA_BACSU MINOR TEICHOIC ACIDS BIOSYNTHESIS | |
| | | | PROTEIN GGAA pir\|\|D69631 galactosamine-containing minor teichoic acid | |
| | | | biosynthesis ggaA - Bacillus subtilis gb\|AAA73512.1\| (U13979) ggaA | |
| | | | [Bacillus subtilis] emb\|CAB15586.1\| (Z99122) memb | |
| SEQ ID N^{o} 630 | LI-4218.1 | | No Hits found | |
| SEQ ID N^{o} 631 | LI-457.1 | 16 | dbj\|BAA78899.1\| (AP000342) yjcA [Plasmid R100] | Length = 436 |
| SEQ ID N^{o} 632 | LI-1614.1 | 56 | gb\|AAB67970.1\| (U77368) inlD [Listeria monocytogenes] | Length = 567 |
| SEQ ID N^{o} 633 | LI-3815.1 | 29 | pir\|\|B26696 hypothetical protein 1 (CYb-COII intergenic region) - Leishmania | Length = 443 |
| | | | tarentolae mitochondrion (fragment) gb\|AAA96601.1\| (M10126) NH2 terminus | |
| | | | uncertain [Leishmania tarentolae] | |
| SEQ ID N^{o} 634 | LI-5621.1 | 37 | gb\|AAB81298.1\| (U53825) RofA [Streptococcus pyogenes] | Length = 497 |
| SEQ ID N^{o} 635 | LI-3183.1 | | No Hits found | |
| SEQ ID N^{o} 636 | LI-4531.1 | | No Hits found | |
| SEQ ID N^{o} 637 | LI-652.1 | 54 | emb\|CAA07457.1\| (AJ007319) internalin H [Listeria monocytogenes] | Length = 548 |
| SEQ ID N^{o} 638 | LI-6081.1 | | No Hits found | |
| SEQ ID N^{o} 639 | LI-3273.1 | 24 | emb\|CAB01947.1\| (Z79692) ExpA5 [Sinorhizobium meliloti] | Length = 390 |
| SEQ ID N^{o} 640 | LI-6002.1 | 57 | pir\|\|IMBP4 site-specific recombinase for integration and excision - Bacillus | Length = 474 |
| | | | phage phi-105 dbj\|BAA36658.1\| (AB016282) site-specific recombinase for | |
| | | | integration and excision [bacteriophage phi-105] | |
| SEQ ID N^{o} 641 | LI-5600.1 | 68 | sp\|P45917\|YQBA_BACSU HYPOTHETICAL 58.5 KD PROTEIN IN SPOIIIC- | |
| | | | CWLA INTERGENIC REGION pir\|\|D69946 phage-related protein homolog yqbA - | |
| | | | Bacillus subtilis dbj\|BAA06934.1\| (D32216) ORF51 [Bacillus subtilis] | |
| | | | dbj\|BAA12396.1\| (D84432) YqbA [Bacillus subtilis] | |
| SEQ ID N^{o} 642 | LI-1091.1 | 84 | gb\|AAC38346.1\| (AF013165) HsdM [Lactococcus lactis] | Length = 515 |
| SEQ ID N^{o} 643 | LI-4543.1 | | No Hits found | |
| SEQ ID N^{o} 644 | LI-1389.1 | 25 | sp\|P11055\|MYSE_HUMAN MYOSIN HEAVY CHAIN, FAST SKELETAL | Length = 1940 |
| | | | MUSCLE, EMBRYONIC (MUSCLE EMBRYONIC MYOSIN HEAVY | |
| | | | CHAIN) (SMHCE) | |
| SEQ ID N^{o} 645 | LI-3152.1 | 63 | gb\|AAF73090.1\|AF103793_1 (AF103793) peptide binding protein OppA [Listeria | Length = 558 |
| | | | monocytogenes] | |
| SEQ ID N^{o} 646 | LI-6118.1 | 55 | pir\|\|H69299 NADH oxidase (noxA-3) homolog - Archaeoglobus fulgidus | Length = 551 |
| | | | gb\|AAB90837.1\| (AE001077) NADH oxidase (noxA-3) [Archaeoglobus fulgidus] | |
| SEQ ID N^{o} 647 | LI-4879.1 | 77 | sp\|Q59925\|FTHS_STRMU FORMATE —TETRAHYDROFOLATE LIGASE | Length = 556 |
| | | | (FORMYLTETRAHYDROFOLATE SYNTHETASE) (FHS) (FTHFS) | |
| | | | gb\|AAB49329.1\| (U39612) formyl-tetrahydrofolate synthetase [Streptococcus | |
| | | | mutans] | |
| SEQ ID N^{o} 648 | LI-2212.1 | 69 | pir\|\|H64879 probable membrane protein b1309 - Escherichia coli | Length = 568 |
| | | | gb\|AAC74391.1\| (AE000229) putative polysaccharide hydrolase [Escherichia | |
| | | | coli] | |
| SEQ ID N^{o} 649 | LI-4128.1 | 49 | emb\|CAB43191.1\| (AJ011655) replication protein Rep63A [Bacillus thuringiensis] | Lenght = 513 |
| SEQ ID N^{o} 650 | LI-4158.1 | 72 | sp\|O32327\|ATKA_CLOAB POTASSIUM-TRANSPORTING ATPASE A CHAIN | Length = 556 |
| | | | (ATP PHOSPHOHYDROLASE [POTASSIUM-TRANSPORTING] A | |
| | | | CHAIN) gb\|AAC45477.1\| (U44892) KdpA [Clostridium acetobutylicum] | |
| SEQ ID N^{o} 651 | LI-6124.1 | 74 | gb\|AAD51849.1\|AF178758_5 (AF178758) ArsA [Sinorhizobium sp. As4] | Length = 587 |
| SEQ ID N^{o} 652 | LI-5597.1 | 34 | sp\|P45918\|YQBB_BACSU HYPOTHETICAL 34.9 KD PROTEIN IN SPOIIIC- | |
| | | | CWLA INTERGENIC REGION pir\|\|E69946 hypothetical protein yqbB - Bacillus | |
| | | | subtilis dbj\|BAA06935.1\| (D32216) ORF66 [Bacillus subtilis] dbj\|BAA12397.1\| | |
| | | | (D84432) YqbB [Bacillus subtilis] emb\|CAB1 | |
| SEQ ID N^{o} 653 | LI-4033.1 | 38 | gb\|AAF27357.1\|AF198256_11 (AF198256) phage D3 terminase-like protein | Length = 555 |
| | | | [Haemophilus influenzae] | |
| SEQ ID N^{o} 654 | LI-815.1 | 57 | sp\|P25146\|INLA_LISMO INTERNALIN A PRECURSOR pir\|\|S37387 internalin A | Length = 800 |
| | | | precursor - Listeria monocytogenes | |
| SEQ ID N^{o} 655 | LI-5484.1 | 15 | emb\|CAA65738.1\| (X97014) ORF A [Listeria seeligeri] | Length = 902 |
| SEQ ID N^{o} 656 | LI-5317.1 | 45 | emb\|CAB72809.1\| (AL139074) excinuclease ABC subunit A [Campylobacter | Length = 941 |
| | | | jejuni] | |
| SEQ ID N^{o} 657 | LI-5644.1 | 45 | pir\|\|F69848 transcription antiterminator BglG family homolog yjdC - Bacillus | Length = 648 |
| | | | subtilis emb\|CAB13057.1\| (Z99110) similar to transcriptional antiterminator | |
| | | | (BglG family) [Bacillus subtilis] | |
| SEQ ID N^{o} 658 | LI-6016.1 | 27 | pir\|\|T00175 hypothetical protein 41 - Staphylococcus aureus phage phi PVL | Length = 332 |
| | | | dbj\|BAA31915.1\| (AB009866) orf 41 [bacteriophage phi PVL] | |
| SEQ ID N^{o} 659 | LI-648.1 | 14 | gi\|6677945 suppressor of clear, C. elegans, homolog of dbj\|BAA74885.1\| | Length = 582 |
| | | | (AB020669) KIAA0862 protein [Homo sapiens] | |
| SEQ ID N^{o} 660 | LI-4168.1 | 56 | pir\|\|F69869 heavy metal-transporting ATPase homolog ykvW - Bacillus subtilis | Length = 637 |
| | | | emb\|CAB13258.1\| (Z99111) similar to heavy metal-transporting ATPase [Bacillus | |
| | | | subtilis] | |
| SEQ ID N^{o} 661 | LI-1442.1 | 63 | sp\|P25146\|INLA_LISMO INTERNALIN A PRECURSOR pir\|\|S37387 internalin A | Length = 800 |
| | | | precursor - Listeria monocytogenes | |
| SEQ ID N^{o} 662 | LI-17.1 | | No Hits found | |
| SEQ ID N^{o} 663 | LI-1010.1 | 12 | emb\|CAA68916.1\| (Y07639) internalin D [Listeria ivanovii] | Length = 313 |
| SEQ ID N^{o} 664 | LI-2143.1 | 42 | sp\|P09975\|YCF2_MARPO HYPOTHETICAL 259 KD PROTEIN (ORF 2136) | Length = 2136 |
| | | | pir\|\|A05037 hypothetical protein 2136 - liverwort (Marchantia polymorpha) | |
| | | | chloroplast emb\|CAA28078.1\| (X04465) ORF2136 [Marchantia polymorpha] | |
| SEQ ID N^{o} 665 | LI-4157.1 | 82 | sp\|O32328\|ATKB_CLOAB POTASSIUM-TRANSPORTING ATPASE B CHAIN | Length = 685 |
| | | | (ATP PHOSPHOHYDROLASE [POTASSIUM-TRANSPORTING] B | |
| | | | CHAIN) gb\|AAC45478.1\| (U44892) KdpB [Clostridium acetobutylicum] | |
| SEQ ID N^{o} 666 | LI-1021.1 | 21 | emb\|CAA07456.1\| (AJ007319) internalin G [Listeria monocytogenes] | Length = 490 |
| SEQ ID N^{o} 667 | LI-13.1 | | No Hits found | |
| SEQ ID N^{o} 668 | LI-4057.1 | 14 | emb\|CAB52532.1\| (AJ131519) hypothetical protein [Lactobacillus bacteriophage | Length = 241 |
| | | | phi adh] | |
| SEQ ID N^{o} 669 | LI-4019.1 | 65 | emb\|CAB63672.1\| (AJ251789) hypothetical protein [Lactobacillus casei | Length = 770 |
| | | | bacteriophage A2] | |
| SEQ ID N^{o} 670 | LI-4102.1 | 25 | dbj\|BAA87672.1\| (AB016260) Hypothetical gene, methylase gene homolog | Length = 1693 |
| | | | [Agrobacterium tumefaciens] | |
| SEQ ID N^{o} 671 | LI-12.1 | 53 | pir\|\|S49240 hypothetical protein 3 (capsulation locus) - Haemophilus influenzae | Length = 789 |
| | | | (strain RM107) emb\|CAA85752.1\| (Z37516) orf3 [Haemophilus influenzae] | |
| SEQ ID N^{o} 672 | LI-20.1 | 39 | gb\|AAC45605.1\| (U82488) AMI [Listeria monocytogenes] | Length = 917 |
| SEQ ID N^{o} 673 | LI-805.1 | 74 | emb\|CAA65738.1\| (X97014) ORF A [Listeria seeligeri] | Length = 902 |
| SEQ ID N^{o} 674 | LI-1188.1 | 9 | dbj\|BAA84909.1\| (AB024946) orf74 [Escherichia coli] | Length = 873 |
| SEQ ID N^{o} 675 | LI-1313.1 | 76 | pir\|\|F69681 phosphoenolpyruvate synthase pps - Bacillus subtilis | Length = 866 |
| | | | gb\|AAB84457.1\| (AF027868) PEP synthase [Bacillus subtilis] emb\|CAB13775.1\| | |
| | | | (Z99114) phosphoenolpyruvate synthase [Bacillus subtilis] | |
| SEQ ID N^{o} 676 | LI-4164.1 | 62 | sp\|P94608\|KDPD_CLOAB SENSOR PROTEIN KDPD gb\|AAB39095.1\| | Length = 900 |
| | | | (U39673) KdpD [Clostridium acetobutylicum] | |
| SEQ ID N^{o} 677 | LI-5773.1 | 27 | gb\|AAB67968.1\| (U77367) internalin [Listeria monocytogenes] | Length = 821 |
| SEQ ID N^{o} 678 | LI-1096.1 | 85 | gb\|AAC38345.1\| (AF013165) HsdR [Lactococcus lactis] | Length = 995 |
| SEQ ID N^{o} 679 | LI-4178.1 | 27 | pir\|\|D72308 conserved hypothetical protein - Thermotoga maritima (strain MSB8) | Length = 967 |
| | | | gb\|AAD36069.1\|AE001761_1 (AE001761) conserved hypothetical protein | |
| | | | [Thermotoga maritima] | |
| SEQ ID N^{o} 680 | LI-4052.1 | 35 | pir\|\|T03323 gene 116 protein - Lactococcus phage bIL170 gb\|AAC27195.1\| | Length = 916 |
| | | | (AF009630) I16 [bacteriophage bIL170] | |
| SEQ ID N^{o} 681 | LI-4232.1 | 15 | sp\|P25146\|INLA_LISMO INTERNALIN A PRECURSOR pir\|\|S37387 internalin A | Length = 800 |
| | | | precursor - Listeria monocytogenes | |
| SEQ ID N^{o} 682 | LI-5575.1 | 20 | sp\|P54334\|XKDO_BACSU PHAGE-LIKE ELEMENT PBSX PROTEIN XKDO | |
| | | | pir\|\|F69732 PBSX prophage ORF xkdO - Bacillus subtilis emb\|CAA94037.1\| | |
| | | | (Z70177) homologous to yqbO of the skin element [Bacillus subtilis] | |
| | | | emb\|CAB13125.1\| (Z99110) PBSX prophage [Bacillus subtil | |
| SEQ ID N^{o} 683 | LI-1720.1 | 18 | emb\|CAB83920.1\| (AL162753) hypothetical protein NMA0631 [Neisseria | Length = 1082 |
| | | | meningitidis] | |
| SEQ ID N^{o} 684 | LI-526.1 | 30 | gb\|AAD39085.1\|AF091393_1 (AF091393) surface protein R28 [Streptococcus | Length = 1260 |
| | | | pyogenes] | |
| SEQ ID N^{o} 685 | LI-4192.1 | 58 | emb\|CAB44655.1\| (Y18605) hypothetical protein RvD1-Rv2024c′ | Length = 1606 |
| | | | [Mycobacterium bovis BCG] | |
| SEQ ID N^{o} 686 | LI-2958.1 | 39 | pir\|\|T13216 minor capsid protein 1608 - Lactobacillus phage phi-gle | Length = 1608 |
| | | | emb\|CAA66745.1\| (X98106) minor capsid protein [Bacteriophage phig1e] | |
| SEQ ID N^{o} 687 | LI-3752.1 | 27 | pir\|\|T28679 fibrinogen-binding protein homolog - Staphylococcus aureus | Length = 1315 |
| | | | emb\|CAA06651.1\| (AJ005646) sdrD [Staphylococcus aureus] | |
| SEQ ID N^{o} 688 | LI-6059.1 | 11 | emb\|CAB63691.1\| (AJ251790) hypothetical protein [Lactobacillus casei | Length = 465 |
| | | | bacteriophage A2] | |
| SEQ ID N^{o} 689 | LI-1259.1 | 48 | sp\|Q07833\|WAPA_BACSU WALL-ASSOCIATED PROTEIN PRECURSOR | |
| | | | pir\|\|S32920 cell wall-associated protein precursor wapA - Bacillus subtilis | |
| | | | gb\|AAA22883.1\| (L05634) wall-associated protein [Bacillus subtilis] | |
| | | | dbj\|BAA06656.1\| (D31856) WapA protein [Bacillus subti | |

| | | | Blastp result on non-redondant protein bank. | | |
|---|---|---|---|---|---|
| SEQ ID | Prot N^{o} | COMMENTS | homology/ | Comments/ | % |
| SEQ ID N^{o} 690 | LM-1002.1 | Unknown, similar to internalin protein | 23 | gi\|2347104\|gb\|AAB67969.1\|(U77368) internalin [Listeria | Length = 548 |
| | | | | monocytogenes] | |
| SEQ ID N^{o} 691 | LM-1005.1 | Unknown, similar to putative NAD(P)- | 53 | gi\|1361424\|pir\|\|S57559 strU protein - Streptomyces glaucescens | |
| | | dependent oxidoreductase | | | |
| SEQ ID N^{o} 692 | LM-1037.1 | Unknown | 15 | gi\|7459761\|pir\|\|C69520 hypothetical protein AF2163 - Archaeoglobus | |
| | | | | fulgidus | |
| SEQ ID N^{o} 693 | LM-1043.1 | Unknown, similar to efflux proteins | 52 | gi\|7474437\|pir\|\|B70065 antibiotic resistance protein homolog ywoG - Bacillus subtilis | |
| SEQ ID N^{o} 694 | LM-1050.1 | Unknown, LPXTG protein withh LRR repeats | 12 | gi\|5059350\|gb\|AAD38982.1\|AF153770_1 (AF153770) immunoreactive 47 kDa | Length = 428 |
| | | | | antigen PG97 [Porphyromonas gingivalis] | |
| SEQ ID N^{o} 695 | LM-1067.2 | Unknown, hypothetical secreted protein | | No Hits found | |
| SEQ ID N^{o} 696 | LM-1074.1 | internalin | 98 | gi\|2347102\|gb\|AAB67968.1\|(U77367) internalin [Listeria | Length = 821 |
| | | | | monocytogenes] | |
| SEQ ID N^{o} 697 | LM-1112.2 | Unknown, similar to B. subtilis transcription | 61 | gi\|1170575\|sp\|P46337\|IOLR_BACSU DNA-BINDING PROTEIN IOLR | |
| | | repressor of myo-inositol catabolism operon | | | |
| | | lolR | | | |
| SEQ ID N^{o} 698 | LM-1114.1 | Unknown | | No Hits found | |
| SEQ ID N^{o} 699 | LM-1115.3 | Unknown | | No Hits found | |
| SEQ ID N^{o} 700 | LM-1123.1 | Unknown | | No Hits found | |
| SEQ ID N^{o} 701 | LM-1127.1 | Unknown | | No Hits found | |
| SEQ ID N^{o} 702 | LM-1132.1 | Unknown, similar to transcription regulator | 25 | gi\|7481541\|pir\|\|T36904 probable transcription regulator - Streptomyces | |
| | | | | coelicolor | |
| SEQ ID N^{o} 703 | LM-1153.2 | Unknown, weakly similar to a bile acid 7-alpha | | No Hits found | |
| | | dehydratase | | | |
| SEQ ID N^{o} 704 | LM-1154.1 | unknown, similar to transcription regulator | 43 | gi\|7521744\|pir\|\|A70344 transcription regulator Crp/Fnr family - Aquifex | |
| | | Crp/Fnr family | | aeolicus | |
| SEQ ID N^{o} 705 | LM-1155.1 | Unknown, weakly similar to a putative | 35 | gi\|7474715\|pir\|\|C69971 conserved hypothetical protein yraK - Bacillus | |
| | | haloacetate dehalogenase | | subtilis | |
| SEQ ID N^{o} 706 | LM-1156.1 | Unknown | | No Hits found | |
| SEQ ID N^{o} 707 | LM-1157.1 | Unknown | | No Hits found | |
| SEQ ID N^{o} 708 | LM-1158.1 | Unknown | | No Hits found | |
| SEQ ID N^{o} 709 | LM-1159.1 | unknown | | No Hits found | |
| SEQ ID N^{o} 710 | LM-1160.1 | Unknown, hypothetical | | No Hits found | |
| SEQ ID N^{o} 711 | LM-1169.1 | Unknown, similar to 6-phospho-beta- | 61 | gi\|6851034\|emb\|CAB71151.1\|(AJ250202) beta-glucosidase | Length = 486 |
| | | glucosidase | | [Lactobacillus plantarum] | |
| SEQ ID N^{o} 712 | LM-1171.1 | Unknown, similar to phosphotransferase | 63 | gi\|6851033\|emb\|CAB71150.1\|(AJ250202) permease [Lactobacillus | Length = 577 |
| | | system (PTS) beta-glucoside-specific enzyme | | plantarum] | |
| | | IIABC component | | | |
| SEQ ID N^{o} 713 | LM-1172.1 | Unknown | | No Hits found | |
| SEQ ID N^{o} 714 | LM-1173.1 | Unknown, similar to ribose 5-phosphate | 70 | gi\|731980\|sp\|P37351\|RPIB_ECOLI RIBOSE 5-PHOSPHATE ISOMERASE B | |
| | | isomerase | | (PHOSPHORIBOISOMERASE B) | |
| SEQ ID N^{o} 715 | LM-1174.1 | Unknown, similar to Ribulose-5-Phosphate 3- | 58 | gi\|2829613\|sp\|P74061\|RPE_SYNY3 RIBULOSE-PHOSPHATE 3-EPIMERASE | |
| | | Epimerase | | (PENTOSE-5-PHOSPHATE 3-EPIMERASE) (PPE) (R5P3E) | |
| SEQ ID N^{o} 716 | LM-1175.1 | Unknown, similar to transcriptional regulator | 51 | gi\|1173387\|sp\|P43472\|SCRR_PEDPE SUCROSE OPERON REPRESSOR | |
| | | (Lacl family) | | (SCR OPERON REGULATORY PROTEIN) | |
| SEQ ID N^{o} 717 | LM-1176.1 | Unknown, similar to transcription regulator | 24 | gi\|2127359\|pir\|\|I40868 hypothetical protein 3 nanH region - Clostridium | |
| | | | | perfringens | |
| SEQ ID N^{o} 718 | LM-1228.1 | Unknown, similar to B. subtilis YxjH and YxjG | 60 | gi\|2828504\|sp\|P42319\|YXJH_BACSU HYPOTHETICAL 38.3 KD PROTEIN IN | |
| | | proteins | | PEPT-KATB INTERGENIC REGION | |
| SEQ ID N^{o} 719 | LM-1243.1 | HEXOSE PHOSPHATE TRANSPORT | 60 | gi\|136773\|sp\|P27670\|UHPT_SALTY HEXOSE PHOSPHATE TRANSPORT | |
| | | PROTEIN | | PROTEIN | |
| SEQ ID N^{o} 720 | LM-1248.1 | unknown, surface protein (LPXTG motif) | 29 | gi\|417315\|sp\|P32653\|MRP_STRSU MURAMIDASE-RELEASED PROTEIN | |
| | | | | PRECURSOR (136 KD SURFACE PROTEIN) | |
| SEQ ID N^{o} 721 | LM-1249.1 | unknown | | No Hits found | |
| SEQ ID N^{o} 722 | LM-1258.1 | unknown, similar to transposases | 57 | gi\|7474337\|pir\|\|H59102 hypothetical protein pXO1-96 - Bacillus anthracis | |
| | | | | virulence plasmid pXO1 | |
| SEQ ID N^{o} 723 | LM-1259.1 | unknown, similar to transposases | 62 | gi\|929968\|gb\|AAA74024.1\|(U30713) ORFA [Bacillus anthracis] | |
| SEQ ID N^{o} 724 | LM-1261 .1 | Unknown, similar to transporter | 52 | gi\|7514408\|pir\|\|H71283 conserved hypothetical integral membrane | |
| | | | | protein TP0771 - syphilis spirochete | |
| SEQ ID N^{o} 725 | LM-1267.1 | unknown | | No Hits found | |
| SEQ ID N^{o} 726 | LM-1306.1 | unknown | | No Hits found | |
| SEQ ID N^{o} 727 | LM-1343.2 | unknown | 39 | gi\|6322945\|ref\|NP_013018.1\|nucleolar protein that is immunologically | |
| | | | | and structurally related to rat Nopp140, a nonribosomal protein of the | |
| | | | | nucleolus and coiled bodies.; Srp40p | |
| SEQ ID N^{o} 728 | LM-1353.1 | unknown, similar to membrane and transport | 61 | gi\|7473695\|pir\|\|A75272 probable transport protein - Deinococcus | |
| | | proteins | | radiodurans (strain R1) | |
| SEQ ID N^{o} 729 | LM-1354.1 | unknown, similar to ABC transporter | 67 | gi\|7445714\|pir\|\|E69762 ABC transporter (permease) homolog yclH - | |
| | | | | Bacillus subtilis | |
| SEQ ID N^{o} 730 | LM-1357.1 | unknown, conserved hypothetical protein | 58 | gi\|7475935\|pir\|\|F69762 transporter homolog ycll - Bacillus subtilis | |
| SEQ ID N^{o} 731 | LM-1358.1 | unknown, similar to sensor protein histidine | 33 | gi\|2500765\|sp\|Q47745\|VANS_ENTFA SENSOR PROTEIN VANSB | |
| | | kinases (2 components regulatory systems) | | (VANCOMYCIN B-TYPE RESISTANCE PROTEIN VANSB) | |
| | | | | (VANCOMYCIN HISTIDINE PROTEIN KINASE) | |
| SEQ ID N^{o} 732 | LM-1359.1 | unknown, similar to transcription response | 65 | gi\|2500744\|sp\|Q47744\|VANR_ENTFA REGULATORY PROTEIN VANRB | |
| | | regulator | | | |
| SEQ ID N^{o} 733 | LM-1390.1 | unknown | | No Hits found | |
| SEQ ID N^{o} 734 | LM-1391.1 | unknown, similar to phosphotransferase | 66 | gi\|1100076\|gb\|AAC05713.1\|(L49336) PTS-dependent enzyme II | Length = 616 |
| | | system (PTS) beta-glucoside-specific enzyme | | [Clostridium longisporum] | |
| | | IIABC | | | |
| SEQ ID N^{o} 735 | LM-1392.1 | unknown, similar to glycerol kinase | 58 | gi\|6685472\|sp\|Q9X049\|GLK1_THEMA GLYCEROL KINASE 1 | |
| | | | | (ATP: GLYCEROL 3-PHOSPHOTRANSFERASE 1) (GLYCEROKINASE | |
| | | | | 1) (GK 1) | |
| SEQ ID N^{o} 736 | LM-1394.1 | unknown, similar to transketolase | 54 | gi\|2833526\|sp\|Q58092\|TKTC_METJA PUTATIVE TRANSKETOLASE C- | |
| | | | | TERMINAL SECTION (TK) | |
| SEQ ID N^{o} 737 | LM-1396.1 | unknown, similar to transketolase | 64 | gi\|2833528\|sp\|Q58094\|TKTN_METJA PUTATIVE TRANSKETOLASE N- | |
| | | | | TERMINAL SECTION (TK) | |
| SEQ ID N^{o} 738 | LM-1398.3 | unknown, similar to hypothetical proteins | 42 | gi\|7462474\|pir\|\|B72314 hypothetical protein - Thermotoga maritima | |
| | | | | (strain MSB8) | |
| SEQ ID N^{o} 739 | LM-1438.1 | Unknwon | 89 | gi\|466144\|sp\|P33383\|YORX_LISMO HYPOTHETICAL 12.2 KD PROTEIN IN | |
| | | | | PLCB-LDH INTERGENIC REGION PRECURSOR (ORFX) | |
| SEQ ID N^{o} 740 | LM-1439.1 | phospholipase C | 95 | gi\|464374\|sp\|P33378\|PHLC_LISMO PHOSPHOLIPASE C PRECURSOR (PLC) | |
| | | | | (PHOSPHATIDYLCHOLINE CHOLINEPHOSPHOHYDROLASE) | |
| | | | | (LECITHINASE) | |
| SEQ ID N^{o} 741 | LM-1440.1 | | | No Hits found | |
| SEQ ID N^{o} 742 | LM-1441.1 | | 16 | gi\|6649606\|gb\|AAF21477.1\|U91654_1 (U91654) merozoite surface | Length = 233 |
| | | | | antigen 2 [Plasmodium falciparum] | |
| SEQ ID N^{o} 743 | LM-1442.1 | actin-assembly inducing protein precursor | 99 | gi\|461463\|sp\|P33379\|ACTA_LISMO ACTIN-ASSEMBLY INDUCING PROTEIN | |
| | | | | PRECURSOR | |
| SEQ ID N^{o} 744 | LM-1444.1 | Zinc metalloproteinase precursor | 98 | gi\|130985\|sp\|P23224\|PRO1_LISMO ZINC METALLOPROTEINASE | |
| | | | | PRECURSOR | |
| SEQ ID N^{o} 745 | LM-1445.1 | listeriolysin O precursor | 98 | gi\|887028\|gb\|AAA69528.1\|(U25446) listeriolysin O [Listeria | |
| | | | | monocytogenes] | |
| SEQ ID N^{o} 746 | LM-1446.1 | phosphatidylinositol-specific phospholipase c | 97 | gi\|464403\|sp\|P34024\|PLC_LISMO 1-PHOSPHATIDYLINOSITOL | |
| | | | | PHOSPHODIESTERASE PRECURSOR (PHOSPHATIDYLINOSITOL- | |
| | | | | SPECIFIC PHOSPHOLIPASE C) (PI-PLC) | |
| SEQ ID N^{o} 747 | LM-1447.1 | listeriolysin positive regulatory protein | 99 | gi\|464460\|sp\|P22262\|PRFA_LISMO LISTERIOLYSIN REGULATORY | |
| | | | | PROTEIN | |
| SEQ ID N^{o} 748 | LM-1518.1 | Unknown, similar to transcription antiterminator | 42 | gi\|7475883\|pir\|\|F69848 transcription antiterminator BgIG family homolog | |
| | | BgIG family | | yjdC - Bacillus subtilis | |
| SEQ ID N^{o} 749 | LM-1519.1 | Unknown, similar to PTS system, fructose- | 51 | gi\|7450499\|pir\|\|H69626 PTS fructose-specific enzyme IIBC component | |
| | | specific IIA component | | fruA - Bacillus subtilis | |
| SEQ ID N^{o} 750 | LM-1520.1 | Unknown, similar to PTS system, fructose- | 69 | gi\|418517\|sp\|P32672\|PTWC_ECOLI PTS SYSTEM, FRUCTOSE-LIKE-2 IIC | |
| | | specific IIC component | | COMPONENT (PHOSPHOTRANSFERASE ENZYME II, C COMPONENT) | |
| SEQ ID N^{o} 751 | LM-1521.1 | Unknown, similar to PTS system, fructose- | 72 | gi\|418518\|sp\|P32673\|PTWB_ECOLI PTS SYSTEM, FRUCTOSE-LIKE-2 IIB | |
| | | specific IIB component | | COMPONENT 1 (PHOSPHOTRANSFERASE ENZYME II, B COMPONENT) | |
| SEQ ID N^{o} 752 | LM-1523.1 | Unknown, similar to an E. coli putative | 36 | gi\|2507030\|sp\|P37191\|GATZ_ECOLI PUTATIVE TAGATOSE δ-PHOSPHATE | |
| | | tagatose 6-phosphate kinase | | KINASE GATZ | |
| SEQ ID N^{o} 753 | LM-1528.1 | Unknown | | No Hits found | |
| SEQ ID N^{o} 754 | LM-1538.1 | unknown | | No Hits found | |
| SEQ ID N^{o} 755 | LM-1547.1 | Internalin B | 98 | gi\|98684\|pir\|\|C39930 hypothetical protein (internalin region) - Listeria | Length = 630 |
| | | | | monocytogenes | |
| SEQ ID N^{o} 756 | LM-1549.1 | internalin A | 95 | gi\|3123226\|sp\|P25146\|INLA_LISMO INTERNALIN A PRECURSOR | |
| SEQ ID N^{o} 757 | LM-156.1 | unknown BEST-BLASTP = | | No Hits found | |
| SEQ ID N^{o} 758 | LM-1571.1 | Unknown, conserved hypothetical protein | 59 | gi\|7388460\|sp\|P75809\|YBJI_ECOLI HYPOTHETICAL 30.2 KDA PROTEIN IN | Length = 271 |
| | | | | CMR-GRXA INTERGENIC REGION | |
| SEQ ID N^{o} 759 | LM-1572.1 | Unknown, similar to unknown protein | 48 | gi\|7482419\|pir\|\|F69065 hypothetical protein MTH1490 - | |
| | | | | Methanobacterium thermoautotrophicum (strain Delta H) | |
| SEQ ID N^{o} 760 | LM-1586.1 | unknown, similar to B. subtilis YkcC protein | 82 | gi\|3123150\|sp\|O34319\|YKCC_BACSU HYPOTHETICAL 37.4 KDA PROTEIN | |
| | | | | IN SPOIISA-HTRA INTERGENIC REGION | |
| SEQ ID N^{o} 761 | LM-1624.2 | Unknown | 19 | gi\|4959401\|gb\|AAD34335.1\|AF115391_4 (AF115391) LaaC [Lactobacillus | Length = 81 |
| | | | | sakei] | |
| SEQ ID N^{o} 762 | LM-1625.2 | Unknown | | No Hits found | |
| SEQ ID N^{o} 763 | LM-1627.1 | Unknown | | No Hits found | |
| SEQ ID N^{o} 764 | LM-1631.1 | Unknown, conserved hypothetical protein | 48 | gi\|2851530\|sp\|P32399\|YHGE_BACSU HYPOTHETICAL 84.1 KD PROTEIN IN | |
| | | | | HEMY-GLTT INTERGENIC REGION (ORFB) | |
| SEQ ID N^{o} 765 | LM-1632.1 | Unknown, similar to transcription regulator | 40 | gi\|6470204\|gb\|AAF13658.1\|AF188935_56 (AF188935) pXO2-53 [Bacillus | Length = 482 |
| | | | | anthracis] | |
| SEQ ID N^{o} 766 | LM-1634.1 | Unknown, similar to penicillin acylase | 61 | gi\|1731309\|sp\|P54948\|YXEI_BACSU HYPOTHETICAL 37.2 KD PROTEIN IN IDH-DEOR | |
| | | | | INTERGENIC REGION | |
| SEQ ID N^{o} 767 | LM-1655.1 | Unknown, similar to transcription regulator | 23 | gi\|482361\|pir\|\|A42730 trans-acting positive regulator Mry - Streptococcus | Length = 530 |
| | | (VirR from Streptococcus pyogenes) | | pyogenes (type M6, strain D471) | |
| SEQ ID N^{o} 768 | LM-1656.1 | Unknown, putative membrane associated | 19 | gi\|6731239\|gb\|AAF27178.1\|AF072716_1 (AF072716) membrane associated | Length = 445 |
| | | lipoprotein | | lipoprotein precursor [Mycoplasma mycoides mycoides SC] | |
| SEQ ID N^{o} 769 | LM-1658.1 | Unknown | | No Hits found | |
| SEQ ID N^{o} 770 | LM-1659.3 | Unknown | | No Hits found | |
| SEQ ID N^{o} 771 | LM-1660.3 | Unknown | | No Hits found | |
| SEQ ID N^{o} 772 | LM-1713.1 | Unknown, similar to transcriptional regulator | 44 | gi\|2996626\|gb\|AAC46441.1\|(AF009224) LysR-type transcriptional | Length = 304 |
| | | (LysR family) | | activator [Acinetobacter sp. ADP1] | |
| SEQ ID N^{o} 773 | LM-1714.1 | Unknown, similar to acylase | 43 | gi\|7476683\|pir\|\|E70610 hypothetical protein Rv1215c - Mycobacterium | |
| | | | | tuberculosis (strain H37RV) | |
| SEQ ID N^{o} 774 | LM-1718.2 | Unknown, similar to sugar transferase | 22 | gi\|4580634\|gb\|AAD24457.1\|AF118389_14 (AF118389) Cps2K | Length = 276 |
| | | | | [Streptococcus suis] | |
| SEQ ID N^{o} 775 | LM-1760.1 | Unknown, similar to unknown proteins | 26 | gi\|7322701\|gb\|AAF59460.1\|(AC024760) contains similarity to TR: Q10466 | Length = 6677 |
| | | | | [Caenorhabditis elegans] | |
| SEQ ID N^{o} 776 | LM-1776.3 | Unknown | 51 | gi\|7475903\|pir\|\|C69931 transcription regulator homolog yozG - Bacillus | |
| | | | | subtilis | |
| SEQ ID N^{o} 777 | LM-1778.1 | Unknown, putative peptidoglycan bound | 24 | gi\|5327234\|emb\|CAB46338.1\|(AJ133114) R5 protein [Streptococcus | Length = 979 |
| | | protein (LPXTG motif) | | agalactiae] | |
| SEQ ID N^{o} 778 | LM-18.1 | Unknwon, similar to hypothetical PTS enzyme | 40 | gi\|732028\|sp\|P39302\|PTXB_ECOLI UNKNOWN PENTITOL | |
| | | IIB component | | PHOSPHOTRANSFERASE ENZYME II, B COMPONENT | |
| SEQ ID N^{o} 779 | LM-1814.1 | Unknown, similar to putative peptidoglycan | 87 | gi\|1730252\|sp\|P54482\|GCPE_BACSU GCPE PROTEIN HOMOLOG | |
| | | acetylation protein | | | |
| SEQ ID N^{o} 780 | LM-1827.1 | Unknown, similar to E. coli LytB protein | 75 | gi\|1731004\|sp\|P54473\|YQFP_BACSU LYTB PROTEIN HOMOLOG | |
| SEQ ID N^{o} 781 | LM-1838.1 | internalin E | 97 | gi\|3980136\|emb\|CAA07458.1\|(AJ007319) internalin E [Listeria | Length = 499 |
| | | | | monocytogenes] | |
| SEQ ID N^{o} 782 | LM-1840.1 | internalin H | 95 | gi\|3980135\|emb\|CAA07457.1\|(AJ007319) internalin H [Listeria | Length = 548 |
| | | | | monocytogenes] | |
| SEQ ID N^{o} 783 | LM-1842.1 | internalin G | 98 | gi\|3980134\|emb\|CAA07456.1\|(AJ007319) internalin G [Listeria | Length = 490 |
| | | | | monocytogenes] | |
| SEQ ID N^{o} 784 | LM-1856.2 | Unknown, similar to unknown protein | 97 | gi\|6002200\|emb\|CAB56705.1\|(Y16468) hypothetical protein [Listeria | Length = 392 |
| | | | | monocytogenes] | |
| SEQ ID N^{o} 785 | LM-1858.1 | Unknown, similar to unknown protein | 47 | gi\|466146\|sp\|P33385\|YORZ_LISMO HYPOTHETICAL 16.9 KD LIPOPROTEIN | |
| | | | | IN PLCB-LDH INTERGENIC REGION PRECURSOR (ORFZ) | |
| SEQ ID N^{o} 786 | LM-1859.2 | Unknown | | No Hits found | |
| SEQ ID N^{o} 787 | LM-1861.1 | | 76 | gi\|3122592\|sp\|O32797\|PFL_LACLA FORMATE ACETYLTRANSFERASE | |
| | | | | (PYRUVATE FORMATE-LYASE) | |
| SEQ ID N^{o} 788 | LM-1972.3 | Unknown, similar to internalin | 19 | gi\|2230998\|emb\|CAA65738.1\|(X97014) ORF A [Listeria seeligeri] | Length = 902 |
| SEQ ID N^{o} 789 | LM-1974.3 | Unknown, similar to internalin | 31 | gi\|2230998\|emb\|CAA65738.1\|(X97014) ORF A [Listeria seeligeri] | Length = 902 |
| SEQ ID N^{o} 790 | LM-198.1 | diol dehydratase-reactivating factor small chain - | 58 | gi\|7467210\|pir\|\|T08598 probable diol dehydratase-reactivating factor | |
| | | Klebsiella oxytoca | | small chain - Klebsiella oxytoca | |
| SEQ ID N^{o} 791 | LM-1997.1 | unknown | | No Hits found | |
| SEQ ID N^{o} 792 | LM-2009.1 | Unknown | | No Hits found | |
| SEQ ID N^{o} 793 | LM-2037.1 | Unknwon, similar to drug-export protein | 66 | gi\|7430060\|pir\|\|F69763 multidrug resistance protein homolog ycnB - | |
| | | | | Bacillus subtilis | |
| SEQ ID N^{o} 794 | LM-2049.3 | Unknown | | No Hits found | |
| SEQ ID N^{o} 795 | LM-2050.1 | Unknown | | No Hits found | |
| SEQ ID N^{o} 796 | LM-208.1 | | | No Hits found | |
| SEQ ID N^{o} 797 | LM-2106.1 | | 95 | gi\|4336793\|gb\|AAD17954.1\|(AF105341) pyrimidine nucleoside | Length = 419 |
| | | | | phosphorylase [Listeria monocytogenes] | |
| SEQ ID N^{o} 798 | LM-2116.1 | Unknown | | No Hits found | |
| SEQ ID N^{o} 799 | LM-2130.1 | unknown | | No Hits found | |
| SEQ ID N^{o} 800 | LM-2137.2 | Unknown, similar to internalin | 19 | gi\|2230998\|emb\|CAA65738.1\|(X97014) ORF A [Listeria seeligeri] | Length = 902 |
| SEQ ID N^{o} 801 | LM-2138.1 | unknown | | No Hits found | |
| SEQ ID N^{o} 802 | LM-2141.1 | Unknown | 27 | gi\|5912390\|emb\|CAB56115.1\|(Y18096) coiled-coil protein [Sulfolobus | Length = 464 |
| | | | | solfataricus] | |
| SEQ ID N^{o} 803 | LM-2142.1 | Unknown | | No Hits found | |
| SEQ ID N^{o} 804 | LM-2145.1 | Unknown | 45 | gi\|548467\|sp\|P36496\|PEDB_PEDAC PEDIOCIN PA-1 IMMUNITY PROTEIN | |
| | | | | (PEDIOCIN ACH IMMUNITY PROTEIN) | |
| SEQ ID N^{o} 805 | LM-2161.2 | Unknwon, similar to toxin components | 17 | gi\|6730537\|pdb\|1QS2\|A Chain A, Crystal Structure Of Vip2 With Nad | Length = 401 |
| SEQ ID N^{o} 806 | LM-221.1 | unknown | 42 | gi\|7479622\|pir\|\|T35326 hypothetical protein SC5H1.10c - Streptomyces | |
| | | | | coelicolor | |
| SEQ ID N^{o} 807 | LM-2215.1 | unknown | | No Hits found | |
| SEQ ID N^{o} 808 | LM-223.1 | unknown | | No Hits found | |
| SEQ ID N^{o} 809 | LM-2244.1 | | 53 | gi\|6165970\|gb\|AAF04740.1\|(AF102169) multidrug resistance-like protein | Length = 228 |
| | | | | [Listeria monocytogenes] | |
| SEQ ID N^{o} 810 | LM-230.1 | unknown | | No Hits found | |
| SEQ ID N^{o} 811 | LM-231.1 | unknown, similar to regulatory proteins | 54 | gi\|7635981\|emb\|CAB88816.1\|(AL353832) putative MerR-family | Length = 135 |
| | | | | transcriptional regulator. [Streptomyces coelicolor A3(2)] | |
| SEQ ID N^{o} 812 | LM-232.1 | unknown, similar to B. subtilis YjcS protein | 36 | gi\|7475112\|pir\|\|C69848 hypothetical protein yjcS - Bacillus subtilis | |
| SEQ ID N^{o} 813 | LM-2323.1 | Unknown, similar to surface protein | 16 | gi\|120457\|sp\|P14738\|FNBA_STAAU FIBRONECTIN-BINDING PROTEIN | |
| | | | | PRECURSOR (FNBP) | |
| SEQ ID N^{o} 814 | LM-2334.1 | Unknown, conserved hypothetical protein | 49 | gi\|7471580\|pir\|\|E75635 conserved hypothetical protein - Deinococcus | |
| | | | | radiodurans (strain R1) | |
| SEQ ID N^{o} 815 | LM-2335.2 | Unknown, similar to unknown protein | 34 | gi\|7472782\|pir\|\|F75635 hypothetical protein - Deinococcus radiodurans | |
| | | | | (strain R1) | |
| SEQ ID N^{o} 816 | LM-2336.3 | Unknown | | No Hits found | |
| SEQ ID N^{o} 817 | LM-2358.1 | unknown, similar to efflux transporter | 44 | gi\|4467970\|emb\|CAB37973.1\|(X76640) hypothetical protein | Length = 507 |
| | | | | [Myxococcus xanthus] | |
| SEQ ID N^{o} 818 | LM-2391.1 | Unknwon | | No Hits found | |
| SEQ ID N^{o} 819 | LM-2392.1 | unknwon | | No Hits found | |
| SEQ ID N^{o} 820 | LM-2393.1 | | | No Hits found | |
| SEQ ID N^{o} 821 | LM-2394.1 | unknown | | No Hits found | |
| SEQ ID N^{o} 822 | LM-2395.1 | Unknwon | | No Hits found | |
| SEQ ID N^{o} 823 | LM-2397.1 | Unknown | | No Hits found | |
| SEQ ID N^{o} 824 | LM-2398.1 | unknown | | No Hits found | |
| SEQ ID N^{o} 825 | LM-2400.1 | unknown | | No Hits found | |
| SEQ ID N^{o} 826 | LM-2401.1 | Unknwon | | No Hits found | |
| SEQ ID N^{o} 827 | LM-2404.1 | Unknwon | | No Hits found | |
| SEQ ID N^{o} 828 | LM-241.2 | unknown | 50 | gi\|7380228\|emb\|CAB84814.1\|(AL162756) hypothetical protein [Neisseria | Length = 181 |
| | | | | meningitidis] | |
| SEQ ID N^{o} 829 | LM-2438.1 | unknwon, surface anchored protein | 21 | gi\|139687\|sp\|P11000\|WAPA_STRMU WALL-ASSOCIATED PROTEIN | |
| | | | | PRECURSOR | |
| SEQ ID N^{o} 830 | LM-2448.2 | autolysin; amidase | 99 | gi\|2653655\|gb\|AAC46384.1\|(AF035424) autolysin; amidase [Listeria | Length = 917 |
| | | | | monocytogenes] | |
| SEQ ID N^{o} 831 | LM-245.1 | unknown | | No Hits found | |
| SEQ ID N^{o} 832 | LM-246.1 | unknown | | No Hits found | |
| SEQ ID N^{o} 833 | LM-247.3 | unknown, similar to methylases | 42 | gi\|7469765\|pir\|\|S76841 hypothetical protein - Synechocystis sp. (strain | |
| | | | | PCC 6803) | |
| SEQ ID N^{o} 834 | LM-2474.1 | Unknown | 49 | gi\|7592633\|dbj\|BAA94339.1\|(AB033763) hypothetical protein | |
| | | | | [Staphylococcus aureus] | |
| SEQ ID N^{o} 835 | LM-2475.1 | Unknown, putaive secreted and Lysin rich | 26 | gi\|5921146\|dbj\|BAA84590.1\|(AB003084) RNA polymerase sigma 70 | Length = 675 |
| | | protein | | [Helicobacter pylori] | |
| SEQ ID N^{o} 836 | LM-2489.2 | Unknown, similar to phospho-N- | 74 | gi\|417313\|sp\|Q03521\|MRAY_BACSU PHOSPHO-N- | |
| | | acetylmuramoyl-pentapeptide transferase | | ACETYLMURAMOYL-PENTAPEPTIDE-TRANSFERASE (UDP- | |
| | | | | MURNAC-PENTAPEPTIDE PHOSPHOTRANSFERASE) | |
| SEQ ID N^{o} 837 | LM-2495.1 | cell-division protein FtsA | 74 | gi\|120567\|sp\|P28264\|FTSA_BACSU CELL DIVISION PROTEIN FTSA | |
| SEQ ID N^{o} 838 | LM-2503.1 | Unknown, similar to internalin protein | 35 | gi\|2347102\|gb\|AAB67968.1\|(U77367) internalin [Listeria | Length = 821 |
| | | | | monocytogenes] | |
| SEQ ID N^{o} 839 | LM-2504.1 | Unknown, putative peptidoglycan bound | 8 | gi\|7485517\|pir\|\|T41744 hypothetical protein F15J1.40 - Arabidopsis | |
| | | protein (LPXTG motif) | | thaliana | |
| SEQ ID N^{o} 840 | LM-2513.1 | | | No Hits found | |
| SEQ ID N^{o} 841 | LM-2521.1 | creatinine amidohydrolase | 46 | gi\|7479569\|pir\|\|T35153 hypothetical protein SC5A7.04c SC5A7.04c - | |
| | | | | Streptomyces coelicolor | |
| SEQ ID N^{o} 842 | LM-2522.1 | | 52 | gi\|7436634\|pir\|\|F72422 2-dehydro-3-deoxyphosphogluconate aldolase/4- | |
| | | | | hydroxy-2-oxoglutarate aldolase - Thermotoga maritima (strain MSB8) | |
| SEQ ID N^{o} 843 | LM-2523.1 | | 54 | gi\|1176259\|sp\|P45548\|PHP_ECOLI PHOSPHOTRIESTERASE HOMOLOGY | |
| | | | | PROTEIN | |
| SEQ ID N^{o} 844 | LM-2524.1 | | 55 | gi\|7480860\|pir\|\|T37066 probable integral membrane protein - | |
| | | | | Streptomyces coelicolor | |
| SEQ ID N^{o} 845 | LM-2527.1 | | 50 | gi\|2851672\|sp\|P39303\|PTXA_ECOLI UNKNOWN PENTITOL | |
| | | | | PHOSPHOTRANSFERASE ENZYME II, A COMPONENT | |
| SEQ ID N^{o} 846 | LM-2528.1 | | 50 | gi\|7443060\|pir\|\|D70044 transcription regulator GntR family homolog yvoA - | |
| | | | | Bacillus subtilis | |
| SEQ ID N^{o} 847 | LM-2597.3 | unknown, similar to MINOR TEICHOIC ACIDS | 22 | gi\|2198542\|emb\|CAA59781.1\|(X85787) ss-1,3-N- | Length = 306 |
| | | BIOSYNTHESIS PROTEIN GGAB | | acetylglucosaminyltransferase [Streptococcus pneumoniae] | |
| SEQ ID N^{o} 848 | LM-2598.3 | | 86 | gi\|3608394\|gb\|AAC35920.1\|(AF071085) putative glucose-1-phosphate | Length = 288 |
| | | | | thymidyl transferase [Enterococcus faecalis] | |
| SEQ ID N^{o} 849 | LM-2599.1 | | 74 | gi\|3608395\|gb\|AAC35921.1\|(AF071085) dTDP-4-dehydrorhamnose 3,5- | Length = 190 |
| | | | | epimerase [Enterococcus faecalis] | |
| SEQ ID N^{o} 850 | LM-2600.1 | | 75 | gi\|7437238\|pir\|\|H69105 dTDP-glucose 4,6-dehydratase - | |
| | | | | Methanobacterium thermoautotrophicum (strain Delta H) | |
| SEQ ID N^{o} 851 | LM-2601.1 | unknown, similar to DTDP-L-RHAMNOSE | 68 | gi\|3608397\|gb\|AAC35923.1\|(AF071085) putative dTDP-4-keto-L- | Length = 299 |
| | | SYNTHASE | | rhamnose reductase [Enterococcus faecalis] | |
| SEQ ID N^{o} 852 | LM-2602.1 | unknown, similar to TEICHOIC ACID | 34 | gi\|1074220\|pir\|\|S49240 hypothetical protein 3 (capsulation locus) - | |
| | | BIOSYNTHESIS PROTEIN B PRECURSOR | | Haemophilus influenzae (strain RM107) | |
| SEQ ID N^{o} 853 | LM-2606.1 | TEICHOIC ACID BIOSYNTHESIS PROTEIN B | 58 | gi\|135272\|sp\|P27621\|TAGB_BACSU TEICHOIC ACID BIOSYNTHESIS | |
| | | PRECURSOR. | | PROTEIN B PRECURSOR | |
| SEQ ID N^{o} 854 | LM-2608.1 | unknown, similar to glycosyltransferase | 46 | gi\|4580634\|gb\|AAD24457.1\|AF118389_14 (AF118389) Cps2K | Length = 276 |
| | | | | [Streptococcus suis] | |
| SEQ ID N^{o} 855 | LM-2609.1 | unknown, siumilar to glysosyltransferase | 30 | gi\|6983732\|emb\|CAB75371.1\|(AL139298) putative glycosyltransferase | Length = 1135 |
| | | | | [Streptomyces coelicolor A3(2)] | |
| SEQ ID N^{o} 856 | LM-2618.1 | integrase | 46 | gi\|7475580\|pir\|\|A69774 integrase homolog ydcL - Bacillus subtilis | |
| SEQ ID N^{o} 857 | LM-2619.1 | unknown, similar to a protein encoded by | 53 | gi\|3243181\|gb\|AAC34795.1\|(AF063010) unknown [Enterococcus faecium] | Length = 143 |
| | | Tn916 | | | |
| SEQ ID N^{o} 858 | LM-2650.2 | Similar to heme A farnesyltransferase | 67 | gi\|1841876\|dbj\|BAA11110.1\|(D70843) heme O synthetase [Bacillus | Length = 307 |
| | | | | stearothermophilus] | |
| SEQ ID N^{o} 859 | LM-2664.2 | | 73 | gi\|399058\|sp\|P31104\|AROC_BACSU CHORISMATE SYNTHASE (5- | |
| | | | | ENOLPYRUVYLSHIKIMATE-3-PHOSPHATE PHOSPHOLYASE) | |
| | | | | (VEGETATIVE PROTEIN 216) (VEG216) | |
| SEQ ID N^{o} 860 | LM-2689.1 | unknown, conserved hypothetical protein, | 60 | gi\|418459\|sp\|P32726\|YLXS_BACSU HYPOTHETICAL 17.6 KDA PROTEIN IN | |
| | | similar to B. subtilis YlxS protein | | NUSA 5′REGION (P15A) (ORF1) | |
| SEQ ID N^{o} 861 | LM-270.1 | unknown | 37 | gi\|7445062\|pir\|\|E72278 transcription regulator, RpiR family - Thermotoga | |
| | | | | maritima (strain MSB8) | |
| SEQ ID N^{o} 862 | LM-272.1 | Unknown, similar to PTS system, fructose- | 50 | gi\|1708300\|sp\|P54745\|HRSA_ECOLI HRSA PROTEIN | |
| | | specific IIABC component | | | |
| SEQ ID N^{o} 863 | LM-273.1 | Unknown, weakly similar to sugar hydrolase | 49 | gi\|2506621\|sp\|P54746\|YBGG_ECOLI HYPOTHETICAL 100.0 KDA PROTEIN IN | |
| | | | | HRSA-CYDA NTERGENIC REGION | |
| SEQ ID N^{o} 864 | LM-274.1 | Unknown, similar to Sucrose phosphorylase | 59 | gi\|7466753\|pir\|\|H64879 probable membrane protein b1309 - Escherichia | |
| | | | | coli | |
| SEQ ID N^{o} 865 | LM-275.1 | Unknown, conserved hypothetical protein | 64 | gi\|1176152\|sp\|P44507\|YHAD_HAEIN HYPOTHETICAL PROTEIN HI0091 | |
| SEQ ID N^{o} 866 | LM-2761.1 | | | No Hits found | |
| SEQ ID N^{o} 867 | LM-284.1 | unknown | | No Hits found | |
| SEQ ID N^{o} 868 | LM-2928.1 | Unknown, similar to oxidoreductase | 51 | gi\|7499000\|pir\|\|T16059 hypothetical protein F13D11.4 - Caenorhabditis | |
| | | | | elegans | |
| SEQ ID N^{o} 869 | LM-2929.1 | Unknown, similar to transcriptional regulator | 41 | gi\|7444887\|pir\|\|B69970 transcription regulator MerR family homolog yraB - | |
| | | (MerR family) | | Bacillus subtilis | |
| SEQ ID N^{o} 870 | LM-3009.3 | unknown BEST-BLASTP = | 34 | gi\|7470965\|pir\|\|T28679 fibrinogen-binding protein homolog - | |
| | | | | Staphylococcus aureus | |
| SEQ ID N^{o} 871 | LM-3010.1 | unknown, highly similar to TN916 ORF23 | 79 | gi\|532556\|gb\|AAB60010.1\|(U09422) ORF23 [Enterococcus faecalis] | |
| SEQ ID N^{o} 872 | LM-3011.1 | unknown, highly similar to TN916 ORF22 | 82 | gi\|532555\|gb\|AAB60011.1\|(U09422) ORF22 [Enterococcus faecalis] | |
| SEQ ID N^{o} 873 | LM-3012.1 | unknown, highly similar to TN916 ORF21 | 80 | gi\|532554\|gb\|AAB60012.1\|(U09422) ORF21 [Enterococcus faecalis] | |
| SEQ ID N^{o} 874 | LM-3013.1 | unknown, highly similar to TN916 ORF20 | 68 | gi\|532553\|gb\|AAB60013.1\|(U09422) ORF20 [Enterococcus faecalis] | |
| SEQ ID N^{o} 875 | LM-3014.1 | unknown, similar to B. subtilis YddA protein | 47 | gi\|7459823\|pir\|\|B69775 hypothetical protein yddA - Bacillus subtilis | |
| | | BEST-BLASTP = | | | |
| SEQ ID N^{o} 876 | LM-3016.1 | unknown, highly similar to TN916 ORF18 | 56 | gi\|532551\|gb\|AAB60015.1\|(U09422) ORF18 [Enterococcus faecalis] | |
| SEQ ID N^{o} 877 | LM-3017.1 | unknown, highly similar to TN916 ORF17 | 73 | gi\|532550\|gb\|AAB60016.1\|(U09422) ORF17 [Enterococcus faecalis] | |
| SEQ ID N^{o} 878 | LM-3018.1 | unknown, highly similar to TN916 ORF16 | 92 | gi\|532549\|gb\|AAB60017.1\|(U09422) ORF16 [Enterococcus faecalis] | |
| SEQ ID N^{o} 879 | LM-3020.1 | unknown, highly similar to TN916 ORF15 | 70 | gi\|532548\|gb\|AAB60018.1\|(U09422) ORF15 [Enterococcus faecalis] | |
| SEQ ID N^{o} 880 | LM-3022.1 | unknown, highly similar to TN916 ORF14 and | 82 | gi\|532547\|gb\|AAB60019.1\|(U09422) ORF14 [Enterococcus faecalis] | |
| | | to L. monocytogenes P60 protein | | | |
| SEQ ID N^{o} 881 | LM-3023.1 | unknown, highly similar to TN916 ORF13 | 71 | gi\|532546\|gb\|AAB60020.1\|(U09422) ORF13 [Enterococcus faecalis] | |
| SEQ ID N^{o} 882 | LM-3024.1 | CADMIUM EFFLUX SYSTEM ACCESSORY | 69 | gi\|3121831\|sp\|Q56405\|CADC_LISMO CADMIUM EFFLUX SYSTEM | |
| | | PROTEIN. | | ACCESSORY PROTEIN | |
| SEQ ID N^{o} 883 | LM-3056.1 | Unknown, similar to plsX protein involved in | 75 | gi\|6686325\|sp\|P71018\|PLSX_BACSU FATTY ACID/PHOSPHOLIPID SYNTHESIS | |
| | | fatty acid/phospholipid synthesis | | PROTEIN PLSX | |
| SEQ ID N^{o} 884 | LM-3101.1 | Unknown, similar to B. subtilis comG operon | 30 | gi\|3287181\|emb\|CAA75315.1\|(Y15043) homology to ComYD from | Length = 150 |
| | | protein 6 | | Streptcoccus gordonii, and ComGD from Bacillus subtilis [Lactococcus | |
| | | | | lactis subsp. cremoris] | |
| SEQ ID N^{o} 885 | LM-3116.2 | unknown, similar to hypothetical proteins | 63 | gi\|1730957\|sp\|P50839\|YPSB_BACSU HYPOTHETICAL 11.6 KD PROTEIN IN | |
| | | | | COTD-KDUD INTERGENIC REGION | |
| SEQ ID N^{o} 886 | LM-3169.1 | | 83 | gi\|466194\|sp\|P35163\|RESD_BACSU TRANSCRIPTIONAL REGULATORY | |
| | | | | PROTEIN RESD | |
| SEQ ID N^{o} 887 | LM-3181.2 | | 73 | gi\|1710383\|sp\|P46352\|RIPX_BACSU PROBABLE | |
| | | | | INTEGRASE/RECOMBINASE RIPX | |
| SEQ ID N^{o} 888 | LM-3244.3 | unknown, similar to carbonic anhydrase | 40 | gi\|4389447\|pdb\|1KOQ\|B Chain B, Neisseria Gonorrhoeae Carbonic | Length = 221 |
| | | | | Anhydrase | |
| SEQ ID N^{o} 889 | LM-3284.1 | Unknown, putative secreted protein | 49 | gi\|140696\|sp\|P10024\|YGl3_BACTU HYPOTHETICAL 13.4 KD PROTEIN (ORF3) | |
| SEQ ID N^{o} 890 | LM-3285.1 | Unknown, putative secreted protein | 34 | gi\|140696\|sp\|P10024\|YGI3_BACTU HYPOTHETICAL 13.4 KD PROTEIN (ORF3) | |
| SEQ ID N^{o} 891 | LM-3286.1 | Unknown, putative secreted protein | 50 | gi\|140696\|sp\|P10024\|YGI3_BACTU HYPOTHETICAL 13.4 KD PROTEIN (ORF3) | |
| SEQ ID N^{o} 892 | LM-3330.2 | Unknown, similar to oxetanocin A resistance | 70 | gi\|7474545\|pir\|\|F69784 conserved hypothetical protein ydhJ - Bacillus | |
| | | protein oxrB | | subtilis | |
| SEQ ID N^{o} 893 | LM-3331.1 | Unknown | | No Hits found | |
| SEQ ID N^{o} 894 | LM-3332.1 | Unknown | | No Hits found | |
| SEQ ID N^{o} 895 | LM-3333.1 | Unknown | | No Hits found | |
| SEQ ID N^{o} 896 | LM-3334.1 | Unknown | | No Hits found | |
| SEQ ID N^{o} 897 | LM-3335.2 | Unknown | 31 | gi\|7479713\|pir\|\|T35598 hypothetical protein SC6G9.01c - Streptomyces | |
| | | | | coelicolor (fragment) | |
| SEQ ID N^{o} 898 | LM-3336.3 | Unknown, weakly similar to site-specific DNA- | 24 | gi\|1769991\|emb\|CAA65779.1\| (X97069) site-specific DNA- | Length = 259 |
| | | methyltransferase | | methyltransferase [Bacillus stearothermophilus] | |
| SEQ ID N^{o} 899 | LM-3337.3 | Unknown | 18 | gi\|7463337\|pir\|\|G70163 hypothetical protein BB0512 - Lyme disease | |
| | | | | spirochete | |
| SEQ ID N^{o} 900 | LM-3344.1 | Unknown | 11 | gi\|7494294\|pir\|\|D71614 hypothetical protein PFB0460c - malaria parasite | |
| | | | | (Plasmodium falciparum) | |
| SEQ ID N^{o} 901 | LM-3361.2 | | 35 | gi\|1730885\|sp\|P50728\|YPBB_BACSU HYPOTHETICAL 40.7 KD PROTEIN IN FER-RECQ | |
| | | | | INTERGENIC REGION | |
| SEQ ID N^{o} 902 | LM-338.1 | Unknown, similar to lipase | 39 | gi\|7448882\|pir\|\|C69464 carboxylesterase (estA) homolog - | |
| | | | | Archaeoglobus fulgidus | |
| SEQ ID N^{o} 903 | LM-3398.1 | cysteinyl-tRNA synthetase | 76 | gi\|549024\|sp\|Q06752\|SYC_BACSU CYSTEINYL-TRNA SYNTHETASE | |
| | | | | (CYSTEINE--TRNA LIGASE) (CYSRS) | |
| SEQ ID N^{o} 904 | LM-3418.2 | unknown, peptidoglycan linked protein | 13 | gi\|2230998\|emb\|CAA65738.1\| (X97014) ORF A [Listeria seeligeri] | Length = 902 |
| | | (LPxTG) | | | |
| SEQ ID N^{o} 905 | LM-345.1 | Unknown, similar to transcription regulator | 46 | gi\|7442876\|pir\|\|F70203 xylose operon regulatory protein (xyIR-2) | |
| | | | | homolog - Lyme disease spirochete | |
| SEQ ID N^{o} 906 | LM-3463.2 | unknown | | No Hits found | |
| SEQ ID N^{o} 907 | LM-3469.2 | Unknown | | No Hits found | |
| SEQ ID N^{o} 908 | LM-3477.1 | Unknown, similar to internalin | 42 | gi\|2347102\|gb\|AAB67968.1\| (U77367) internalin [Listeria | Length = 821 |
| | | | | monocytogenes] | |
| SEQ ID N^{o} 909 | LM-3480.3 | Unknown | 54 | gi\|7475781\|pir\|\|A70002 protein kinase homolog ytvA - Bacillus subtilis | |
| SEQ ID N^{o} 910 | LM-3494.3 | unknown, similar to B. subtilis protein YkvS | 53 | gi\|7475155\|pir\|\|B69869 hypothetical protein ykvS - Bacillus subtilis | |
| SEQ ID N^{o} 911 | LM-3512.1 | Unknown, similar to conjugated bile acid | 82 | gi\|729058\|sp\|Q06115\|CBH_LACPL CHOLOYLGLYCINE HYDROLASE | |
| | | hydrolase | | (CONJUGATED BILE ACID HYDROLASE) (CBAH) (BILE SALT | |
| | | | | HYDROLASE) | |
| SEQ ID N^{o} 912 | LM-3517.2 | Unknown, similar to unknown proteins | 73 | gi\|7429544\|pir\|\|E69879 conserved hypothetical protein yloV - Bacillus | |
| | | | | subtilis | |
| SEQ ID N^{o} 913 | LM-3528.2 | Unknown, similar to heme O oxygenase | 71 | gi\|4584149\|emb\|CAB40605.1\| (AJ010111) cytochrome aa3 controlling | Length = 311 |
| | | | | protein [Bacillus cereus] | |
| SEQ ID N^{o} 914 | LM-3538.3 | unknown, similar to transcriptional regulator, | 50 | gi\|7442860\|pir\|\|D69834 transcription regulator Lacl family homolog yhjM - | |
| | | Lacl family | | Bacillus subtilis | |
| SEQ ID N^{o} 915 | LM-3562.2 | | | No Hits found | |
| SEQ ID N^{o} 916 | LM-3582.1 | Unknown, similar to transcription regulator | 43 | gi\|7521750\|pir\|\|C70487 transcription regulator TetR/AcrR family - Aquifex | |
| | | TetR/AcrR family | | aeolicus | |
| SEQ ID N^{o} 917 | LM-360.1 | unknown | | No Hits found | |
| SEQ ID N^{o} 918 | LM-3609.1 | unknown, similar to AUTOLYSIN (EC 3.5.1.28) | 24 | gi\|584748\|sp\|P37710\|ALYS_ENTFA AUTOLYSIN (N- | |
| | | (N-ACETYLMURAMOYL-L-ALANINE | | ACETYLMURAMOYL-L-ALANINE AMIDASE) | |
| | | AMIDASE) | | | |
| SEQ ID N^{o} 919 | LM-3612.1 | unknown, similar to TEICHOIC ACID | 35 | gi\|1074220\|pir\|\|S49240 hypothetical protein 3 (capsulation locus) - | |
| | | BIOSYNTHESIS PROTEIN B PRECURSOR | | Haemophilus influenzae (strain RM107) | |
| SEQ ID N^{o} 920 | LM-3614.3 | unknown, similar to B. subtilis YfhO protein | 40 | gi\|7475003\|pir\|\|G69801 hypothetical protein yfhO - Bacillus subtilis | |
| SEQ ID N^{o} 921 | LM-3676.2 | | 65 | gi\|1706797\|sp\|P49937\|FHUG_BACSU FERRICHROME TRANSPORT | |
| | | | | SYSTEM PERMEASE PROTEIN FHUG | |
| SEQ ID N^{o} 922 | LM-3681.2 | unknown | | No Hits found | |
| SEQ ID N^{o} 923 | LM-3691.2 | Unknown, putative peptdoglycan bound protein | 31 | gi\|7470965\|pir\|\|T28679 fibrinogen-binding protein homolog - | |
| | | (LPXTG motif) BEST-BLASTP = | | Staphylococcus aureus | |
| SEQ ID N^{o} 924 | LM-3700.2 | Unknwon, peptidoglycan anchored protein | 22 | gi\|7470965\|pir\|\|T28679 fibrinogen-binding protein homolog - | |
| | | | | Staphylococcus aureus | |
| SEQ ID N^{o} 925 | LM-3728.1 | unknown | | No Hits found | |
| SEQ ID N^{o} 926 | LM-3746.2 | Unknown, similar to N-acetyltransferase | 29 | gi\|7427903\|pir\|\|B70064 probable phosphinothricin N-acetyltransferase | |
| | | | | (EC 2.3.1.-) ywnH - Bacillus subtilis | |
| SEQ ID N^{o} 927 | LM-375.1 | Unknwon, similar to oxidoreductases | 68 | gi\|7447923\|pir\|\|H72307 oxidoreductase, aldo/keto reductase family - | |
| | | | | Thermotoga maritima (strain MSB8) | |
| SEQ ID N^{o} 928 | LM-3750.2 | Unknown, similar to transposase | 57 | gi\|7474337\|pir\|\|H59102 hypothetical protein pXO1-96 - Bacillus anthracis | |
| | | | | virulence plasmid pXO1 | |
| SEQ ID N^{o} 929 | LM-3754.2 | | | No Hits found | |
| SEQ ID N^{o} 930 | LM-376.1 | Unknown, similar to transcription regulator | 60 | gi\|7444887\|pir\|\|B69970 transcription regulator MerR family homolog yraB - | |
| | | (merR family) | | Bacillus subtilis | |
| SEQ ID N^{o} 931 | LM-377.1 | Unknwon | | No Hits found | |
| SEQ ID N^{o} 932 | LM-3779.3 | internalin C | 92 | gi\|1546905\|emb\|CAA65088.1\| (X95822) internalin family protein [Listeria | |
| | | | | monocytogenes] | |
| SEQ ID N^{o} 933 | LM-378.1 | Unknwon | | No Hits found | |
| SEQ ID N^{o} 934 | LM-379.1 | Unknwon | | No Hits found | |
| SEQ ID N^{o} 935 | LM-380.1 | Unknwon | | No Hits found | |
| SEQ ID N^{o} 936 | LM-3811.3 | Unknown | 20 | gi\|2128791\|pir\|\|A64465 hypothetical protein MJ1322 - Methanococcus | |
| | | | | jannaschii | |
| SEQ ID N^{o} 937 | LM-3836.1 | | | No Hits found | |
| SEQ ID N^{o} 938 | LM-3848.1 | riboflavin kinase and FAD synthase | 66 | gi\|7444638\|pir\|\|D69692 riboflavin kinase/FAD synthase ribC - Bacillus | |
| | | | | subtilis | |
| SEQ ID N^{o} 939 | LM-3853.2 | Unknown, weakly similar to transposase | 45 | gi\|7474193\|pir\|\|T09011 probable transposase TnpA - Streptococcus | |
| | | | | pyogenes (fragment) | |
| SEQ ID N^{o} 940 | LM-387.1 | Unknown | | No Hits found | |
| SEQ ID N^{o} 941 | LM-388.1 | unknown | | No Hits found | |
| SEQ ID N^{o} 942 | LM-3887.1 | unknown | | No Hits found | |
| SEQ ID N^{o} 943 | LM-389.1 | unknown | | No Hits found | |
| SEQ ID N^{o} 944 | LM-3890.1 | Unknown | 78 | gi\|6226126\|sp\|O32233\|SECG_BACSU PROBABLE PROTEIN-EXPORT | |
| | | | | MEMBRANE PROTEIN SECG | |
| SEQ ID N^{o} 945 | LM-390.3 | unknown | | No Hits found | |
| SEQ ID N^{o} 946 | LM-3905.2 | Unknown | | No Hits found | |
| SEQ ID N^{o} 947 | LM-392.3 | unknown | | No Hits found | |
| SEQ ID N^{o} 948 | LM-3929.1 | unknown | | No Hits found | |
| SEQ ID N^{o} 949 | LM-3934.1 | Unknown, similar to transposase | 62 | gi\|929968\|gb\|AAA74024.1\| (U30713) ORFA [Bacillus anthracis] | |
| SEQ ID N^{o} 950 | LM-395.2 | Unknown, similar to dinitrogenase reductase | 47 | gi\|6136600\|sp\|Q58588\|YB87_METJA HYPOTHETICAL PROTEIN MJ1187 | |
| | | ADP-ribosylation system | | | |
| SEQ ID N^{o} 951 | LM-3951.1 | Unknown | 14 | gi\|1389737\|gb\|AAB03089.1\| (U55187) arabinosidase [Butyrivibrio | Length = 789 |
| | | | | fibrisolvens] | |
| SEQ ID N^{o} 952 | LM-3953.1 | Hypothetical orf | | No Hits found | |
| SEQ ID N^{o} 953 | LM-3954.2 | Unknown | | No Hits found | |
| SEQ ID N^{o} 954 | LM-3958.1 | | 58 | gi\|5929908\|gb\|AAD56637.1\|AF174588_1 (AF174588) ComK [Listeria | Length = 190 |
| | | | | monocytogenes] | |
| SEQ ID N^{o} 955 | LM-3973.2 | LIPOPROTEIN SIGNAL PEPTIDASE (EC | 56 | gi\|400202\|sp\|P31024\|LSPA_STAAU LIPOPROTEIN SIGNAL PEPTIDASE | |
| | | 3.4.23.36) (PROLIPOPROTEIN SIGNAL | | (PROLIPOPROTEIN SIGNAL PEPTIDASE) (SIGNAL PEPTIDASE II) | |
| | | PEPTIDASE) (SIGNAL PEPTIDASE II) | | (SPASE II) | |
| | | (SPASE II). | | | |
| SEQ ID N^{o} 956 | LM-3976.4 | Unknown, similar to repressor (penicilinase | 53 | sp\|P06555\|BLAI_BACLI PENICILLINASE REPRESSOR (REGULATORY | |
| | | repressor) | | PROTEIN BLAI) (BETA-LACTAMASE REPRESSOR PROTEIN) | |
| SEQ ID N^{o} 957 | LM-3995.1 | unknown | | No Hits found | |
| SEQ ID N^{o} 958 | LM-4013.2 | unknown, similar to transcriptional regulator | 56 | gi\|1730943\|sp\|P54182\|YPOP_BACSU HYPOTHETICAL TRANSCRIPTIONAL | |
| | | (MarR family) | | REGULATOR IN UVRX-ILVA INTERGENIC REGION | |
| SEQ ID N^{o} 959 | LM-402.1 | Unknown | | No Hits found | |
| SEQ ID N^{o} 960 | LM-4040.1 | Unknown | | No Hits found | |
| SEQ ID N^{o} 961 | LM-4065.1 | | 47 | gi\|7480201\|pir\|\|T37067 hypothetical protein SCJ21.18c - Streptomyces | |
| | | | | coelicolor (fragment) | |
| SEQ ID N^{o} 962 | LM-4096.1 | Unknown | | No Hits found | |
| SEQ ID N^{o} 963 | LM-4097.1 | Unknown | | No Hits found | |
| SEQ ID N^{o} 964 | LM-4106.1 | Unknown, similar to putative transcription | 72 | gi\|7474280\|pir\|\|H59095 hypothetical protein pXO1-40 - Bacillus anthracis | |
| | | regulator | | virulence plasmid pXO1 | |
| SEQ ID N^{o} 965 | LM-4118.1 | Unknown | | No Hits found | |
| SEQ ID N^{o} 966 | LM-4119.1 | unknown | | No Hits found | |
| 5EQ ID N^{o} 967 | LM-4121.1 | unknown | | No Hits found | |
| SEQ ID N^{o} 968 | LM-4137.1 | unknown | | No Hits found | |
| SEQ ID N^{o} 969 | LM-4147.1 | unknown, highly similar to TN916 ORF8 | 61 | gi\|431129\|gb\|AAC36979.1\| (L15633) [Conjugative transposon Tn916 | |
| | | | | (from Enterococcus faecalis, DS16), 3′ end.], gene products [Transposon | |
| | | | | Tn916] | |
| SEQ ID N^{o} 970 | LM-4148.1 | unknown, highly similar to TN916 ORF19 | 89 | gi\|532552\|gb\|AAB60014.1\| (U09422) ORF19 [Enterococcus faecalis] | |
| SEQ ID N^{o} 971 | LM-4149.2 | unknown | 18 | gi\|6325248\|ref\|NP_015316.1\|YpI009cp | |
| SEQ ID N^{o} 972 | LM-4152.2 | unknown, similar to transcriptional regulator | | No Hits found | |
| SEQ ID N^{o} 973 | LM-4166.1 | Unknown, hypothetical gene | | No Hits found | |
| SEQ ID N^{o} 974 | LM-417.1 | hosphoribosylaminoimidazole carboxylase I | 84 | gi\|131626\|sp\|P12044\|PUR6_BACSU PHOSPHORIBOSYLAMINOIMIDAZOLE | |
| | | | | CARBOXYLASE CATALYTIC SUBUNIT (AIR CARBOXYLASE) (AIRC) | |
| SEQ ID N^{o} 975 | LM-4174.1 | unknown | | No Hits found | |
| SEQ ID N^{o} 976 | LM-4175.1 | unknown | 53 | gi\|7475018\|pir\|\|F69808 hypothetical protein yfkK - Bacillus subtilis | |
| SEQ ID N^{o} 977 | LM-4188.1 | | | No Hits found | |
| SEQ ID N^{o} 978 | LM-4192.1 | unknown | | No Hits found | |
| SEQ ID N^{o} 979 | LM-4193.1 | unknown | | No Hits found | |
| SEQ ID N^{o} 980 | LM-4195.1 | Unknown | | No Hits found | |
| SEQ ID N^{o} 981 | LM-4197.1 | Unknown | | No Hits found | |
| SEQ ID N^{o} 982 | LM-4200.1 | unknown | | No Hits found | |
| SEQ ID N^{o} 983 | LM-4203.1 | | 49 | gi\|7475129\|pir\|\|G69854 hypothetical protein yjzD - Bacillus subtilis | |
| SEQ ID N^{o} 984 | LM-4207.1 | unknwon | 48 | gi\|5730320\|emb\|CAB52541.1\| (AJ131519) hypothetical protein | Length = 61 |
| | | | | [Lactobacillus bacteriophage phi adh] | |
| SEQ ID N^{o} 985 | LM-4209.1 | Bacteriophage A118 gp65 protein | 69 | gi\|5823667\|emb\|CAB53855.1\| (AJ242593) gp65 [Bacteriophage A118] | Length = 54 |
| SEQ ID N^{o} 986 | LM-4211.1 | | | No Hits found | |
| SEQ ID N^{o} 987 | LM-4213.1 | Unknown | | No Hits found | |
| SEQ ID N^{o} 988 | LM-4214.1 | Unknown | | No Hits found | |
| SEQ ID N^{o} 989 | LM-4215.1 | gp44 [Bacteriophage A118] | 57 | gi\|5823646\|emb\|CAB53834.1\| (AJ242593) gp44 [Bacteriophage A118] | Length = 72 |
| SEQ ID N^{o} 990 | LM-4216.1 | Unknown, similar to transcription regulator | 53 | gi\|1176725\|sp\|P45903\|YQAF_BACSU HYPOTHETICAL TRANSCRIPTIONAL | |
| | | | | REGULATOR IN SPOIIIC-CWLA INTERGENIC REGION (ORF8) | |
| SEQ ID N^{o} 991 | LM-4226.1 | | | No Hits found | |
| SEQ ID N^{o} 992 | LM-4227.1 | Unknown, similar to repressor protein | 67 | gi\|7521352\|pir\|\|G75183 probable repressor protein PAB7155 - | |
| | | | | Pyrococcus abyssi (strain Orsay) | |
| SEQ ID N^{o} 993 | LM-4236.1 | unknown | | No Hits found | |
| SEQ ID N^{o} 994 | LM-4246.1 | Unknwon, hypothetical protein | | No Hits found | |
| SEQ ID N^{o} 995 | LM-4251.1 | Unknown, Hypothetical | | No Hits found | |
| SEQ ID N^{o} 996 | LM-4262.2 | Unknown, similar to penicillinase antirepressor | 44 | sp\|P12287\|BLAR_BACLI REGULATORY PROTEIN BLAR1 | |
| SEQ ID N^{o} 997 | LM-4267.1 | unknown, similar to regulatory proteins | 18 | gi\|2495368\|sp\|Q56070\|MARA_SALTY MULTIPLE ANTIBIOTIC RESISTANCE | |
| | | | | PROTEIN MARA | |
| SEQ ID N^{o} 998 | LM-4268.1 | unknown | | No Hits found | |
| SEQ ID N^{o} 999 | LM-4295.1 | | | No Hits found | |
| SEQ ID N^{o} 1000 | LM-4342.1 | | 46 | pir\|\|H70081 hypothetical protein yxIE - Bacillus subtilis dbj\|BAA11736.1\| | Length = 62 |
| | | | | (D83026) hypothetical [Bacillus subtilis] emb\|CAB15893.1\| (Z99123) yxIE | |
| | | | | [Bacillus subtilis] | |
| SEQ ID N^{o} 1001 | LM-4351.1 | | | No Hits found | |
| SEQ ID N^{o} 1002 | LM-48.1 | Unknown, conserved hypothetical protein | 40 | gi\|4895134\|gb\|AAD32741.1\| (AF127374) MmcQ [Streptomyces lavendulae] | Length = 123 |
| SEQ ID N^{o} 1003 | LM-49.1 | Unknown | | No Hits found | |
| SEQ ID N^{o} 1004 | LM-494.1 | Unknown, similar to transcriptional regulator | 29 | gi\|7478115\|pir\|\|H70940 probable helix-turn-helix motif at aa 18-39 - | |
| | | | | Mycobacterium tuberculosis (strain H37RV) | |
| SEQ ID N^{o} 1005 | LM-497.1 | Unknown, weakly similar to | 12 | gi\|5823630\|emb\|CAB53818.1\| (AJ242593) gp32 [Bacteriophage A118] | Length = 246 |
| | | gp32_Bacteriophage A118 protein | | | |
| SEQ ID N^{o} 1006 | LM-501.1 | | | No Hits found | |
| SEQ ID N^{o} 1007 | LM-502.1 | Unknown | | No Hits found | |
| SEQ ID N^{o} 1008 | LM-506.1 | Unknown, similar to anti-repressor | 83 | gi\|5823644\|emb\|CAB53832.1\| (AJ242593) putative anti-repressor | Length = 262 |
| | | [Bacteriophage A118] BEST-BLASTP = | | promoter [Bacteriophage A118] | |
| SEQ ID N^{o} 1009 | LM-509.1 | Unknown, similar to bacteriophage proteins | 48 | gi\|6599320\|emb\|CAB63666.1\| (AJ251789) hypothetical protein | Length = 163 |
| | | | | [Lactobacillus casei bacteriophage A2] | |
| SEQ ID N^{o} 1010 | LM-51.1 | Unknown, weakly similar to AraC-like | 17 | gi\|7387707\|sp\|O87389\|GLXA_RHIME TRANSCRIPTIONAL REGULATOR | |
| | | transcription regulator | | GLXA | |
| SEQ ID N^{o} 1011 | LM-510.1 | Unknown | 26 | gi\|6015511\|emb\|CAA63097.1\| (X92187) p22 erf-like protein [unidentified] | Length = 207 |
| SEQ ID N^{o} 1012 | LM-512.1 | Unknown, similar to protein gp49 | 63 | gi\|5823651\|emb\|CAB53839.1\| (AJ242593) gp49 [Bacteriophage A118] | Length = 310 |
| | | [Bacteriophage A118] | | | |
| SEQ ID N^{o} 1013 | LM-514.1 | Unknown, similar to protein gp51 | 59 | gi\|5823653\|emb\|CAB53841.1\| (AJ242593) gp51 [Bacteriophage A118] | Length = 186 |
| | | [Bacteriophage A118] | | | |
| SEQ ID N^{o} 1014 | LM-517.1 | Unknown, similar to a bacteriophage protein | 34 | gi\|5001708\|gb\|AAD37108.1\|AF109874_14 (AF109874) unknown | Length = 131 |
| | | | | [Bacteriophage Tuc2009] | |
| SEQ ID N^{o} 1015 | LM-518.1 | Unknown | 26 | gi\|6226483\|sp\|Q52118\|YMO3_ERWST HYPOTHETICAL 31.4 KDA PROTEIN | |
| | | | | IN MOBD 3′REGION | |
| SEQ ID N^{o} 1016 | LM-520.1 | Unknown | | No Hits found | |
| SEQ ID N^{o} 1017 | LM-527.1 | | | No Hits found | |
| SEQ ID N^{o} 1018 | LM-529.1 | | 37 | gi\|5823599\|emb\|CAB53787.1\| (AJ242593) putative terminase small | Length = 180 |
| | | | | subunit [Bacteriophage A118] | |
| SEQ ID N^{o} 1019 | LM-535.1 | | 56 | gi\|2120257\|pir\|\|S58142 coat protein - phage SPP1 | |
| SEQ ID N^{o} 1020 | LM-541.1 | | 71 | gi\|5823611\|emb\|CAB53799.1\| (AJ242593) gp13 [Bacteriophage A118] | Length = 110 |
| SEQ ID N^{o} 1021 | LM-549.1 | | 94 | gi\|5823615\|emb\|CAB53803.1\| (AJ242593) gp17 [Bacteriophage A118] | Length = 272 |
| SEQ ID N^{o} 1022 | LM-552.1 | | 99 | gi\|5823617\|emb\|CAB53805.1\| (AJ242593) gp19 [Bacteriophage A118] | Length = 342 |
| SEQ ID N^{o} 1023 | LM-553.1 | | 91 | gi\|5823618\|emb\|CAB53806.1\| (AJ242593) gp20 [Bacteriophage A118] | Length = 357 |
| SEQ ID N^{o} 1024 | LM-554.1 | | 86 | gi\|5823619\|emb\|CAB53807.1\| (AJ242593) gp21 [Bacteriophage A118] | Length = 105 |
| SEQ ID N^{o} 1025 | LM-558.1 | | 99 | gi\|2801778\|gb\|AAC38580.1\| (AF042193) peptidoglycan lytic enzyme | Length = 281 |
| | | | | [Listeria monocytogenes] | |
| SEQ ID N^{o} 1026 | LM-559.1 | | | No Hits found | |
| SEQ ID N^{o} 1027 | LM-56.1 | Unknown, similar to hydrolase (esterase) | 43 | gi\|7474811\|pir\|\|E70010 dihydrolipoamide S-acetyltransferase homolog | |
| | | | | yugF - Bacillus subtilis | |
| SEQ ID N^{o} 1028 | LM-560.1 | | 47 | gi\|7460036\|pir\|\|T13226 hypothetical protein R232 - Lactobacillus phage | |
| | | | | phi-gle | |
| SEQ ID N^{o} 1029 | LM-561.1 | | | No Hits found | |
| SEQ ID N^{o} 1030 | LM-587.1 | Unknown, similar to Bacillus anthracis CapA | 63 | gi\|4584121\|emb\|CAB40617.1\| (AJ007788) related sequence M24150 | Length = 367 |
| | | protein (polyglutamate capsule biosynthesis) | | [Bacillus cereus] | |
| SEQ ID N^{o} 1031 | LM-611.1 | Unknown, similar to ornithine | 58 | gi\|6685709\|sp\|O93656\|OTC_PYRAB ORNITHINE | |
| | | carbamoyltransferase | | CARBAMOYLTRANSFERASE (OTCASE) | |
| SEQ ID N^{o} 1032 | LM-613.1 | Unknown, similar to amino acid transporter | 42 | gi\|6009438\|dbj\|BAA84897.1\| (AB024946) orf62 [Escherichia coli] | Length = 486 |
| SEQ ID N^{o} 1033 | LM-614.1 | Unknown, conserved hypothetical protein | 61 | gi\|5712716\|gb\|AAD47622.1\| (AF153708) unknown [Pseudomonas sp. | Length = 376 |
| | | | | BG33R] | |
| SEQ ID N^{o} 1034 | LM-615.1 | carbamate kinase | 76 | gi\|6980398\|pdb\|1B7B\|A Chain A, Carbamate Kinase From Enterococcus | |
| | | | | Faecium | |
| SEQ ID N^{o} 1035 | LM-616.1 | Unknown, conserved hypothetical protein | 58 | gi\|5712716\|gb\|AAD47622.1\| (AF153708) unknown [Pseudomonas sp. | Length = 376 |
| | | | | BG33R] | |
| SEQ ID N^{o} 1036 | LM-617.1 | Unknown, conserved hypothetical protein, | 43 | gi\|4206184\|gb\|AAD11507.1\| (U60828) Unknown [Lactococcus lactis] | Length = 244 |
| | | hypothetical regulator | | | |
| SEQ ID N^{o} 1037 | LM-653.1 | Unknown, similar to flagellar motor switch | 53 | gi\|2120517\|pir\|\|JC4505 flagellar motor switch protein fliY - syphilis | |
| | | protein fliY C-terminal part | | spirochete | |
| SEQ ID N^{o} 1038 | LM-692.1 | Unknown | | No Hits found | |
| SEQ ID N^{o} 1039 | LM-710.1 | | 67 | gi\|2833406\|sp\|Q46829\|BGLA_ECOLI 6-PHOSPHO-BETA-GLUCOSIDASE | |
| | | | | BGLA | |
| SEQ ID N^{o} 1040 | LM-711.2 | | 67 | gi\|730418\|sp\|P40739\|PTBA_BACSU PTS SYSTEM, BETA-GLUCOSIDES- | |
| | | | | SPECIFIC IIABC COMPONENT (EIIABC-BGL) (BETA-GLUCOSIDES- | |
| | | | | PERMEASE IIABC COMPONENT) (PHOSPHOTRANSFERASE | |
| | | | | ENZYME II, ABC COMPONENT) (EII-BGL) | |
| SEQ ID N^{o} 1041 | LM-712.2 | | 70 | gi\|729940\|sp\|P39805\|LICT_BACSU TRANSCRIPTION ANTITERMINATOR | |
| | | | | LICT | |
| SEQ ID N^{o} 1042 | LM-716.1 | | | No Hits found | |
| SEQ ID N^{o} 1043 | LM-721.1 | | 51 | gi\|7434480\|pir\|\|B69785 cellobiose phosphotransferase system enzym | |
| | | | | homolog ydhN - Bacillus subtilis | |
| SEQ ID N^{o} 1044 | LM-723.1 | | 63 | gi\|6002243\|emb\|CAB56688.1\| (AL121596) Beta-glucosidase (EC | Length = 762 |
| | | | | 3.2.1.21) [Streptomyces coelicolor A3(2)] | |
| SEQ ID N^{o} 1045 | LM-724.1 | | 62 | gi\|7450520\|pir\|\|\|A69785 cellobiose phosphotransferase system enzym | |
| | | | | homolog ydhM - Bacillus subtilis | |
| SEQ ID N^{o} 1046 | LM-725.1 | Unknown, similar to cellobiose | 55 | gi\|7449992\|pir\|\|C69785 cellobiose phosphotransferase system enzym | |
| | | phosphotransferase system enzyme IIC | | homolog ydhO - Bacillus subtilis | |
| SEQ ID N^{o} 1047 | LM-726.1 | Unknown, similar to lichenan operon | 55 | gi\|1168885\|sp\|P46321\|CELR_BACSU PUTATIVE CEL OPERON REGULATOR | |
| | | transcription antiterminator licR | | | |
| SEQ ID N^{o} 1048 | LM-728.1 | Unknown | 97 | gi\|4138150\|emb\|CAA07718.1\| (AJ007877) ADP ribosyl glycohydrolase | Length = 327 |
| | | | | [Listeria monocytogenes] | |
| SEQ ID N^{o} 1049 | LM-730.1 | beta-glucoside-specific phosphotransferase | 94 | gi\|4138149\|emb\|CAA07717.1\| (AJ007877) PTS enzyme II [Listeria | Length = 640 |
| | | enzyme II | | monocytogenes] | |
| SEQ ID N^{o} 1050 | LM-731.2 | transcription antiterminator | 96 | gi\|4138148\|emb\|CAA07716.1\| (AJ007877) antiterminator [Listeria | Length = 270 |
| | | | | monocytogenes] | |
| SEQ ID N^{o} 1051 | LM-757.1 | Unknown, similar to internalin proteins | 38 | gi\|2347102\|gb\|AAB67968.1\| (U77367) internalin [Listeria | Length = 821 |
| | | | | monocytogenes] | |
| SEQ ID N^{o} 1052 | LM-80.1 | unknown | | No Hits found | |
| SEQ ID N^{o} 1053 | LM-81 2.1 | Unknown, similar to unknown protein | 43 | gi\|4033715\|gb\|AAC97152.1\| (U49397) unknown [Streptococcus pyogenes] | Length = 591 |
| SEQ ID N^{o} 1054 | LM-842.1 | Unknown | 57 | gi\|1044888\|emb\|CAA63151.1\| (X92423) sepA [Listeria monocytogenes] | Length = 391 |
| SEQ ID N^{o} 1055 | LM-843.1 | Unknown | | No Hits found | |
| SEQ ID N^{o} 1056 | LM-857.1 | Unknown, similar to transcription regulator | 48 | gi\|7443060\|pir\|\|D70044 transcription regulator GntR family homolog yvoA - | |
| | | GntR family | | Bacillus subtilis | |
| SEQ ID N^{o} 1057 | LM-858.1 | Unknown, weakly similar to mannose-6- | 40 | gi\|7480238\|pir\|\|T37128 hypothetical protein SCJ4.45c - Streptomyces | |
| | | phosphate isomerase | | coelicolor | |
| SEQ ID N^{o} 1058 | LM-879.1 | Unknown | 49 | gi\|3786190\|emb\|CAA71106.1\| (Y09988) hypothetical protein [Listeria | Length = 171 |
| | | | | ivanovii] | |
| SEQ ID N^{o} 1059 | LM-880.1 | Unknown | | No Hits found | |
| SEQ ID N^{o} 1060 | LM-881.1 | unknown | | No Hits found | |
| SEQ ID N^{o} 1061 | LM-896.1 | | | No Hits found | |
| SEQ ID N^{o} 1062 | LM-906.1 | Unknown, conserved hypothetical protein | 34 | gi\|7476217\|pir\|\|B70617 hypothetical protein Rv0143c - Mycobacterium | |
| | | | | tuberculosis (strain H37RV) | |
| SEQ ID N^{o} 1063 | LM-924.1 | ABC-transporter ATP binding protein | 60 | gi\|7479118\|pir\|\|T34822 ABC-transporter ATP binding protein - | |
| | | | | Streptomyces coelicolor | |
| SEQ ID N^{o} 1064 | LM-968.1 | Unknown, similar to transposase | 62 | gi\|929968\|gb\|AAA74024.1\| (U30713) ORFA [Bacillus anthracis] | |
| SEQ ID N^{o} 1065 | LM-969.1 | Unknown, similar to transposase (N-terminal | 49 | gi\|7474337\|pir\|\|H59102 hypothetical protein pXO1-96 - Bacillus anthracis | |
| | | part) | | virulence plasmid pXO1 | |
| SEQ ID N^{o} 1066 | LM-970.1 | Unknown, similar to transposase C-terminal | 59 | gi\|7474337\|pir\|\|H59102 hypothetical protein pXO1-96 - Bacillus anthracis | |
| | | part | | virulence plasmid pXO1 | |
| SEQ ID N^{o} 1067 | LM-973.1 | Unknwon, similar to internalin proteins | 24 | gi\|2347105\|gb\|AAB67970.1\| (U77368) inID [Listeria monocytogenes] | Length = 567 |

Table VI: Legends

SEQ ID Nos. 2059-2601: nucleotide sequences of 543 genes specific for Listeria innocua Clip11262; with, in the first column, the SEQ ID identifier, in the second column the name of the gene, in the third column the IPF number (<<Pasteur Institute>> identifier number abbreviation, making it possible to correlate the sequence with the sequences of Table V) and in the last column the corresponding annotation.

TABLE VI

| SEQ ID | Name | IPF ID | Function |
|---|---|---|---|
| SEQ ID N° 2059 | pli0001 | 4106.2 | Unknown, similar to insertion sequence ATP binding protein |
| SEQ ID N^{o} 2060 | pli0002 | 6602.1 | Unknown |
| SEQ ID N^{o} 2061 | pli0003 | 4103.1 | Unknown, similar to transposase |
| SEQ ID N^{o} 2062 | pli0004 | 4102.1 | Unknown, similar to DNA methylase |
| SEQ ID N^{o} 2063 | pli0005 | 4099.1 | Unknown |
| SEQ ID N^{o} 2064 | pli0006 | 4098.1 | Unknown |
| SEQ ID N^{o} 2065 | pli0007 | 4097.1 | Unknown |
| SEQ ID N^{o} 2066 | pli0008 | 4095.1 | Unknown, similar to unknown protein |
| SEQ ID N^{o} 2067 | pli0009 | 4092.1 | Unknown, similar to unknown protein |
| SEQ ID N^{o} 2068 | pli0010 | 4088.1 | Unknown |
| SEQ ID N^{o} 2069 | pli0011 | 4086.1 | Unknown, hypothetical gene |
| SEQ ID N^{o} 2070 | pli0012 | 4084.1 | Transposase |
| SEQ ID N^{o} 2071 | pli0013 | 4081.1 | Unknown, similar to unknown protein |
| SEQ ID N^{o} 2072 | pli0014 | 4079.1 | Unknown, similar to unknown protein |
| SEQ ID N^{o} 2073 | pli0015 | 4077.1 | Unknown, similar to plasmid replication protein |
| SEQ ID N^{o} 2074 | pli0016 | 6241.1 | Unknown, similar to transposase C-terminal part |
| SEQ ID N^{o} 2075 | pli0017 | 4073.1 | Unknown |
| SEQ ID N^{o} 2076 | pli0018 | 4195.3 | transposase (truncated) |
| SEQ ID N^{o} 2077 | pli0019 | 4194.3 | Transposase, truncated |
| SEQ ID N^{o} 2078 | pli0020 | 4193.1 | Unknown, similar to transposase |
| SEQ ID N^{o} 2079 | pli0021 | 4192.1 | Unknown, similar to putative helicase |
| SEQ ID N^{o} 2080 | pli0022 | 4184.1 | Unknown, similar to plasmid replication protein B |
| SEQ ID N^{o} 2081 | pli0023 | 4183.1 | unknown, similar to plasmid replication initiation protein |
| SEQ ID N^{o} 2082 | pli0024 | 4182.1 | Unknown, similar to transposase |
| SEQ ID N^{o} 2083 | pli0025 | 4181.1 | Unknown, similar to transposase |
| SEQ ID N^{o} 2084 | pli0026 | 4179.1 | Unknown, similar to gram positive plasmid replication protein B |
| SEQ ID N^{o} 2085 | pli0027 | 4178.1 | Unknown, similar to helicase |
| SEQ ID N^{o} 2086 | pli0028 | 6113.2 | Transposase |
| SEQ ID N^{o} 2087 | pli0029 | 6111.1 | Unknown, similar to DNA methyltransferase |
| SEQ ID N^{o} 2088 | pli0030 | 6109.1 | Unknown |
| SEQ ID N^{o} 2089 | pli0031 | 6606.1 | Unknown |
| SEQ ID N^{o} 2090 | pli0032 | 6106.2 | Transposase |
| SEQ ID N^{o} 2091 | pli0033 | 6128.2 | Pseudogene, similar to C-terminal part of arsenite-translocating ATPase |
| SEQ ID N^{o} 2092 | pli0034 | 6127.1 | Unknown, similar to arsenical resistance operon repressor |
| SEQ ID N^{o} 2093 | pli0035 | 6126.1 | unknown, similar to arsenical resistance operon trans-acting repressor ArsD |
| SEQ ID N^{o} 2094 | pli0036 | 6125.1 | Unknown, similar to arsenical resistance operon repressor |
| SEQ ID N^{o} 2095 | pli0037 | 6124.1 | Unknown, similar to arsenical pump-driving ATPase |
| SEQ ID N^{o} 2096 | pli0038 | 6122.1 | unknown, similar to possible arsenic resistance membrane transport protein ArsB |
| SEQ ID N^{o} 2097 | pli0039 | 6120.1 | Unknown, similar to heavy metal membrane efflux protein |
| SEQ ID N^{o} 2098 | pli0040 | 6118.1 | Unknown, similar to flavoprotein oxidoreductase |
| SEQ ID N^{o} 2099 | pli0041 | 6115.1 | Unknown, similar to ABC transporter ATP-binding protein |
| SEQ ID N^{o} 2100 | pli0042 | 6605.1 | Similar to transposase, N-terminal part |
| SEQ ID N^{o} 2101 | pli0043 | 4177.2 | Transposase |
| SEQ ID N^{o} 2102 | pli0044 | 4175.1 | Unknown, similar to NADH peroxidase |
| SEQ ID N^{o} 2103 | pli0045 | 4174.1 | Pseudogene, similar to glycine-betaine binding protein (ABC transporter) |
| SEQ ID N^{o} 2104 | pli0046 | 4170.1 | Unknown, hypothetical protein |
| SEQ ID N^{o} 2105 | pli0047 | 4169.1 | Unknown |
| SEQ ID N^{o} 2106 | pli0048 | 4168.1 | Unknown, similar to heavy metal-transporting ATPase |
| SEQ lD N^{o} 2107 | pli0049 | 4166.1 | Transposase |
| SEQ ID N^{o} 2108 | pli0050 | 4164.1 | Unknown, similar to the two components sensor protein kdpD |
| SEQ ID N^{o} 2109 | pli0051 | 4160.1 | Unknown, similar to the two components response regulator KdpE |
| SEQ ID N^{o} 2110 | pli0052 | 4158.1 | Unknown, similar to potassium-transporting atpase a chain |
| SEQ ID N^{o} 2111 | pli0053 | 4157.1 | Unknown, similar to potassium-transporting atpase b chain |
| SEQ ID N^{o} 2112 | pli0054 | 4154.1 | Unknown |
| SEQ ID N^{o} 2113 | pli0055 | 4153.1 | Unknown, similar to potassium-transporting atpase c chain |
| SEQ ID N^{o} 2114 | pli0056 | 4150.1 | Unknown, similar to resolvase/integrase |
| SEQ ID N^{o} 2115 | pli0057 | 4148.1 | Unknown, similar to DNA transposition protein |
| SEQ ID N^{o} 2116 | pli0058 | 4147.1 | Unknown, similar to transposase |
| SEQ ID N^{o} 2117 | pli0059 | 4146.1 | Unknown, similar to invertase |
| SEQ ID N^{o} 2118 | pli0060 | 4145.1 | Unknown, similar to cadmium resistance accessory protein |
| SEQ ID N^{o} 2119 | pli0062 | 4140.1 | Unknown, similar to unknown protein |
| SEQ ID N^{o} 2120 | pli0063 | 4139.1 | Unknown, similar to transposase |
| SEQ ID N^{o} 2121 | pli0064 | 4138.1 | Unknown, similar to unknown protein |
| SEQ ID N^{o} 2122 | pli0065 | 4137.1 | Unknown |
| SEQ ID N^{o} 2123 | pli0066 | 4136.1 | Unknown |
| SEQ ID N^{o} 2124 | pli0067 | 4135.1 | Unknown, similar to UV-damage repair protein |
| SEQ ID N^{o} 2125 | pli0068 | 4132.1 | Unknown, similar to unknown protein |
| SEQ ID N^{o} 2126 | pli0069 | 4130.1 | Unknown, similar to plasmid copy control protein repB |
| SEQ ID N^{o} 2127 | pli0070 | 4128.1 | Unknown, similar to plasmid replication protein |
| SEQ ID N^{o} 2128 | pli0071 | 4124.1 | Unknown, similar to transposase |
| SEQ ID N^{o} 2129 | pli0072 | 4123.1 | Unknown, similar to transposase |
| SEQ ID N^{o} 2130 | pli0073 | 4122.1 | unknown |
| SEQ ID N^{o} 2131 | pli0074 | 4121.1 | Unknown |
| SEQ ID N^{o} 2132 | pli0075 | 4120.1 | Unknown |
| SEQ ID N^{o} 2133 | pli0076 | 4116.1 | Transposase |
| SEQ ID N^{o} 2134 | pli0077 | 4113.1 | Unknown, similar to transposase N-terminal part |
| SEQ ID N^{o} 2135 | pli0078 | 4112.1 | Unknown, similar to enolase (phosphopyruvate hydratase), truncated C-terminal end |
| SEQ ID N^{o} 2136 | pli0079 | 4111.1 | Unknown, similar to Na+/H+ antiporter |
| SEQ ID N^{o} 2137 | pli0080 | 4107.2 | Unknown |
| SEQ ID N^{o} 2138 | Lin0059 | 4543.1 | Unknown |
| SEQ ID N^{o} 2139 | Lin0060 | 4535.1 | unknown |
| SEQ ID N^{o} 2140 | Lin0061 | 4533.1 | unknown, hypothetical protein |
| SEQ ID N^{o} 2141 | Lin0062 | 4532.1 | unknown, hypothetical protein |
| SEQ ID N^{o} 2142 | Lin0063 | 4531.1 | Unknown, pseudogene |
| SEQ ID N^{o} 2143 | Lin0064 | 4522.1 | unknown |
| SEQ ID N^{o} 2144 | Lin0065 | 4521.1 | unknown |
| SEQ ID N^{o} 2145 | Lin0066 | 4520.1 | Unknwon |
| SEQ ID N^{o} 2146 | Lin0071 | 4508.1 | Unknown, similar to integrase |
| SEQ ID N^{o} 2147 | Lin0072 | 4506.1 | unknown |
| SEQ ID N^{o} 2148 | Lin0073 | 4503.2 | Unknown, similar to a putative repressor protein [Bacteriophage A118] |
| SEQ ID N^{o} 2149 | Lin0074 | 6149.2 | unknown, highly similar to gp37 [Bacteriophage A118] |
| SEQ ID N^{o} 2150 | Lin0075 | 6148.2 | unknown, highly similar to gp37-1 [Bacteriophage A118] |
| SEQ ID N^{o} 2151 | Lin0076 | 6544.1 | Unknown |
| SEQ ID N^{o} 2152 | Lin0077 | 6317.1 | unknown, identical to gp40 [Bacteriophage A118] |
| SEQ ID N^{o} 2153 | Lin0079 | 6145.1 | unknown |
| SEQ ID N^{o} 2154 | Lin0080 | 6144.1 | unknown, similar to similar to anti-repressor [Bacteriophage A118] |
| SEQ ID N^{o} 2155 | Lin0081 | 6142.1 | unknown, highly similar to gp43 [Bacteriophage A118] |
| SEQ ID N^{o} 2156 | Lin0082 | 6453.2 | Unknown |
| SEQ ID N^{o} 2157 | Lin0083 | 6139.1 | unknown, highly similar to gp45 [Bacteriophage A118] |
| SEQ ID N^{o} 2158 | Lin0084 | 6138.3 | unknown, highly similar to gp47 [Bacteriophage A118] |
| SEQ ID N^{o} 2159 | Lin0085 | 6136.2 | putative recombinase [Bacteriophage A118] |
| SEQ ID N^{o} 2160 | Lin0086 | 3386.1 | Unknown, similar to protein gp49 |
| SEQ ID N^{o} 2161 | Lin0087 | 6543.1 | Unknown, similar to phage protein |
| SEQ ID N^{o} 2162 | Lin0089 | 3379.1 | unknown, similar to gp51 [Bacteriophage A118] |
| SEQ ID N^{o} 2163 | Lin0090 | 3378.1 | unknown, |
| SEQ ID N^{o} 2164 | Lin0091 | 3375.1 | unknown, similar to phage proteins |
| SEQ ID N^{o} 2165 | Lin0092 | 6542.1 | Unknown |
| SEQ ID N^{o} 2166 | Lin0093 | 3374.1 | unknown |
| SEQ ID N^{o} 2167 | Lin0094 | 3373.1 | unknown, highly similar to gp55 [Bacteriophage A118] |
| SEQ ID N^{o} 2168 | Lin0095 | 3371.1 | unknown, highly similar to gp59 [Bacteriophage A118] |
| SEQ ID N^{o} 2169 | Lin0096 | 3370.1 | unknown |
| SEQ ID N^{o} 2170 | Lin0102 | 3363.1 | unknown |
| SEQ ID N^{o} 2171 | Lin0103 | 3362.1 | unknown |
| SEQ ID N^{o} 2172 | Lin0104 | 3361.1 | unknown, highly similar to putative terminase small subunit [Bacteriophage A118] |
| SEQ ID N^{o} 2173 | Lin0109 | 3354.1 | unknown, highly similar to major capsid protein [Bacteriophage A118] |
| SEQ ID N^{o} 2174 | Lin0110 | 6540.1 | Protein gp7 [Bacteriophage A118] |
| SEQ ID N^{o} 2175 | Lin0116 | 3345.1 | unknown, highly similar to gp13 [Bacteriophage A118](truncated, C-terminal end) |
| SEQ ID N^{o} 2176 | Lin0120 | 3333.1 | unknown, highly similar to gp17 [Bacteriophage A118] |
| SEQ ID N^{o} 2177 | Lin0121 | 3330.1 | unknown, highly similar to gp18 [Bacteriophage A118] |
| SEQ ID N^{o} 2178 | Lin0122 | 3329.1 | unknown, highly similar to gp19 [Bacteriophage A118] |
| SEQ ID N^{o} 2179 | Lin0123 | 3326.1 | unknown, similar to gp20 [Bacteriophage A118] |
| SEQ ID N^{o} 2180 | Lin0124 | 3325.1 | Unknown |
| SEQ ID N^{o} 2181 | Lin0125 | 6539.1 | Unknown |
| SEQ ID N^{o} 2182 | Lin0128 | 3320.1 | L-alanoyl-D-glutamate peptidase [Bacteriophage A500 from Listeria] |
| SEQ ID N^{o} 2183 | Lin0129 | 3319.1 | Unknown |
| SEQ ID N^{o} 2184 | Lin0140 | 3291.1 | unknown |
| SEQ ID N^{o} 2185 | Lin0141 | 3290.1 | Unknown, putative peptidoglycan bound protein (LPXTG motif) |
| SEQ ID N^{o} 2186 | Lin0142 | 3283.1 | unknown |
| SEQ ID N^{o} 2187 | Lin0148 | 3273.1 | Unknown, similar to unknown protein |
| SEQ ID N^{o} 2188 | Lin0154 | 3255.1 | Unknown, similar to unknown protein |
| SEQ ID N^{o} 2189 | Lin0167 | 3232.1 | Unknown |
| SEQ ID N^{o} 2190 | Lin0187 | 3183.1 | Unknown |
| SEQ ID N^{o} 2191 | Lin0188 | 3180.1 | Unknown |
| SEQ ID N^{o} 2192 | Lin0189 | 6538.1 | Unknown |
| SEQ ID N^{o} 2193 | Lin0197 | 3160.1 | unknown, similar to chloromuconate cycloisomerase ykfB of B. subtilis |
| SEQ ID N^{o} 2194 | Lin0198 | 3159.1 | unknown, P45 related protein |
| SEQ ID N^{o} 2195 | Lin0199 | 3157.1 | unknown, some similarities to probable beta-lactamase |
| SEQ ID N^{o} 2196 | Lin0200 | 3152.1 | Unknown, similar to ABC transporter oligopeptide-binding protein |
| SEQ ID N^{o} 2197 | Lin0201 | 3148.1 | Unknown, similar to dipeptide ABC transporter |
| SEQ ID N^{o} 2198 | Lin0202 | 3147.1 | unknwon, surface anchored protein (LPXTG motif) |
| SEQ ID N^{o} 2199 | Lin0290 | 1614.1 | unknown, internalin like protein (LPXTG motif) |
| SEQ ID N^{o} 2200 | Lin0295 | 1601.1 | Unknown, similar to internalin proteins |
| SEQ ID N^{o} 2201 | Lin0307 | 1578.1 | unknown, similar to ABC transporters (ATP-binding protein) |
| SEQ ID N^{o} 2202 | Lin0308 | 1577.1 | unknown, similar to hypothetical proteins |
| SEQ ID N^{o} 2203 | Lin0332 | 1526.1 | unknown, similar to putative permeases |
| SEQ ID N^{o} 2204 | Lin0338 | 1513.1 | unknown |
| SEQ ID N^{o} 2205 | Lin0345 | 1492.1 | unknown |
| SEQ ID N^{o} 2206 | Lin0349 | 1484.1 | Unknown |
| SEQ ID N^{o} 2207 | Lin0357 | 1466.1 | Unknown |
| SEQ ID N^{o} 2208 | Lin0372 | 1442.1 | unknown, probable cell surface protein (LPXTG motif) |
| SEQ ID N^{o} 2209 | Lin0397 | 1390.1 | unknown |
| SEQ ID N^{o} 2210 | Lin0398 | 1389.1 | unknown |
| SEQ ID N^{o} 2211 | Lin0399 | 1388.1 | unknown |
| SEQ ID N^{o} 2212 | Lin0415 | 1348.1 | unknown, probable cell surface protein (LPXTG motif) |
| SEQ ID N^{o} 2213 | Lin0416 | 1346.1 | Unknown, similar to transcription regulator |
| SEQ ID N^{o} 2214 | Lin0417 | 1345.1 | Unknown, similar to unknown protein |
| SEQ ID N^{o} 2215 | Lin0418 | 1344.1 | Unknown, similar to ABC transporter, ATP-binding protein |
| SEQ ID N^{o} 2216 | Lin0419 | 1342.1 | Unknown, similar to ABC transporter, ATP-binding protein |
| SEQ ID N^{o} 2217 | Lin0426 | 1323.1 | Unknown |
| SEQ ID N^{o} 2218 | Lin0453 | 1260.1 | unknown |
| SEQ ID N^{o} 2219 | Lin0454 | 1259.1 | unknown, similar to cell wall-associated protein precursor wapA (B. subtilis) |
| SEQ ID N^{o} 2220 | Lin0455 | 1239.1 | unknown |
| SEQ ID N^{o} 2221 | Lin0456 | 1236.1 | unknown |
| SEQ ID N^{o} 2222 | Lin0464 | 1216.1 | Unknown, similar to putative transcription regulator |
| SEQ ID N^{o} 2223 | Lin0465 | 1215.1 | unknown, conserved hypothetical protein, similar to yoaZ B. subtilis |
| SEQ ID N^{o} 2224 | Lin0476 | 1191.1 | unknown |
| SEQ ID N^{o} 2225 | Lin0477 | 1188.1 | unknown |
| SEQ ID N^{o} 2226 | Lin0478 | 1186.1 | unknown |
| SEQ ID N^{o} 2227 | Lin0479 | 1183.1 | Unknown |
| SEQ ID N^{o} 2228 | Lin0480 | 6504.1 | Unknown, putative secreted protein |
| SEQ ID N^{o} 2229 | Lin0481 | 6525.1 | pseudogene |
| SEQ ID N^{o} 2230 | Lin0486 | 1171.1 | unknown, similar to unknown proteins |
| SEQ ID N^{o} 2231 | Lin0510 | 1125.1 | Unknown |
| SEQ ID N^{o} 2232 | Lin0521 | 1096.1 | Unknown, similar to HsdR type IC restriction subunit |
| SEQ ID N^{o} 2233 | Lin0522 | 1091.1 | Unknown, similar to HsdM type IC modification subunit |
| SEQ ID N^{o} 2234 | Lin0523 | 1088.1 | Unknown, similar to specificity determinant HsdS |
| SEQ ID N^{o} 2235 | Lin0524 | 1087.1 | Unknown, similar to bacteriophage integrase |
| SEQ ID N^{o} 2236 | Lin0525 | 1086.1 | Unknown, similar to specificity determinant HsdS |
| SEQ ID N^{o} 2237 | Lin0553 | 1021.1 | Unknown, similar to internalin protein |
| SEQ ID N^{o} 2238 | Lin0554 | 1018.1 | unknown, probable cell surface protein (LPXTG motif) |
| SEQ ID N^{o} 2239 | Lin0557 | 1012.1 | Unknown |
| SEQ ID N^{o} 2240 | Lin0558 | 1010.1 | Unknown, similar to internalin protein |
| SEQ ID N^{o} 2241 | Lin0559 | 1007.1 | unknown, probable cell surface protein (LPXTG motif) |
| SEQ ID N^{o} 2242 | Lin0560 | 1005.1 | Unknown |
| SEQ ID N^{o} 2243 | Lin0561 | 1004.1 | Unknown, similar to unknown protein |
| SEQ ID N^{o} 2244 | Lin0661 | 815.1 | unknown, internalin like protein (LPXTG motif) |
| SEQ ID N^{o} 2245 | Lin0665 | 805.1 | unknown, highly similar to ORFA of Listeria seeligeri, (LPXTG motif) |
| SEQ ID N^{o} 2246 | Lin0677 | 781.1 | unknown, conserved hypothetical protein |
| SEQ ID N^{o} 2247 | Lin0678 | 779.1 | Unknown |
| SEQ ID N^{o} 2248 | Lin0679 | 776.1 | Unknown, similar to unknown protein |
| SEQ ID N^{o} 2249 | Lin0739 | 652.1 | unknown, internalin like protein (LPXTG) |
| SEQ ID N^{o} 2250 | Lin0740 | 648.1 | unknown, probable cell surface protein (LPXTG motif) |
| SEQ ID N^{o} 2251 | Lin0746 | 639.1 | Unknown, similar to ABC transporter, ATP-binding protein (truncated, N-terminal part) |
| SEQ ID N^{o} 2252 | Lin0772 | 589.1 | unknown |
| SEQ ID N^{o} 2253 | Lin0801 | 529.1 | unknown, similar to two-component response regulators |
| SEQ ID N^{o} 2254 | Lin0802 | 528.2 | unknown, similar to two-component sensor histidine kinases |
| SEQ ID N^{o} 2255 | Lin0803 | 526.1 | Unknown, surface protein (LPXTG motif) |
| SEQ ID N^{o} 2256 | Lin0804 | 523.1 | Unknown, similar to unknown protein |
| SEQ ID N^{o} 2257 | Lin0805 | 522.1 | Unknown, similar to oxidoreductase |
| SEQ ID N^{o} 2258 | Lin0806 | 521.1 | Unknown, similar to transcriptional regulator, MerR family |
| SEQ ID N^{o} 2259 | Lin0822 | 485.1 | Unknown, similar to transport protein (Truncated, N-terminal part) |
| SEQ ID N^{o} 2260 | Lin0823 | 484.1 | Unknown, similar to transport protein (truncated, C-terminal part) |
| SEQ ID N^{o} 2261 | Lin0827 | 472.1 | unknown, similar to transposase |
| SEQ ID N^{o} 2262 | Lin0833 | 457.1 | unknown |
| SEQ ID N^{o} 2263 | Lin0834 | 455.1 | unknown, some similarities to hypothetical proteins |
| SEQ ID N^{o} 2264 | Lin0835 | 454.1 | unknown |
| SEQ ID N^{o} 2265 | Lin0842 | 440.1 | Unknown, similar to amidases |
| SEQ ID N^{o} 2266 | Lin0864 | 395.1 | Unknown, similar to transcription regulator |
| SEQ ID N^{o} 2267 | Lin0865 | 394.1 | unknown, hypothetical protein |
| SEQ ID N^{o} 2268 | Lin0866 | 392.1 | unknown, similar to ABC transporters, ATP-binding protein homologue |
| SEQ ID N^{o} 2269 | Lin0867 | 391.1 | unknown |
| SEQ ID N^{o} 2270 | Lin0868 | 390.1 | unknown |
| SEQ ID N^{o} 2271 | Lin0877 | 372.1 | unknown (truncated, N-terminal part) |
| SEQ ID N^{o} 2272 | Lin0878 | 371.1 | unknown (truncated, C-terminal part) |
| SEQ ID N^{o} 2273 | Lin0903 | 325.1 | Unknown, truncated N-terminal part |
| SEQ ID N^{o} 2274 | Lin0904 | 324.1 | Unknown (truncated, C-terminal end) |
| SEQ ID N^{o} 2275 | Lin0915 | 302.1 | Unknown, similar to phosphotransferase system enzyme IIC (truncated, N-terminal end) |
| SEQ ID N^{o} 2276 | Lin0916 | 301.1 | Unknown, similar to phosphotransferase system enzyme IIC (truncated, C-terminal end) |
| SEQ ID N^{o} 2277 | Lin0940 | 6518.1 | unknown, similar to heat shock protein HtpG (truncated, C-terminal part) |
| SEQ ID N^{o} 2278 | Lin1056 | 36.1 | unknown |
| SEQ ID N^{o} 2279 | Lin1064 | 20.1 | unknown, similar to autolysin (amidase) |
| SEQ ID N^{o} 2280 | Lin1065 | 17.3 | Unknown |
| SEQ ID N^{o} 2281 | Lin1066 | 14.4 | unknown, similar to dolichol phosphate mannose synthase |
| SEQ ID N^{o} 2282 | Lin1067 | 13.1 | unknown |
| SEQ ID N^{o} 2283 | Lin1068 | 12.1 | unknown, similar to hypothetical protein 3 (capsulation locus) of Haemophilus influenzae |
| SEQ ID N^{o} 2284 | Lin1069 | 9.1 | unknown |
| SEQ ID N^{o} 2285 | Lin1073 | 3.1 | unknown, similar to galactosamine-containing minor teichoic acid biosynthesis protein GgaA |
| SEQ ID N^{o} 2286 | Lin1074 | 2.2 | Unknown, similar to B. subtilis TagF protein (probable CDPglycerol glycerophosphotransferase) |
| SEQ ID N^{o} 2287 | Lin1075 | 4502.2 | unknown, similar to teichoic acid biosynthesis protein B precursor |
| SEQ ID N^{o} 2288 | Lin1082 | 4486.1 | unknown |
| SEQ ID N^{o} 2289 | Lin1083 | 4485.1 | unknown |
| SEQ ID N^{o} 2290 | Lin1084 | 4484.1 | Unknown |
| SEQ ID N^{o} 2291 | Lin1085 | 4483.1 | unknown |
| SEQ ID N^{o} 2292 | Lin1086 | 4482.1 | unknown |
| SEQ ID N^{o} 2293 | Lin1090 | 4478.1 | unknown |
| SEQ ID N^{o} 2294 | Lin1091 | 4477.1 | unknown |
| SEQ ID N^{o} 2295 | Lin1099 | 4460.1 | unknown |
| SEQ ID N^{o} 2296 | Lin1100 | 4459.1 | unknown |
| SEQ ID N^{o} 2297 | Lin1177 | 4309.1 | unknown |
| SEQ ID N^{o} 2298 | Lin1204 | 4232.1 | unknown, similar to internalin proteins (LPXTG motif) |
| SEQ ID N^{o} 2299 | Lin1209 | 4220.1 | unknown |
| SEQ ID N^{o} 2300 | Lin1210 | 4219.1 | unknown |
| SEQ ID N^{o} 2301 | Lin1211 | 4218.1 | unknown |
| SEQ ID N^{o} 2302 | Lin1212 | 4217.1 | unknown |
| SEQ ID N^{o} 2303 | Lin1220 | 6513.1 | Unknown |
| SEQ ID N^{o} 2304 | Lin1231 | 6002.1 | Unknown, similar to site-specific recombinase for integration and excision [bacteriophage phi-105] |
| SEQ ID N^{o} 2305 | Lin1232 | 6003.1 | Unknown, similar to unknown protein |
| SEQ ID N^{o} 2306 | Lin1233 | 6005.1 | Unknown, similar to bacteriophage phi-105 ORF2 protein |
| SEQ ID N^{o} 2307 | Lin1234 | 6006.4 | Unknown, similar to immunity repressor [bacteriophage phi-105] |
| SEQ ID N^{o} 2308 | Lin1235 | 6007.4 | Unknown, similar to transcription regulator |
| SEQ ID N^{o} 2309 | Lin1236 | 6189.4 | Unknown |
| SEQ ID N^{o} 2310 | Lin1237 | 6512.2 | Unknown |
| SEQ ID N^{o} 2311 | Lin1238 | 6511.1 | Unknown |
| SEQ ID N^{o} 2312 | Lin1239 | 6188.1 | Unknown |
| SEQ ID N^{o} 2313 | Lin1240 | 6186.1 | Unknown |
| SEQ ID N^{o} 2314 | Lin1241 | 6185.5 | Unknown, similar to bacteriophage protein |
| SEQ ID N^{o} 2315 | Lin1242 | 6658.1 | unknown, some similarities to phage related proteins |
| SEQ ID N^{o} 2316 | Lin1243 | 6659.1 | unknown, similar to hypothetical protein 44 - Staphylococcus aureus phage phi PVL |
| SEQ ID N^{o} 2317 | Lin1244 | 6660.1 | unknown |
| SEQ ID N^{o} 2318 | Lin1245 | 6661.1 | unknown, similarities Staphylococcus aureus prophage phiPV83 |
| SEQ ID N^{o} 2319 | Lin1246 | 6662.1 | unknown |
| SEQ ID N^{o} 2320 | Lin1247 | 6663.1 | unknown |
| SEQ ID N^{o} 2321 | Lin1248 | 6664.1 | unknown, similar to hypothetical protein, Staphylococcus aureus phage phi PVL |
| SEQ ID N^{o} 2322 | Lin1249 | 6665.1 | unknown |
| SEQ ID N^{o} 2323 | Lin1250 | 6666.1 | unknown |
| SEQ ID N^{o} 2324 | Lin1251 | 6667.1 | Unknown |
| SEQ ID N^{o} 2325 | Lin1252 | 6668.1 | Unknown |
| SEQ ID N^{o} 2326 | Lin1253 | 6669.1 | Unknown |
| SEQ ID N^{o} 2327 | Lin1254 | 6670.1 | unknown, similar to phage intagrase proteins |
| SEQ ID N^{o} 2328 | Lin1255 | 6671.1 | unknown |
| SEQ ID N^{o} 2329 | Lin1256 | 6040.3 | Unknown |
| SEQ ID N^{o} 2330 | Lin1257 | 2999.2 | Unknown |
| SEQ ID N^{o} 2331 | Lin1258 | 2998.1 | Unknown |
| SEQ ID N^{o} 2332 | Lin1259 | 2997.1 | Unknown, similar to protein gp66 [Bacteriophage A118] |
| SEQ ID N^{o} 2333 | Lin1260 | 6202.2 | unknown, similar to probable antirepressor - Bacillus subtilis |
| | | | phage SPBc2 |
| SEQ ID N^{o} 2334 | Lin1261 | 5611.4 | Unknown |
| SEQ ID N^{o} 2335 | Lin1262 | 5609.3 | Unknown |
| SEQ ID N^{o} 2336 | Lin1263 | 6588.1 | Unknown |
| SEQ ID N^{o} 2337 | Lin1264 | 5606.1 | Unknown |
| SEQ ID N^{o} 2338 | Lin1265 | 5605.1 | Unknown |
| SEQ ID N^{o} 2339 | Lin1266 | 5604.1 | Unknown, similar to phage protein |
| SEQ ID N^{o} 2340 | Lin1267 | 5602.1 | Unknown, similar to a B. subtilis PBSX phage protein |
| SEQ ID N^{o} 2341 | Lin1268 | 5600.1 | Unknown, similar to a B. subtilis PBSX phage protein |
| SEQ ID N^{o} 2342 | Lin1269 | 5597.1 | Unknown, similar to unknown protein |
| SEQ ID N^{o} 2343 | Lin1270 | 5593.1 | Unknown, similar to a B. subtilis PBSX phage protein |
| SEQ ID N^{o} 2344 | Lin1271 | 5590.1 | Unknown, similar to a B. subtilis PBSX phage protein |
| SEQ ID N^{o} 2345 | Lin1272 | 6394.1 | unknown |
| SEQ ID N^{o} 2346 | Lin1273 | 5587.1 | Unknown, similar to unknown protein |
| SEQ ID N^{o} 2347 | Lin1274 | 5586.1 | Unknown, similar to a B. subtilis PBSX phage protein |
| SEQ ID N^{o} 2348 | Lin1275 | 5584.1 | Unknown, similar to a B. subtilis PBSX phage protein |
| SEQ ID N^{o} 2349 | Lin1276 | 5583.1 | Unknown, similar to a B. subtilis PBSX phage protein |
| SEQ ID N^{o} 2350 | Lin1277 | 5580.1 | unknown |
| SEQ ID N^{o} 2351 | Lin1278 | 5579.1 | Unknown, similar to a B. subtilis PBSX phage protein |
| SEQ ID N^{o} 2352 | Lin1279 | 5577.1 | Unknown, similar to a B. subtilis PBSX phage protein |
| SEQ ID N^{o} 2353 | Lin1280 | 5576.1 | Unknown, similar to a B. subtilis PBSX phage protein |
| SEQ ID N^{o} 2354 | Lin1281 | 6636.1 | Unknown, similar to phage protein (truncated, C-terminal end) |
| SEQ ID N^{o} 2355 | Lin1282 | 5575.1 | Unknown, similar to phage proteins |
| SEQ ID N^{o} 2356 | Lin1283 | 5569.1 | Unknown, similar to a B. subtilis PBSX prophage protein |
| SEQ ID N^{o} 2357 | Lin1284 | 5568.1 | Unknown, similar to a B. subtilis PBSX prophage protein |
| SEQ ID N^{o} 2358 | Lin1285 | 5567.1 | Unknown, similar to a B. subtilis PBSX prophage protein |
| SEQ ID N^{o} 2359 | Lin1286 | 5565.1 | Unknown, similar to a B. subtilis PBSX prophage protein |
| SEQ ID N^{o} 2360 | Lin1287 | 5564.1 | Unknown, similar to a B. subtilis PBSX prophage protein |
| SEQ ID N^{o} 2361 | Lin1288 | 5561.1 | Unknown, similar to a B. subtilis PBSX prophage protein |
| SEQ ID N^{o} 2362 | Lin1289 | 5560.1 | Unknown, similar to unknown protein |
| SEQ ID N^{o} 2363 | Lin1290 | 5558.1 | Unknown |
| SEQ ID N^{o} 2364 | Lin1291 | 5556.1 | Unknown |
| SEQ ID N^{o} 2365 | Lin1292 | 6589.1 | Unknown |
| SEQ ID N^{o} 2366 | Lin1293 | 5555.1 | Unknown |
| SEQ ID N^{o} 2367 | Lin1294 | 5553.1 | Unknown |
| SEQ ID N^{o} 2368 | Lin1295 | 5551.1 | Unknown, similar to holin |
| SEQ ID N^{o} 2369 | Lin1296 | 5550.1 | unknown, similar to hypothetical protein - phage SPP1 |
| SEQ ID N^{o} 2370 | Lin1297 | 6590.1 | Unknown, similar to Portein gp28 [Bacteriophage A118] |
| SEQ ID N^{o} 2371 | Lin1298 | 5546.1 | unknown |
| SEQ ID N^{o} 2372 | Lin1299 | 5545.1 | unknown |
| SEQ ID N^{o} 2373 | Lin1300 | 5543.1 | unknown |
| SEQ ID N^{o} 2374 | Lin1301 | 5541.1 | Unknown |
| SEQ ID N^{o} 2375 | Lin1302 | 6591.1 | Unknown |
| SEQ ID N^{o} 2376 | Lin1328 | 5484.1 | unknown, internalin like protein (LPXTG motif) |
| SEQ ID N^{o} 2377 | Lin1378 | 6432.2 | Unknown, weakly similar to B. subtilis comG |
| | | | operon protein 7 (comGG) |
| SEQ ID N^{o} 2378 | Lin1379 | 2316.2 | Unknown, similar to B. subtilis comG operon protein 6 |
| SEQ ID N^{o} 2379 | Lin1381 | 2318.1 | Unknown, similar to comG operon protein 4 (comGD) |
| SEQ ID N^{o} 2380 | Lin1450 | 2451.1 | Unknown |
| SEQ ID N^{o} 2381 | Lin1451 | 2455.1 | Unknwon |
| SEQ ID N^{o} 2382 | Lin1452 | 2456.1 | unknown |
| SEQ ID N^{o} 2383 | Lin1618 | 2777.1 | Unknown, similar to a protein encoded by Th916 |
| SEQ ID N^{o} 2384 | Lin1619 | 2778.1 | Unknown |
| SEQ ID N^{o} 2385 | Lin1620 | 2779.1 | Unknown, similar to putative iron-sulfur flavoprotein |
| SEQ ID N^{o} 2386 | Lin1621 | 2781.1 | unknown, similar to ketoacyl reductases |
| SEQ ID N^{o} 2387 | Lin1622 | 2783.1 | Unknown, similar to transcriptional regulator (MerR family) |
| SEQ ID N^{o} 2388 | Lin1623 | 2785.1 | Unknown, similar to site-specific recombinase tnpX - Clostridium perfringens transposon Tn4451 |
| | | | (N terminal part) |
| SEQ ID N^{o} 2389 | Lin1624 | 2788.1 | unknown, similar to site-specific recombinase tnpX - Clostridium perfringens transposon Tn4451 |
| SEQ ID N^{o} 2390 | Lin1695 | 2923.1 | Unknown, similar to unknown protein |
| SEQ ID N^{o} 2391 | Lin1696 | 2924.1 | unknown |
| SEQ ID N^{o} 2392 | Lin1700 | 2934.1 | Unknown, similar to N-acetylmuramoyl-L-alanine amidase (N-terminal part) and to L-alanoyl-D- |
| | | | glutamate peptidase (C-terminal part) |
| SEQ ID N^{o} 2393 | Lin1701 | 6597.1 | Unknown |
| SEQ ID N^{o} 2394 | Lin1702 | 6598.1 | Unknown similar to holin from bacteriophage |
| SEQ ID N^{o} 2395 | Lin1703 | 2937.1 | unknown |
| SEQ ID N^{o} 2396 | Lin1704 | 2939.1 | Unknown |
| SEQ ID N^{o} 2397 | Lin1705 | 2940.1 | Unknown |
| SEQ ID N^{o} 2398 | Lin1706 | 6599.1 | Unknown, similar to protein gp22 [Bacteriophage A118] |
| SEQ ID N^{o} 2399 | Lin1707 | 2941.1 | Unknown |
| SEQ ID N^{o} 2400 | Lin1708 | 2942.1 | Unknown |
| SEQ ID N^{o} 2401 | Lin1709 | 2943.1 | Unknown |
| SEQ ID N^{o} 2402 | Lin1710 | 2944.2 | Unknown, similar to unknown protein |
| SEQ ID N^{o} 2403 | Lin1711 | 2945.2 | Unknown |
| SEQ ID N^{o} 2404 | Lin1712 | 2946.2 | Unknown |
| SEQ ID N^{o} 2405 | Lin1713 | 2947.1 | Unknown |
| SEQ ID N^{o} 2406 | Lin1714 | 2949.1 | Unknown |
| SEQ ID N^{o} 2407 | Lin1715 | 2950.1 | Unknown |
| SEQ ID N^{o} 2408 | Lin1716 | 2958.1 | Unknown, similar to minor capsid protein 1608 - Lactobacillus phage phi-gle |
| SEQ ID N^{o} 2409 | Lin1717 | 2959.1 | Unknown |
| SEQ ID N^{o} 2410 | Lin1718 | 2961.1 | unknown |
| SEQ ID N^{o} 2411 | Lin1719 | 2964.1 | unknown |
| SEQ ID N^{o} 2412 | Lin1720 | 2966.1 | unknown, weakly similar to hypothetical protein of bacteriophage Felix 01 |
| SEQ ID N^{o} 2413 | Lin1721 | 2967.1 | unknown |
| SEQ ID N^{o} 2414 | Lin1722 | 2968.1 | unknown |
| SEQ ID N^{o} 2415 | Lin1723 | 2969.1 | unknown |
| SEQ ID N^{o} 2416 | Lin1724 | 2970.1 | unknown |
| SEQ ID N^{o} 2417 | Lin1725 | 2973.1 | unknown |
| SEQ ID N^{o} 2418 | Lin1726 | 2975.1 | unknown, similar to hypothetical proteins |
| SEQ ID N^{o} 2419 | Lin1727 | 2976.1 | unknown |
| SEQ ID N^{o} 2420 | Lin1728 | 2978.1 | unknown, similar to hypothetical proteins |
| SEQ ID N^{o} 2421 | Lin1729 | 6600.1 | Unknown |
| SEQ ID N^{o} 2422 | Lin1730 | 2981.1 | unknown, some similarities to plasmid-related proteins |
| SEQ ID N^{o} 2423 | Lin1731 | 2982.1 | unknown, some similarities to conserved hypothetical proteins |
| SEQ ID N^{o} 2424 | Lin1732 | 2985.1 | unknown, some similarities to phage related proteins |
| SEQ ID N^{o} 2425 | Lin1733 | 2987.1 | unknown, weakly similar to phage related proteins |
| SEQ ID N^{o} 2426 | Lin1734 | 2988.1 | unknown |
| SEQ ID N^{o} 2427 | Lin1735 | 2989.1 | Unknown |
| SEQ ID N^{o} 2428 | Lin1736 | 6601.1 | Unknown |
| SEQ ID N^{o} 2429 | Lin1737 | 2993.1 | unknown, weakly similar to methyltransferases |
| SEQ ID N^{o} 2430 | Lin1738 | 2995.3 | Unknown, similar to a putative antirepressor [Bacteriophage SPBc2] |
| SEQ ID N^{o} 2431 | Lin1739 | 6045.4 | unknown, similar to protein gp66 of Bacteriophage A118 |
| SEQ ID N^{o} 2432 | Lin1740 | 6043.1 | unknown, similar to hypothetical protein of Lactobacillus phage phi-gle |
| SEQ ID N^{o} 2433 | Lin1741 | 6042.1 | unknown, similar to hypothetical protein from phage P2 |
| SEQ ID N^{o} 2434 | Lin1742 | 6038.1 | unknown |
| SEQ ID N^{o} 2435 | Lin1743 | 6037.1 | unknown, similar to phage intagrase proteins |
| SEQ ID N^{o} 2436 | Lin1744 | 6035.1 | unknown |
| SEQ ID N^{o} 2437 | Lin1745 | 6032.1 | unknown |
| SEQ ID N^{o} 2438 | Lin1746 | 6574.1 | Unknown |
| SEQ ID N^{o} 2439 | Lin1747 | 6030.1 | unknown |
| SEQ ID N^{o} 2440 | Lin1748 | 6029.1 | unknown |
| SEQ ID N^{o} 2441 | Lin1749 | 6028.1 | unknown, similar to hypothetical protein, Staphylococcus aureus phage phi PVL |
| SEQ ID N^{o} 2442 | Lin1750 | 6027.1 | unknown |
| SEQ ID N^{o} 2443 | Lin1751 | 6026.1 | unknown |
| SEQ ID N^{o} 2444 | Lin1752 | 6025.1 | unknown, similarities Staphylococcus aureus prophage phiPV83 |
| SEQ ID N^{o} 2445 | Lin1753 | 6021.1 | unknown |
| SEQ ID N^{o} 2446 | Lin1754 | 6019.1 | unknown, similar to hypothetical protein 44 - Staphylococcus aureus phage phi PVL |
| SEQ ID N^{o} 2447 | Lin1755 | 6018.1 | unknown, some similarities to phage related proteins |
| SEQ ID N^{o} 2448 | Lin1756 | 6016.1 | unknown, similar to hypothetical protein of Staphylococcus aureus phage phi PVL |
| SEQ ID N^{o} 2449 | Lin1757 | 6015.1 | unknown |
| SEQ ID N^{o} 2450 | Lin1758 | 6575.1 | Unknown |
| SEQ ID N^{o} 2451 | Lin1759 | 6012.1 | unknown |
| SEQ ID N^{o} 2452 | Lin1760 | 6009.4 | Unknown, similar to protein gp43 [Bacteriophage A118] |
| SEQ ID N^{o} 2453 | Lin1761 | 6291.1 | Unknown, similar to transcription regulator |
| SEQ ID N^{o} 2454 | Lin1762 | 5937.2 | Unknown, similar to immunity repressor protein - Bacillus phage phi-105 |
| SEQ ID N^{o} 2455 | Lin1763 | 5935.2 | Unknown, similar to ORF2 [bacteriophage phi-105] |
| SEQ ID N^{o} 2456 | Lin1764 | 5934.1 | Unknown, Listeria prophage protein |
| SEQ ID N^{o} 2457 | Lin1765 | 5933.1 | Unknown, similar to integrase |
| SEQ ID N^{o} 2458 | Lin1768 | 5926.1 | unknown, similar to Antigen C |
| SEQ ID N^{o} 2459 | Lin1811 | 5313.1 | unknown, similar to unknown proteins |
| SEQ ID N^{o} 2460 | Lin1812 | 5315.1 | Unknown, similar to excinuclease ABC (subunit A) (truncated, C-terminal end) |
| SEQ ID N^{o} 2461 | Lin1813 | 5317.1 | Unknown, similar to excinuclease ABC subunit A |
| SEQ ID N^{o} 2462 | Lin1814 | 5319.1 | unknown, similar to putative AraC-type regulators |
| SEQ ID N^{o} 2463 | Lin1898 | 4696.1 | Unknown, similar to putative NAD(P)H oxidoreductase |
| SEQ ID N^{o} 2464 | Lin1899 | 4697.1 | Unknown, similar to unknown protein |
| SEQ ID N^{o} 2465 | Lin1955 | 4817.1 | unknown |
| SEQ ID N^{o} 2466 | Lin2001 | 4896.2 | Unknown, hypothetical CDS |
| SEQ ID N^{o} 2467 | Lin2100 | 5275.3 | unknown, similar to p60-related proteins |
| SEQ ID N^{o} 2468 | Lin2210 | 6700.1 | Unknown |
| SEQ ID N^{o} 2469 | Lin2281 | 3752.1 | unknown, probable cell surface protein (LPXTG motif) |
| SEQ ID N^{o} 2470 | Lin2344 | 5982.1 | Unknown, similar to O6-methylguanine-DNA methyltransferase |
| SEQ ID N^{o} 2471 | Lin2371 | 4069.2 | Unknown, similar to competence transcription factor ComK, N terminal part |
| SEQ ID N^{o} 2472 | Lin2373 | 4899.3 | Unknown, similar to AbiD phage protein |
| SEQ ID N^{o} 2473 | Lin2374 | 4066.1 | Unknown, similar to N-acetylmuramoyl-L-alanine amidase (N-terminal part) and to L-alanoyl-D- |
| | | | glutamate peptidase (C-terminal part) |
| SEQ ID N^{o} 2474 | Lin2375 | 4065.1 | unknown, similar to phage related proteins |
| SEQ ID N^{o} 2475 | Lin2376 | 4062.1 | Unknown |
| SEQ ID N^{o} 2476 | Lin2377 | 6565.1 | Unknown |
| SEQ ID N^{o} 2477 | Lin2378 | 6199.6 | Unknown |
| SEQ ID N^{o} 2478 | Lin2379 | 6046.2 | Unknown, similar to protein gp20 [Bacteriophage A118] |
| SEQ ID N^{o} 2479 | Lin2380 | 6050.1 | Unknown, similar to protein gp19 [Bacteriophage A118] |
| SEQ ID N^{o} 2480 | Lin2381 | 6051.1 | Unknown, similar to protein R372 - Lactobacillus phage phi-gle |
| SEQ ID N^{o} 2481 | Lin2382 | 6052.1 | Unknown, similar to gp17 [Bacteriophage A118] |
| SEQ ID N^{o} 2482 | Lin2383 | 6059.1 | unknown, similar to hypothetical protein [Lactobacillus casei bacteriophage A2] |
| SEQ ID N^{o} 2483 | Lin2384 | 6063.1 | Unknown |
| SEQ ID N^{o} 2484 | Lin2385 | 6066.1 | Unknown, similar to protein gp13 [Bacteriophage A118] |
| SEQ ID N^{o} 2485 | Lin2386 | 6068.1 | Unknown |
| SEQ ID N^{o} 2486 | Lin2387 | 6070.1 | Unknown |
| SEQ ID N^{o} 2487 | Lin2388 | 6071.1 | Unknown |
| SEQ ID N^{o} 2488 | Lin2389 | 6572.1 | Unknown |
| SEQ ID N^{o} 2489 | Lin2390 | 6075.1 | Unknown, similar to main capsid protein Gp34 - Lactobacillus delbrueckii subsp. |
| | | | bulgaricus phage mv4 |
| SEQ ID N^{o} 2490 | Lin2391 | 6076.1 | Unknown |
| SEQ ID N^{o} 2491 | Lin2392 | 6078.1 | Unknown, similar to protein gp4 [Bacteriophage A118] |
| SEQ ID N^{o} 2492 | Lin2393 | 6081.1 | unknown |
| SEQ ID N^{o} 2493 | Lin2394 | 6084.1 | unknown |
| SEQ ID N^{o} 2494 | Lin2395 | 6085.1 | unknown, some similarities to phage-related terminase small subunit homolog yqaS |
| SEQ ID N^{o} 2495 | Lin2396 | 6086.1 | Unknown |
| SEQ ID N^{o} 2496 | Lin2397 | 6087.1 | unknown, similar to sigma factor-like positive control protein of B. subtilis |
| SEQ ID N^{o} 2497 | Lin2398 | 6570.1 | Unknown, hypothetical gene |
| SEQ ID N^{o} 2498 | Lin2399 | 6090.1 | unknown |
| SEQ ID N^{o} 2499 | Lin2400 | 6092.1 | Unknown, similar to Lactococcus lactis prophage pi3 protein 45 |
| SEQ ID N^{o} 2500 | Lin2401 | 6569.1 | Unknown |
| SEQ ID N^{o} 2501 | Lin2402 | 6093.1 | unknown, similar to single-stranded DNA-binding protein |
| SEQ ID N^{o} 2502 | Lin2403 | 6096.1 | unknown |
| SEQ ID N^{o} 2503 | Lin2404 | 6099.1 | unknown |
| SEQ ID N^{o} 2504 | Lin2405 | 6101.1 | unknown |
| SEQ ID N^{o} 2505 | Lin2406 | 6502.1 | Protein gp52 [Bacteriophage A118] |
| SEQ ID N^{o} 2506 | Lin2407 | 6104.2 | pseudogene |
| SEQ ID N^{o} 2507 | Lin2408 | 6698.1 | Unknown |
| SEQ ID N^{o} 2508 | Lin2409 | 3995.1 | unknown, similar to intrgase proteins |
| SEQ ID N^{o} 2509 | Lin2410 | 6131.2 | unknown, similar to phage related proteins |
| SEQ ID N^{o} 2510 | Lin2411 | 6132.1 | unknown |
| SEQ ID N^{o} 2511 | Lin2412 | 6135.1 | unknown, highly similar to gp49 [Bacteriophage A118] |
| SEQ ID N^{o} 2512 | Lin2413 | 3387.3 | unknown, highly similar to putative recombinase [Bacteriophage A118] |
| SEQ ID N^{o} 2513 | Lin2414 | 5720.2 | gp47 [Bacteriophage A118] |
| SEQ ID N^{o} 2514 | Lin2415 | 6697.1 | Unknown |
| SEQ ID N^{o} 2515 | Lin2418 | 5715.1 | Unknown, similar to anti-repressor |
| SEQ ID N^{o} 2516 | Lin2419 | 6620.2 | Unknown |
| SEQ ID N^{o} 2517 | Lin2420 | 5710.1 | Unknwon, similar to Bacteriophage A118 protein gp40 |
| SEQ ID N^{o} 2518 | Lin2421 | 5709.1 | Unknwon |
| SEQ ID N^{o} 2519 | Lin2422 | 5708.1 | Unknown, similar to Bacteriophage A118 putative repressor protein |
| SEQ ID N^{o} 2520 | Lin2423 | 5706.1 | Unknown, similar to Bacteriophage A118 protein gp34 |
| SEQ ID N^{o} 2521 | Lin2425 | 5704.1 | unknown |
| SEQ ID N^{o} 2522 | Lin2454 | 5645.1 | Unknown, similar to 6-phospho-beta-glucosidase |
| SEQ ID N^{o} 2523 | Lin2455 | 5644.1 | Unknown, similar to transcription antiterminator BgIG family |
| SEQ ID N^{o} 2524 | Lin2456 | 5643.1 | Unknown, similar to unknown proteins |
| SEQ ID N^{o} 2525 | Lin2457 | 5642.1 | Unknown, similar to PTS system, cellobiose-specific enzyme IIA component |
| SEQ ID N^{o} 2526 | Lin2458 | 5640.1 | Unknown, similar to PTS system, cellobiose-specific enzyme IIB component |
| SEQ ID N^{o} 2527 | Lin2459 | 5638.1 | Unknown, similar to PTS system, cellobiose-specific enzyme IIC component |
| SEQ ID N^{o} 2528 | Lin2487 | 5052.1 | Unknown, similar to unknown proteins |
| SEQ ID N^{o} 2529 | Lin2494 | 5039.1 | unknown, hypothetical protein |
| SEQ ID N^{o} 2530 | Lin2537 | 4955.1 | Unknown, similar to internalin proteins |
| SEQ ID N^{o} 2531 | Lin2561 | 6688.1 | Unknown, similar to unknown protein |
| SEQ ID N^{o} 2532 | Lin2562 | 4067.2 | Unknown, similar to unknown protein |
| SEQ ID N^{o} 2533 | Lin2563 | 6704.1 | Unknown, similar to N-acetylmuramoyl-L-alanine amidase (N-terminal part) and to L-alanoyl-D- |
| | | | glutamate peptidase (C-terminal part) |
| SEQ ID N^{o} 2534 | Lin2564 | 4061.3 | Unknown |
| SEQ ID N^{o} 2535 | Lin2565 | 4060.1 | Unknown, similar to protein gp20 [Bacteriophage A118] |
| SEQ ID N^{o} 2536 | Lin2566 | 4057.1 | Unknown, similar to endopeptidase [bacteriophage bIL285] |
| SEQ ID N^{o} 2537 | Lin2567 | 4054.1 | Unknown, similar to Orf53 [bacteriophage bIL285] |
| SEQ ID N^{o} 2538 | Lin2568 | 4052.1 | Unknown, similar to tail protein [bacteriophage bIL285] |
| SEQ ID N^{o} 2539 | Lin2569 | 6564.1 | Unknown, similar to Lactococcus lactis prophage pi2 protein 41 |
| SEQ ID N^{o} 2540 | Lin2570 | 4047.1 | Unknown, similar to Orf51 [bacteriophage bIL285] |
| SEQ ID N^{o} 2541 | Lin2571 | 4046.1 | Unknown, similar to Orf50 [bacteriophage bIL285] |
| SEQ ID N^{o} 2542 | Lin2572 | 4044.1 | Unknown, similar to Orf49 [bacteriophage bIL285] |
| SEQ ID N^{o} 2543 | Lin2573 | 4042.1 | Unknown, similar to Orf48 [bacteriophage bIL285] |
| SEQ ID N^{o} 2544 | Lin2574 | 4041.1 | Unknown, similar to Orf47 [bacteriophage bIL285] |
| SEQ ID N^{o} 2545 | Lin2575 | 4040.1 | Unknown, similar to Orf46 [bacteriophage bIL285] |
| SEQ ID N^{o} 2546 | Lin2576 | 4039.1 | Unknown, similar to capsid protein [bacteriophage bIL285] |
| SEQ ID N^{o} 2547 | Lin2577 | 4037.1 | Unknown, similar to protease [bacteriophage bIL285] |
| SEQ ID N^{o} 2548 | Lin2578 | 4036.1 | Unknown, similar to portal protein [bacteriophage bIL285] |
| SEQ ID N^{o} 2549 | Lin2579 | 4033.1 | Unknown, similar to terminase [bacteriophage bIL285] |
| SEQ ID N^{o} 2550 | Lin2580 | 4030.1 | Unknown, similar to bacteriophage protein |
| SEQ ID N^{o} 2551 | Lin2581 | 4028.1 | Unknown, similar to bacteriophage protein |
| SEQ ID N^{o} 2552 | Lin2582 | 4026.1 | Unknown |
| SEQ ID N^{o} 2553 | Lin2583 | 4023.1 | Unknown |
| SEQ ID N^{o} 2554 | Lin2584 | 4022.1 | Unknown |
| SEQ ID N^{o} 2555 | Lin2585 | 4021.1 | Unknown |
| SEQ ID N^{o} 2556 | Lin2586 | 4020.1 | Unknown, similar to bacteriophage protein |
| SEQ ID N^{o} 2557 | Lin2587 | 4019.1 | Unknown, similar to bacteriophage protein |
| SEQ ID N^{o} 2558 | Lin2588 | 4016.1 | Unknown, similar to bacteriophage protein |
| SEQ ID N^{o} 2559 | Lin2589 | 4015.1 | Unknown, similar to DEAH-family helicase |
| SEQ ID N^{o} 2560 | Lin2590 | 4013.1 | Unknown, similar to bacteriophage protein |
| SEQ ID N^{o} 2561 | Lin2591 | 4012.1 | Unknown, similar to bacteriophage protein |
| SEQ ID N^{o} 2562 | Lin2592 | 6696.1 | Unknown |
| SEQ ID N^{o} 2563 | Lin2593 | 4008.1 | Hypothetical gene |
| SEQ ID N^{o} 2564 | Lin2594 | 4006.1 | Unknown |
| SEQ ID N^{o} 2565 | Lin2595 | 4005.1 | Unknown |
| SEQ ID N^{o} 2566 | Lin2596 | 4003.1 | Unknown |
| SEQ ID N^{o} 2567 | Lin2597 | 4001.1 | Unknown |
| SEQ ID N^{o} 2568 | Lin2598 | 6559.1 | Unknown |
| SEQ ID N^{o} 2569 | Lin2599 | 3998.1 | Unknown |
| SEQ ID N^{o} 2570 | Lin2600 | 3996.2 | unknown |
| SEQ ID N^{o} 2571 | Lin2601 | 6130.2 | Unknown, similar to bacteriophage integrase |
| SEQ ID N^{o} 2572 | Lin2602 | 3994.2 | Unknown, similar to phage protein |
| SEQ ID N^{o} 2573 | Lin2603 | 3993.1 | unknown |
| SEQ ID N^{o} 2574 | Lin2604 | 3990.1 | unknown |
| SEQ ID N^{o} 2575 | Lin2605 | 6557.1 | Unknown |
| SEQ ID N^{o} 2576 | Lin2606 | 6556.1 | Unknown |
| SEQ ID N^{o} 2577 | Lin2607 | 3989.1 | Unknown, similar to a putative repressor protein [Bacteriophage A118] |
| SEQ ID N^{o} 2578 | Lin2608 | 3988.1 | Unknwon, similar to protein gp35 from Bacteriophage A118 |
| SEQ ID N^{o} 2579 | Lin2609 | 3987.1 | Unknown |
| SEQ ID N^{o} 2580 | Lin2610 | 3985.1 | unknown, similar to integrases |
| SEQ ID N^{o} 2581 | Lin2656 | 3889.1 | unknown, similar to late competence protein comFC |
| SEQ ID N^{o} 2582 | Lin2693 | 3815.1 | Unknown |
| SEQ ID N^{o} 2583 | Lin2703 | 5735.1 | autolysin, amidase |
| SEQ ID N^{o} 2584 | Lin2723 | 5767.1 | Unknwon, conserved hypothetical protein |
| SEQ ID N^{o} 2585 | Lin2724 | 5773.1 | unknown, internalin-like protein (LPXTG motif) |
| SEQ ID N^{o} 2586 | Lin2735 | 6183.3 | Unknown |
| SEQ ID N^{o} 2587 | Lin2736 | 6181.1 | Unknown |
| SEQ ID N^{o} 2588 | Lin2741 | 1716.1 | unknown |
| SEQ ID N^{o} 2589 | Lin2742 | 1717.1 | Unknown |
| SEQ ID N^{o} 2590 | Lin2743 | 1718.1 | unknown |
| SEQ ID N^{o} 2591 | Lin2744 | 1720.1 | unknown, similar to hypothetical proteins |
| SEQ ID N^{o} 2592 | Lin2824 | 6550.1 | Unknown, similar to hydrolase (esterase) (truncated, C-terminal end) |
| SEQ ID N^{o} 2593 | Lin2825 | 1892.1 | Unknown, similar to hydrolase (esterase) (truncated, N-terminal end) |
| SEQ ID N^{o} 2594 | Lin2839 | 1935.1 | unknown |
| SEQ ID N^{o} 2595 | Lin2918 | 2102.1 | unknown |
| SEQ ID N^{o} 2596 | Lin2921 | 6547.1 | Unknown, similar to unknown protein |
| SEQ ID N^{o} 2597 | Lin2940 | 2142.1 | unknown, similar to abortive phage resistance mechanism [Lactococcus lactis ] |
| SEQ ID N^{o} 2598 | Lin2941 | 2143.1 | unknown |
| SEQ ID N^{o} 2599 | Lin2945 | 2151.1 | unknown |
| SEQ ID N^{o} 2600 | Lin2958 | 2180.1 | Unknown, similar to efflux proteins (truncated, N-terminal end) |
| SEQ ID N^{o} 2601 | Lin2959 | 2182.1 | Unknown, similar to efflux proteins (truncated, N-terminal end) |

Table VII: Legends

SEQ ID Nos. 2602-2871: nucleotide sequences of the 270 genes specific for Listeria monocytogenes EGDe; with, in the first column, the SEQ ID identifier, in the second column the name of the gene, in the third column the IPF number (<<Pasteur Institute>> identifier No. making it possible to correlate the sequence with the sequences of Table V) and in the final column the corresponding annotation.

TABLE VII

| SEQ ID | Name | IPF ID | Function |
|---|---|---|---|
| SEQ ID N^{o} 2602 | Imo0017 | 587.1 | Unknown, similar to Bacillus anthracis CapA protein (polyglutamate capsule biosynthesis) |
| SEQ ID N^{o} 2603 | Imo0036 | 611.1 | Unknown, similar to ornithine carbamoyltransferase |
| SEQ ID N^{o} 2604 | Imo0037 | 613.1 | Unknown, similar to amino acid transporter |
| SEQ ID N^{o} 2605 | Imo0038 | 614.1 | Unknown, conserved hypothetical protein |
| SEQ ID N^{o} 2606 | Imo0039 | 615.1 | Unknown, similar to carbamate kinase |
| SEQ ID N^{o} 2607 | Imo0040 | 616.1 | Unknown, conserved hypothetical protein |
| SEQ ID N^{o} 2608 | Imo0041 | 617.1 | Unknown, conserved hypothetical protein, hypothetical regulator |
| SEQ ID N^{o} 2609 | Imo0066 | 2161.2 | Unknwon, similar to toxin components |
| SEQ ID N^{o} 2610 | Imo0067 | 395.2 | Unknown, similar to dinitrogenase reductase ADP-ribosylation system |
| SEQ ID N^{o} 2611 | Imo0069 | 392.3 | unknown |
| SEQ ID N^{o} 2612 | Imo0070 | 390.3 | unknown |
| SEQ ID N^{o} 2613 | Imo0071 | 389.1 | unknown |
| SEQ ID N^{o} 2614 | Imo0072 | 4251.1 | Unknown, Hypothetical |
| SEQ ID N^{o} 2615 | Imo0073 | 388.1 | unknown |
| SEQ ID N^{o} 2616 | Imo0074 | 387.1 | Unknown |
| SEQ ID N^{o} 2617 | Imo0079 | 380.1 | Unknown |
| SEQ ID N^{o} 2618 | Imo0080 | 379.1 | Unknown |
| SEQ ID N^{o} 2619 | Imo0081 | 378.1 | Unknown |
| SEQ ID N^{o} 2620 | Imo0082 | 377.1 | Unknown |
| SEQ ID N^{o} 2621 | Imo0083 | 376.1 | Unknown, similar to transcription regulator (merR family) |
| SEQ ID N^{o} 2622 | Imo0084 | 375.1 | Unknwon, similar to oxidoreductases |
| SEQ ID N^{o} 2623 | Imo0106 | 345.1 | Unknown, similar to transcription regulator |
| SEQ ID N^{o} 2624 | Imo0110 | 338.1 | Unknown, similar to lipase |
| SEQ ID N^{o} 2625 | Imo0140 | 2401.1 | Unknwon |
| SEQ ID N^{o} 2626 | Imo0141 | 3995.1 | unknown |
| SEQ ID N^{o} 2627 | Imo0142 | 2400.1 | unknown |
| SEQ ID N^{o} 2628 | Imo0143 | 2398.1 | unknown |
| SEQ ID N^{o} 2629 | Imo0144 | 2397.1 | Unknown |
| SEQ ID N^{o} 2630 | Imo0145 | 4247.1 | Unknwon, hypothetical protein |
| SEQ ID N^{o} 2631 | Imo0146 | 4246.1 | Unknwon, hypothetical protein |
| SEQ ID N^{o} 2632 | Imo0147 | 2395.1 | Unknown |
| SEQ ID N^{o} 2633 | Imo0148 | 2394.1 | unknown |
| SEQ ID N^{o} 2634 | Imo0149 | 2393.1 | |
| SEQ ID N^{o} 2635 | Imo0150 | 2392.1 | unknown |
| SEQ ID N^{o} 2636 | Imo0151 | 2391.1 | Unknown |
| SEQ ID N^{o} 2637 | Imo0171 | 973.1 | Unknwon, similar to internalin proteins, putative peptidoglycan bound protein (LPXTG motif) |
| SEQ ID N^{o} 2638 | Imo0172 | 970.1 | Unknown, similar to transposase C-terminal part |
| SEQ ID N^{o} 2639 | Imo0173 | 969.1 | Unknown, similar to transposase (N-terminal part) |
| SEQ ID N^{o} 2640 | Imo0174 | 968.1 | Unknown, similar to transposase |
| SEQ ID N^{o} 2641 | prfA | 1447.1 | listeriolysin positive regulatory protein |
| SEQ ID N^{o} 2642 | plcA | 1446.1 | phosphatidylinositol-specific phospholipase c |
| SEQ ID N^{o} 2643 | hly | 1445.1 | listeriolysin O precursor |
| SEQ ID N^{o} 2644 | mpl | 1444.1 | Zinc metalloproteinase precursor |
| SEQ ID N^{o} 2645 | actA | 1442.1 | actin-assembly inducing protein precursor |
| SEQ ID N^{o} 2646 | plcB | 1439.1 | phospholipase C |
| SEQ ID N^{o} 2647 | Imo0206 | 1438.1 | Unknwon |
| SEQ ID N^{o} 2648 | Imo0252 | 3976.4 | Unknown, similar to repressor (penicilinase repressor) |
| SEQ ID N^{o} 2649 | Imo0253 | 4262.2 | Unknown, similar to penicillinase antirepressor |
| SEQ ID N^{o} 2650 | Imo0254 | 1859.2 | Unknown |
| SEQ ID N^{o} 2651 | Imo0255 | 1858.1 | Unknown, similar to unknown protein |
| SEQ ID N^{o} 2652 | Imo0257 | 1856.2 | Unknown, similar to unknown protein |
| SEQ ID N^{o} 2653 | inIG | 1842.1 | internalin G |
| SEQ ID N^{o} 2654 | inIH | 1840.1 | internalin H |
| SEQ ID N^{o} 2655 | inIE | 1838.1 | internalin E |
| SEQ ID N^{o} 2656 | Imo0304 | 2474.1 | Unknown |
| SEQ ID N^{o} 2657 | Imo0310 | 3811.3 | Unknown |
| SEQ ID N^{o} 2658 | Imo0311 | 2336.3 | Unknown |
| SEQ ID N^{o} 2659 | Imo0312 | 2335.2 | Unknown, similar to unknown proteins |
| SEQ ID N^{o} 2660 | Imo0313 | 2334.1 | Unknown, conserved hypothetical protein |
| SEQ ID N^{o} 2661 | Imo0320 | 2323.1 | Unknown, similar to surface protein (peptidoglycan bound, LPXTG motif) |
| SEQ ID N^{o} 2662 | Imo0329 | 3934.1 | Unknown, similar to transposase |
| SEQ ID N^{o} 2663 | Imo0330 | 3750.2 | Unknown, similar to transposase |
| SEQ ID N^{o} 2664 | Imo0332 | 3754.2 | Unknown |
| SEQ ID N^{o} 2665 | Imo0333 | 2137.2 | Unknown, similar to internalin proteins, putative peptidoglycan bound protein (LPXTG motif) |
| SEQ ID N^{o} 2666 | Imo0334 | 2138.1 | unknown |
| SEQ ID N^{o} 2667 | Imo0337 | 2141.1 | Unknown |
| SEQ ID N^{o} 2668 | Imo0338 | 2142.1 | Unknown |
| SEQ ID N^{o} 2669 | Imo0340 | 4097.1 | Unknown |
| SEQ ID N^{o} 2670 | Imo0378 | 2050.1 | Unknown |
| SEQ ID N^{o} 2671 | Imo0379 | 2049.3 | Unknown |
| SEQ ID N^{o} 2672 | Imo0380 | 1115.3 | Unknown |
| SEQ ID N^{o} 2673 | Imo0381 | 1114.1 | Unknown |
| SEQ ID N^{o} 2674 | Imo0409 | 1074.1 | Unknown, similar to internalin, peptidoglycan bound protein (LPxTG motif) |
| SEQ ID N^{o} 2675 | Imo0419 | 1572.1 | Unknown, similar to unknown protein |
| SEQ ID N^{o} 2676 | inIA | 1549.1 | Internalin A |
| SEQ ID N^{o} 2677 | inIB | 1547.1 | Internalin B |
| SEQ ID N^{o} 2678 | Imo0438 | 1538.1 | unknown |
| SEQ ID N^{o} 2679 | Imo0440 | 1625.2 | Unknown |
| SEQ ID N^{o} 2680 | Imo0444 | 1631.1 | Unknown, conserved hypothetical protein |
| SEQ ID N^{o} 2681 | Imo0445 | 1632.1 | Unknown, similar to transcription regulator |
| SEQ ID N^{o} 2682 | Imo0446 | 1634.1 | Unknown, similar to penicillin acylase and to conjugated bile acid hydrolase |
| SEQ ID N^{o} 2683 | Imo0447 | 1635.1 | Unknown, similar to glutamate decarboxylase |
| SEQ ID N^{o} 2684 | Imo0448 | 1636.1 | Unknown, similar to amino acid antiporter |
| SEQ ID N^{o} 2685 | Imo0459 | 1655.1 | Unknown, similar to transcription regulator (VirR from Streptococcus pyogenes) |
| SEQ ID N^{o} 2686 | Imo0460 | 1656.1 | Unknown, putative membrane associated lipoprotein |
| SEQ ID N^{o} 2687 | Imo0461 | 1658.1 | Unknown |
| SEQ ID N^{o} 2688 | Imo0462 | 1659.3 | Unknown |
| SEQ ID N^{o} 2689 | Imo0463 | 1660.3 | Unknown |
| SEQ ID N^{o} 2690 | Imo0464 | 3853.2 | Unknown, weakly similar to transposase |
| SEQ ID N^{o} 2691 | Imo0465 | 3953.1 | Hypothetical orf |
| SEQ ID N^{o} 2692 | Imo0466 | 3905.2 | Unknown |
| SEQ ID N^{o} 2693 | Imo0467 | 3954.2 | Unknown |
| SEQ ID N^{o} 2694 | Imo0468 | 4040.1 | Unknown |
| SEQ ID N^{o} 2695 | Imo0469 | 3337.3 | Unknown |
| SEQ ID N^{o} 2696 | Imo0470 | 3336.3 | Unknown, weakly similar to site-specific DNA-methyltransferase |
| SEQ ID N^{o} 2697 | Imo0471 | 3335.2 | Unknown |
| SEQ ID N^{o} 2698 | Imo0472 | 3334.1 | Unknown |
| SEQ ID N^{o} 2699 | Imo0473 | 3333.1 | Unknown |
| SEQ ID N^{o} 2700 | Imo0474 | 3332.1 | Unknown |
| SEQ ID N^{o} 2701 | Imo0475 | 3331.1 | Unknown |
| SEQ ID N^{o} 2702 | Imo0476 | 3330.2 | Unknown, similar to oxetanocin A resistance protein oxrB |
| SEQ ID N^{o} 2703 | Imo0477 | 3284.1 | Unknown, putative secreted protein |
| SEQ ID N^{o} 2704 | Imo0478 | 3285.1 | Unknown, putative secreted protein |
| SEQ ID N^{o} 2705 | Imo0479 | 3286.1 | Unknown, putative secreted protein |
| SEQ ID N^{o} 2706 | Imo0492 | 1713.1 | Unknown, similar to transcriptional regulator (LysR family) |
| SEQ ID N^{o} 2707 | Imo0493 | 1714.1 | Unknown, similar to acylase |
| SEQ ID N^{o} 2708 | Imo0497 | 1718.2 | Unknown, similar to sugar transferase |
| SEQ ID N^{o} 2709 | Imo0525 | 1037.1 | Unknown |
| SEQ ID N^{o} 2710 | Imo0630 | 1518.1 | Unknown, similar to transcription antiterminator BgIG family |
| SEQ ID N^{o} 2711 | Imo0631 | 1519.1 | Unknown, similar to PTS system, fructose-specific IIA component |
| SEQ ID N^{o} 2712 | Imo0632 | 1520.1 | Unknown, similar to PTS system, fructose-specific IIC component |
| SEQ ID N^{o} 2713 | Imo0633 | 1521.1 | Unknown, similar to PTS system, fructose-specific IIB component |
| SEQ ID N^{o} 2714 | Imo0634 | 1523.1 | Unknown, similar to an E. coli putative tagatose 6-phosphate kinase |
| SEQ ID N^{o} 2715 | Imo0638 | 1528.1 | Unknown |
| SEQ ID N^{o} 2716 | Imo0726 | 4124.1 | Hypothetical CDS |
| SEQ ID N^{o} 2717 | Imo0733 | 1176.1 | Unknown, similar to transcription regulator |
| SEQ ID N^{o} 2718 | Imo0734 | 1175.1 | Unknown, similar to transcriptional regulator (Lacl family) |
| SEQ ID N^{o} 2719 | Imo0735 | 1174.1 | Unknown, similar to Ribulose-5-Phosphate 3-Epimerase |
| SEQ ID N^{o} 2720 | Imo0736 | 1173.1 | Unknown, similar to ribose 5-phosphate isomerase |
| SEQ ID N^{o} 2721 | Imo0737 | 1172.1 | Unknown |
| SEQ ID N^{o} 2722 | Imo0738 | 1171.1 | Unknown, similar to phosphotransferase system (PTS) beta-glucoside-specific enzyme IIABC componei |
| SEQ ID N^{o} 2723 | Imo0739 | 1169.1 | Unknown, similar to 6-phospho-beta-glucosidase |
| SEQ ID N^{o} 2724 | Imo0746 | 1160.1 | Unknown, hypothetical |
| SEQ ID N^{o} 2725 | Imo0747 | 1159.1 | unknown |
| SEQ ID N^{o} 2726 | Imo0748 | 1158.1 | Unknown |
| SEQ ID N^{o} 2727 | Imo0749 | 4361.1 | |
| SEQ ID N^{o} 2728 | Imo0750 | 1157.1 | Unknown |
| SEQ ID N^{o} 2729 | Imo0751 | 1156.1 | Unknown |
| SEQ ID N^{o} 2730 | Imo0752 | 1155.1 | Unknown, weakly similar to a putative haloacetate dehalogenase |
| SEQ ID N^{o} 2731 | Imo0753 | 1154.1 | unknown, similar to transcription regulator Crp/Fnr family |
| SEQ ID N^{o} 2732 | Imo0754 | 1153.2 | Unknown, weakly similar to a bile acid 7-alpha dehydratase |
| SEQ ID N^{o} 2733 | Imo0780 | 1123.1 | Unknown |
| SEQ ID N^{o} 2734 | Imo0801 | 3477.1 | Unknown, similar to internalin, putative peptidoglycan bound protein (LPXTG motif) |
| SEQ ID N^{o} 2735 | Imo0804 | 3469.2 | Unknown |
| SEQ ID N^{o} 2736 | Imo0805 | 3929.1 | unknown |
| SEQ ID N^{o} 2737 | Imo0826 | 1261.1 | Unknown, similar to transport protein |
| SEQ ID N^{o} 2738 | Imo0827 | 1259.1 | unknown, similar to transposases |
| SEQ ID N^{o} 2739 | Imo0828 | 1258.1 | unknown, similar to transposases |
| SEQ ID N^{o} 2740 | Imo0833 | 1250.1 | Unknown, similar to transcriptional regulator |
| SEQ ID N^{o} 2741 | Imo0834 | 1249.1 | unknown |
| SEQ ID N^{o} 2742 | Imo0835 | 1248.1 | Unknown, putative peptidoglycan bound protein (LPXTG motif) |
| SEQ ID N^{o} 2743 | uhpT | 1243.1 | unknown, highly similar to hexose phosphate transport protein |
| SEQ ID N^{o} 2744 | Imo0842 | 1235.1 | Unknown, putative peptidoglycan bound protein (LPXTG motif) |
| SEQ ID N^{o} 2745 | Imo0904 | 1624.2 | Unknown |
| SEQ ID N^{o} 2746 | Imo0933 | 1586.1 | unknown, similar to sugar transferase |
| SEQ ID N^{o} 2747 | Imo0940 | 1580.1 | Unknown |
| SEQ ID N^{o} 2748 | Imo1030 | 3538.3 | unknown, similar to transcriptional regulator, Lacl family |
| SEQ ID N^{o} 2749 | Imo1031 | 1398.3 | unknown, similar to hypothetical proteins |
| SEQ ID N^{o} 2750 | Imo1032 | 1396.1 | unknown, similar to transketolase |
| SEQ ID N^{o} 2751 | Imo1033 | 1394.1 | unknown, similar to transketolase |
| SEQ ID N^{o} 2752 | Imo1034 | 1392.1 | unknown, similar to glycerol kinase |
| SEQ ID N^{o} 2753 | Imo1035 | 1391.1 | unknown, similar to phosphotransferase system (PTS) beta-glucoside-specific enzyme IIABC |
| SEQ ID N^{o} 2754 | Imo1036 | 1390.1 | unknown |
| SEQ ID N^{o} 2755 | Imo1060 | 1359.1 | unknown, similar to transcription response regulator |
| SEQ ID N^{o} 2756 | Imo1061 | 1358.1 | Unknown, similar to two-component sensor histidine kinase |
| SEQ ID N^{o} 2757 | Imo1062 | 1357.1 | unknown, similar to ABC transporters (permease protein) |
| SEQ ID N^{o} 2758 | Imo1063 | 1354.1 | unknown, similar to ABC transporter (ATP binding protein) |
| SEQ ID N^{o} 2759 | Imo1076 | 3609.1 | unknown, similar to autolysin (EC 3.5.1.28) (N-acetylmuramoyl-L-alanine amidase) |
| SEQ ID N^{o} 2760 | Imo1077 | 3612.1 | unknown, similar to teichoic acid biosynthesis protein B |
| SEQ ID N^{o} 2761 | Imo1079 | 3614.3 | unknown, similar to B. subtilis YfhO protein |
| SEQ ID N^{o} 2762 | Imo1080 | 2597.3 | unknown, similar to B. subtilis minor teichoic acids biosynthesis protein GgaB |
| SEQ ID N^{o} 2763 | Imo1081 | 2598.3 | Unknown, similar to glucose-1-phosphate thymidyl transferase |
| SEQ ID N^{o} 2764 | Imo1082 | 2599.1 | Unknown, similar to dTDP-sugar epimerase |
| SEQ ID N^{o} 2765 | Imo1083 | 2600.1 | Unknown, similar to dTDP-D-glucose 4,6-dehydratase |
| SEQ ID N^{o} 2766 | Imo1084 | 2601.1 | unknown, similar to DTDP-L-rhamnose synthetase |
| SEQ ID N^{o} 2767 | Imo1085 | 2602.1 | unknown, similar to teichoic acid biosynthesis protein B |
| SEQ ID N^{o} 2768 | Imo1090 | 2608.1 | unknown, similar to glycosyltransferases |
| SEQ ID N^{o} 2769 | Imo1091 | 2609.1 | unknown, similar to glysosyltransferases |
| SEQ ID N^{o} 2770 | Imo1097 | 2618.1 | unknown, similar to integrases |
| SEQ ID N^{o} 2771 | Imo1098 | 4147.1 | unknown, highly similar to TN916 ORF8 |
| SEQ ID N^{o} 2772 | Imo1099 | 2619.1 | unknown, similar to a protein encoded by Tn916 |
| SEQ ID N^{o} 2773 | cadA | 2621.4 | cadmium resistance protein |
| SEQ ID N^{o} 2774 | Imo1101 | 3973.2 | Unknown, similar to lipoprotein signal peptidase |
| SEQ ID N^{o} 2775 | Imo1102 | 3024.1 | unknown, similar to cadmium efflux system accessory proteins |
| SEQ ID N^{o} 2776 | Imo1103 | 3023.1 | unknown, highly similar to TN916 ORF13 |
| SEQ ID N^{o} 2777 | Imo1104 | 3022.1 | unknown, highly similar to TN916 ORF14 and to L. monocytogenes P60 protein |
| SEQ ID N^{o} 2778 | Imo1105 | 3020.1 | unknown, highly similar to TN916 ORF15 |
| SEQ ID N^{o} 2779 | Imo1106 | 3018.1 | unknown, highly similar to TN916 ORF16 |
| SEQ ID N^{o} 2780 | Imo1107 | 3017.1 | unknown, highly similar to TN916 ORF17 |
| SEQ ID N^{o} 2781 | Imo1108 | 3016.1 | unknown, highly similar to TN916 ORF18 |
| SEQ ID N^{o} 2782 | Imo1109 | 4148.1 | unknown, highly similar to TN916 ORF19 |
| SEQ ID N^{o} 2783 | Imo1110 | 3014.1 | unknown, similar to unknown proteins |
| SEQ ID N^{o} 2784 | Imo1111 | 3013.1 | unknown, highly similar to TN916 ORF20 |
| SEQ ID N^{o} 2785 | Imo1112 | 3012.1 | unknown, highly similar to TN916 ORF21 |
| SEQ ID N^{o} 2786 | Imo1113 | 3011.1 | unknown, highly similar to TN916 ORF22 |
| SEQ ID N^{o} 2787 | Imo1114 | 3010.1 | unknown, highly similar to TN916 ORF23 |
| SEQ ID N^{o} 2788 | Imo1115 | 3009.3 | unknown, similar to fibrinogen-binding protein (LPXTG motif) |
| SEQ ID N^{o} 2789 | Imo1116 | 4267.1 | unknown, similar to regulatory proteins |
| SEQ ID N^{o} 2790 | Imo1117 | 4268.1 | unknown |
| SEQ ID N^{o} 2791 | Imo1118 | 4149.2 | unknown |
| SEQ ID N^{o} 2792 | Imo1119 | 247.3 | unknown, similar to methylases |
| SEQ ID N^{o} 2793 | Imo1120 | 246.1 | unknown |
| SEQ ID N^{o} 2794 | Imo1121 | 245.1 | unknown |
| SEQ ID N^{o} 2795 | Imo1125 | 241.2 | unknown |
| SEQ ID N^{o} 2796 | Imo1133 | 232.1 | unknown, similar to B. subtilis YjcS protein |
| SEQ ID N^{o} 2797 | Imo1134 | 231.1 | unknown, similar to regulatory proteins |
| SEQ ID N^{o} 2798 | Imo1135 | 230.1 | unknown |
| SEQ ID N^{o} 2799 | Imo1139 | 223.1 | unknown |
| SEQ ID N^{o} 2800 | Imo1188 | 156.1 | unknown |
| SEQ ID N^{o} 2801 | Imo1247 | 2296.1 | unknown |
| SEQ ID N^{o} 2802 | Imo1263 | 4152.2 | unknown, similar to transcriptional regulator |
| SEQ ID N^{o} 2803 | Imo1290 | 1974.3 | Unknown, similar to internalin proteins, putative peptidoglycan bound protein (LPXTG motif) |
| SEQ ID N^{o} 2804 | Imo1307 | 1997.1 | unknown |
| SEQ ID N^{o} 2805 | Imo1413 | 1778.1 | Unknown, putative peptidoglycan bound protein (LPXTG motif) |
| SEQ ID N^{o} 2806 | Imo1441 | 1814.1 | Unknown, similar to putative peptidoglycan acetylation protein |
| SEQ ID N^{o} 2807 | Imo1451 | 1827.1 | Unknown, similar to E. coli LytB protein |
| SEQ ID N^{o} 2808 | Imo1477 | 2928.1 | Unknown, similar to oxidoreductase |
| SEQ ID N^{o} 2809 | Imo1478 | 2929.1 | Unknown, similar to transcriptional regulator (MerR family) |
| SEQ ID N^{o} 2810 | Imo1597 | 3951.1 | Unknown |
| SEQ ID N^{o} 2811 | Imo1648 | 2130.1 | unknown |
| SEQ ID N^{o} 2812 | Imo1656 | 3728.1 | unknown |
| SEQ ID N^{o} 2813 | Imo1659 | 3681.2 | unknown |
| SEQ ID N^{o} 2814 | Imo1666 | 3418.2 | unknown, peptidoglycan linked protein (LPxTG) |
| SEQ ID N^{o} 2815 | Imo1714 | 3463.2 | unknown |
| SEQ ID N^{o} 2816 | inlC | 3779.3 | internalin C |
| SEQ ID N^{o} 2817 | Imo1968 | 2521.1 | unknown, similar to creatinine amidohydrolases |
| SEQ ID N^{o} 2818 | Imo1969 | 2522.1 | Unknown, similar to 2-keto-3-deoxygluconate-6-phosphate aldolase |
| SEQ ID N^{o} 2819 | Imo1970 | 2523.1 | Unknown, similar to putative phosphotriesterase related proteins |
| SEQ ID N^{o} 2820 | Imo1971 | 2524.1 | Unknown, similar to pentitol PTS system enzyme II C component |
| SEQ ID N^{o} 2821 | Imo1972 | 4065.1 | Unknown, similar to pentitol PTS system enzyme II B component |
| SEQ ID N^{o} 2822 | Imo1973 | 2527.1 | Unknown, similar to PTS system enzyme II A component |
| SEQ ID N^{o} 2823 | Imo1974 | 2528.1 | Unknown, similar to transcription regulators, (GntR family) |
| SEQ ID N^{o} 2824 | Imo2026 | 2504.1 | Unknown, putative peptidoglycan bound protein (LPXTG motif) |
| SEQ ID N^{o} 2825 | Imo2027 | 2503.1 | Unknown, putative cell surface protein, similar to internalin proteins |
| SEQ ID N^{o} 2826 | Imo2067 | 3512.1 | Unknown, similar to conjugated bile acid hydrolase |
| SEQ ID N^{o} 2827 | Imo2085 | 3691.2 | Unknown, putative peptidoglycan bound protein (LPXTG motif) |
| SEQ ID N^{o} 2828 | Imo2093 | 4359.1 | Unknown |
| SEQ ID N^{o} 2829 | Imo2143 | 858.1 | Unknown, weakly similar to mannose-6-phosphate isomerase |
| SEQ ID N^{o} 2830 | Imo2144 | 857.1 | Unknown, similar to transcription regulator GntR family |
| SEQ ID N^{o} 2831 | sepA | 842.1 | Unknown |
| SEQ ID N^{o} 2832 | Imo2197 | 3539.1 | Unknown |
| SEQ ID N^{o} 2833 | Imo2228 | 4342.1 | Unknown, similar to unknown protein |
| SEQ ID N^{o} 2834 | Imo2257 | 2761.1 | Unknown, hypothetical CDS |
| SEQ ID N^{o} 2835 | Imo2364 | 452.1 | Hypothetical protein |
| SEQ ID N^{o} 2836 | Imo2387 | 906.1 | Unknown, conserved hypothetical protein |
| SEQ ID N^{o} 2837 | Imo2395 | 4226.1 | Unknown |
| SEQ ID N^{o} 2838 | Imo2407 | 881.1 | unknown |
| SEQ ID N^{o} 2839 | Imo2408 | 4227.1 | Unknown, similar to repressor protein |
| SEQ ID N^{o} 2840 | Imo2409 | 880.1 | Unknown |
| SEQ ID N^{o} 2841 | Imo2410 | 879.1 | Unknown |
| SEQ ID N^{o} 2842 | Imo2420 | 4358.1 | unknown |
| SEQ ID N^{o} 2843 | Imo2443 | 2215.1 | unknown |
| SEQ ID N^{o} 2844 | Imo2470 | 757.1 | Unknown, similar to internalin proteins |
| SEQ ID N^{o} 2845 | Imo2576 | 3700.2 | Unknwon, peptidoglycan anchored protein (LPXTG motif) |
| SEQ ID N^{o} 2846 | Imo2594 | 1759.1 | Unknown |
| SEQ ID N^{o} 2847 | Imo2595 | 1760.1 | Unknown, similar to unknown proteins |
| SEQ ID N^{o} 2848 | Imo2671 | 49.1 | Unknown |
| SEQ ID N^{o} 2849 | Imo2672 | 51.1 | Unknown, weakly similar to transcription regulator |
| SEQ ID N^{o} 2850 | Imo2686 | 80.1 | unknown |
| SEQ ID N^{o} 2851 | Imo2732 | 270.1 | unknown |
| SEQ ID N^{o} 2852 | Imo2733 | 272.1 | Unknown, similar to PTS system, fructose-specific IIABC component |
| SEQ ID N^{o} 2853 | Imo2734 | 273.1 | Unknown, weakly similar to sugar hydrolase |
| SEQ ID N^{o} 2854 | Imo2735 | 274.1 | Unknown, similar to Sucrose phosphorylase |
| SEQ ID N^{o} 2855 | Imo2736 | 275.1 | Unknown, conserved hypothetical protein |
| SEQ ID N^{o} 2856 | Imo2771 | 710.1 | Unknown, similar to beta-glucosidase |
| SEQ ID N^{o} 2857 | Imo2772 | 711.2 | Unknown, similar to beta-glucoside-specific enzyme IIABC |
| SEQ ID N^{o} 2858 | Imo2773 | 712.2 | Unknwon, similar to transcription antiterminator |
| SEQ ID N^{o} 2859 | Imo2776 | 716.1 | Unknown |
| SEQ ID N^{o} 2860 | Imo2780 | 721.1 | Unknown, similar to cellobiose PTS enzyme IIA |
| SEQ ID N^{o} 2861 | Imo2781 | 723.1 | Unknown, similar to beta-glucosidase |
| SEQ ID N^{o} 2862 | Imo2782 | 724.1 | Unknown, similar to PTS, cellobiose-specific IIB component |
| SEQ ID N^{o} 2863 | Imo2783 | 725.1 | Unknown, similar to cellobiose phosphotransferase system enzyme IIC |
| SEQ ID N^{o} 2864 | Imo2784 | 726.1 | Unknown, similar to lichenan operon transcription antiterminator licR |
| SEQ ID N^{o} 2865 | bvrC | 728.1 | Unknown |
| SEQ ID N^{o} 2866 | bvrB | 730.1 | beta-glucoside-specific phosphotransferase enzyme II ABC component |
| SEQ ID N^{o} 2867 | bvrA | 731.2 | transcription antiterminator |
| SEQ ID N^{o} 2868 | Imo2807 | 3431.2 | Unknown, hypothetical secreted protein |
| SEQ ID N^{o} 2869 | Imo2809 | 1067.2 | Unknown, hypothetical secreted protein |
| SEQ ID N^{o} 2870 | Imo2821 | 1050.1 | Unknown, similar to internalin, Unknown, putative peptidoglycan bound protein (LPXTG motif) |
| SEQ ID N^{o} 2871 | Imo2841 | 1206.1 | unknown, weakly similar to sucrose phosphorylase |

Table VIII: Legends

SEQ ID Nos. 2872-3891: sequences of 1020 Contigs derived from the assembly of 13 919 sequences of Listeria monocytogenes 4b.

In these sequences, the undetermined bases are marked by an “N”. Some of these contigs contain the 974 former contigs of Lm4b SEQ ID Nos. 1068 to 2041; with, in the first column, the SEQ ID identifier, in the second column the contig number and the number(s) of the corresponding sequences SEQ ID Nos. 1068 to 2041 of table V.

TABLE VIII

| | |
|---|---|
| SEQ ID N^{o} 2872 | Listeria monocytogenes 4b Contig1 |
| SEQ ID N^{o} 2873 | Listeria monocytogenes 4b Contig2 |
| SEQ ID N^{o} 2874 | Listeria monocytogenes 4b Contig3 |
| SEQ ID N^{o} 2875 | Listeria monocytogenes 4b Contig4 |
| SEQ ID N^{o} 2876 | Listeria monocytogenes 4b Contig5 |
| | Corresponding to the former SEQ ID n^{o} 1069 |
| SEQ ID N^{o} 2877 | Listeria monocytogenes 4b Contig6 |
| SEQ ID N^{o} 2878 | Listeria monocytogenes 4b Contig7 |
| SEQ ID N^{o} 2879 | Listeria monocytogenes 4b Contig8 |
| | Corresponding to the former SEQ ID n^{o} 1076 |
| SEQ ID N^{o} 2880 | Listeria monocytogenes 4b Contig9 |
| SEQ ID N^{o} 2881 | Listeria monocytogenes 4b Contig10 |
| SEQ ID N^{o} 2882 | Listeria monocytogenes 4b Contig11 |
| SEQ ID N^{o} 2883 | Listeria monocytogenes 4b Contig12 |
| | Corresponding to the former SEQ ID n^{o} 1075 |
| SEQ ID N^{o} 2884 | Listeria monocytogenes 4b Contig13 |
| SEQ ID N^{o} 2885 | Listeria monocytogenes 4b Contig14 |
| SEQ ID N^{o} 2886 | Listeria monocytogenes 4b Contig15 |
| SEQ ID N^{o} 2887 | Listeria monocytogenes 4b Contig16 |
| SEQ ID N^{o} 2888 | Listeria monocytogenes 4b Contig17 |
| SEQ ID N^{o} 2889 | Listeria monocytogenes 4b Contig18 |
| SEQ ID N^{o} 2890 | Listeria monocytogenes 4b Contig19 |
| SEQ ID N^{o} 2891 | Listeria monocytogenes 4b Contig20 |
| SEQ ID N^{o} 2892 | Listeria monocytogenes 4b Contig21 |
| SEQ ID N^{o} 2893 | Listeria monocytogenes 4b Contig22 |
| SEQ ID N^{o} 2894 | Listeria monocytogenes 4b Contig23 |
| SEQ ID N^{o} 2895 | Listeria monocytogenes 4b Contig24 |
| SEQ ID N^{o} 2896 | Listeria monocytogenes 4b Contig25 |
| SEQ ID N^{o} 2897 | Listeria monocytogenes 4b Contig26 |
| SEQ ID N^{o} 2898 | Listeria monocytogenes 4b Contig27 |
| SEQ ID N^{o} 2899 | Listeria monocytogenes 4b Contig28 |
| SEQ ID N^{o} 2900 | Listeria monocytogenes 4b Contig29 |
| SEQ ID N^{o} 2901 | Listeria monocytogenes 4b Contig30 |
| SEQ ID N^{o} 2902 | Listeria monocytogenes 4b Contig31 |
| SEQ ID N^{o} 2903 | Listeria monocytogenes 4b Contig32 |
| SEQ ID N^{o} 2904 | Listeria monocytogenes 4b Contig33 |
| SEQ ID N^{o} 2905 | Listeria monocytogenes 4b Contig34 |
| SEQ ID N^{o} 2906 | Listeria monocytogenes 4b Contig35 |
| SEQ ID N^{o} 2907 | Listeria monocytogenes 4b Contig36 |
| SEQ ID N^{o} 2908 | Listeria monocytogenes 4b Contig37 |
| | Corresponding to the former SEQ ID n^{o} 1363 |
| SEQ ID N^{o} 2909 | Listeria monocytogenes 4b Contig38 |
| SEQ ID N^{o} 2910 | Listeria monocytogenes 4b Contig39 |
| SEQ ID N^{o} 2911 | Listeria monocytogenes 4b Contig40 |
| | Corresponding to the former SEQ ID n^{o} 1126 |
| SEQ ID N^{o} 2912 | Listeria monocytogenes 4b Contig41 |
| SEQ ID N^{o} 2913 | Listeria monocytogenes 4b Contig42 |
| SEQ ID N^{o} 2914 | Listeria monocytogenes 4b Contig43 |
| SEQ ID N^{o} 2915 | Listeria monocytogenes 4b Contig44 |
| | Corresponding to the former SEQ ID n^{o} 1096 |
| SEQ ID N^{o} 2916 | Listeria monocytogenes 4b Contig45 |
| SEQ ID N^{o} 2917 | Listeria monocytogenes 4b Contig46 |
| SEQ ID N^{o} 2918 | Listeria monocytogenes 4b Contig47 |
| SEQ ID N^{o} 2919 | Listeria monocytogenes 4b Contig48 |
| SEQ ID N^{o} 2920 | Listeria monocytogenes 4b Contig49 |
| SEQ ID N^{o} 2921 | Listeria monocytogenes 4b Contig50 |
| SEQ ID N^{o} 2922 | Listeria monocytogenes 4b Contig51 |
| | Corresponding to the former SEQ ID n^{o} 1145 |
| SEQ ID N^{o} 2923 | Listeria monocytogenes 4b Contig52 |
| SEQ ID N^{o} 2924 | Listeria monocytogenes 4b Contig53 |
| SEQ ID N^{o} 2925 | Listeria monocytogenes 4b Contig54 |
| SEQ ID N^{o} 2926 | Listeria monocytogenes 4b Contig55 |
| | Corresponding to the former SEQ ID n^{o} 1209 |
| SEQ ID N^{o} 2927 | Listeria monocytogenes 4b Contig56 |
| SEQ ID N^{o} 2928 | Listeria monocytogenes 4b Contig57 |
| SEQ ID N^{o} 2929 | Listeria monocytogenes 4b Contig58 |
| SEQ ID N^{o} 2930 | Listeria monocytogenes 4b Contig59 |
| | Corresponding to the former SEQ ID n^{o} 1379 |
| SEQ ID N^{o} 2931 | Listeria monocytogenes 4b Contig60 |
| SEQ ID N^{o} 2932 | Listeria monocytogenes 4b Contig61 |
| SEQ ID N^{o} 2933 | Listeria monocytogenes 4b Contig62 |
| SEQ ID N^{o} 2934 | Listeria monocytogenes 4b Contig63 |
| SEQ ID N^{o} 2935 | Listeria monocytogenes 4b Contig64 |
| | Corresponding to the former SEQ ID n^{o} 1082 |
| SEQ ID N^{o} 2936 | Listeria monocytogenes 4b Contig65 |
| SEQ ID N^{o} 2937 | Listeria monocytogenes 4b Contig66 |
| | Corresponding to the former SEQ ID n^{o} 1345 |
| SEQ ID N^{o} 2938 | Listeria monocytogenes 4b Contig67 |
| SEQ ID N^{o} 2939 | Listeria monocytogenes 4b Contig68 |
| SEQ ID N^{o} 2940 | Listeria monocytogenes 4b Contig69 |
| SEQ ID N^{o} 2941 | Listeria monocytogenes 4b Contig70 |
| | Corresponding to the former SEQ ID n^{o} 1332 |
| SEQ ID N^{o} 2942 | Listeria monocytogenes 4b Contig71 |
| SEQ ID N^{o} 2943 | Listeria monocytogenes 4b Contig72 |
| SEQ ID N^{o} 2944 | Listeria monocytogenes 4b Contig73 |
| SEQ ID N^{o} 2945 | Listeria monocytogenes 4b Contig74 |
| SEQ ID N^{o} 2946 | Listeria monocytogenes 4b Contig75 |
| SEQ ID N^{o} 2947 | Listeria monocytogenes 4b Contig76 |
| SEQ ID N^{o} 2948 | Listeria monocytogenes 4b Contig77 |
| SEQ ID N^{o} 2949 | Listeria monocytogenes 4b Contig78 |
| | Corresponding to the former SEQ ID n^{o} 1194 |
| SEQ ID N^{o} 2950 | Listeria monocytogenes 4b Contig79 |
| SEQ ID N^{o} 2951 | Listeria monocytogenes 4b Contig80 |
| SEQ ID N^{o} 2952 | Listeria monocytogenes 4b Contig81 |
| SEQ ID N^{o} 2953 | Listeria monocytogenes 4b Contig82 |
| | Corresponding to the former SEQ ID n^{o} 1161 |
| SEQ ID N^{o} 2954 | Listeria monocytogenes 4b Contig83 |
| SEQ ID N^{o} 2955 | Listeria monocytogenes 4b Contig84 |
| | Corresponding to the former SEQ ID n^{o} 1179 |
| SEQ ID N^{o} 2956 | Listeria monocytogenes 4b Contig85 |
| SEQ ID N^{o} 2957 | Listeria monocytogenes 4b Contig86 |
| SEQ ID N^{o} 2958 | Listeria monocytogenes 4b Contig87 |
| SEQ ID N^{o} 2959 | Listeria monocytogenes 4b Contig88 |
| | Corresponding to the former SEQ ID n^{o} 1100 |
| SEQ ID N^{o} 2960 | Listeria monocytogenes 4b Contig89 |
| SEQ ID N^{o} 2961 | Listeria monocytogenes 4b Contig90 |
| | Corresponding to the former SEQ ID n^{o} 1294 |
| SEQ ID N^{o} 2962 | Listeria monocytogenes 4b Contig91 |
| | Corresponding to the former SEQ ID n^{o} 1136 |
| SEQ ID N^{o} 2963 | Listeria monocytogenes 4b Contig92 |
| SEQ ID N^{o} 2964 | Listeria monocytogenes 4b Contig93 |
| SEQ ID N^{o} 2965 | Listeria monocytogenes 4b Contig94 |
| SEQ ID N^{o} 2966 | Listeria monocytogenes 4b Contig95 |
| SEQ ID N^{o} 2967 | Listeria monocytogenes 4b Contig96 |
| SEQ ID N^{o} 2968 | Listeria monocytogenes 4b Contig97 |
| SEQ ID N^{o} 2969 | Listeria monocytogenes 4b Contig98 |
| SEQ ID N^{o} 2970 | Listeria monocytogenes 4b Contig99 |
| SEQ ID N^{o} 2971 | Listeria monocytogenes 4b Contig100 |
| SEQ ID N^{o} 2972 | Listeria monocytogenes 4b Contig101 |
| SEQ ID N^{o} 2973 | Listeria monocytogenes 4b Contig102 |
| SEQ ID N^{o} 2974 | Listeria monocytogenes 4b Contig103 |
| SEQ ID N^{o} 2975 | Listeria monocytogenes 4b Contig104 |
| SEQ ID N^{o} 2976 | Listeria monocytogenes 4b Contig105 |
| SEQ ID N^{o} 2977 | Listeria monocytogenes 4b Contig106 |
| SEQ ID N^{o} 2978 | Listeria monocytogenes 4b Contig107 |
| SEQ ID N^{o} 2979 | Listeria monocytogenes 4b Contig108 |
| | Corresponding to the former SEQ ID n^{o} 1256 |
| SEQ ID N^{o} 2980 | Listeria monocytogenes 4b Contig109 |
| | Corresponding to the former SEQ ID n^{o} 1210 |
| SEQ ID N^{o} 2981 | Listeria monocytogenes 4b Contig110 |
| | Corresponding to the former SEQ ID n^{o} 1220 |
| SEQ ID N^{o} 2982 | Listeria monocytogenes 4b Contig111 |
| | Corresponding to the former SEQ ID n^{o} 1406 |
| SEQ ID N^{o} 2983 | Listeria monocytogenes 4b Contig112 |
| | Corresponding to the former SEQ ID n^{o} 1355 |
| SEQ ID N^{o} 2984 | Listeria monocytogenes 4b Contig113 |
| SEQ ID N^{o} 2985 | Listeria monocytogenes 4b Contig114 |
| SEQ ID N^{o} 2986 | Listeria monocytogenes 4b Contig115 |
| | Corresponding to the former SEQ ID n^{o} 1183 |
| SEQ ID N^{o} 2987 | Listeria monocytogenes 4b Contig116 |
| SEQ ID N^{o} 2988 | Listeria monocytogenes 4b Contig117 |
| | Corresponding to the former SEQ ID n^{o} 1235 |
| SEQ ID N^{o} 2989 | Listeria monocytogenes 4b Contig118 |
| SEQ ID N^{o} 2990 | Listeria monocytogenes 4b Contig119 |
| SEQ ID N^{o} 2991 | Listeria monocytogenes 4b Contig120 |
| SEQ ID N^{o} 2992 | Listeria monocytogenes 4b Contig121 |
| SEQ ID N^{o} 2993 | Listeria monocytogenes 4b Contig122 |
| SEQ ID N^{o} 2994 | Listeria monocytogenes 4b Contig123 |
| SEQ ID N^{o} 2995 | Listeria monocytogenes 4b Contig124 |
| | Corresponding to the former SEQ ID n^{o} 1138 |
| SEQ ID N^{o} 2996 | Listeria monocytogenes 4b Contig125 |
| SEQ ID N^{o} 2997 | Listeria monocytogenes 4b Contig126 |
| SEQ ID N^{o} 2998 | Listeria monocytogenes 4b Contig127 |
| SEQ ID N^{o} 2999 | Listeria monocytogenes 4b Contig128 |
| SEQ ID N^{o} 3000 | Listeria monocytogenes 4b Contig129 |
| | Corresponding to the former SEQ ID n^{o} 1092 |
| SEQ ID N^{o} 3001 | Listeria monocytogenes 4b Contig130 |
| | Corresponding to the former SEQ ID n^{o} 1085 |
| SEQ ID N^{o} 3002 | Listeria monocytogenes 4b Contig131 |
| | Corresponding to the former SEQ ID n^{o} 1290 |
| SEQ ID N^{o} 3003 | Listeria monocytogenes 4b Contig132 |
| SEQ ID N^{o} 3004 | Listeria monocytogenes 4b Contig133 |
| SEQ ID N^{o} 3005 | Listeria monocytogenes 4b Contig134 |
| | Corresponding to the former SEQ ID n^{o} 1108 |
| SEQ ID N^{o} 3006 | Listeria monocytogenes 4b Contig135 |
| | Corresponding to the former SEQ ID n^{o} 1417 |
| SEQ ID N^{o} 3007 | Listeria monocytogenes 4b Contig136 |
| SEQ ID N^{o} 3008 | Listeria monocytogenes 4b Contig137 |
| SEQ ID N^{o} 3009 | Listeria monocytogenes 4b Contig138 |
| SEQ ID N^{o} 3010 | Listeria monocytogenes 4b Contig139 |
| SEQ ID N^{o} 3011 | Listeria monocytogenes 4b Contig140 |
| | Corresponding to the former SEQ ID n^{o} 1098 |
| SEQ ID N^{o} 3012 | Listeria monocytogenes 4b Contig141 |
| SEQ ID N^{o} 3013 | Listeria monocytogenes 4b Contig142 |
| SEQ ID N^{o} 3014 | Listeria monocytogenes 4b Contig143 |
| SEQ ID N^{o} 3015 | Listeria monocytogenes 4b Contig144 |
| SEQ ID N^{o} 3016 | Listeria monocytogenes 4b Contig145 |
| SEQ ID N^{o} 3017 | Listeria monocytogenes 4b Contig146 |
| SEQ ID N^{o} 3018 | Listeria monocytogenes 4b Contig147 |
| SEQ ID N^{o} 3019 | Listeria monocytogenes 4b Contig148 |
| SEQ ID N^{o} 3020 | Listeria monocytogenes 4b Contig149 |
| SEQ ID N^{o} 3021 | Listeria monocytogenes 4b Contig150 |
| SEQ ID N^{o} 3022 | Listeria monocytogenes 4b Contig151 |
| | Corresponding to the former SEQ ID n^{o} 1584 |
| SEQ ID N^{o} 3023 | Listeria monocytogenes 4b Contig152 |
| | Corresponding to the former SEQ ID n^{o} 1455 |
| SEQ ID N^{o} 3024 | Listeria monocytogenes 4b Contig153 |
| SEQ ID N^{o} 3025 | Listeria monocytogenes 4b Contig154 |
| SEQ ID N^{o} 3026 | Listeria monocytogenes 4b Contig155 |
| SEQ ID N^{o} 3027 | Listeria monocytogenes 4b Contig156 |
| SEQ ID N^{o} 3028 | Listeria monocytogenes 4b Contig157 |
| | Corresponding to the former SEQ ID n^{o} 1107 |
| SEQ ID N^{o} 3029 | Listeria monocytogenes 4b Contig158 |
| SEQ ID N^{o} 3030 | Listeria monocytogenes 4b Contig159 |
| SEQ ID N^{o} 3031 | Listeria monocytogenes 4b Contig160 |
| SEQ ID N^{o} 3032 | Listeria monocytogenes 4b Contig161 |
| | Corresponding to the former SEQ ID n^{o} 1485 |
| SEQ ID N^{o} 3033 | Listeria monocytogenes 4b Contig162 |
| SEQ ID N^{o} 3034 | Listeria monocytogenes 4b Contig163 |
| SEQ ID N^{o} 3035 | Listeria monocytogenes 4b Contig164 |
| SEQ ID N^{o} 3036 | Listeria monocytogenes 4b Contig165 |
| | Corresponding to the former SEQ ID n^{o} 1538 |
| SEQ ID N^{o} 3037 | Listeria monocytogenes 4b Contig166 |
| | Corresponding to the former SEQ ID n^{o} 1409 |
| SEQ ID N^{o} 3038 | Listeria monocytogenes 4b Contig167 |
| SEQ ID N^{o} 3039 | Listeria monocytogenes 4b Contig168 |
| SEQ ID N^{o} 3040 | Listeria monocytogenes 4b Contig169 |
| SEQ ID N^{o} 3041 | Listeria monocytogenes 4b Contig170 |
| SEQ ID N^{o} 3042 | Listeria monocytogenes 4b Contig171 |
| | Corresponding to the former SEQ ID n^{o} 1246 |
| SEQ ID N^{o} 3043 | Listeria monocytogenes 4b Contig172 |
| SEQ ID N^{o} 3044 | Listeria monocytogenes 4b Contig173 |
| SEQ ID N^{o} 3045 | Listeria monocytogenes 4b Contig174 |
| SEQ ID N^{o} 3046 | Listeria monocytogenes 4b Contig175 |
| | Corresponding to the former SEQ ID n^{o} 1445 |
| SEQ ID N^{o} 3047 | Listeria monocytogenes 4b Contig176 |
| SEQ ID N^{o} 3048 | Listeria monocytogenes 4b Contig177 |
| SEQ ID N^{o} 3049 | Listeria monocytogenes 4b Contig178 |
| SEQ ID N^{o} 3050 | Listeria monocytogenes 4b Contig179 |
| | Corresponding to the former SEQ ID n^{o} 1326 |
| SEQ ID N^{o} 3051 | Listeria monocytogenes 4b Contig180 |
| | Corresponding to the former SEQ ID n^{o} 1590 |
| SEQ ID N^{o} 3052 | Listeria monocytogenes 4b Contig181 |
| SEQ ID N^{o} 3053 | Listeria monocytogenes 4b Contig182 |
| | Corresponding to the former SEQ ID n^{o} 1152 |
| SEQ ID N^{o} 3054 | Listeria monocytogenes 4b Contig183 |
| SEQ ID N^{o} 3055 | Listeria monocytogenes 4b Contig184 |
| SEQ ID N^{o} 3056 | Listeria monocytogenes 4b Contig185 |
| SEQ ID N^{o} 3057 | Listeria monocytogenes 4b Contig186 |
| | Corresponding to the former SEQ ID n^{o} 1550 |
| SEQ ID N^{o} 3058 | Listeria monocytogenes 4b Contig187 |
| SEQ ID N^{o} 3059 | Listeria monocytogenes 4b Contig188 |
| SEQ ID N^{o} 3060 | Listeria monocytogenes 4b Contig189 |
| SEQ ID N^{o} 3061 | Listeria monocytogenes 4b Contig190 |
| SEQ ID N^{o} 3062 | Listeria monocytogenes 4b Contig191 |
| SEQ ID N^{o} 3063 | Listeria monocytogenes 4b Contig192 |
| SEQ ID N^{o} 3064 | Listeria monocytogenes 4b Contig193 |
| SEQ ID N^{o} 3065 | Listeria monocytogenes 4b Contig194 |
| | Corresponding to the former SEQ ID n^{o} 1535 |
| SEQ ID N^{o} 3066 | Listeria monocytogenes 4b Contig195 |
| SEQ ID N^{o} 3067 | Listeria monocytogenes 4b Contig196 |
| | Corresponding to the former SEQ ID n^{o} 1630 |
| SEQ ID N^{o} 3068 | Listeria monocytogenes 4b Contig197 |
| SEQ ID N^{o} 3069 | Listeria monocytogenes 4b Contig198 |
| SEQ ID N^{o} 3070 | Listeria monocytogenes 4b Contig199 |
| SEQ ID N^{o} 3071 | Listeria monocytogenes 4b Contig200 |
| SEQ ID N^{o} 3072 | Listeria monocytogenes 4b Contig201 |
| SEQ ID N^{o} 3073 | Listeria monocytogenes 4b Contig202 |
| | Corresponding to the former SEQ ID n^{o} 1436 |
| SEQ ID N^{o} 3074 | Listeria monocytogenes 4b Contig203 |
| SEQ ID N^{o} 3075 | Listeria monocytogenes 4b Contig204 |
| SEQ ID N^{o} 3076 | Listeria monocytogenes 4b Contig205 |
| | Corresponding to the former SEQ ID n^{o} 1267 |
| SEQ ID N^{o} 3077 | Listeria monocytogenes 4b Contig206 |
| | Corresponding to the former SEQ ID n^{o} 1461 |
| SEQ ID N^{o} 3078 | Listeria monocytogenes 4b Contig207 |
| SEQ ID N^{o} 3079 | Listeria monocytogenes 4b Contig208 |
| | Corresponding to the former SEQ ID n^{o} 1168 |
| SEQ ID N^{o} 3080 | Listeria monocytogenes 4b Contig209 |
| SEQ ID N^{o} 3081 | Listeria monocytogenes 4b Contig210 |
| SEQ ID N^{o} 3082 | Listeria monocytogenes 4b Contig211 |
| | Corresponding to the former SEQ ID n^{o} 1159 |
| SEQ ID N^{o} 3083 | Listeria monocytogenes 4b Contig212 |
| SEQ ID N^{o} 3084 | Listeria monocytogenes 4b Contig213 |
| SEQ ID N^{o} 3085 | Listeria monocytogenes 4b Contig214 |
| SEQ ID N^{o} 3086 | Listeria monocytogenes 4b Contig215 |
| SEQ ID N^{o} 3087 | Listeria monocytogenes 4b Contig216 |
| SEQ ID N^{o} 3088 | Listeria monocytogenes 4b Contig217 |
| SEQ ID N^{o} 3089 | Listeria monocytogenes 4b Contig218 |
| | Corresponding to the former SEQ ID n^{o} 1206 |
| SEQ ID N^{o} 3090 | Listeria monocytogenes 4b Contig219 |
| | Corresponding to the former SEQ ID n^{o} 1618 |
| SEQ ID N^{o} 3091 | Listeria monocytogenes 4b Contig220 |
| | Corresponding to the former SEQ ID n^{o} 1540 |
| SEQ ID N^{o} 3092 | Listeria monocytogenes 4b Contig221 |
| | Corresponding to the former SEQ ID n^{o} 1568 |
| SEQ ID N^{o} 3093 | Listeria monocytogenes 4b Contig222 |
| | Corresponding to the former SEQ ID n^{o} 1084 |
| SEQ ID N^{o} 3094 | Listeria monocytogenes 4b Contig223 |
| SEQ ID N^{o} 3095 | Listeria monocytogenes 4b Contig224 |
| SEQ ID N^{o} 3096 | Listeria monocytogenes 4b Contig225 |
| SEQ ID N^{o} 3097 | Listeria monocytogenes 4b Contig226 |
| | Corresponding to the former SEQ ID n^{o} 1464 |
| SEQ ID N^{o} 3098 | Listeria monocytogenes 4b Contig227 |
| SEQ ID N^{o} 3099 | Listeria monocytogenes 4b Contig228 |
| SEQ ID N^{o} 3100 | Listeria monocytogenes 4b Contig229 |
| SEQ ID N^{o} 3101 | Listeria monocytogenes 4b Contig230 |
| SEQ ID N^{o} 3102 | Listeria monocytogenes 4b Contig231 |
| SEQ ID N^{o} 3103 | Listeria monocytogenes 4b Contig232 |
| | Corresponding to the former SEQ ID n^{o} 1401 |
| SEQ ID N^{o} 3104 | Listeria monocytogenes 4b Contig233 |
| SEQ ID N^{o} 3105 | Listeria monocytogenes 4b Contig234 |
| | Corresponding to the former SEQ ID n^{o} 1337 |
| SEQ ID N^{o} 3106 | Listeria monocytogenes 4b Contig235 |
| SEQ ID N^{o} 3107 | Listeria monocytogenes 4b Contig236 |
| | Corresponding to the former SEQ ID n^{o} 1471 |
| SEQ ID N^{o} 3108 | Listeria monocytogenes 4b Contig237 |
| SEQ ID N^{o} 3109 | Listeria monocytogenes 4b Contig238 |
| SEQ ID N^{o} 3110 | Listeria monocytogenes 4b Contig239 |
| SEQ ID N^{o} 3111 | Listeria monocytogenes 4b Contig240 |
| SEQ ID N^{o} 3112 | Listeria monocytogenes 4b Contig241 |
| | Corresponding to the former SEQ ID n^{o} 1621 |
| SEQ ID N^{o} 3113 | Listeria monocytogenes 4b Contig242 |
| SEQ ID N^{o} 3114 | Listeria monocytogenes 4b Contig243 |
| SEQ ID N^{o} 3115 | Listeria monocytogenes 4b Contig244 |
| SEQ ID N^{o} 3116 | Listeria monocytogenes 4b Contig245 |
| SEQ ID N^{o} 3117 | Listeria monocytogenes 4b Contig246 |
| SEQ ID N^{o} 3118 | Listeria monocytogenes 4b Contig247 |
| | Corresponding to the former SEQ ID n^{o} 1661 |
| SEQ ID N^{o} 3119 | Listeria monocytogenes 4b Contig248 |
| SEQ ID N^{o} 3120 | Listeria monocytogenes 4b Contig249 |
| SEQ ID N^{o} 3121 | Listeria monocytogenes 4b Contig250 |
| SEQ ID N^{o} 3122 | Listeria monocytogenes 4b Contig251 |
| SEQ ID N^{o} 3123 | Listeria monocytogenes 4b Contig252 |
| SEQ ID N^{o} 3124 | Listeria monocytogenes 4b Contig253 |
| SEQ ID N^{o} 3125 | Listeria monocytogenes 4b Contig254 |
| | Corresponding to the former SEQ ID n^{o} 1428 |
| SEQ ID N^{o} 3126 | Listeria monocytogenes 4b Contig255 |
| SEQ ID N^{o} 3127 | Listeria monocytogenes 4b Contig256 |
| SEQ ID N^{o} 3128 | Listeria monocytogenes 4b Contig257 |
| SEQ ID N^{o} 3129 | Listeria monocytogenes 4b Contig258 |
| | Corresponding to the former SEQ ID n^{o} 1147 |
| SEQ ID N^{o} 3130 | Listeria monocytogenes 4b Contig259 |
| SEQ ID N^{o} 3131 | Listeria monocytogenes 4b Contig260 |
| | Corresponding to the former SEQ ID n^{o} 1260 |
| SEQ ID N^{o} 3132 | Listeria monocytogenes 4b Contig261 |
| SEQ ID N^{o} 3133 | Listeria monocytogenes 4b Contig262 |
| | Corresponding to the former SEQ ID n^{o} 1465 |
| SEQ ID N^{o} 3134 | Listeria monocytogenes 4b Contig263 |
| | Corresponding to the former SEQ ID n^{o} 1131 |
| SEQ ID N^{o} 3135 | Listeria monocytogenes 4b Contig264 |
| SEQ ID N^{o} 3136 | Listeria monocytogenes 4b Contig265 |
| | Corresponding to the former SEQ ID n^{o} 1512 |
| SEQ ID N^{o} 3137 | Listeria monocytogenes 4b Contig266 |
| SEQ ID N^{o} 3138 | Listeria monocytogenes 4b Contig267 |
| SEQ ID N^{o} 3139 | Listeria monocytogenes 4b Contig268 |
| SEQ ID N^{o} 3140 | Listeria monocytogenes 4b Contig269 |
| SEQ ID N^{o} 3141 | Listeria monocytogenes 4b Contig270 |
| | Corresponding to the former SEQ ID n^{o} 1710 |
| SEQ ID N^{o} 3142 | Listeria monocytogenes 4b Contig271 |
| SEQ ID N^{o} 3143 | Listeria monocytogenes 4b Contig272 |
| | Corresponding to the former SEQ ID n^{o} 1450 |
| SEQ ID N^{o} 3144 | Listeria monocytogenes 4b Contig273 |
| | Corresponding to the former SEQ ID n^{o} 1472 |
| SEQ ID N^{o} 3145 | Listeria monocytogenes 4b Contig274 |
| | Corresponding to the former SEQ ID n^{o} 1276 |
| SEQ ID N^{o} 3146 | Listeria monocytogenes 4b Contig275 |
| SEQ ID N^{o} 3147 | Listeria monocytogenes 4b Contig276 |
| SEQ ID N^{o} 3148 | Listeria monocytogenes 4b Contig277 |
| | Corresponding to the former SEQ ID n^{o} 1524 |
| SEQ ID N^{o} 3149 | Listeria monocytogenes 4b Contig278 |
| SEQ ID N^{o} 3150 | Listeria monocytogenes 4b Contig279 |
| SEQ ID N^{o} 3151 | Listeria monocytogenes 4b Contig280 |
| | Corresponding to the former SEQ ID n^{o} 1171 |
| SEQ ID N^{o} 3152 | Listeria monocytogenes 4b Contig281 |
| | Corresponding to the former SEQ ID n^{o} 1528 |
| SEQ ID N^{o} 3153 | Listeria monocytogenes 4b Contig282 |
| SEQ ID N^{o} 3154 | Listeria monocytogenes 4b Contig283 |
| SEQ ID N^{o} 3155 | Listeria monocytogenes 4b Contig284 |
| | Corresponding to the former SEQ ID n^{o} 1570 |
| SEQ ID N^{o} 3156 | Listeria monocytogenes 4b Contig285 |
| SEQ ID N^{o} 3157 | Listeria monocytogenes 4b Contig286 |
| SEQ ID N^{o} 3158 | Listeria monocytogenes 4b Contig287 |
| | Corresponding to the former SEQ ID n^{o} 1435 |
| SEQ ID N^{o} 3159 | Listeria monocytogenes 4b Contig288 |
| SEQ ID N^{o} 3160 | Listeria monocytogenes 4b Contig289 |
| | Corresponding to the former SEQ ID n^{o} 1659 |
| SEQ ID N^{o} 3161 | Listeria monocytogenes 4b Contig290 |
| SEQ ID N^{o} 3162 | Listeria monocytogenes 4b Contig291 |
| SEQ ID N^{o} 3163 | Listeria monocytogenes 4b Contig292 |
| SEQ ID N^{o} 3164 | Listeria monocytogenes 4b Contig293 |
| SEQ ID N^{o} 3165 | Listeria monocytogenes 4b Contig294 |
| SEQ ID N^{o} 3166 | Listeria monocytogenes 4b Contig295 |
| SEQ ID N^{o} 3167 | Listeria monocytogenes 4b Contig296 |
| SEQ ID N^{o} 3168 | Listeria monocytogenes 4b Contig297 |
| | Corresponding to the former SEQ ID n^{o} 1500 |
| SEQ ID N^{o} 3169 | Listeria monocytogenes 4b Contig298 |
| SEQ ID N^{o} 3170 | Listeria monocytogenes 4b Contig299 |
| | Corresponding to the former SEQ ID n^{o} 1668 |
| SEQ ID N^{o} 3171 | Listeria monocytogenes 4b Contig300 |
| SEQ ID N^{o} 3172 | Listeria monocytogenes 4b Contig301 |
| | Corresponding to the former SEQ ID n^{o} 1091 |
| SEQ ID N^{o} 3173 | Listeria monocytogenes 4b Contig302 |
| SEQ ID N^{o} 3174 | Listeria monocytogenes 4b Contig303 |
| | Corresponding to the former SEQ ID n^{o} 1338 |
| SEQ ID N^{o} 3175 | Listeria monocytogenes 4b Contig304 |
| | Corresponding to the former SEQ ID n^{o} 1109 |
| SEQ ID N^{o} 3176 | Listeria monocytogenes 4b Contig305 |
| SEQ ID N^{o} 3177 | Listeria monocytogenes 4b Contig306 |
| SEQ ID N^{o} 3178 | Listeria monocytogenes 4b Contig307 |
| SEQ ID N^{o} 3179 | Listeria monocytogenes 4b Contig308 |
| | Corresponding to the former SEQ ID n^{o} 1380 |
| SEQ ID N^{o} 3180 | Listeria monocytogenes 4b Contig309 |
| SEQ ID N^{o} 3181 | Listeria monocytogenes 4b Contig310 |
| SEQ ID N^{o} 3182 | Listeria monocytogenes 4b Contig311 |
| SEQ ID N^{o} 3183 | Listeria monocytogenes 4b Contig312 |
| | Corresponding to the former SEQ ID n^{o} 1099 |
| SEQ ID N^{o} 3184 | Listeria monocytogenes 4b Contig313 |
| SEQ ID N^{o} 3185 | Listeria monocytogenes 4b Contig314 |
| SEQ ID N^{o} 3186 | Listeria monocytogenes 4b Contig315 |
| SEQ ID N^{o} 3187 | Listeria monocytogenes 4b Contig316 |
| SEQ ID N^{o} 3188 | Listeria monocytogenes 4b Contig317 |
| | Corresponding to the former SEQ ID n^{o} 1199 |
| SEQ ID N^{o} 3189 | Listeria monocytogenes 4b Contig318 |
| | Corresponding to the former SEQ ID n^{o} 1506 |
| SEQ ID N^{o} 3190 | Listeria monocytogenes 4b Contig319 |
| SEQ ID N^{o} 3191 | Listeria monocytogenes 4b Contig320 |
| | Corresponding to the former SEQ ID n^{o} 1239 |
| SEQ ID N^{o} 3192 | Listeria monocytogenes 4b Contig321 |
| SEQ ID N^{o} 3193 | Listeria monocytogenes 4b Contig322 |
| | Corresponding to the former SEQ ID n^{o} 1424 |
| SEQ ID N^{o} 3194 | Listeria monocytogenes 4b Contig323 |
| | Corresponding to the former SEQ ID n^{o} 1576 |
| SEQ ID N^{o} 3195 | Listeria monocytogenes 4b Contig324 |
| SEQ ID N^{o} 3196 | Listeria monocytogenes 4b Contig325 |
| | Corresponding to the former SEQ ID n^{o} 1392 |
| SEQ ID N^{o} 3197 | Listeria monocytogenes 4b Contig326 |
| | Corresponding to the former SEQ ID n^{o} 1492 |
| SEQ ID N^{o} 3198 | Listeria monocytogenes 4b Contig327 |
| SEQ ID N^{o} 3199 | Listeria monocytogenes 4b Contig328 |
| | Corresponding to the former SEQ ID n^{o} 1122 |
| SEQ ID N^{o} 3200 | Listeria monocytogenes 4b Contig329 |
| SEQ ID N^{o} 3201 | Listeria monocytogenes 4b Contig330 |
| | Corresponding to the former SEQ ID n^{o} 1231 |
| SEQ ID N^{o} 3202 | Listeria monocytogenes 4b Contig331 |
| | Corresponding to the former SEQ ID n^{o} 1583 |
| SEQ ID N^{o} 3203 | Listeria monocytogenes 4b Contig332 |
| SEQ ID N^{o} 3204 | Listeria monocytogenes 4b Contig333 |
| SEQ ID N^{o} 3205 | Listeria monocytogenes 4b Contig334 |
| | Corresponding to the former SEQ ID n^{o} 1125 |
| SEQ ID N^{o} 3206 | Listeria monocytogenes 4b Contig335 |
| SEQ ID N^{o} 3207 | Listeria monocytogenes 4b Contig336 |
| SEQ ID N^{o} 3208 | Listeria monocytogenes 4b Contig337 |
| SEQ ID N^{o} 3209 | Listeria monocytogenes 4b Contig338 |
| | Corresponding to the former SEQ ID n^{o} 1105 |
| SEQ ID N^{o} 3210 | Listeria monocytogenes 4b Contig339 |
| | Corresponding to the former SEQ ID n^{o} 1840 |
| SEQ ID N^{o} 3211 | Listeria monocytogenes 4b Contig340 |
| | Corresponding to the former SEQ ID n^{o} 1262 |
| SEQ ID N^{o} 3212 | Listeria monocytogenes 4b Contig341 |
| SEQ ID N^{o} 3213 | Listeria monocytogenes 4b Contig342 |
| SEQ ID N^{o} 3214 | Listeria monocytogenes 4b Contig343 |
| SEQ ID N^{o} 3215 | Listeria monocytogenes 4b Contig344 |
| | Corresponding to the former SEQ ID n^{o} 1662 |
| SEQ ID N^{o} 3216 | Listeria monocytogenes 4b Contig345 |
| | Corresponding to the former SEQ ID n^{o} 1215 |
| SEQ ID N^{o} 3217 | Listeria monocytogenes 4b Contig346 |
| | Corresponding to the former SEQ ID n^{o} 1350 |
| SEQ ID N^{o} 3218 | Listeria monocytogenes 4b Contig347 |
| | Corresponding to the former SEQ ID n^{o} 1101 |
| SEQ ID N^{o} 3219 | Listeria monocytogenes 4b Contig348 |
| | Corresponding to the former SEQ ID n^{o} 1162 |
| SEQ ID N^{o} 3220 | Listeria monocytogenes 4b Contig349 |
| | Corresponding to the former SEQ ID n^{o} 1251 |
| SEQ ID N^{o} 3221 | Listeria monocytogenes 4b Contig350 |
| | Corresponding to the former SEQ ID n^{o} 1696 |
| SEQ ID N^{o} 3222 | Listeria monocytogenes 4b Contig351 |
| SEQ ID N^{o} 3223 | Listeria monocytogenes 4b Contig352 |
| SEQ ID N^{o} 3224 | Listeria monocytogenes 4b Contig353 |
| SEQ ID N^{o} 3225 | Listeria monocytogenes 4b Contig354 |
| SEQ ID N^{o} 3226 | Listeria monocytogenes 4b Contig355 |
| SEQ ID N^{o} 3227 | Listeria monocytogenes 4b Contig356 |
| | Corresponding to the former SEQ ID n^{o} 1248 |
| SEQ ID N^{o} 3228 | Listeria monocytogenes 4b Contig357 |
| | Corresponding to the former SEQ ID n^{o} 1849 |
| SEQ ID N^{o} 3229 | Listeria monocytogenes 4b Contig358 |
| | Corresponding to the former SEQ ID n^{o} 1229 |
| SEQ ID N^{o} 3230 | Listeria monocytogenes 4b Contig359 |
| | Corresponding to the former SEQ ID n^{o} 1858 |
| SEQ ID N^{o} 3231 | Listeria monocytogenes 4b Contig360 |
| | Corresponding to the former SEQ ID n^{o} 1270 |
| SEQ ID N^{o} 3232 | Listeria monocytogenes 4b Contig361 |
| SEQ ID N^{o} 3233 | Listeria monocytogenes 4b Contig362 |
| | Corresponding to the former SEQ ID n^{o} 1862 |
| SEQ ID N^{o} 3234 | Listeria monocytogenes 4b Contig363 |
| | Corresponding to the former SEQ ID n^{o} 1078 |
| SEQ ID N^{o} 3235 | Listeria monocytogenes 4b Contig364 |
| | Corresponding to the former SEQ ID n^{o} 1090 |
| SEQ ID N^{o} 3236 | Listeria monocytogenes 4b Contig365 |
| SEQ ID N^{o} 3237 | Listeria monocytogenes 4b Contig366 |
| SEQ ID N^{o} 3238 | Listeria monocytogenes 4b Contig367 |
| | Corresponding to the former SEQ ID n^{o} 1115 |
| SEQ ID N^{o} 3239 | Listeria monocytogenes 4b Contig368 |
| SEQ ID N^{o} 3240 | Listeria monocytogenes 4b Contig369 |
| SEQ ID N^{o} 3241 | Listeria monocytogenes 4b Contig370 |
| | Corresponding to the former SEQ ID n^{o} 1741 |
| SEQ ID N^{o} 3242 | Listeria monocytogenes 4b Contig371 |
| | Corresponding to the former SEQ ID n^{o} 1340 |
| SEQ ID N^{o} 3243 | Listeria monocytogenes 4b Contig372 |
| | Corresponding to the former SEQ ID n^{o} 1822 |
| SEQ ID N^{o} 3244 | Listeria monocytogenes 4b Contig373 |
| SEQ ID N^{o} 3245 | Listeria monocytogenes 4b Contig374 |
| | Corresponding to the former SEQ ID n^{o} 1426 |
| SEQ ID N^{o} 3246 | Listeria monocytogenes 4b Contig375 |
| | Corresponding to the former SEQ ID n^{o} 1614 |
| SEQ ID N^{o} 3247 | Listeria monocytogenes 4b Contig376 |
| SEQ ID N^{o} 3248 | Listeria monocytogenes 4b Contig377 |
| SEQ ID N^{o} 3249 | Listeria monocytogenes 4b Contig378 |
| SEQ ID N^{o} 3250 | Listeria monocytogenes 4b Contig379 |
| SEQ ID N^{o} 3251 | Listeria monocytogenes 4b Contig380 |
| | Corresponding to the former SEQ ID n^{o} 1679 |
| SEQ ID N^{o} 3252 | Listeria monocytogenes 4b Contig381 |
| SEQ ID N^{o} 3253 | Listeria monocytogenes 4b Contig382 |
| SEQ ID N^{o} 3254 | Listeria monocytogenes 4b Contig383 |
| SEQ ID N^{o} 3255 | Listeria monocytogenes 4b Contig384 |
| SEQ ID N^{o} 3256 | Listeria monocytogenes 4b Contig385 |
| | Corresponding to the former SEQ ID n^{o} 1394 |
| SEQ ID N^{o} 3257 | Listeria monocytogenes 4b Contig386 |
| | Corresponding to the former SEQ ID n^{o} 1722 |
| SEQ ID N^{o} 3258 | Listeria monocytogenes 4b Contig387 |
| SEQ ID N^{o} 3259 | Listeria monocytogenes 4b Contig388 |
| | Corresponding to the former SEQ ID n^{o} 1609 |
| SEQ ID N^{o} 3260 | Listeria monocytogenes 4b Contig389 |
| | Corresponding to the former SEQ ID n^{o} 1777 |
| SEQ ID N^{o} 3261 | Listeria monocytogenes 4b Contig390 |
| | Corresponding to the former SEQ ID n^{o} 1396 |
| SEQ ID N^{o} 3262 | Listeria monocytogenes 4b Contig391 |
| | Corresponding to the former SEQ ID n^{o} 1546 |
| SEQ ID N^{o} 3263 | Listeria monocytogenes 4b Contig392 |
| | Corresponding to the former SEQ ID n^{o} 1438 |
| SEQ ID N^{o} 3264 | Listeria monocytogenes 4b Contig393 |
| | Corresponding to the former SEQ ID n^{o} 1440 |
| SEQ ID N^{o} 3265 | Listeria monocytogenes 4b Contig394 |
| | Corresponding to the former SEQ ID n^{o} 1582 |
| SEQ ID N^{o} 3266 | Listeria monocytogenes 4b Contig395 |
| SEQ ID N^{o} 3267 | Listeria monocytogenes 4b Contig396 |
| | Corresponding to the former SEQ ID n^{o} 1449 |
| SEQ ID N^{o} 3268 | Listeria monocytogenes 4b Contig397 |
| | Corresponding to the former SEQ ID n^{o} 1651 |
| SEQ ID N^{o} 3269 | Listeria monocytogenes 4b Contig398 |
| | Corresponding to the former SEQ ID n^{o} 1325 |
| SEQ ID N^{o} 3270 | Listeria monocytogenes 4b Contig399 |
| | Corresponding to the former SEQ ID n^{o} 1703 |
| SEQ ID N^{o} 3271 | Listeria monocytogenes 4b Contig400 |
| SEQ ID N^{o} 3272 | Listeria monocytogenes 4b Contig401 |
| | Corresponding to the former SEQ ID n^{o} 1483 |
| SEQ ID N^{o} 3273 | Listeria monocytogenes 4b Contig402 |
| SEQ ID N^{o} 3274 | Listeria monocytogenes 4b Contig403 |
| | Corresponding to the former SEQ ID n^{o} 1448 |
| SEQ ID N^{o} 3275 | Listeria monocytogenes 4b Contig404 |
| SEQ ID N^{o} 3276 | Listeria monocytogenes 4b Contig405 |
| | Corresponding to the former SEQ ID n^{o} 1653 |
| SEQ ID N^{o} 3277 | Listeria monocytogenes 4b Contig406 |
| | Corresponding to the former SEQ ID n^{o} 1725 |
| SEQ ID N^{o} 3278 | Listeria monocytogenes 4b Contig407 |
| SEQ ID N^{o} 3279 | Listeria monocytogenes 4b Contig408 |
| | Corresponding to the former SEQ ID n^{o} 1505 |
| SEQ ID N^{o} 3280 | Listeria monocytogenes 4b Contig409 |
| SEQ ID N^{o} 3281 | Listeria monocytogenes 4b Contig410 |
| | Corresponding to the former SEQ ID n^{o} 1669 |
| SEQ ID N^{o} 3282 | Listeria monocytogenes 4b Contig411 |
| SEQ ID N^{o} 3283 | Listeria monocytogenes 4b Contig412 |
| | Corresponding to the former SEQ ID n^{o} 1282 |
| SEQ ID N^{o} 3284 | Listeria monocytogenes 4b Contig413 |
| SEQ ID N^{o} 3285 | Listeria monocytogenes 4b Contig414 |
| SEQ ID N^{o} 3286 | Listeria monocytogenes 4b Contig415 |
| SEQ ID N^{o} 3287 | Listeria monocytogenes 4b Contig416 |
| | Corresponding to the former SEQ ID n^{o} 1693 |
| SEQ ID N^{o} 3288 | Listeria monocytogenes 4b Contig417 |
| | Corresponding to the former SEQ ID n^{o} 1242 |
| SEQ ID N^{o} 3289 | Listeria monocytogenes 4b Contig418 |
| | Corresponding to the former SEQ ID n^{o} 1527 |
| SEQ ID N^{o} 3290 | Listeria monocytogenes 4b Contig419 |
| | Corresponding to the former SEQ ID n^{o} 1683 |
| SEQ ID N^{o} 3291 | Listeria monocytogenes 4b Contig420 |
| | Corresponding to the former SEQ ID n^{o} 1166 |
| SEQ ID N^{o} 3292 | Listeria monocytogenes 4b Contig421 |
| SEQ ID N^{o} 3293 | Listeria monocytogenes 4b Contig422 |
| | Corresponding to the former SEQ ID n^{o} 1676 |
| SEQ ID N^{o} 3294 | Listeria monocytogenes 4b Contig423 |
| | Corresponding to the former SEQ ID n^{o} 1675 |
| SEQ ID N^{o} 3295 | Listeria monocytogenes 4b Contig424 |
| SEQ ID N^{o} 3296 | Listeria monocytogenes 4b Contig425 |
| | Corresponding to the former SEQ ID n^{o} 1299 |
| SEQ ID N^{o} 3297 | Listeria monocytogenes 4b Contig426 |
| SEQ ID N^{o} 3298 | Listeria monocytogenes 4b Contig427 |
| | Corresponding to the former SEQ ID n^{o} 1578 |
| SEQ ID N^{o} 3299 | Listeria monocytogenes 4b Contig428 |
| SEQ ID N^{o} 3300 | Listeria monocytogenes 4b Contig429 |
| | Corresponding to the former SEQ ID n^{o} 1176 |
| SEQ ID N^{o} 3301 | Listeria monocytogenes 4b Contig430 |
| SEQ ID N^{o} 3302 | Listeria monocytogenes 4b Contig431 |
| SEQ ID N^{o} 3303 | Listeria monocytogenes 4b Contig432 |
| | Corresponding to the former SEQ ID n^{o} 1263 |
| SEQ ID N^{o} 3304 | Listeria monocytogenes 4b Contig433 |
| | Corresponding to the former SEQ ID n^{o} 1637 |
| SEQ ID N^{o} 3305 | Listeria monocytogenes 4b Contig434 |
| | Corresponding to the former SEQ ID n^{o} 1665 |
| SEQ ID N^{o} 3306 | Listeria monocytogenes 4b Contig435 |
| | Corresponding to the former SEQ ID n^{o} 1243 |
| | Corresponding to the former SEQ ID n^{o} 1431 |
| SEQ ID N^{o} 3307 | Listeria monocytogenes 4b Contig436 |
| SEQ ID N^{o} 3308 | Listeria monocytogenes 4b Contig437 |
| SEQ ID N^{o} 3309 | Listeria monocytogenes 4b Contig438 |
| SEQ ID N^{o} 3310 | Listeria monocytogenes 4b Contig439 |
| | Corresponding to the former SEQ ID n^{o} 1615 |
| SEQ ID N^{o} 3311 | Listeria monocytogenes 4b Contig440 |
| | Corresponding to the former SEQ ID n^{o} 1074 |
| | Corresponding to the former SEQ ID n^{o} 1518 |
| SEQ ID N^{o} 3312 | Listeria monocytogenes 4b Contig441 |
| | Corresponding to the former SEQ ID n^{o} 1118 |
| SEQ ID N^{o} 3313 | Listeria monocytogenes 4b Contig442 |
| | Corresponding to the former SEQ ID n^{o} 1692 |
| SEQ ID N^{o} 3314 | Listeria monocytogenes 4b Contig443 |
| | Corresponding to the former SEQ ID n^{o} 1255 |
| SEQ ID N^{o} 3315 | Listeria monocytogenes 4b Contig444 |
| SEQ ID N^{o} 3316 | Listeria monocytogenes 4b Contig445 |
| | Corresponding to the former SEQ ID n^{o} 1283 |
| SEQ ID N^{o} 3317 | Listeria monocytogenes 4b Contig446 |
| | Corresponding to the former SEQ ID n^{o} 1387 |
| SEQ ID N^{o} 3318 | Listeria monocytogenes 4b Contig447 |
| SEQ ID N^{o} 3319 | Listeria monocytogenes 4b Contig448 |
| | Corresponding to the former SEQ ID n^{o} 1953 |
| SEQ ID N^{o} 3320 | Listeria monocytogenes 4b Contig449 |
| SEQ ID N^{o} 3321 | Listeria monocytogenes 4b Contig450 |
| SEQ ID N^{o} 3322 | Listeria monocytogenes 4b Contig451 |
| | Corresponding to the former SEQ ID n^{o} 1714 |
| SEQ ID N^{o} 3323 | Listeria monocytogenes 4b Contig452 |
| SEQ ID N^{o} 3324 | Listeria monocytogenes 4b Contig453 |
| SEQ ID N^{o} 3325 | Listeria monocytogenes 4b Contig454 |
| SEQ ID N^{o} 3326 | Listeria monocytogenes 4b Contig455 |
| SEQ ID N^{o} 3327 | Listeria monocytogenes 4b Contig456 |
| SEQ ID N^{o} 3328 | Listeria monocytogenes 4b Contig457 |
| SEQ ID N^{o} 3329 | Listeria monocytogenes 4b Contig458 |
| | Corresponding to the former SEQ ID n^{o} 1208 |
| SEQ ID N^{o} 3330 | Listeria monocytogenes 4b Contig459 |
| | Corresponding to the former SEQ ID n^{o} 1403 |
| SEQ ID N^{o} 3331 | Listeria monocytogenes 4b Contig460 |
| | Corresponding to the former SEQ ID n^{o} 1443 |
| | Corresponding to the former SEQ ID n^{o} 1558 |
| SEQ ID N^{o} 3332 | Listeria monocytogenes 4b Contig461 |
| | Corresponding to the former SEQ ID n^{o} 1323 |
| SEQ ID N^{o} 3333 | Listeria monocytogenes 4b Contig462 |
| | Corresponding to the former SEQ ID n^{o} 1184 |
| SEQ ID N^{o} 3334 | Listeria monocytogenes 4b Contig463 |
| SEQ ID N^{o} 3335 | Listeria monocytogenes 4b Contig464 |
| SEQ ID N^{o} 3336 | Listeria monocytogenes 4b Contig465 |
| | Corresponding to the former SEQ ID n^{o} 1274 |
| SEQ ID N^{o} 3337 | Listeria monocytogenes 4b Contig466 |
| | Corresponding to the former SEQ ID n^{o} 1815 |
| SEQ ID N^{o} 3338 | Listeria monocytogenes 4b Contig467 |
| | Corresponding to the former SEQ ID n^{o} 1607 |
| SEQ ID N^{o} 3339 | Listeria monocytogenes 4b Contig468 |
| | Corresponding to the former SEQ ID n^{o} 1573 |
| SEQ ID N^{o} 3340 | Listeria monocytogenes 4b Contig469 |
| SEQ ID N^{o} 3341 | Listeria monocytogenes 4b Contig470 |
| | Corresponding to the former SEQ ID n^{o} 1737 |
| SEQ ID N^{o} 3342 | Listeria monocytogenes 4b Contig471 |
| | Corresponding to the former SEQ ID n^{o} 1404 |
| SEQ ID N^{o} 3343 | Listeria monocytogenes 4b Contig472 |
| | Corresponding to the former SEQ ID n^{o} 1344 |
| SEQ ID N^{o} 3344 | Listeria monocytogenes 4b Contig473 |
| | Corresponding to the former SEQ ID n^{o} 1197 |
| | Corresponding to the former SEQ ID n^{o} 1490 |
| SEQ ID N^{o} 3345 | Listeria monocytogenes 4b Contig474 |
| SEQ ID N^{o} 3346 | Listeria monocytogenes 4b Contig475 |
| | Corresponding to the former SEQ ID n^{o} 1346 |
| SEQ ID N^{o} 3347 | Listeria monocytogenes 4b Contig476 |
| | Corresponding to the former SEQ ID n^{o} 1112 |
| SEQ ID N^{o} 3348 | Listeria monocytogenes 4b Contig477 |
| SEQ ID N^{o} 3349 | Listeria monocytogenes 4b Contig478 |
| SEQ ID N^{o} 3350 | Listeria monocytogenes 4b Contig479 |
| SEQ ID N^{o} 3351 | Listeria monocytogenes 4b Contig480 |
| SEQ ID N^{o} 3352 | Listeria monocytogenes 4b Contig481 |
| | Corresponding to the former SEQ ID n^{o} 1213 |
| SEQ ID N^{o} 3353 | Listeria monocytogenes 4b Contig482 |
| | Corresponding to the former SEQ ID n^{o} 1124 |
| SEQ ID N^{o} 3354 | Listeria monocytogenes 4b Contig483 |
| | Corresponding to the former SEQ ID n^{o} 1250 |
| SEQ ID N^{o} 3355 | Listeria monocytogenes 4b Contig484 |
| SEQ ID N^{o} 3356 | Listeria monocytogenes 4b Contig485 |
| | Corresponding to the former SEQ ID n^{o} 1362 |
| SEQ ID N^{o} 3357 | Listeria monocytogenes 4b Contig486 |
| | Corresponding to the former SEQ ID n^{o} 1408 |
| SEQ ID N^{o} 3358 | Listeria monocytogenes 4b Contig487 |
| SEQ ID N^{o} 3359 | Listeria monocytogenes 4b Contig488 |
| | Corresponding to the former SEQ ID n^{o} 1331 |
| SEQ ID N^{o} 3360 | Listeria monocytogenes 4b Contig489 |
| SEQ ID N^{o} 3361 | Listeria monocytogenes 4b Contig490 |
| | Corresponding to the former SEQ ID n^{o} 1596 |
| SEQ ID N^{o} 3362 | Listeria monocytogenes 4b Contig491 |
| | Corresponding to the former SEQ ID n^{o} 1537 |
| SEQ ID N^{o} 3363 | Listeria monocytogenes 4b Contig492 |
| SEQ ID N^{o} 3364 | Listeria monocytogenes 4b Contig493 |
| | Corresponding to the former SEQ ID n^{o} 1503 |
| SEQ ID N^{o} 3365 | Listeria monocytogenes 4b Contig494 |
| | Corresponding to the former SEQ ID n^{o} 1915 |
| SEQ ID N^{o} 3366 | Listeria monocytogenes 4b Contig495 |
| SEQ ID N^{o} 3367 | Listeria monocytogenes 4b Contig496 |
| SEQ ID N^{o} 3368 | Listeria monocytogenes 4b Contig497 |
| SEQ ID N^{o} 3369 | Listeria monocytogenes 4b Contig498 |
| | Corresponding to the former SEQ ID n^{o} 1476 |
| SEQ ID N^{o} 3370 | Listeria monocytogenes 4b Contig499 |
| | Corresponding to the former SEQ ID n^{o} 1871 |
| SEQ ID N^{o} 3371 | Listeria monocytogenes 4b Contig500 |
| | Corresponding to the former SEQ ID n^{o} 1071 |
| | Corresponding to the former SEQ ID n^{o} 1526 |
| SEQ ID N^{o} 3372 | Listeria monocytogenes 4b Contig501 |
| | Corresponding to the former SEQ ID n^{o} 1324 |
| SEQ ID N^{o} 3373 | Listeria monocytogenes 4b Contig502 |
| | Corresponding to the former SEQ ID n^{o} 1709 |
| SEQ ID N^{o} 3374 | Listeria monocytogenes 4b Contig503 |
| SEQ ID N^{o} 3375 | Listeria monocytogenes 4b Contig504 |
| | Corresponding to the former SEQ ID n^{o} 1172 |
| SEQ ID N^{o} 3376 | Listeria monocytogenes 4b Contig505 |
| SEQ ID N^{o} 3377 | Listeria monocytogenes 4b Contig506 |
| | Corresponding to the former SEQ ID n^{o} 1077 |
| SEQ ID N^{o} 3378 | Listeria monocytogenes 4b Contig507 |
| | Corresponding to the former SEQ ID n^{o} 1329 |
| SEQ ID N^{o} 3379 | Listeria monocytogenes 4b Contig508 |
| | Corresponding to the former SEQ ID n^{o} 1416 |
| SEQ ID N^{o} 3380 | Listeria monocytogenes 4b Contig509 |
| | Corresponding to the former SEQ ID n^{o} 1763 |
| SEQ ID N^{o} 3381 | Listeria monocytogenes 4b Contig510 |
| | Corresponding to the former SEQ ID n^{o} 1916 |
| SEQ ID N^{o} 3382 | Listeria monocytogenes 4b Contig511 |
| | Corresponding to the former SEQ ID n^{o} 1285 |
| SEQ ID N^{o} 3383 | Listeria monocytogenes 4b Contig512 |
| | Corresponding to the former SEQ ID n^{o} 1298 |
| SEQ ID N^{o} 3384 | Listeria monocytogenes 4b Contig513 |
| SEQ ID N^{o} 3385 | Listeria monocytogenes 4b Contig514 |
| | Corresponding to the former SEQ ID n^{o} 1237 |
| SEQ ID N^{o} 3386 | Listeria monocytogenes 4b Contig515 |
| SEQ ID N^{o} 3387 | Listeria monocytogenes 4b Contig516 |
| SEQ ID N^{o} 3388 | Listeria monocytogenes 4b Contig517 |
| | Corresponding to the former SEQ ID n^{o} 1658 |
| SEQ ID N^{o} 3389 | Listeria monocytogenes 4b Contig518 |
| SEQ ID N^{o} 3390 | Listeria monocytogenes 4b Contig519 |
| | Corresponding to the former SEQ ID n^{o} 1454 |
| SEQ ID N^{o} 3391 | Listeria monocytogenes 4b Contig520 |
| | Corresponding to the former SEQ ID n^{o} 1351 |
| SEQ ID N^{o} 3392 | Listeria monocytogenes 4b Contig521 |
| | Corresponding to the former SEQ ID n^{o} 1148 |
| SEQ ID N^{o} 3393 | Listeria monocytogenes 4b Contig522 |
| | Corresponding to the former SEQ ID n^{o} 1456 |
| SEQ ID N^{o} 3394 | Listeria monocytogenes 4b Contig523 |
| | Corresponding to the former SEQ ID n^{o} 1899 |
| SEQ ID N^{o} 3395 | Listeria monocytogenes 4b Contig524 |
| | Corresponding to the former SEQ ID n^{o} 1847 |
| SEQ ID N^{o} 3396 | Listeria monocytogenes 4b Contig525 |
| | Corresponding to the former SEQ ID n^{o} 1639 |
| SEQ ID N^{o} 3397 | Listeria monocytogenes 4b Contig526 |
| | Corresponding to the former SEQ ID n^{o} 1663 |
| SEQ ID N^{o} 3398 | Listeria monocytogenes 4b Contig527 |
| | Corresponding to the former SEQ ID n^{o} 1093 |
| SEQ ID N^{o} 3399 | Listeria monocytogenes 4b Contig528 |
| | Corresponding to the former SEQ ID n^{o} 1193 |
| SEQ ID N^{o} 3400 | Listeria monocytogenes 4b Contig529 |
| | Corresponding to the former SEQ ID n^{o} 1280 |
| SEQ ID N^{o} 3401 | Listeria monocytogenes 4b Contig530 |
| | Corresponding to the former SEQ ID n^{o} 1143 |
| SEQ ID N^{o} 3402 | Listeria monocytogenes 4b Contig531 |
| | Corresponding to the former SEQ ID n^{o} 1499 |
| SEQ ID N^{o} 3403 | Listeria monocytogenes 4b Contig532 |
| | Corresponding to the former SEQ ID n^{o} 1627 |
| SEQ ID N^{o} 3404 | Listeria monocytogenes 4b Contig533 |
| SEQ ID N^{o} 3405 | Listeria monocytogenes 4b Contig534 |
| SEQ ID N^{o} 3406 | Listeria monocytogenes 4b Contig535 |
| | Corresponding to the former SEQ ID n^{o} 1133 |
| SEQ ID N^{o} 3407 | Listeria monocytogenes 4b Contig536 |
| | Corresponding to the former SEQ ID n^{o} 1163 |
| SEQ ID N^{o} 3408 | Listeria monocytogenes 4b Contig537 |
| | Corresponding to the former SEQ ID n^{o} 1135 |
| SEQ ID N^{o} 3409 | Listeria monocytogenes 4b Contig538 |
| SEQ ID N^{o} 3410 | Listeria monocytogenes 4b Contig539 |
| | Corresponding to the former SEQ ID n^{o} 1838 |
| SEQ ID N^{o} 3411 | Listeria monocytogenes 4b Contig540 |
| | Corresponding to the former SEQ ID n^{o} 1217 |
| SEQ ID N^{o} 3412 | Listeria monocytogenes 4b Contig541 |
| | Corresponding to the former SEQ ID n^{o} 1297 |
| SEQ ID N^{o} 3413 | Listeria monocytogenes 4b Contig542 |
| SEQ ID N^{o} 3414 | Listeria monocytogenes 4b Contig543 |
| | Corresponding to the former SEQ ID n^{o} 1765 |
| SEQ ID N^{o} 3415 | Listeria monocytogenes 4b Contig544 |
| | Corresponding to the former SEQ ID n^{o} 1228 |
| SEQ ID N^{o} 3416 | Listeria monocytogenes 4b Contig545 |
| | Corresponding to the former SEQ ID n^{o} 1638 |
| SEQ ID N^{o} 3417 | Listeria monocytogenes 4b Contig546 |
| | Corresponding to the former SEQ ID n^{o} 1965 |
| SEQ ID N^{o} 3418 | Listeria monocytogenes 4b Contig547 |
| | Corresponding to the former SEQ ID n^{o} 1211 |
| SEQ ID N^{o} 3419 | Listeria monocytogenes 4b Contig548 |
| | Corresponding to the former SEQ ID n^{o} 1327 |
| SEQ ID N^{o} 3420 | Listeria monocytogenes 4b Contig549 |
| | Corresponding to the former SEQ ID n^{o} 1502 |
| SEQ ID N^{o} 3421 | Listeria monocytogenes 4b Contig550 |
| | Corresponding to the former SEQ ID n^{o} 1252 |
| SEQ ID N^{o} 3422 | Listeria monocytogenes 4b Contig551 |
| | Corresponding to the former SEQ ID n^{o} 1721 |
| SEQ ID N^{o} 3423 | Listeria monocytogenes 4b Contig552 |
| | Corresponding to the former SEQ ID n^{o} 1349 |
| | Corresponding to the former SEQ ID n^{o} 1728 |
| SEQ ID N^{o} 3424 | Listeria monocytogenes 4b Contig553 |
| | Corresponding to the former SEQ ID n^{o} 1691 |
| SEQ ID N^{o} 3425 | Listeria monocytogenes 4b Contig554 |
| | Corresponding to the former SEQ ID n^{o} 1227 |
| | Corresponding to the former SEQ ID n^{o} 1399 |
| SEQ ID N^{o} 3426 | Listeria monocytogenes 4b Contig555 |
| | Corresponding to the former SEQ ID n^{o} 1555 |
| SEQ ID N^{o} 3427 | Listeria monocytogenes 4b Contig556 |
| SEQ ID N^{o} 3428 | Listeria monocytogenes 4b Contig557 |
| | Corresponding to the former SEQ ID n^{o} 1114 |
| | Corresponding to the former SEQ ID n^{o} 1366 |
| SEQ ID N^{o} 3429 | Listeria monocytogenes 4b Contig558 |
| SEQ ID N^{o} 3430 | Listeria monocytogenes 4b Contig559 |
| | Corresponding to the former SEQ ID n^{o} 1805 |
| SEQ ID N^{o} 3431 | Listeria monocytogenes 4b Contig560 |
| | Corresponding to the former SEQ ID n^{o} 1852 |
| SEQ ID N^{o} 3432 | Listeria monocytogenes 4b Contig561 |
| SEQ ID N^{o} 3433 | Listeria monocytogenes 4b Contig562 |
| | Corresponding to the former SEQ ID n^{o} 1923 |
| SEQ ID N^{o} 3434 | Listeria monocytogenes 4b Contig563 |
| | Corresponding to the former SEQ ID n^{o} 1395 |
| SEQ ID N^{o} 3435 | Listeria monocytogenes 4b Contig564 |
| | Corresponding to the former SEQ ID n^{o} 1561 |
| SEQ ID N^{o} 3436 | Listeria monocytogenes 4b Contig565 |
| SEQ ID N^{o} 3437 | Listeria monocytogenes 4b Contig566 |
| | Corresponding to the former SEQ ID n^{o} 1643 |
| SEQ ID N^{o} 3438 | Listeria monocytogenes 4b Contig567 |
| | Corresponding to the former SEQ ID n^{o} 1820 |
| SEQ ID N^{o} 3439 | Listeria monocytogenes 4b Contig568 |
| | Corresponding to the former SEQ ID n^{o} 1177 |
| SEQ ID N^{o} 3440 | Listeria monocytogenes 4b Contig569 |
| SEQ ID N^{o} 3441 | Listeria monocytogenes 4b Contig570 |
| | Corresponding to the former SEQ ID n^{o} 1501 |
| SEQ ID N^{o} 3442 | Listeria monocytogenes 4b Contig571 |
| | Corresponding to the former SEQ ID n^{o} 1195 |
| SEQ ID N^{o} 3443 | Listeria monocytogenes 4b Contig572 |
| | Corresponding to the former SEQ ID n^{o} 1556 |
| SEQ ID N^{o} 3444 | Listeria monocytogenes 4b Contig573 |
| SEQ ID N^{o} 3445 | Listeria monocytogenes 4b Contig574 |
| | Corresponding to the former SEQ ID n^{o} 1888 |
| SEQ ID N^{o} 3446 | Listeria monocytogenes 4b Contig575 |
| | Corresponding to the former SEQ ID n^{o} 1730 |
| SEQ ID N^{o} 3447 | Listeria monocytogenes 4b Contig576 |
| | Corresponding to the former SEQ ID n^{o} 1629 |
| SEQ ID N^{o} 3448 | Listeria monocytogenes 4b Contig577 |
| | Corresponding to the former SEQ ID n^{o} 1688 |
| SEQ ID N^{o} 3449 | Listeria monocytogenes 4b Contig578 |
| | Corresponding to the former SEQ ID n^{o} 1549 |
| SEQ ID N^{o} 3450 | Listeria monocytogenes 4b Contig579 |
| | Corresponding to the former SEQ ID n^{o} 1673 |
| SEQ ID N^{o} 3451 | Listeria monocytogenes 4b Contig580 |
| | Corresponding to the former SEQ ID n^{o} 1273 |
| SEQ ID N^{o} 3452 | Listeria monocytogenes 4b Contig581 |
| | Corresponding to the former SEQ ID n^{o} 1415 |
| SEQ ID N^{o} 3453 | Listeria monocytogenes 4b Contig582 |
| SEQ ID N^{o} 3454 | Listeria monocytogenes 4b Contig583 |
| | Corresponding to the former SEQ ID n^{o} 1281 |
| SEQ ID N^{o} 3455 | Listeria monocytogenes 4b Contig584 |
| | Corresponding to the former SEQ ID n^{o} 1572 |
| SEQ ID N^{o} 3456 | Listeria monocytogenes 4b Contig585 |
| SEQ ID N^{o} 3457 | Listeria monocytogenes 4b Contig586 |
| | Corresponding to the former SEQ ID n^{o} 1949 |
| SEQ ID N^{o} 3458 | Listeria monocytogenes 4b Contig587 |
| | Corresponding to the former SEQ ID n^{o} 1625 |
| SEQ ID N^{o} 3459 | Listeria monocytogenes 4b Contig588 |
| SEQ ID N^{o} 3460 | Listeria monocytogenes 4b Contig589 |
| | Corresponding to the former SEQ ID n^{o} 1622 |
| SEQ ID N^{o} 3461 | Listeria monocytogenes 4b Contig590 |
| | Corresponding to the former SEQ ID n^{o} 1086 |
| SEQ ID N^{o} 3462 | Listeria monocytogenes 4b Contig591 |
| | Corresponding to the former SEQ ID n^{o} 1781 |
| SEQ ID N^{o} 3463 | Listeria monocytogenes 4b Contig592 |
| | Corresponding to the former SEQ ID n^{o} 1304 |
| SEQ ID N^{o} 3464 | Listeria monocytogenes 4b Contig593 |
| | Corresponding to the former SEQ ID n^{o} 1489 |
| SEQ ID N^{o} 3465 | Listeria monocytogenes 4b Contig594 |
| | Corresponding to the former SEQ ID n^{o} 1770 |
| SEQ ID N^{o} 3466 | Listeria monocytogenes 4b Contig595 |
| | Corresponding to the former SEQ ID n^{o} 1377 |
| | Corresponding to the former SEQ ID n^{o} 1689 |
| SEQ ID N^{o} 3467 | Listeria monocytogenes 4b Contig596 |
| | Corresponding to the former SEQ ID n^{o} 1225 |
| | Corresponding to the former SEQ ID n^{o} 1759 |
| SEQ ID N^{o} 3468 | Listeria monocytogenes 4b Contig597 |
| SEQ ID N^{o} 3469 | Listeria monocytogenes 4b Contig598 |
| SEQ ID N^{o} 3470 | Listeria monocytogenes 4b Contig599 |
| | Corresponding to the former SEQ ID n^{o} 1477 |
| SEQ ID N^{o} 3471 | Listeria monocytogenes 4b Contig600 |
| | Corresponding to the former SEQ ID n^{o} 1903 |
| SEQ ID N^{o} 3472 | Listeria monocytogenes 4b Contig601 |
| | Corresponding to the former SEQ ID n^{o} 1961 |
| SEQ ID N^{o} 3473 | Listeria monocytogenes 4b Contig602 |
| | Corresponding to the former SEQ ID n^{o} 1754 |
| SEQ ID N^{o} 3474 | Listeria monocytogenes 4b Contig603 |
| | Corresponding to the former SEQ ID n^{o} 1188 |
| SEQ ID N^{o} 3475 | Listeria monocytogenes 4b Contig604 |
| SEQ ID N^{o} 3476 | Listeria monocytogenes 4b Contig605 |
| | Corresponding to the former SEQ ID n^{o} 1913 |
| SEQ ID N^{o} 3477 | Listeria monocytogenes 4b Contig606 |
| SEQ ID N^{o} 3478 | Listeria monocytogenes 4b Contig607 |
| SEQ ID N^{o} 3479 | Listeria monocytogenes 4b Contig608 |
| | Corresponding to the former SEQ ID n^{o} 1439 |
| | Corresponding to the former SEQ ID n^{o} 1545 |
| SEQ ID N^{o} 3480 | Listeria monocytogenes 4b Contig609 |
| | Corresponding to the former SEQ ID n^{o} 1794 |
| SEQ ID N^{o} 3481 | Listeria monocytogenes 4b Contig610 |
| | Corresponding to the former SEQ ID n^{o} 1798 |
| SEQ ID N^{o} 3482 | Listeria monocytogenes 4b Contig611 |
| | Corresponding to the former SEQ ID n^{o} 1200 |
| SEQ ID N^{o} 3483 | Listeria monocytogenes 4b Contig612 |
| | Corresponding to the former SEQ ID n^{o} 1808 |
| SEQ ID N^{o} 3484 | Listeria monocytogenes 4b Contig613 |
| | Corresponding to the former SEQ ID n^{o} 1894 |
| SEQ ID N^{o} 3485 | Listeria monocytogenes 4b Contig614 |
| | Corresponding to the former SEQ ID n^{o} 1812 |
| SEQ ID N^{o} 3486 | Listeria monocytogenes 4b Contig615 |
| | Corresponding to the former SEQ ID n^{o} 1205 |
| SEQ ID N^{o} 3487 | Listeria monocytogenes 4b Contig616 |
| SEQ ID N^{o} 3488 | Listeria monocytogenes 4b Contig617 |
| SEQ ID N^{o} 3489 | Listeria monocytogenes 4b Contig618 |
| | Corresponding to the former SEQ ID n^{o} 1352 |
| SEQ ID N^{o} 3490 | Listeria monocytogenes 4b Contig619 |
| | Corresponding to the former SEQ ID n^{o} 1142 |
| | Corresponding to the former SEQ ID n^{o} 1601 |
| SEQ ID N^{o} 3491 | Listeria monocytogenes 4b Contig620 |
| | Corresponding to the former SEQ ID n^{o} 1575 |
| SEQ ID N^{o} 3492 | Listeria monocytogenes 4b Contig621 |
| | Corresponding to the former SEQ ID n^{o} 1670 |
| SEQ ID N^{o} 3493 | Listeria monocytogenes 4b Contig622 |
| | Corresponding to the former SEQ ID n^{o} 1890 |
| SEQ ID N^{o} 3494 | Listeria monocytogenes 4b Contig623 |
| | Corresponding to the former SEQ ID n^{o} 1333 |
| SEQ ID N^{o} 3495 | Listeria monocytogenes 4b Contig624 |
| | Corresponding to the former SEQ ID n^{o} 1789 |
| SEQ ID N^{o} 3496 | Listeria monocytogenes 4b Contig625 |
| | Corresponding to the former SEQ ID n^{o} 1508 |
| SEQ ID N^{o} 3497 | Listeria monocytogenes 4b Contig626 |
| SEQ ID N^{o} 3498 | Listeria monocytogenes 4b Contig627 |
| | Corresponding to the former SEQ ID n^{o} 1775 |
| SEQ ID N^{o} 3499 | Listeria monocytogenes 4b Contig628 |
| | Corresponding to the former SEQ ID n^{o} 1391 |
| SEQ ID N^{o} 3500 | Listeria monocytogenes 4b Contig629 |
| | Corresponding to the former SEQ ID n^{o} 1657 |
| SEQ ID N^{o} 3501 | Listeria monocytogenes 4b Contig630 |
| | Corresponding to the former SEQ ID n^{o} 1851 |
| SEQ ID N^{o} 3502 | Listeria monocytogenes 4b Contig631 |
| SEQ ID N^{o} 3503 | Listeria monocytogenes 4b Contig632 |
| | Corresponding to the former SEQ ID n^{o} 1711 |
| SEQ ID N^{o} 3504 | Listeria monocytogenes 4b Contig633 |
| | Corresponding to the former SEQ ID n^{o} 1169 |
| SEQ ID N^{o} 3505 | Listeria monocytogenes 4b Contig634 |
| | Corresponding to the former SEQ ID n^{o} 1660 |
| SEQ ID N^{o} 3506 | Listeria monocytogenes 4b Contig635 |
| SEQ ID N^{o} 3507 | Listeria monocytogenes 4b Contig636 |
| SEQ ID N^{o} 3508 | Listeria monocytogenes 4b Contig637 |
| | Corresponding to the former SEQ ID n^{o} 1767 |
| SEQ ID N^{o} 3509 | Listeria monocytogenes 4b Contig638 |
| SEQ ID N^{o} 3510 | Listeria monocytogenes 4b Contig639 |
| SEQ ID N^{o} 3511 | Listeria monocytogenes 4b Contig640 |
| | Corresponding to the former SEQ ID n^{o} 1992 |
| SEQ ID N^{o} 3512 | Listeria monocytogenes 4b Contig641 |
| | Corresponding to the former SEQ ID n^{o} 1413 |
| | Corresponding to the former SEQ ID n^{o} 1515 |
| SEQ ID N^{o} 3513 | Listeria monocytogenes 4b Contig642 |
| | Corresponding to the former SEQ ID n^{o} 1140 |
| | Corresponding to the former SEQ ID n^{o} 1373 |
| SEQ ID N^{o} 3514 | Listeria monocytogenes 4b Contig643 |
| | Corresponding to the former SEQ ID n^{o} 1498 |
| SEQ ID N^{o} 3515 | Listeria monocytogenes 4b Contig644 |
| SEQ ID N^{o} 3516 | Listeria monocytogenes 4b Contig645 |
| SEQ ID N^{o} 3517 | Listeria monocytogenes 4b Contig646 |
| | Corresponding to the former SEQ ID n^{o} 1496 |
| SEQ ID N^{o} 3518 | Listeria monocytogenes 4b Contig647 |
| | Corresponding to the former SEQ ID n^{o} 1934 |
| SEQ ID N^{o} 3519 | Listeria monocytogenes 4b Contig648 |
| | Corresponding to the former SEQ ID n^{o} 1650 |
| SEQ ID N^{o} 3520 | Listeria monocytogenes 4b Contig649 |
| | Corresponding to the former SEQ ID n^{o} 1233 |
| | Corresponding to the former SEQ ID n^{o} 1671 |
| SEQ ID N^{o} 3521 | Listeria monocytogenes 4b Contig650 |
| | Corresponding to the former SEQ ID n^{o} 1950 |
| SEQ ID N^{o} 3522 | Listeria monocytogenes 4b Contig651 |
| | Corresponding to the former SEQ ID n^{o} 1889 |
| SEQ ID N^{o} 3523 | Listeria monocytogenes 4b Contig652 |
| | Corresponding to the former SEQ ID n^{o} 1922 |
| SEQ ID N^{o} 3524 | Listeria monocytogenes 4b Contig653 |
| | Corresponding to the former SEQ ID n^{o} 1313 |
| | Corresponding to the former SEQ ID n^{o} 1453 |
| SEQ ID N^{o} 3525 | Listeria monocytogenes 4b Contig654 |
| | Corresponding to the former SEQ ID n^{o} 1185 |
| SEQ ID N^{o} 3526 | Listeria monocytogenes 4b Contig655 |
| | Corresponding to the former SEQ ID n^{o} 1562 |
| SEQ ID N^{o} 3527 | Listeria monocytogenes 4b Contig656 |
| SEQ ID N^{o} 3528 | Listeria monocytogenes 4b Contig657 |
| SEQ ID N^{o} 3529 | Listeria monocytogenes 4b Contig658 |
| SEQ ID N^{o} 3530 | Listeria monocytogenes 4b Contig659 |
| | Corresponding to the former SEQ ID n^{o} 1348 |
| SEQ ID N^{o} 3531 | Listeria monocytogenes 4b Contig660 |
| | Corresponding to the former SEQ ID n^{o} 1293 |
| SEQ ID N^{o} 3532 | Listeria monocytogenes 4b Contig661 |
| | Corresponding to the former SEQ ID n^{o} 1165 |
| | Corresponding to the former SEQ ID n^{o} 1762 |
| SEQ ID N^{o} 3533 | Listeria monocytogenes 4b Contig662 |
| | Corresponding to the former SEQ ID n^{o} 1640 |
| SEQ ID N^{o} 3534 | Listeria monocytogenes 4b Contig663 |
| SEQ ID N^{o} 3535 | Listeria monocytogenes 4b Contig664 |
| SEQ ID N^{o} 3536 | Listeria monocytogenes 4b Contig665 |
| | Corresponding to the former SEQ ID n^{o} 1764 |
| SEQ ID N^{o} 3537 | Listeria monocytogenes 4b Contig666 |
| | Corresponding to the former SEQ ID n^{o} 1543 |
| SEQ ID N^{o} 3538 | Listeria monocytogenes 4b Contig667 |
| | Corresponding to the former SEQ ID n^{o} 1844 |
| SEQ ID N^{o} 3539 | Listeria monocytogenes 4b Contig668 |
| | Corresponding to the former SEQ ID n^{o} 1560 |
| SEQ ID N^{o} 3540 | Listeria monocytogenes 4b Contig669 |
| | Corresponding to the former SEQ ID n^{o} 1744 |
| SEQ ID N^{o} 3541 | Listeria monocytogenes 4b Contig670 |
| | Corresponding to the former SEQ ID n^{o} 1796 |
| SEQ ID N^{o} 3542 | Listeria monocytogenes 4b Contig671 |
| | Corresponding to the former SEQ ID n^{o} 1776 |
| SEQ ID N^{o} 3543 | Listeria monocytogenes 4b Contig672 |
| | Corresponding to the former SEQ ID n^{o} 1897 |
| SEQ ID N^{o} 3544 | Listeria monocytogenes 4b Contig673 |
| | Corresponding to the former SEQ ID n^{o} 1704 |
| SEQ ID N^{o} 3545 | Listeria monocytogenes 4b Contig674 |
| | Corresponding to the former SEQ ID n^{o} 1713 |
| SEQ ID N^{o} 3546 | Listeria monocytogenes 4b Contig675 |
| | Corresponding to the former SEQ ID n^{o} 1295 |
| | Corresponding to the former SEQ ID n^{o} 1353 |
| SEQ ID N^{o} 3547 | Listeria monocytogenes 4b Contig676 |
| | Corresponding to the former SEQ ID n^{o} 1303 |
| SEQ ID N^{o} 3548 | Listeria monocytogenes 4b Contig677 |
| SEQ ID N^{o} 3549 | Listeria monocytogenes 4b Contig678 |
| SEQ ID N^{o} 3550 | Listeria monocytogenes 4b Contig679 |
| SEQ ID N^{o} 3551 | Listeria monocytogenes 4b Contig680 |
| SEQ ID N^{o} 3552 | Listeria monocytogenes 4b Contig681 |
| | Corresponding to the former SEQ ID n^{o} 1212 |
| | Corresponding to the former SEQ ID n^{o} 1521 |
| SEQ ID N^{o} 3553 | Listeria monocytogenes 4b Contig682 |
| SEQ ID N^{o} 3554 | Listeria monocytogenes 4b Contig683 |
| | Corresponding to the former SEQ ID n^{o} 1694 |
| SEQ ID N^{o} 3555 | Listeria monocytogenes 4b Contig684 |
| | Corresponding to the former SEQ ID n^{o} 1939 |
| SEQ ID N^{o} 3556 | Listeria monocytogenes 4b Contig685 |
| | Corresponding to the former SEQ ID n^{o} 1717 |
| SEQ ID N^{o} 3557 | Listeria monocytogenes 4b Contig686 |
| | Corresponding to the former SEQ ID n^{o} 1626 |
| SEQ ID N^{o} 3558 | Listeria monocytogenes 4b Contig687 |
| | Corresponding to the former SEQ ID n^{o} 1585 |
| SEQ ID N^{o} 3559 | Listeria monocytogenes 4b Contig688 |
| | Corresponding to the former SEQ ID n^{o} 1491 |
| SEQ ID N^{o} 3560 | Listeria monocytogenes 4b Contig689 |
| | Corresponding to the former SEQ ID n^{o} 1314 |
| | Corresponding to the former SEQ ID n^{o} 1481 |
| SEQ ID N^{o} 3561 | Listeria monocytogenes 4b Contig690 |
| | Corresponding to the former SEQ ID n^{o} 1155 |
| SEQ ID N^{o} 3562 | Listeria monocytogenes 4b Contig691 |
| | Corresponding to the former SEQ ID n^{o} 1149 |
| | Corresponding to the former SEQ ID n^{o} 1747 |
| SEQ ID N^{o} 3563 | Listeria monocytogenes 4b Contig692 |
| | Corresponding to the former SEQ ID n^{o} 1364 |
| SEQ ID N^{o} 3564 | Listeria monocytogenes 4b Contig693 |
| | Corresponding to the former SEQ ID n^{o} 1594 |
| SEQ ID N^{o} 3565 | Listeria monocytogenes 4b Contig694 |
| | Corresponding to the former SEQ ID n^{o} 1398 |
| | Corresponding to the former SEQ ID n^{o} 1771 |
| SEQ ID N^{o} 3566 | Listeria monocytogenes 4b Contig695 |
| | Corresponding to the former SEQ ID n^{o} 1178 |
| | Corresponding to the former SEQ ID n^{o} 1684 |
| SEQ ID N^{o} 3567 | Listeria monocytogenes 4b Contig696 |
| | Corresponding to the former SEQ ID n^{o} 1433 |
| | Corresponding to the former SEQ ID n^{o} 1756 |
| SEQ ID N^{o} 3568 | Listeria monocytogenes 4b Contig697 |
| | Corresponding to the former SEQ ID n^{o} 1774 |
| SEQ ID N^{o} 3569 | Listeria monocytogenes 4b Contig698 |
| SEQ ID N^{o} 3570 | Listeria monocytogenes 4b Contig699 |
| | Corresponding to the former SEQ ID n^{o} 1300 |
| SEQ ID N^{o} 3571 | Listeria monocytogenes 4b Contig700 |
| SEQ ID N^{o} 3572 | Listeria monocytogenes 4b Contig701 |
| | Corresponding to the former SEQ ID n^{o} 1547 |
| SEQ ID N^{o} 3573 | Listeria monocytogenes 4b Contig702 |
| | Corresponding to the former SEQ ID n^{o} 1788 |
| SEQ ID N^{o} 3574 | Listeria monocytogenes 4b Contig703 |
| SEQ ID N^{o} 3575 | Listeria monocytogenes 4b Contig704 |
| | Corresponding to the former SEQ ID n^{o} 1872 |
| SEQ ID N^{o} 3576 | Listeria monocytogenes 4b Contig705 |
| | Corresponding to the former SEQ ID n^{o} 1861 |
| SEQ ID N^{o} 3577 | Listeria monocytogenes 4b Contig706 |
| | Corresponding to the former SEQ ID n^{o} 1932 |
| SEQ ID N^{o} 3578 | Listeria monocytogenes 4b Contig707 |
| | Corresponding to the former SEQ ID n^{o} 1553 |
| SEQ ID N^{o} 3579 | Listeria monocytogenes 4b Contig708 |
| | Corresponding to the former SEQ ID n^{o} 1473 |
| SEQ ID N^{o} 3580 | Listeria monocytogenes 4b Contig709 |
| | Corresponding to the former SEQ ID n^{o} 1328 |
| | Corresponding to the former SEQ ID n^{o} 1745 |
| SEQ ID N^{o} 3581 | Listeria monocytogenes 4b Contig710 |
| | Corresponding to the former SEQ ID n^{o} 1557 |
| SEQ ID N^{o} 3582 | Listeria monocytogenes 4b Contig711 |
| | Corresponding to the former SEQ ID n^{o} 1773 |
| SEQ ID N^{o} 3583 | Listeria monocytogenes 4b Contig712 |
| | Corresponding to the former SEQ ID n^{o} 1444 |
| SEQ ID N^{o} 3584 | Listeria monocytogenes 4b Contig713 |
| | Corresponding to the former SEQ ID n^{o} 1826 |
| SEQ ID N^{o} 3585 | Listeria monocytogenes 4b Contig714 |
| | Corresponding to the former SEQ ID n^{o} 1356 |
| | Corresponding to the former SEQ ID n^{o} 1612 |
| SEQ ID N^{o} 3586 | Listeria monocytogenes 4b Contig715 |
| | Corresponding to the former SEQ ID n^{o} 1952 |
| SEQ ID N^{o} 3587 | Listeria monocytogenes 4b Contig716 |
| | Corresponding to the former SEQ ID n^{o} 1874 |
| SEQ ID N^{o} 3588 | Listeria monocytogenes 4b Contig717 |
| | Corresponding to the former SEQ ID n^{o} 1095 |
| | Corresponding to the former SEQ ID n^{o} 1173 |
| SEQ ID N^{o} 3589 | Listeria monocytogenes 4b Contig718 |
| | Corresponding to the former SEQ ID n^{o} 1800 |
| SEQ ID N^{o} 3590 | Listeria monocytogenes 4b Contig719 |
| | Corresponding to the former SEQ ID n^{o} 1320 |
| | Corresponding to the former SEQ ID n^{o} 1832 |
| SEQ ID N^{o} 3591 | Listeria monocytogenes 4b Contig720 |
| | Corresponding to the former SEQ ID n^{o} 1160 |
| | Corresponding to the former SEQ ID n^{o} 1641 |
| SEQ ID N^{o} 3592 | Listeria monocytogenes 4b Contig721 |
| SEQ ID N^{o} 3593 | Listeria monocytogenes 4b Contig722 |
| | Corresponding to the former SEQ ID n^{o} 1144 |
| | Corresponding to the former SEQ ID n^{o} 1216 |
| SEQ ID N^{o} 3594 | Listeria monocytogenes 4b Contig723 |
| SEQ ID N^{o} 3595 | Listeria monocytogenes 4b Contig724 |
| | Corresponding to the former SEQ ID n^{o} 2026 |
| SEQ ID N^{o} 3596 | Listeria monocytogenes 4b Contig725 |
| | Corresponding to the former SEQ ID n^{o} 1226 |
| | Corresponding to the former SEQ ID n^{o} 1588 |
| SEQ ID N^{o} 3597 | Listeria monocytogenes 4b Contig726 |
| | Corresponding to the former SEQ ID n^{o} 1804 |
| SEQ ID N^{o} 3598 | Listeria monocytogenes 4b Contig727 |
| | Corresponding to the former SEQ ID n^{o} 1393 |
| SEQ ID N^{o} 3599 | Listeria monocytogenes 4b Contig728 |
| | Corresponding to the former SEQ ID n^{o} 1223 |
| SEQ ID N^{o} 3600 | Listeria monocytogenes 4b Contig729 |
| | Corresponding to the former SEQ ID n^{o} 1973 |
| SEQ ID N^{o} 3601 | Listeria monocytogenes 4b Contig730 |
| | Corresponding to the former SEQ ID n^{o} 1743 |
| SEQ ID N^{o} 3602 | Listeria monocytogenes 4b Contig731 |
| | Corresponding to the former SEQ ID n^{o} 1860 |
| SEQ ID N^{o} 3603 | Listeria monocytogenes 4b Contig732 |
| | Corresponding to the former SEQ ID n^{o} 1203 |
| SEQ ID N^{o} 3604 | Listeria monocytogenes 4b Contig733 |
| | Corresponding to the former SEQ ID n^{o} 1690 |
| | Corresponding to the former SEQ ID n^{o} 1701 |
| SEQ ID N^{o} 3605 | Listeria monocytogenes 4b Contig734 |
| | Corresponding to the former SEQ ID n^{o} 1525 |
| SEQ ID N^{o} 3606 | Listeria monocytogenes 4b Contig735 |
| | Corresponding to the former SEQ ID n^{o} 1272 |
| SEQ ID N^{o} 3607 | Listeria monocytogenes 4b Contig736 |
| SEQ ID N^{o} 3608 | Listeria monocytogenes 4b Contig737 |
| | Corresponding to the former SEQ ID n^{o} 1986 |
| SEQ ID N^{o} 3609 | Listeria monocytogenes 4b Contig738 |
| | Corresponding to the former SEQ ID n^{o} 1799 |
| SEQ ID N^{o} 3610 | Listeria monocytogenes 4b Contig739 |
| SEQ ID N^{o} 3611 | Listeria monocytogenes 4b Contig740 |
| | Corresponding to the former SEQ ID n^{o} 1070 |
| | Corresponding to the former SEQ ID n^{o} 1783 |
| SEQ ID N^{o} 3612 | Listeria monocytogenes 4b Contig741 |
| SEQ ID N^{o} 3613 | Listeria monocytogenes 4b Contig742 |
| SEQ ID N^{o} 3614 | Listeria monocytogenes 4b Contig743 |
| | Corresponding to the former SEQ ID n^{o} 1437 |
| SEQ ID N^{o} 3615 | Listeria monocytogenes 4b Contig744 |
| | Corresponding to the former SEQ ID n^{o} 1094 |
| | Corresponding to the former SEQ ID n^{o} 1523 |
| SEQ ID N^{o} 3616 | Listeria monocytogenes 4b Contig745 |
| | Corresponding to the former SEQ ID n^{o} 1929 |
| SEQ ID N^{o} 3617 | Listeria monocytogenes 4b Contig746 |
| | Corresponding to the former SEQ ID n^{o} 1383 |
| | Corresponding to the former SEQ ID n^{o} 1486 |
| SEQ ID N^{o} 3618 | Listeria monocytogenes 4b Contig747 |
| SEQ ID N^{o} 3619 | Listeria monocytogenes 4b Contig748 |
| | Corresponding to the former SEQ ID n^{o} 1957 |
| SEQ ID N^{o} 3620 | Listeria monocytogenes 4b Contig749 |
| SEQ ID N^{o} 3621 | Listeria monocytogenes 4b Contig750 |
| | Corresponding to the former SEQ ID n^{o} 1859 |
| | Corresponding to the former SEQ ID n^{o} 1963 |
| SEQ ID N^{o} 3622 | Listeria monocytogenes 4b Contig751 |
| SEQ ID N^{o} 3623 | Listeria monocytogenes 4b Contig752 |
| SEQ ID N^{o} 3624 | Listeria monocytogenes 4b Contig753 |
| | Corresponding to the former SEQ ID n^{o} 1971 |
| SEQ ID N^{o} 3625 | Listeria monocytogenes 4b Contig754 |
| | Corresponding to the former SEQ ID n^{o} 1189 |
| | Corresponding to the former SEQ ID n^{o} 1289 |
| | Corresponding to the former SEQ ID n^{o} 1619 |
| SEQ ID N^{o} 3626 | Listeria monocytogenes 4b Contig755 |
| SEQ ID N^{o} 3627 | Listeria monocytogenes 4b Contig756 |
| | Corresponding to the former SEQ ID n^{o} 1883 |
| SEQ ID N^{o} 3628 | Listeria monocytogenes 4b Contig757 |
| | Corresponding to the former SEQ ID n^{o} 1316 |
| | Corresponding to the former SEQ ID n^{o} 1460 |
| SEQ ID N^{o} 3629 | Listeria monocytogenes 4b Contig758 |
| SEQ ID N^{o} 3630 | Listeria monocytogenes 4b Contig759 |
| | Corresponding to the former SEQ ID n^{o} 1389 |
| SEQ ID N^{o} 3631 | Listeria monocytogenes 4b Contig760 |
| SEQ ID N^{o} 3632 | Listeria monocytogenes 4b Contig761 |
| | Corresponding to the former SEQ ID n^{o} 1397 |
| SEQ ID N^{o} 3633 | Listeria monocytogenes 4b Contig762 |
| | Corresponding to the former SEQ ID n^{o} 1261 |
| | Corresponding to the former SEQ ID n^{o} 1531 |
| SEQ ID N^{o} 3634 | Listeria monocytogenes 4b Contig763 |
| | Corresponding to the former SEQ ID n^{o} 1563 |
| SEQ ID N^{o} 3635 | Listeria monocytogenes 4b Contig764 |
| | Corresponding to the former SEQ ID n^{o} 1945 |
| SEQ ID N^{o} 3636 | Listeria monocytogenes 4b Contig765 |
| | Corresponding to the former SEQ ID n^{o} 1306 |
| SEQ ID N^{o} 3637 | Listeria monocytogenes 4b Contig766 |
| | Corresponding to the former SEQ ID n^{o} 1253 |
| SEQ ID N^{o} 3638 | Listeria monocytogenes 4b Contig767 |
| | Corresponding to the former SEQ ID n^{o} 1116 |
| | Corresponding to the former SEQ ID n^{o} 1154 |
| SEQ ID N^{o} 3639 | Listeria monocytogenes 4b Contig768 |
| | Corresponding to the former SEQ ID n^{o} 1807 |
| SEQ ID N^{o} 3640 | Listeria monocytogenes 4b Contig769 |
| | Corresponding to the former SEQ ID n^{o} 1580 |
| SEQ ID N^{o} 3641 | Listeria monocytogenes 4b Contig770 |
| | Corresponding to the former SEQ ID n^{o} 1234 |
| | Corresponding to the former SEQ ID n^{o} 1951 |
| SEQ ID N^{o} 3642 | Listeria monocytogenes 4b Contig771 |
| | Corresponding to the former SEQ ID n^{o} 1933 |
| SEQ ID N^{o} 3643 | Listeria monocytogenes 4b Contig772 |
| | Corresponding to the former SEQ ID n^{o} 1186 |
| | Corresponding to the former SEQ ID n^{o} 1462 |
| SEQ ID N^{o} 3644 | Listeria monocytogenes 4b Contig773 |
| | Corresponding to the former SEQ ID n^{o} 1073 |
| | Corresponding to the former SEQ ID n^{o} 1167 |
| | Corresponding to the former SEQ ID n^{o} 1322 |
| SEQ ID N^{o} 3645 | Listeria monocytogenes 4b Contig774 |
| | Corresponding to the former SEQ ID n^{o} 1686 |
| SEQ ID N^{o} 3646 | Listeria monocytogenes 4b Contig775 |
| | Corresponding to the former SEQ ID n^{o} 2005 |
| SEQ ID N^{o} 3647 | Listeria monocytogenes 4b Contig776 |
| | Corresponding to the former SEQ ID n^{o} 1385 |
| | Corresponding to the former SEQ ID n^{o} 1685 |
| SEQ ID N^{o} 3648 | Listeria monocytogenes 4b Contig777 |
| | Corresponding to the former SEQ ID n^{o} 1277 |
| | Corresponding to the former SEQ ID n^{o} 1785 |
| SEQ ID N^{o} 3649 | Listeria monocytogenes 4b Contig778 |
| SEQ ID N^{o} 3650 | Listeria monocytogenes 4b Contig779 |
| | Corresponding to the former SEQ ID n^{o} 1946 |
| SEQ ID N^{o} 3651 | Listeria monocytogenes 4b Contig780 |
| | Corresponding to the former SEQ ID n^{o} 1388 |
| | Corresponding to the former SEQ ID n^{o} 1732 |
| SEQ ID N^{o} 3652 | Listeria monocytogenes 4b Contig781 |
| | Corresponding to the former SEQ ID n^{o} 1652 |
| | Corresponding to the former SEQ ID n^{o} 1697 |
| SEQ ID N^{o} 3653 | Listeria monocytogenes 4b Contig782 |
| | Corresponding to the former SEQ ID n^{o} 1244 |
| | Corresponding to the former SEQ ID n^{o} 1286 |
| SEQ ID N^{o} 3654 | Listeria monocytogenes 4b Contig783 |
| | Corresponding to the former SEQ ID n^{o} 1275 |
| | Corresponding to the former SEQ ID n^{o} 1421 |
| SEQ ID N^{o} 3655 | Listeria monocytogenes 4b Contig784 |
| | Corresponding to the former SEQ ID n^{o} 1240 |
| SEQ ID N^{o} 3656 | Listeria monocytogenes 4b Contig785 |
| | Corresponding to the former SEQ ID n^{o} 1129 |
| | Corresponding to the former SEQ ID n^{o} 1384 |
| | Corresponding to the former SEQ ID n^{o} 1469 |
| SEQ ID N^{o} 3657 | Listeria monocytogenes 4b Contig786 |
| | Corresponding to the former SEQ ID n^{o} 1610 |
| | Corresponding to the former SEQ ID n^{o} 1857 |
| SEQ ID N^{o} 3658 | Listeria monocytogenes 4b Contig787 |
| | Corresponding to the former SEQ ID n^{o} 1484 |
| SEQ ID N^{o} 3659 | Listeria monocytogenes 4b Contig788 |
| | Corresponding to the former SEQ ID n^{o} 1081 |
| | Corresponding to the former SEQ ID n^{o} 1117 |
| | Corresponding to the former SEQ ID n^{o} 1196 |
| SEQ ID N^{o} 3660 | Listeria monocytogenes 4b Contig789 |
| | Corresponding to the former SEQ ID n^{o} 1175 |
| SEQ ID N^{o} 3661 | Listeria monocytogenes 4b Contig790 |
| | Corresponding to the former SEQ ID n^{o} 1727 |
| SEQ ID N^{o} 3662 | Listeria monocytogenes 4b Contig791 |
| | Corresponding to the former SEQ ID n^{o} 1925 |
| SEQ ID N^{o} 3663 | Listeria monocytogenes 4b Contig792 |
| | Corresponding to the former SEQ ID n^{o} 1134 |
| | Corresponding to the former SEQ ID n^{o} 1157 |
| | Corresponding to the former SEQ ID n^{o} 1779 |
| SEQ ID N^{o} 3664 | Listeria monocytogenes 4b Contig793 |
| | Corresponding to the former SEQ ID n^{o} 1795 |
| SEQ ID N^{o} 3665 | Listeria monocytogenes 4b Contig794 |
| | Corresponding to the former SEQ ID n^{o} 1988 |
| SEQ ID N^{o} 3666 | Listeria monocytogenes 4b Contig795 |
| | Corresponding to the former SEQ ID n^{o} 1616 |
| SEQ ID N^{o} 3667 | Listeria monocytogenes 4b Contig796 |
| SEQ ID N^{o} 3668 | Listeria monocytogenes 4b Contig797 |
| | Corresponding to the former SEQ ID n^{o} 1103 |
| SEQ ID N^{o} 3669 | Listeria monocytogenes 4b Contig798 |
| | Corresponding to the former SEQ ID n^{o} 1511 |
| SEQ ID N^{o} 3670 | Listeria monocytogenes 4b Contig799 |
| | Corresponding to the former SEQ ID n^{o} 1446 |
| SEQ ID N^{o} 3671 | Listeria monocytogenes 4b Contig800 |
| | Corresponding to the former SEQ ID n^{o} 1264 |
| | Corresponding to the former SEQ ID n^{o} 2017 |
| SEQ ID N^{o} 3672 | Listeria monocytogenes 4b Contig801 |
| | Corresponding to the former SEQ ID n^{o} 1151 |
| | Corresponding to the former SEQ ID n^{o} 1577 |
| SEQ ID N^{o} 3673 | Listeria monocytogenes 4b Contig802 |
| | Corresponding to the former SEQ ID n^{o} 1520 |
| | Corresponding to the former SEQ ID n^{o} 1755 |
| SEQ ID N^{o} 3674 | Listeria monocytogenes 4b Contig803 |
| | Corresponding to the former SEQ ID n^{o} 1291 |
| | Corresponding to the former SEQ ID n^{o} 1305 |
| | Corresponding to the former SEQ ID n^{o} 1589 |
| SEQ ID N^{o} 3675 | Listeria monocytogenes 4b Contig804 |
| | Corresponding to the former SEQ ID n^{o} 1207 |
| SEQ ID N^{o} 3676 | Listeria monocytogenes 4b Contig805 |
| | Corresponding to the former SEQ ID n^{o} 1987 |
| SEQ ID N^{o} 3677 | Listeria monocytogenes 4b Contig806 |
| | Corresponding to the former SEQ ID n^{o} 1912 |
| SEQ ID N^{o} 3678 | Listeria monocytogenes 4b Contig807 |
| | Corresponding to the former SEQ ID n^{o} 1432 |
| | Corresponding to the former SEQ ID n^{o} 1681 |
| SEQ ID N^{o} 3679 | Listeria monocytogenes 4b Contig808 |
| | Corresponding to the former SEQ ID n^{o} 1955 |
| SEQ ID N^{o} 3680 | Listeria monocytogenes 4b Contig809 |
| | Corresponding to the former SEQ ID n^{o} 1382 |
| SEQ ID N^{o} 3681 | Listeria monocytogenes 4b Contig810 |
| | Corresponding to the former SEQ ID n^{o} 1885 |
| SEQ ID N^{o} 3682 | Listeria monocytogenes 4b Contig811 |
| SEQ ID N^{o} 3683 | Listeria monocytogenes 4b Contig812 |
| | Corresponding to the former SEQ ID n^{o} 1451 |
| | Corresponding to the former SEQ ID n^{o} 1592 |
| SEQ ID N^{o} 3684 | Listeria monocytogenes 4b Contig813 |
| | Corresponding to the former SEQ ID n^{o} 1402 |
| | Corresponding to the former SEQ ID n^{o} 1647 |
| | Corresponding to the former SEQ ID n^{o} 1768 |
| SEQ ID N^{o} 3685 | Listeria monocytogenes 4b Contig814 |
| | Corresponding to the former SEQ ID n^{o} 1522 |
| SEQ ID N^{o} 3686 | Listeria monocytogenes 4b Contig815 |
| | Corresponding to the former SEQ ID n^{o} 1984 |
| SEQ ID N^{o} 3687 | Listeria monocytogenes 4b Contig816 |
| | Corresponding to the former SEQ ID n^{o} 1335 |
| SEQ ID N^{o} 3688 | Listeria monocytogenes 4b Contig817 |
| | Corresponding to the former SEQ ID n^{o} 2007 |
| SEQ ID N^{o} 3689 | Listeria monocytogenes 4b Contig818 |
| | Corresponding to the former SEQ ID n^{o} 1121 |
| SEQ ID N^{o} 3690 | Listeria monocytogenes 4b Contig819 |
| | Corresponding to the former SEQ ID n^{o} 1182 |
| SEQ ID N^{o} 3691 | Listeria monocytogenes 4b Contig820 |
| | Corresponding to the former SEQ ID n^{o} 1554 |
| SEQ ID N^{o} 3692 | Listeria monocytogenes 4b Contig821 |
| | Corresponding to the former SEQ ID n^{o} 1153 |
| | Corresponding to the former SEQ ID n^{o} 1458 |
| SEQ ID N^{o} 3693 | Listeria monocytogenes 4b Contig822 |
| | Corresponding to the former SEQ ID n^{o} 1254 |
| | Corresponding to the former SEQ ID n^{o} 1864 |
| SEQ ID N^{o} 3694 | Listeria monocytogenes 4b Contig823 |
| | Corresponding to the former SEQ ID n^{o} 1407 |
| | Corresponding to the former SEQ ID n^{o} 1736 |
| SEQ ID N^{o} 3695 | Listeria monocytogenes 4b Contig824 |
| | Corresponding to the former SEQ ID n^{o} 1412 |
| | Corresponding to the former SEQ ID n^{o} 1482 |
| SEQ ID N^{o} 3696 | Listeria monocytogenes 4b Contig825 |
| | Corresponding to the former SEQ ID n^{o} 1360 |
| | Corresponding to the former SEQ ID n^{o} 1375 |
| | Corresponding to the former SEQ ID n^{o} 1645 |
| SEQ ID N^{o} 3697 | Listeria monocytogenes 4b Contig826 |
| | Corresponding to the former SEQ ID n^{o} 1164 |
| SEQ ID N^{o} 3698 | Listeria monocytogenes 4b Contig827 |
| | Corresponding to the former SEQ ID n^{o} 1459 |
| | Corresponding to the former SEQ ID n^{o} 1772 |
| SEQ ID N^{o} 3699 | Listeria monocytogenes 4b Contig828 |
| | Corresponding to the former SEQ ID n^{o} 1334 |
| | Corresponding to the former SEQ ID n^{o} 1533 |
| | Corresponding to the former SEQ ID n^{o} 1750 |
| SEQ ID N^{o} 3700 | Listeria monocytogenes 4b Contig829 |
| | Corresponding to the former SEQ ID n^{o} 1586 |
| SEQ ID N^{o} 3701 | Listeria monocytogenes 4b Contig830 |
| | Corresponding to the former SEQ ID n^{o} 2021 |
| SEQ ID N^{o} 3702 | Listeria monocytogenes 4b Contig831 |
| | Corresponding to the former SEQ ID n^{o} 1374 |
| | Corresponding to the former SEQ ID n^{o} 1819 |
| SEQ ID N^{o} 3703 | Listeria monocytogenes 4b Contig832 |
| | Corresponding to the former SEQ ID n^{o} 1425 |
| | Corresponding to the former SEQ ID n^{o} 1598 |
| SEQ ID N^{o} 3704 | Listeria monocytogenes 4b Contig833 |
| | Corresponding to the former SEQ ID n^{o} 1198 |
| | Corresponding to the former SEQ ID n^{o} 1296 |
| SEQ ID N^{o} 3705 | Listeria monocytogenes 4b Contig834 |
| | Corresponding to the former SEQ ID n^{o} 1893 |
| SEQ ID N^{o} 3706 | Listeria monocytogenes 4b Contig835 |
| | Corresponding to the former SEQ ID n^{o} 1119 |
| | Corresponding to the former SEQ ID n^{o} 1720 |
| SEQ ID N^{o} 3707 | Listeria monocytogenes 4b Contig836 |
| | Corresponding to the former SEQ ID n^{o} 1457 |
| SEQ ID N^{o} 3708 | Listeria monocytogenes 4b Contig837 |
| | Corresponding to the former SEQ ID n^{o} 1655 |
| | Corresponding to the former SEQ ID n^{o} 1880 |
| SEQ ID N^{o} 3709 | Listeria monocytogenes 4b Contig838 |
| | Corresponding to the former SEQ ID n^{o} 1191 |
| SEQ ID N^{o} 3710 | Listeria monocytogenes 4b Contig839 |
| | Corresponding to the former SEQ ID n^{o} 1358 |
| SEQ ID N^{o} 3711 | Listeria monocytogenes 4b Contig840 |
| | Corresponding to the former SEQ ID n^{o} 1187 |
| | Corresponding to the former SEQ ID n^{o} 1495 |
| | Corresponding to the former SEQ ID n^{o} 1856 |
| SEQ ID N^{o} 3712 | Listeria monocytogenes 4b Contig841 |
| | Corresponding to the former SEQ ID n^{o} 1130 |
| SEQ ID N^{o} 3713 | Listeria monocytogenes 4b Contig842 |
| | Corresponding to the former SEQ ID n^{o} 1141 |
| | Corresponding to the former SEQ ID n^{o} 1292 |
| | Corresponding to the former SEQ ID n^{o} 1452 |
| SEQ ID N^{o} 3714 | Listeria monocytogenes 4b Contig843 |
| | Corresponding to the former SEQ ID n^{o} 1120 |
| | Corresponding to the former SEQ ID n^{o} 1967 |
| SEQ ID N^{o} 3715 | Listeria monocytogenes 4b Contig844 |
| | Corresponding to the former SEQ ID n^{o} 1954 |
| SEQ ID N^{o} 3716 | Listeria monocytogenes 4b Contig845 |
| | Corresponding to the former SEQ ID n^{o} 1966 |
| SEQ ID N^{o} 3717 | Listeria monocytogenes 4b Contig846 |
| | Corresponding to the former SEQ ID n^{o} 1680 |
| | Corresponding to the former SEQ ID n^{o} 1882 |
| SEQ ID N^{o} 3718 | Listeria monocytogenes 4b Contig847 |
| | Corresponding to the former SEQ ID n^{o} 1474 |
| | Corresponding to the former SEQ ID n^{o} 1842 |
| SEQ ID N^{o} 3719 | Listeria monocytogenes 4b Contig848 |
| | Corresponding to the former SEQ ID n^{o} 1102 |
| | Corresponding to the former SEQ ID n^{o} 1423 |
| | Corresponding to the former SEQ ID n^{o} 1559 |
| SEQ ID N^{o} 3720 | Listeria monocytogenes 4b Contig849 |
| | Corresponding to the former SEQ ID n^{o} 1128 |
| | Corresponding to the former SEQ ID n^{o} 1181 |
| | Corresponding to the former SEQ ID n^{o} 1599 |
| SEQ ID N^{o} 3721 | Listeria monocytogenes 4b Contig850 |
| | Corresponding to the former SEQ ID n^{o} 1269 |
| | Corresponding to the former SEQ ID n^{o} 1791 |
| SEQ ID N^{o} 3722 | Listeria monocytogenes 4b Contig851 |
| | Corresponding to the former SEQ ID n^{o} 1218 |
| | Corresponding to the former SEQ ID n^{o} 1928 |
| SEQ ID N^{o} 3723 | Listeria monocytogenes 4b Contig852 |
| | Corresponding to the former SEQ ID n^{o} 1938 |
| SEQ ID N^{o} 3724 | Listeria monocytogenes 4b Contig853 |
| | Corresponding to the former SEQ ID n^{o} 2028 |
| SEQ ID N^{o} 3725 | Listeria monocytogenes 4b Contig854 |
| | Corresponding to the former SEQ ID n^{o} 1376 |
| | Corresponding to the former SEQ ID n^{o} 1870 |
| SEQ ID N^{o} 3726 | Listeria monocytogenes 4b Contig855 |
| | Corresponding to the former SEQ ID n^{o} 1278 |
| | Corresponding to the former SEQ ID n^{o} 1634 |
| SEQ ID N^{o} 3727 | Listeria monocytogenes 4b Contig856 |
| | Corresponding to the former SEQ ID n^{o} 1312 |
| | Corresponding to the former SEQ ID n^{o} 1705 |
| SEQ ID N^{o} 3728 | Listeria monocytogenes 4b Contig857 |
| | Corresponding to the former SEQ ID n^{o} 1479 |
| | Corresponding to the former SEQ ID n^{o} 1972 |
| SEQ ID N^{o} 3729 | Listeria monocytogenes 4b Contig858 |
| | Corresponding to the former SEQ ID n^{o} 1307 |
| | Corresponding to the former SEQ ID n^{o} 1829 |
| SEQ ID N^{o} 3730 | Listeria monocytogenes 4b Contig859 |
| | Corresponding to the former SEQ ID n^{o} 1123 |
| | Corresponding to the former SEQ ID n^{o} 1687 |
| SEQ ID N^{o} 3731 | Listeria monocytogenes 4b Contig860 |
| | Corresponding to the former SEQ ID n^{o} 1905 |
| SEQ ID N^{o} 3732 | Listeria monocytogenes 4b Contig861 |
| | Corresponding to the former SEQ ID n^{o} 1579 |
| SEQ ID N^{o} 3733 | Listeria monocytogenes 4b Contig862 |
| | Corresponding to the former SEQ ID n^{o} 1080 |
| | Corresponding to the former SEQ ID n^{o} 1146 |
| SEQ ID N^{o} 3734 | Listeria monocytogenes 4b Contig863 |
| | Corresponding to the former SEQ ID n^{o} 1111 |
| | Corresponding to the former SEQ ID n^{o} 1514 |
| SEQ ID N^{o} 3735 | Listeria monocytogenes 4b Contig864 |
| | Corresponding to the former SEQ ID n^{o} 1139 |
| | Corresponding to the former SEQ ID n^{o} 1602 |
| SEQ ID N^{o} 3736 | Listeria monocytogenes 4b Contig865 |
| | Corresponding to the former SEQ ID n^{o} 1221 |
| | Corresponding to the former SEQ ID n^{o} 2010 |
| SEQ ID N^{o} 3737 | Listeria monocytogenes 4b Contig866 |
| | Corresponding to the former SEQ ID n^{o} 1174 |
| | Corresponding to the former SEQ ID n^{o} 1480 |
| | Corresponding to the former SEQ ID n^{o} 1895 |
| SEQ ID N^{o} 3738 | Listeria monocytogenes 4b Contig867 |
| | Corresponding to the former SEQ ID n^{o} 1780 |
| | Corresponding to the former SEQ ID n^{o} 1784 |
| SEQ ID N^{o} 3739 | Listeria monocytogenes 4b Contig868 |
| | Corresponding to the former SEQ ID n^{o} 2009 |
| SEQ ID N^{o} 3740 | Listeria monocytogenes 4b Contig869 |
| | Corresponding to the former SEQ ID n^{o} 1308 |
| | Corresponding to the former SEQ ID n^{o} 1597 |
| SEQ ID N^{o} 3741 | Listeria monocytogenes 4b Contig870 |
| | Corresponding to the former SEQ ID n^{o} 1311 |
| | Corresponding to the former SEQ ID n^{o} 1315 |
| | Corresponding to the former SEQ ID n^{o} 2003 |
| SEQ ID N^{o} 3742 | Listeria monocytogenes 4b Contig871 |
| | Corresponding to the former SEQ ID n^{o} 1493 |
| | Corresponding to the former SEQ ID n^{o} 1707 |
| SEQ ID N^{o} 3743 | Listeria monocytogenes 4b Contig872 |
| | Corresponding to the former SEQ ID n^{o} 1089 |
| | Corresponding to the former SEQ ID n^{o} 1624 |
| SEQ ID N^{o} 3744 | Listeria monocytogenes 4b Contig873 |
| | Corresponding to the former SEQ ID n^{o} 1846 |
| SEQ ID N^{o} 3745 | Listeria monocytogenes 4b Contig874 |
| | Corresponding to the former SEQ ID n^{o} 1603 |
| | Corresponding to the former SEQ ID n^{o} 1921 |
| SEQ ID N^{o} 3746 | Listeria monocytogenes 4b Contig875 |
| | Corresponding to the former SEQ ID n^{o} 1268 |
| | Corresponding to the former SEQ ID n^{o} 1752 |
| SEQ ID N^{o} 3747 | Listeria monocytogenes 4b Contig876 |
| | Corresponding to the former SEQ ID n^{o} 1336 |
| | Corresponding to the former SEQ ID n^{o} 1623 |
| SEQ ID N^{o} 3748 | Listeria monocytogenes 4b Contig877 |
| | Corresponding to the former SEQ ID n^{o} 1259 |
| | Corresponding to the former SEQ ID n^{o} 1551 |
| | Corresponding to the former SEQ ID n^{o} 1866 |
| SEQ ID N^{o} 3749 | Listeria monocytogenes 4b Contig878 |
| | Corresponding to the former SEQ ID n^{o} 1224 |
| SEQ ID N^{o} 3750 | Listeria monocytogenes 4b Contig879 |
| | Corresponding to the former SEQ ID n^{o} 1418 |
| | Corresponding to the former SEQ ID n^{o} 1571 |
| | Corresponding to the former SEQ ID n^{o} 1809 |
| SEQ ID N^{o} 3751 | Listeria monocytogenes 4b Contig880 |
| | Corresponding to the former SEQ ID n^{o} 1420 |
| | Corresponding to the former SEQ ID n^{o} 1664 |
| SEQ ID N^{o} 3752 | Listeria monocytogenes 4b Contig881 |
| | Corresponding to the former SEQ ID n^{o} 1137 |
| | Corresponding to the former SEQ ID n^{o} 1367 |
| SEQ ID N^{o} 3753 | Listeria monocytogenes 4b Contig882 |
| | Corresponding to the former SEQ ID n^{o} 1222 |
| | Corresponding to the former SEQ ID n^{o} 1318 |
| | Corresponding to the former SEQ ID n^{o} 1758 |
| SEQ ID N^{o} 3754 | Listeria monocytogenes 4b Contig883 |
| | Corresponding to the former SEQ ID n^{o} 1978 |
| SEQ ID N^{o} 3755 | Listeria monocytogenes 4b Contig884 |
| | Corresponding to the former SEQ ID n^{o} 1793 |
| | Corresponding to the former SEQ ID n^{o} 1855 |
| SEQ ID N^{o} 3756 | Listeria monocytogenes 4b Contig885 |
| | Corresponding to the former SEQ ID n^{o} 1236 |
| | Corresponding to the former SEQ ID n^{o} 1666 |
| | Corresponding to the former SEQ ID n^{o} 1892 |
| SEQ ID N^{o} 3757 | Listeria monocytogenes 4b Contig886 |
| | Corresponding to the former SEQ ID n^{o} 1466 |
| | Corresponding to the former SEQ ID n^{o} 1825 |
| SEQ ID N^{o} 3758 | Listeria monocytogenes 4b Contig887 |
| | Corresponding to the former SEQ ID n^{o} 1901 |
| SEQ ID N^{o} 3759 | Listeria monocytogenes 4b Contig888 |
| | Corresponding to the former SEQ ID n^{o} 1761 |
| | Corresponding to the former SEQ ID n^{o} 1947 |
| SEQ ID N^{o} 3760 | Listeria monocytogenes 4b Contig889 |
| | Corresponding to the former SEQ ID n^{o} 1517 |
| | Corresponding to the former SEQ ID n^{o} 1943 |
| SEQ ID N^{o} 3761 | Listeria monocytogenes 4b Contig890 |
| | Corresponding to the former SEQ ID n^{o} 1654 |
| | Corresponding to the former SEQ ID n^{o} 1787 |
| SEQ ID N^{o} 3762 | Listeria monocytogenes 4b Contig891 |
| | Corresponding to the former SEQ ID n^{o} 1427 |
| | Corresponding to the former SEQ ID n^{o} 2019 |
| SEQ ID N^{o} 3763 | Listeria monocytogenes 4b Contig892 |
| | Corresponding to the former SEQ ID n^{o} 1441 |
| | Corresponding to the former SEQ ID n^{o} 1974 |
| SEQ ID N^{o} 3764 | Listeria monocytogenes 4b Contig893 |
| | Corresponding to the former SEQ ID n^{o} 1411 |
| | Corresponding to the former SEQ ID n^{o} 1733 |
| SEQ ID N^{o} 3765 | Listeria monocytogenes 4b Contig894 |
| SEQ ID N^{o} 3766 | Listeria monocytogenes 4b Contig895 |
| | Corresponding to the former SEQ ID n^{o} 1994 |
| SEQ ID N^{o} 3767 | Listeria monocytogenes 4b Contig896 |
| | Corresponding to the former SEQ ID n^{o} 1552 |
| SEQ ID N^{o} 3768 | Listeria monocytogenes 4b Contig897 |
| | Corresponding to the former SEQ ID n^{o} 1442 |
| SEQ ID N^{o} 3769 | Listeria monocytogenes 4b Contig898 |
| | Corresponding to the former SEQ ID n^{o} 1150 |
| | Corresponding to the former SEQ ID n^{o} 1937 |
| | Corresponding to the former SEQ ID n^{o} 1980 |
| SEQ ID N^{o} 3770 | Listeria monocytogenes 4b Contig899 |
| | Corresponding to the former SEQ ID n^{o} 1258 |
| | Corresponding to the former SEQ ID n^{o} 1816 |
| SEQ ID N^{o} 3771 | Listeria monocytogenes 4b Contig900 |
| | Corresponding to the former SEQ ID n^{o} 1983 |
| SEQ ID N^{o} 3772 | Listeria monocytogenes 4b Contig901 |
| | Corresponding to the former SEQ ID n^{o} 1422 |
| | Corresponding to the former SEQ ID n^{o} 1726 |
| SEQ ID N^{o} 3773 | Listeria monocytogenes 4b Contig902 |
| | Corresponding to the former SEQ ID n^{o} 1410 |
| | Corresponding to the former SEQ ID n^{o} 1778 |
| SEQ ID N^{o} 3774 | Listeria monocytogenes 4b Contig903 |
| | Corresponding to the former SEQ ID n^{o} 1232 |
| | Corresponding to the former SEQ ID n^{o} 1487 |
| SEQ ID N^{o} 3775 | Listeria monocytogenes 4b Contig904 |
| | Corresponding to the former SEQ ID n^{o} 1898 |
| SEQ ID N^{o} 3776 | Listeria monocytogenes 4b Contig905 |
| | Corresponding to the former SEQ ID n^{o} 1368 |
| | Corresponding to the former SEQ ID n^{o} 1985 |
| SEQ ID N^{o} 3777 | Listeria monocytogenes 4b Contig906 |
| | Corresponding to the former SEQ ID n^{o} 1997 |
| SEQ ID N^{o} 3778 | Listeria monocytogenes 4b Contig907 |
| | Corresponding to the former SEQ ID n^{o} 1321 |
| | Corresponding to the former SEQ ID n^{o} 1542 |
| | Corresponding to the former SEQ ID n^{o} 1843 |
| SEQ ID N^{o} 3779 | Listeria monocytogenes 4b Contig908 |
| | Corresponding to the former SEQ ID n^{o} 1530 |
| | Corresponding to the former SEQ ID n^{o} 1667 |
| SEQ ID N^{o} 3780 | Listeria monocytogenes 4b Contig909 |
| | Corresponding to the former SEQ ID n^{o} 1534 |
| | Corresponding to the former SEQ ID n^{o} 1936 |
| SEQ ID N^{o} 3781 | Listeria monocytogenes 4b Contig910 |
| | Corresponding to the former SEQ ID n^{o} 1567 |
| | Corresponding to the former SEQ ID n^{o} 1587 |
| | Corresponding to the former SEQ ID n^{o} 1642 |
| | Corresponding to the former SEQ ID n^{o} 1674 |
| SEQ ID N^{o} 3782 | Listeria monocytogenes 4b Contig911 |
| | Corresponding to the former SEQ ID n^{o} 2000 |
| SEQ ID N^{o} 3783 | Listeria monocytogenes 4b Contig912 |
| | Corresponding to the former SEQ ID n^{o} 1909 |
| SEQ ID N^{o} 3784 | Listeria monocytogenes 4b Contig913 |
| | Corresponding to the former SEQ ID n^{o} 1106 |
| | Corresponding to the former SEQ ID n^{o} 1365 |
| | Corresponding to the former SEQ ID n^{o} 1734 |
| SEQ ID N^{o} 3785 | Listeria monocytogenes 4b Contig914 |
| | Corresponding to the former SEQ ID n^{o} 1284 |
| | Corresponding to the former SEQ ID n^{o} 1309 |
| | Corresponding to the former SEQ ID n^{o} 1613 |
| SEQ ID N^{o} 3786 | Listeria monocytogenes 4b Contig915 |
| | Corresponding to the former SEQ ID n^{o} 2002 |
| SEQ ID N^{o} 3787 | Listeria monocytogenes 4b Contig916 |
| | Corresponding to the former SEQ ID n^{o} 1271 |
| | Corresponding to the former SEQ ID n^{o} 1941 |
| SEQ ID N^{o} 3788 | Listeria monocytogenes 4b Contig917 |
| | Corresponding to the former SEQ ID n^{o} 1104 |
| | Corresponding to the former SEQ ID n^{o} 2030 |
| SEQ ID N^{o} 3789 | Listeria monocytogenes 4b Contig918 |
| | Corresponding to the former SEQ ID n^{o} 1959 |
| SEQ ID N^{o} 3790 | Listeria monocytogenes 4b Contig919 |
| | Corresponding to the former SEQ ID n^{o} 1266 |
| | Corresponding to the former SEQ ID n^{o} 2020 |
| SEQ ID N^{o} 3791 | Listeria monocytogenes 4b Contig920 |
| | Corresponding to the former SEQ ID n^{o} 1405 |
| | Corresponding to the former SEQ ID n^{o} 1718 |
| | Corresponding to the former SEQ ID n^{o} 1919 |
| SEQ ID N^{o} 3792 | Listeria monocytogenes 4b Contig921 |
| | Corresponding to the former SEQ ID n^{o} 1908 |
| SEQ ID N^{o} 3793 | Listeria monocytogenes 4b Contig922 |
| | Corresponding to the former SEQ ID n^{o} 1786 |
| SEQ ID N^{o} 3794 | Listeria monocytogenes 4b Contig923 |
| | Corresponding to the former SEQ ID n^{o} 1370 |
| | Corresponding to the former SEQ ID n^{o} 1371 |
| | Corresponding to the former SEQ ID n^{o} 1372 |
| | Corresponding to the former SEQ ID n^{o} 1574 |
| SEQ ID N^{o} 3795 | Listeria monocytogenes 4b Contig924 |
| | Corresponding to the former SEQ ID n^{o} 1488 |
| SEQ ID N^{o} 3796 | Listeria monocytogenes 4b Contig925 |
| | Corresponding to the former SEQ ID n^{o} 1532 |
| | Corresponding to the former SEQ ID n^{o} 2008 |
| SEQ ID N^{o} 3797 | Listeria monocytogenes 4b Contig926 |
| | Corresponding to the former SEQ ID n^{o} 1677 |
| | Corresponding to the former SEQ ID n^{o} 1906 |
| SEQ ID N^{o} 3798 | Listeria monocytogenes 4b Contig927 |
| | Corresponding to the former SEQ ID n^{o} 1497 |
| | Corresponding to the former SEQ ID n^{o} 1699 |
| | Corresponding to the former SEQ ID n^{o} 1700 |
| | Corresponding to the former SEQ ID n^{o} 1948 |
| SEQ ID N^{o} 3799 | Listeria monocytogenes 4b Contig928 |
| | Corresponding to the former SEQ ID n^{o} 1891 |
| SEQ ID N^{o} 3800 | Listeria monocytogenes 4b Contig929 |
| | Corresponding to the former SEQ ID n^{o} 1633 |
| | Corresponding to the former SEQ ID n^{o} 1656 |
| SEQ ID N^{o} 3801 | Listeria monocytogenes 4b Contig930 |
| | Corresponding to the former SEQ ID n^{o} 1419 |
| | Corresponding to the former SEQ ID n^{o} 1494 |
| SEQ ID N^{o} 3802 | Listeria monocytogenes 4b Contig931 |
| | Corresponding to the former SEQ ID n^{o} 2027 |
| SEQ ID N^{o} 3803 | Listeria monocytogenes 4b Contig932 |
| | Corresponding to the former SEQ ID n^{o} 1814 |
| | Corresponding to the former SEQ ID n^{o} 1828 |
| SEQ ID N^{o} 3804 | Listeria monocytogenes 4b Contig933 |
| | Corresponding to the former SEQ ID n^{o} 1400 |
| | Corresponding to the former SEQ ID n^{o} 1628 |
| | Corresponding to the former SEQ ID n^{o} 1698 |
| SEQ ID N^{o} 3805 | Listeria monocytogenes 4b Contig934 |
| | Corresponding to the former SEQ ID n^{o} 1513 |
| | Corresponding to the former SEQ ID n^{o} 1695 |
| | Corresponding to the former SEQ ID n^{o} 1960 |
| SEQ ID N^{o} 3806 | Listeria monocytogenes 4b Contig935 |
| | Corresponding to the former SEQ ID n^{o} 1648 |
| | Corresponding to the former SEQ ID n^{o} 2018 |
| SEQ ID N^{o} 3807 | Listeria monocytogenes 4b Contig936 |
| | Corresponding to the former SEQ ID n^{o} 1238 |
| | Corresponding to the former SEQ ID n^{o} 1636 |
| SEQ ID N^{o} 3808 | Listeria monocytogenes 4b Contig937 |
| SEQ ID N^{o} 3809 | Listeria monocytogenes 4b Contig938 |
| | Corresponding to the former SEQ ID n^{o} 1341 |
| | Corresponding to the former SEQ ID n^{o} 1836 |
| | Corresponding to the former SEQ ID n^{o} 1848 |
| SEQ ID N^{o} 3810 | Listeria monocytogenes 4b Contig939 |
| | Corresponding to the former SEQ ID n^{o} 1087 |
| | Corresponding to the former SEQ ID n^{o} 1381 |
| SEQ ID N^{o} 3811 | Listeria monocytogenes 4b Contig940 |
| | Corresponding to the former SEQ ID n^{o} 1288 |
| | Corresponding to the former SEQ ID n^{o} 1386 |
| | Corresponding to the former SEQ ID n^{o} 1881 |
| SEQ ID N^{o} 3812 | Listeria monocytogenes 4b Contig941 |
| | Corresponding to the former SEQ ID n^{o} 1729 |
| | Corresponding to the former SEQ ID n^{o} 2014 |
| SEQ ID N^{o} 3813 | Listeria monocytogenes 4b Contig942 |
| | Corresponding to the former SEQ ID n^{o} 1319 |
| | Corresponding to the former SEQ ID n^{o} 1470 |
| | Corresponding to the former SEQ ID n^{o} 1904 |
| SEQ ID N^{o} 3814 | Listeria monocytogenes 4b Contig943 |
| | Corresponding to the former SEQ ID n^{o} 1447 |
| | Corresponding to the former SEQ ID n^{o} 1810 |
| SEQ ID N^{o} 3815 | Listeria monocytogenes 4b Contig944 |
| | Corresponding to the former SEQ ID n^{o} 1999 |
| SEQ ID N^{o} 3816 | Listeria monocytogenes 4b Contig945 |
| | Corresponding to the former SEQ ID n^{o} 1127 |
| | Corresponding to the former SEQ ID n^{o} 1504 |
| | Corresponding to the former SEQ ID n^{o} 1507 |
| | Corresponding to the former SEQ ID n^{o} 1631 |
| SEQ ID N^{o} 3817 | Listeria monocytogenes 4b Contig946 |
| | Corresponding to the former SEQ ID n^{o} 2011 |
| SEQ ID N^{o} 3818 | Listeria monocytogenes 4b Contig947 |
| | Corresponding to the former SEQ ID n^{o} 1475 |
| | Corresponding to the former SEQ ID n^{o} 1611 |
| | Corresponding to the former SEQ ID n^{o} 1672 |
| SEQ ID N^{o} 3819 | Listeria monocytogenes 4b Contig948 |
| | Corresponding to the former SEQ ID n^{o} 1088 |
| | Corresponding to the former SEQ ID n^{o} 1539 |
| SEQ ID N^{o} 3820 | Listeria monocytogenes 4b Contig949 |
| | Corresponding to the former SEQ ID n^{o} 1204 |
| | Corresponding to the former SEQ ID n^{o} 1347 |
| | Corresponding to the former SEQ ID n^{o} 1845 |
| SEQ ID N^{o} 3821 | Listeria monocytogenes 4b Contig950 |
| | Corresponding to the former SEQ ID n^{o} 1706 |
| | Corresponding to the former SEQ ID n^{o} 1869 |
| | Corresponding to the former SEQ ID n^{o} 1976 |
| SEQ ID N^{o} 3822 | Listeria monocytogenes 4b Contig951 |
| | Corresponding to the former SEQ ID n^{o} 1620 |
| SEQ ID N^{o} 3823 | Listeria monocytogenes 4b Contig952 |
| | Corresponding to the former SEQ ID n^{o} 1886 |
| | Corresponding to the former SEQ ID n^{o} 1935 |
| SEQ ID N^{o} 3824 | Listeria monocytogenes 4b Contig953 |
| | Corresponding to the former SEQ ID n^{o} 1279 |
| | Corresponding to the former SEQ ID n^{o} 1301 |
| | Corresponding to the former SEQ ID n^{o} 1827 |
| SEQ ID N^{o} 3825 | Listeria monocytogenes 4b Contig954 |
| | Corresponding to the former SEQ ID n^{o} 1605 |
| | Corresponding to the former SEQ ID n^{o} 1753 |
| | Corresponding to the former SEQ ID n^{o} 1792 |
| SEQ ID N^{o} 3826 | Listeria monocytogenes 4b Contig955 |
| | Corresponding to the former SEQ ID n^{o} 1998 |
| SEQ ID N^{o} 3827 | Listeria monocytogenes 4b Contig956 |
| | Corresponding to the former SEQ ID n^{o} 1310 |
| | Corresponding to the former SEQ ID n^{o} 1632 |
| | Corresponding to the former SEQ ID n^{o} 1853 |
| SEQ ID N^{o} 3828 | Listeria monocytogenes 4b Contig957 |
| | Corresponding to the former SEQ ID n^{o} 1914 |
| | Corresponding to the former SEQ ID n^{o} 1968 |
| SEQ ID N^{o} 3829 | Listeria monocytogenes 4b Contig958 |
| | Corresponding to the former SEQ ID n^{o} 1569 |
| | Corresponding to the former SEQ ID n^{o} 1801 |
| SEQ ID N^{o} 3830 | Listeria monocytogenes 4b Contig959 |
| | Corresponding to the former SEQ ID n^{o} 1369 |
| | Corresponding to the former SEQ ID n^{o} 1931 |
| SEQ ID N^{o} 3831 | Listeria monocytogenes 4b Contig960 |
| | Corresponding to the former SEQ ID n^{o} 1247 |
| | Corresponding to the former SEQ ID n^{o} 1617 |
| | Corresponding to the former SEQ ID n^{o} 1731 |
| SEQ ID N^{o} 3832 | Listeria monocytogenes 4b Contig961 |
| | Corresponding to the former SEQ ID n^{o} 1302 |
| | Corresponding to the former SEQ ID n^{o} 1920 |
| | Corresponding to the former SEQ ID n^{o} 2012 |
| SEQ ID N^{o} 3833 | Listeria monocytogenes 4b Contig962 |
| | Corresponding to the former SEQ ID n^{o} 1068 |
| | Corresponding to the former SEQ ID n^{o} 1072 |
| | Corresponding to the former SEQ ID n^{o} 1635 |
| SEQ ID N^{o} 3834 | Listeria monocytogenes 4b Contig963 |
| | Corresponding to the former SEQ ID n^{o} 1757 |
| | Corresponding to the former SEQ ID n^{o} 2024 |
| SEQ ID N^{o} 3835 | Listeria monocytogenes 4b Contig964 |
| | Corresponding to the former SEQ ID n^{o} 1509 |
| | Corresponding to the former SEQ ID n^{o} 1831 |
| SEQ ID N^{o} 3836 | Listeria monocytogenes 4b Contig965 |
| | Corresponding to the former SEQ ID n^{o} 1097 |
| | Corresponding to the former SEQ ID n^{o} 1230 |
| | Corresponding to the former SEQ ID n^{o} 1760 |
| SEQ ID N^{o} 3837 | Listeria monocytogenes 4b Contig966 |
| | Corresponding to the former SEQ ID n^{o} 1343 |
| | Corresponding to the former SEQ ID n^{o} 1766 |
| | Corresponding to the former SEQ ID n^{o} 1878 |
| SEQ ID N^{o} 3838 | Listeria monocytogenes 4b Contig967 |
| | Corresponding to the former SEQ ID n^{o} 1593 |
| | Corresponding to the former SEQ ID n^{o} 1604 |
| | Corresponding to the former SEQ ID n^{o} 1979 |
| SEQ ID N^{o} 3839 | Listeria monocytogenes 4b Contig968 |
| | Corresponding to the former SEQ ID n^{o} 1863 |
| | Corresponding to the former SEQ ID n^{o} 1969 |
| SEQ ID N^{o} 3840 | Listeria monocytogenes 4b Contig969 |
| | Corresponding to the former SEQ ID n^{o} 1339 |
| | Corresponding to the former SEQ ID n^{o} 1608 |
| | Corresponding to the former SEQ ID n^{o} 1942 |
| SEQ ID N^{o} 3841 | Listeria monocytogenes 4b Contig970 |
| | Corresponding to the former SEQ ID n^{o} 2038 |
| SEQ ID N^{o} 3842 | Listeria monocytogenes 4b Contig971 |
| | Corresponding to the former SEQ ID n^{o} 1716 |
| | Corresponding to the former SEQ ID n^{o} 1854 |
| SEQ ID N^{o} 3843 | Listeria monocytogenes 4b Contig972 |
| | Corresponding to the former SEQ ID n^{o} 1132 |
| | Corresponding to the former SEQ ID n^{o} 1354 |
| | Corresponding to the former SEQ ID n^{o} 1927 |
| | Corresponding to the former SEQ ID n^{o} 1975 |
| SEQ ID N^{o} 3844 | Listeria monocytogenes 4b Contig973 |
| | Corresponding to the former SEQ ID n^{o} 1541 |
| | Corresponding to the former SEQ ID n^{o} 1564 |
| SEQ ID N^{o} 3845 | Listeria monocytogenes 4b Contig974 |
| | Corresponding to the former SEQ ID n^{o} 1201 |
| | Corresponding to the former SEQ ID n^{o} 1797 |
| | Corresponding to the former SEQ ID n^{o} 1910 |
| | Corresponding to the former SEQ ID n^{o} 1981 |
| SEQ ID N^{o} 3846 | Listeria monocytogenes 4b Contig975 |
| | Corresponding to the former SEQ ID n^{o} 1342 |
| | Corresponding to the former SEQ ID n^{o} 1649 |
| | Corresponding to the former SEQ ID n^{o} 1751 |
| | Corresponding to the former SEQ ID n^{o} 1944 |
| SEQ ID N^{o} 3847 | Listeria monocytogenes 4b Contig976 |
| | Corresponding to the former SEQ ID n^{o} 1742 |
| | Corresponding to the former SEQ ID n^{o} 1896 |
| SEQ ID N^{o} 3848 | Listeria monocytogenes 4b Contig977 |
| | Corresponding to the former SEQ ID n^{o} 1079 |
| | Corresponding to the former SEQ ID n^{o} 1868 |
| | Corresponding to the former SEQ ID n^{o} 1875 |
| SEQ ID N^{o} 3849 | Listeria monocytogenes 4b Contig978 |
| | Corresponding to the former SEQ ID n^{o} 2013 |
| SEQ ID N^{o} 3850 | Listeria monocytogenes 4b Contig979 |
| | Corresponding to the former SEQ ID n^{o} 1818 |
| | Corresponding to the former SEQ ID n^{o} 1884 |
| SEQ ID N^{o} 3851 | Listeria monocytogenes 4b Contig980 |
| | Corresponding to the former SEQ ID n^{o} 1359 |
| | Corresponding to the former SEQ ID n^{o} 1519 |
| | Corresponding to the former SEQ ID n^{o} 1839 |
| SEQ ID N^{o} 3852 | Listeria monocytogenes 4b Contig981 |
| | Corresponding to the former SEQ ID n^{o} 1414 |
| | Corresponding to the former SEQ ID n^{o} 1782 |
| | Corresponding to the former SEQ ID n^{o} 1900 |
| SEQ ID N^{o} 3853 | Listeria monocytogenes 4b Contig982 |
| | Corresponding to the former SEQ ID n^{o} 1566 |
| SEQ ID N^{o} 3854 | Listeria monocytogenes 4b Contig983 |
| | Corresponding to the former SEQ ID n^{o} 2033 |
| SEQ ID N^{o} 3855 | Listeria monocytogenes 4b Contig984 |
| | Corresponding to the former SEQ ID n^{o} 1330 |
| | Corresponding to the former SEQ ID n^{o} 1748 |
| | Corresponding to the former SEQ ID n^{o} 1790 |
| | Corresponding to the former SEQ ID n^{o} 1962 |
| SEQ ID N^{o} 3856 | Listeria monocytogenes 4b Contig985 |
| | Corresponding to the former SEQ ID n^{o} 1682 |
| | Corresponding to the former SEQ ID n^{o} 2031 |
| SEQ ID N^{o} 3857 | Listeria monocytogenes 4b Contig986 |
| | Corresponding to the former SEQ ID n^{o} 1468 |
| | Corresponding to the former SEQ ID n^{o} 1606 |
| | Corresponding to the former SEQ ID n^{o} 1930 |
| SEQ ID N^{o} 3858 | Listeria monocytogenes 4b Contig987 |
| | Corresponding to the former SEQ ID n^{o} 1740 |
| | Corresponding to the former SEQ ID n^{o} 2034 |
| SEQ ID N^{o} 3859 | Listeria monocytogenes 4b Contig988 |
| | Corresponding to the former SEQ ID n^{o} 1156 |
| | Corresponding to the former SEQ ID n^{o} 1241 |
| | Corresponding to the former SEQ ID n^{o} 1715 |
| | Corresponding to the former SEQ ID n^{o} 1958 |
| SEQ ID N^{o} 3860 | Listeria monocytogenes 4b Contig989 |
| | Corresponding to the former SEQ ID n^{o} 1158 |
| | Corresponding to the former SEQ ID n^{o} 1719 |
| | Corresponding to the former SEQ ID n^{o} 2023 |
| SEQ ID N^{o} 3861 | Listeria monocytogenes 4b Contig990 |
| | Corresponding to the former SEQ ID n^{o} 1110 |
| | Corresponding to the former SEQ ID n^{o} 1911 |
| | Corresponding to the former SEQ ID n^{o} 2022 |
| SEQ ID N^{o} 3862 | Listeria monocytogenes 4b Contig991 |
| | Corresponding to the former SEQ ID n^{o} 1190 |
| | Corresponding to the former SEQ ID n^{o} 1735 |
| | Corresponding to the former SEQ ID n^{o} 1823 |
| | Corresponding to the former SEQ ID n^{o} 1824 |
| SEQ ID N^{o} 3863 | Listeria monocytogenes 4b Contig992 |
| | Corresponding to the former SEQ ID n^{o} 1257 |
| | Corresponding to the former SEQ ID n^{o} 1907 |
| | Corresponding to the former SEQ ID n^{o} 1989 |
| SEQ ID N^{o} 3864 | Listeria monocytogenes 4b Contig993 |
| | Corresponding to the former SEQ ID n^{o} 1802 |
| | Corresponding to the former SEQ ID n^{o} 1803 |
| | Corresponding to the former SEQ ID n^{o} 1833 |
| | Corresponding to the former SEQ ID n^{o} 2016 |
| SEQ ID N^{o} 3865 | Listeria monocytogenes 4b Contig994 |
| | Corresponding to the former SEQ ID n^{o} 1879 |
| | Corresponding to the former SEQ ID n^{o} 1924 |
| | Corresponding to the former SEQ ID n^{o} 1977 |
| | Corresponding to the former SEQ ID n^{o} 1993 |
| SEQ ID N^{o} 3866 | Listeria monocytogenes 4b Contig995 |
| | Corresponding to the former SEQ ID n^{o} 1390 |
| | Corresponding to the former SEQ ID n^{o} 1834 |
| | Corresponding to the former SEQ ID n^{o} 1876 |
| SEQ ID N^{o} 3867 | Listeria monocytogenes 4b Contig996 |
| | Corresponding to the former SEQ ID n^{o} 1192 |
| | Corresponding to the former SEQ ID n^{o} 1591 |
| | Corresponding to the former SEQ ID n^{o} 1712 |
| SEQ ID N^{o} 3868 | Listeria monocytogenes 4b Contig997 |
| | Corresponding to the former SEQ ID n^{o} 1964 |
| | Corresponding to the former SEQ ID n^{o} 2032 |
| SEQ ID N^{o} 3869 | Listeria monocytogenes 4b Contig998 |
| | Corresponding to the former SEQ ID n^{o} 1287 |
| | Corresponding to the former SEQ ID n^{o} 1430 |
| | Corresponding to the former SEQ ID n^{o} 1678 |
| | Corresponding to the former SEQ ID n^{o} 1902 |
| | Corresponding to the former SEQ ID n^{o} 1940 |
| SEQ ID N^{o} 3870 | Listeria monocytogenes 4b Contig999 |
| | Corresponding to the former SEQ ID n^{o} 1357 |
| | Corresponding to the former SEQ ID n^{o} 1646 |
| | Corresponding to the former SEQ ID n^{o} 1887 |
| | Corresponding to the former SEQ ID n^{o} 1995 |
| SEQ ID N^{o} 3871 | Listeria monocytogenes 4b Contig1000 |
| | Corresponding to the former SEQ ID n^{o} 1202 |
| | Corresponding to the former SEQ ID n^{o} 1724 |
| | Corresponding to the former SEQ ID n^{o} 2036 |
| SEQ ID N^{o} 3872 | Listeria monocytogenes 4b Contig1001 |
| | Corresponding to the former SEQ ID n^{o} 2039 |
| SEQ ID N^{o} 3873 | Listeria monocytogenes 4b Contig1002 |
| | Corresponding to the former SEQ ID n^{o} 1113 |
| | Corresponding to the former SEQ ID n^{o} 1429 |
| | Corresponding to the former SEQ ID n^{o} 1600 |
| | Corresponding to the former SEQ ID n^{o} 1956 |
| SEQ ID N^{o} 3874 | Listeria monocytogenes 4b Contig1003 |
| | Corresponding to the former SEQ ID n^{o} 1214 |
| | Corresponding to the former SEQ ID n^{o} 1817 |
| SEQ ID N^{o} 3875 | Listeria monocytogenes 4b Contig1004 |
| | Corresponding to the former SEQ ID n^{o} 1249 |
| | Corresponding to the former SEQ ID n^{o} 1378 |
| | Corresponding to the former SEQ ID n^{o} 1463 |
| | Corresponding to the former SEQ ID n^{o} 1708 |
| | Corresponding to the former SEQ ID n^{o} 1749 |
| SEQ ID N^{o} 3876 | Listeria monocytogenes 4b Contig1005 |
| | Corresponding to the former SEQ ID n^{o} 1544 |
| | Corresponding to the former SEQ ID n^{o} 1723 |
| | Corresponding to the former SEQ ID n^{o} 1738 |
| | Corresponding to the former SEQ ID n^{o} 2015 |
| SEQ ID N^{o} 3877 | Listeria monocytogenes 4b Contig1006 |
| | Corresponding to the former SEQ ID n^{o} 1516 |
| | Corresponding to the former SEQ ID n^{o} 1746 |
| | Corresponding to the former SEQ ID n^{o} 1830 |
| SEQ ID N^{o} 3878 | Listeria monocytogenes 4b Contig1007 |
| | Corresponding to the former SEQ ID n^{o} 1467 |
| | Corresponding to the former SEQ ID n^{o} 1806 |
| | Corresponding to the former SEQ ID n^{o} 1811 |
| | Corresponding to the former SEQ ID n^{o} 1837 |
| SEQ ID N^{o} 3879 | Listeria monocytogenes 4b Contig1008 |
| | Corresponding to the former SEQ ID n^{o} 1644 |
| | Corresponding to the former SEQ ID n^{o} 1702 |
| | Corresponding to the former SEQ ID n^{o} 1990 |
| | Corresponding to the former SEQ ID n^{o} 1996 |
| SEQ ID N^{o} 3880 | Listeria monocytogenes 4b Contig1009 |
| | Corresponding to the former SEQ ID n^{o} 1565 |
| | Corresponding to the former SEQ ID n^{o} 1595 |
| | Corresponding to the former SEQ ID n^{o} 1918 |
| SEQ ID N^{o} 3881 | Listeria monocytogenes 4b Contig1010 |
| | Corresponding to the former SEQ ID n^{o} 1361 |
| | Corresponding to the former SEQ ID n^{o} 1478 |
| | Corresponding to the former SEQ ID n^{o} 1510 |
| | Corresponding to the former SEQ ID n^{o} 2006 |
| SEQ ID N^{o} 3882 | Listeria monocytogenes 4b Contig1011 |
| | Corresponding to the former SEQ ID n^{o} 1083 |
| | Corresponding to the former SEQ ID n^{o} 2037 |
| SEQ ID N^{o} 3883 | Listeria monocytogenes 4b Contig1012 |
| | Corresponding to the former SEQ ID n^{o} 1769 |
| | Corresponding to the former SEQ ID n^{o} 1835 |
| | Corresponding to the former SEQ ID n^{o} 1850 |
| | Corresponding to the former SEQ ID n^{o} 1867 |
| SEQ ID N^{o} 3884 | Listeria monocytogenes 4b Contig1013 |
| | Corresponding to the former SEQ ID n^{o} 1982 |
| | Corresponding to the former SEQ ID n^{o} 2035 |
| SEQ ID N^{o} 3885 | Listeria monocytogenes 4b Contig1014 |
| | Corresponding to the former SEQ ID n^{o} 1529 |
| | Corresponding to the former SEQ ID n^{o} 1581 |
| | Corresponding to the former SEQ ID n^{o} 1739 |
| | Corresponding to the former SEQ ID n^{o} 1865 |
| | Corresponding to the former SEQ ID n^{o} 1970 |
| SEQ ID N^{o} 3886 | Listeria monocytogenes 4b Contig1015 |
| | Corresponding to the former SEQ ID n^{o} 1170 |
| | Corresponding to the former SEQ ID n^{o} 1180 |
| | Corresponding to the former SEQ ID n^{o} 1265 |
| | Corresponding to the former SEQ ID n^{o} 1434 |
| | Corresponding to the former SEQ ID n^{o} 1536 |
| | Corresponding to the former SEQ ID n^{o} 1548 |
| | Corresponding to the former SEQ ID n^{o} 1877 |
| SEQ ID N^{o} 3887 | Listeria monocytogenes 4b Contig1016 |
| | Corresponding to the former SEQ ID n^{o} 1219 |
| | Corresponding to the former SEQ ID n^{o} 1917 |
| | Corresponding to the former SEQ ID n^{o} 2040 |
| SEQ ID N^{o} 3888 | Listeria monocytogenes 4b Contig1017 |
| | Corresponding to the former SEQ ID n^{o} 1245 |
| | Corresponding to the former SEQ ID n^{o} 1821 |
| | Corresponding to the former SEQ ID n^{o} 1841 |
| | Corresponding to the former SEQ ID n^{o} 2004 |
| | Corresponding to the former SEQ ID n^{o} 2025 |
| SEQ ID N^{o} 3889 | Listeria monocytogenes 4b Contig1018 |
| | Corresponding to the former SEQ ID n^{o} 1317 |
| | Corresponding to the former SEQ ID n^{o} 1813 |
| | Corresponding to the former SEQ ID n^{o} 1991 |
| | Corresponding to the former SEQ ID n^{o} 2001 |
| | Corresponding to the former SEQ ID n^{o} 2029 |
| SEQ ID N^{o} 3890 | Listeria monocytogenes 4b Contig1019 |
| | Corresponding to the former SEQ ID n^{o} 1926 |
| | Corresponding to the former SEQ ID n^{o} 2041 |
| SEQ ID N^{o} 3891 | Listeria monocytogenes 4b Contig1020 |
| | Corresponding to the former SEQ ID n^{o} 1873 |

Table IX: Legends

SEQ ID Nos. 3892-4025: sequences of 134 Contigs derived from the assembly of 13 919 sequences of Listeria monocytogenes 4b after taking away the sequences of L.monocytogenes EGDe and of L. innocua Clip11262.

TABLE IX

| | |
|---|---|
| SEQ ID N° 3892 | Listeria monocytogenes 4b-specifique Contig6 |
| SEQ ID N° 3893 | Listeria monocytogenes 4b-specifique Contig7 |
| SEQ ID N° 3894 | Listeria monocytogenes 4b-specifique Contig8 |
| SEQ ID N° 3895 | Listeria monocytogenes 4b-specifique Contig9 |
| SEQ ID N° 3896 | Listeria monocytogenes 4b-specifique Contig10 |
| SEQ ID N° 3897 | Listeria monocytogenes 4b-specifique Contig11 |
| SEQ ID N° 3898 | Listeria monocytogenes 4b-specifique Contig12 |
| SEQ ID N° 3899 | Listeria monocytogenes 4b-specifique Contig13 |
| SEQ ID N° 3900 | Listeria monocytogenes 4b-specifique Contig14 |
| SEQ ID N° 3901 | Listeria monocytogenes 4b-specifique Contig15 |
| SEQ ID N° 3902 | Listeria monocytogenes 4b-specifique Contig16 |
| SEQ ID N° 3903 | Listeria monocytogenes 4b-specifique Contig17 |
| SEQ ID N° 3904 | Listeria monocytogenes 4b-specifique Contig18 |
| SEQ ID N° 3905 | Listeria monocytogenes 4b-specifique Contig19 |
| SEQ ID N° 3906 | Listeria monocytogenes 4b-specifique Contig20 |
| SEQ ID N° 3907 | Listeria monocytogenes 4b-specifique Contig21 |
| SEQ ID N° 3908 | Listeria monocytogenes 4b-specifique Contig22 |
| SEQ ID N° 3909 | Listeria monocytogenes 4b-specifique Contig23 |
| SEQ ID N° 3910 | Listeria monocytogenes 4b-specifique Contig24 |
| SEQ ID N° 3911 | Listeria monocytogenes 4b-specifique Contig25 |
| SEQ ID N° 3912 | Listeria monocytogenes 4b-specifique Contig26 |
| SEQ ID N° 3913 | Listeria monocytogenes 4b-specifique Contig27 |
| SEQ ID N° 3914 | Listeria monocytogenes 4b-specifique Contig28 |
| SEQ ID N° 3915 | Listeria monocytogenes 4b-specifique Contig29 |
| SEQ ID N° 3916 | Listeria monocytogenes 4b-specifique Contig30 |
| SEQ ID N° 3917 | Listeria monocytogenes 4b-specifique Contig31 |
| SEQ ID N° 3918 | Listeria monocytogenes 4b-specifique Contig32 |
| SEQ ID N° 3919 | Listeria monocytogenes 4b-specifique Contig33 |
| SEQ ID N° 3920 | Listeria monocytogenes 4b-specifique Contig34 |
| SEQ ID N° 3921 | Listeria monocytogenes 4b-specifique Contig35 |
| SEQ ID N° 3922 | Listeria monocytogenes 4b-specifique Contig36 |
| SEQ ID N° 3923 | Listeria monocytogenes 4b-specifique Contig37 |
| SEQ ID N° 3924 | Listeria monocytogenes 4b-specifique Contig38 |
| SEQ ID N° 3925 | Listeria monocytogenes 4b-specifique Contig39 |
| SEQ ID N° 3926 | Listeria monocytogenes 4b-specifique Contig40 |
| SEQ ID N° 3927 | Listeria monocytogenes 4b-specifique Contig41 |
| SEQ ID N° 3928 | Listeria monocytogenes 4b-specifique Contig42 |
| SEQ ID N° 3929 | Listeria monocytogenes 4b-specifique Contig43 |
| SEQ ID N° 3930 | Listeria monocytogenes 4b-specifique Contig44 |
| SEQ ID N° 3931 | Listeria monocytogenes 4b-specifique Contig45 |
| SEQ ID N° 3932 | Listeria monocytogenes 4b-specifique Contig46 |
| SEQ ID N° 3933 | Listeria monocytogenes 4b-specifique Contig47 |
| SEQ ID N° 3934 | Listeria monocytogenes 4b-specifique Contig48 |
| SEQ ID N° 3935 | Listeria monocytogenes 4b-specifique Contig49 |
| SEQ ID N° 3936 | Listeria monocytogenes 4b-specifique Contig50 |
| SEQ ID N° 3937 | Listeria monocytogenes 4b-specifique Contig51 |
| SEQ ID N° 3938 | Listeria monocytogenes 4b-specifique Contig52 |
| SEQ ID N° 3939 | Listeria monocytogenes 4b-specifique Contig53 |
| SEQ ID N° 3940 | Listeria monocytogenes 4b-specifique Contig54 |
| SEQ ID N° 3941 | Listeria monocytogenes 4b-specifique Contig55 |
| SEQ ID N° 3942 | Listeria monocytogenes 4b-specifique Contig56 |
| SEQ ID N° 3943 | Listeria monocytogenes 4b-specifique Contig57 |
| SEQ ID N° 3944 | Listeria monocytogenes 4b-specifique Contig58 |
| SEQ ID N° 3945 | Listeria monocytogenes 4b-specifique Contig59 |
| SEQ ID N° 3946 | Listeria monocytogenes 4b-specifique Contig60 |
| SEQ ID N° 3947 | Listeria monocytogenes 4b-specifique Contig61 |
| SEQ ID N° 3948 | Listeria monocytogenes 4b-specifique Contig62 |
| SEQ ID N° 3949 | Listeria monocytogenes 4b-specifique Contig63 |
| SEQ ID N° 3950 | Listeria monocytogenes 4b-specifique Contig64 |
| SEQ ID N° 3951 | Listeria monocytogenes 4b-specifique Contig65 |
| SEQ ID N° 3952 | Listeria monocytogenes 4b-specifique Contig66 |
| SEQ ID N° 3953 | Listeria monocytogenes 4b-specifique Contig67 |
| SEQ ID N° 3954 | Listeria monocytogenes 4b-specifique Contig68 |
| SEQ ID N° 3955 | Listeria monocytogenes 4b-specifique Contig69 |
| SEQ ID N° 3956 | Listeria monocytogenes 4b-specifique Contig70 |
| SEQ ID N° 3957 | Listeria monocytogenes 4b-specifique Contig71 |
| SEQ ID N° 3958 | Listeria monocytogenes 4b-specifique Contig72 |
| SEQ ID N° 3959 | Listeria monocytogenes 4b-specifique Contig73 |
| SEQ ID N° 3960 | Listeria monocytogenes 4b-specifique Contig74 |
| SEQ ID N° 3961 | Listeria monocytogenes 4b-specifique Contig75 |
| SEQ ID N° 3962 | Listeria monocytogenes 4b-specifique Contig76 |
| SEQ ID N° 3963 | Listeria monocytogenes 4b-specifique Contig77 |
| SEQ ID N° 3964 | Listeria monocytogenes 4b-specifique Contig78 |
| SEQ ID N° 3965 | Listeria monocytogenes 4b-specifique Contig79 |
| SEQ ID N° 3966 | Listeria monocytogenes 4b-specifique Contig80 |
| SEQ ID N° 3967 | Listeria monocytogenes 4b-specifique Contig81 |
| SEQ ID N° 3968 | Listeria monocytogenes 4b-specifique Contig82 |
| SEQ ID N° 3969 | Listeria monocytogenes 4b-specifique Contig83 |
| SEQ ID N° 3970 | Listeria monocytogenes 4b-specifique Contig84 |
| SEQ ID N° 3971 | Listeria monocytogenes 4b-specifique Contig85 |
| SEQ ID N° 3972 | Listeria monocytogenes 4b-specifique Contig86 |
| SEQ ID N° 3973 | Listeria monocytogenes 4b-specifique Contig87 |
| SEQ ID N° 3974 | Listeria monocytogenes 4b-specifique Contig88 |
| SEQ ID N° 3975 | Listeria monocytogenes 4b-specifique Contig89 |
| SEQ ID N° 3976 | Listeria monocytogenes 4b-specifique Contig90 |
| SEQ ID N° 3977 | Listeria monocytogenes 4b-specifique Contig91 |
| SEQ ID N° 3978 | Listeria monocytogenes 4b-specifique Contig92 |
| SEQ ID N° 3979 | Listeria monocytogenes 4b-specifique Contig93 |
| SEQ ID N° 3980 | Listeria monocytogenes 4b-specifique Contig94 |
| SEQ ID N° 3981 | Listeria monocytogenes 4b-specifique Contig95 |
| SEQ ID N° 3982 | Listeria monocytogenes 4b-specifique Contig96 |
| SEQ ID N° 3983 | Listeria monocytogenes 4b-specifique Contig97 |
| SEQ ID N° 3984 | Listeria monocytogenes 4b-specifique Contig98 |
| SEQ ID N° 3985 | Listeria monocytogenes 4b-specifique Contig99 |
| SEQ ID N° 3986 | Listeria monocytogenes 4b-specifique Contig100 |
| SEQ ID N° 3987 | Listeria monocytogenes 4b-specifique Contig101 |
| SEQ ID N° 3988 | Listeria monocytogenes 4b-specifique Contig102 |
| SEQ ID N° 3989 | Listeria monocytogenes 4b-specifique Contig103 |
| SEQ ID N° 3990 | Listeria monocytogenes 4b-specifique Contig104 |
| SEQ ID N° 3991 | Listeria monocytogenes 4b-specifique Contig105 |
| SEQ ID N° 3992 | Listeria monocytogenes 4b-specifique Contig106 |
| SEQ ID N° 3993 | Listeria monocytogenes 4b-specifique Contig107 |
| SEQ ID N° 3994 | Listeria monocytogenes 4b-specifique Contig108 |
| SEQ ID N° 3995 | Listeria monocytogenes 4b-specifique Contig109 |
| SEQ ID N° 3996 | Listeria monocytogenes 4b-specifique Contig110 |
| SEQ ID N° 3997 | Listeria monocytogenes 4b-specifique Contig111 |
| SEQ ID N° 3998 | Listeria monocytogenes 4b-specifique Contig112 |
| SEQ ID N° 3999 | Listeria monocytogenes 4b-specifique Contig113 |
| SEQ ID N° 4000 | Listeria monocytogenes 4b-specifique Contig114 |
| SEQ ID N° 4001 | Listeria monocytogenes 4b-specifique Contig115 |
| SEQ ID N° 4002 | Listeria monocytogenes 4b-specifique Contig116 |
| SEQ ID N° 4003 | Listeria monocytogenes 4b-specifique Contig117 |
| SEQ ID N° 4004 | Listeria monocytogenes 4b-specifique Contig118 |
| SEQ ID N° 4005 | Listeria monocytogenes 4b-specifique Contig119 |
| SEQ ID N° 4006 | Listeria monocytogenes 4b-specifique Contig120 |
| SEQ ID N° 4007 | Listeria monocytogenes 4b-specifique Contig121 |
| SEQ ID N° 4008 | Listeria monocytogenes 4b-specifique Contig122 |
| SEQ ID N° 4009 | Listeria monocytogenes 4b-specifique Contig123 |
| SEQ ID N° 4010 | Listeria monocytogenes 4b-specifique Contig124 |
| SEQ ID N° 4011 | Listeria monocytogenes 4b-specifique Contig125 |
| SEQ ID N° 4012 | Listeria monocytogenes 4b-specifique Contig126 |
| SEQ ID N° 4013 | Listeria monocytogenes 4b-specifique Contig127 |
| SEQ ID N° 4014 | Listeria monocytogenes 4b-specifique Contig128 |
| SEQ ID N° 4015 | Listeria monocytogenes 4b-specifique Contig129 |
| SEQ ID N° 4016 | Listeria monocytogenes 4b-specifique Contig130 |
| SEQ ID N° 4017 | Listeria monocytogenes 4b-specifique Contig131 |
| SEQ ID N° 4018 | Listeria monocytogenes 4b-specifique Contig132 |
| SEQ ID N° 4019 | Listeria monocytogenes 4b-specifique Contig133 |
| SEQ ID N° 4020 | Listeria monocytogenes 4b-specifique Contig134 |
| SEQ ID N° 4021 | Listeria monocytogenes 4b-specifique Contig135 |
| SEQ ID N° 4022 | Listeria monocytogenes 4b-specifique Contig136 |
| SEQ ID N° 4023 | Listeria monocytogenes 4b-specifique Contig137 |
| SEQ ID N° 4024 | Listeria monocytogenes 4b-specifique Contig138 |
| SEQ ID N° 4025 | Listeria monocytogenes 4b-specifique Contig139 |

1. A method for identifying nucleotide sequences specific for the genome of a strain of bacterium of the genus Listeria, in particular specific for a strain of L. innocua or L. monocytogenes, such as the strain L. monocytogenes EGDe or L. monocytogenes 4b.

2. The method for identifying nucleotide sequences as claimed in claim 1, characterized in that the sequences specific for:
L. innocua compared to L. monocytogenes, in particular compared to L. monocytogenes EGDe and/or L. monocytogenes 4b;
L. monocytogenes, in particular L. monocytogenes EGDe or L. monocytogenes 4b, compared to L. innocua;
L. monocytogenes EGDe compared to L. innocua and/or L. monocytogenes 4b; or
L. monocytogenes 4b compared to L. innocua and/or L. monocytogenes EGDe are identified.

3. The method for identifying nucleotide sequences as claimed in claim 1 or 2, characterized in that it comprises at least the following steps:
a) aligning the nucleotide sequences of L. monocytogenes, in particular those of L. monocytogenes EGDe and/or L. monocytogenes 4b, and those of L. innocua as claimed in claims 5 to 8, 10 to 17 and 21; and
b) processing the data obtained with this alignment in order to isolate said specific sequences.

4. The method for identifying nucleotide sequences as claimed in one of claims 1 to 3, characterized in that the nucleotide sequences specific for L. innocua or L. monocytogenes, in particular those for L. monocytogenes EGDe and/or L. monocytogenes 4b, hybridize, under high stringency conditions, with respectively the nucleotide sequences, or the sequence complementary thereto, of L. innocua or L. monocytogenes, in particular those of L. monocytogenes EGDe and/or L. monocytogenes 4b.

5. A nucleotide sequence derived from Listeria innocua, characterized in that it corresponds to a sequence chosen from SEQ ID No. 1 to SEQ ID No. 11, SEQ ID No. 2057 and SEQ ID No. 2058.

6. A nucleotide sequence derived from Listeria innocua, characterized in that it is chosen from
a) a nucleotide sequence comprising at least 75% identity with a sequence chosen from SEQ ID No. 1 to SEQ ID No. 11, SEQ ID No. 2057 and SEQ ID No. 2058;
b) a nucleotide sequence which hybridizes, under high stringency conditions, with a sequence chosen from SEQ ID No. 1 to SEQ ID No. 11, SEQ ID No. 2057 and SEQ ID No. 2058;
c) a nucleotide sequence complementary to a sequence chosen from SEQ ID No. 1 to SEQ ID No. 11, SEQ ID No. 2057 and SEQ ID No. 2058, or complementary to a nucleotide sequence as defined in a) or b), or a nucleotide sequence of the RNA corresponding to either of the sequences a) and b);
d) a nucleotide sequence of a representative fragment of a sequence chosen from SEQ ID No. 1 to SEQ ID No. 11, SEQ ID No. 2057 and SEQ ID No. 2058, or of a representative fragment of a nucleotide sequence as defined in a), b) or c);
e) a nucleotide sequence comprising a sequence as defined in a), b), c) or d); and
f) a nucleotide sequence as defined in a), b), c), d) or e) which has been modified.

7. The nucleotide sequence as claimed in claim 6, characterized in that it is a sequence derived from a sequence chosen from SEQ ID No. 1 to SEQ ID No. 11, SEQ ID No. 2057 and SEQ ID No. 2058, and in that it encodes a polypeptide, said nucleotide sequence preferably being chosen from the sequences SEQ ID No. 12 to SEQ ID No. 689, SEQ ID No. 2053 to SEQ ID No. 2056 and SEQ ID No. 2059 to SEQ ID No. 2601.

8. A nucleotide sequence characterized in that it comprises a nucleotide sequence chosen from:
a) a nucleotide sequence as claimed in claim 7;
b) a nucleotide sequence comprising at least 75% identity with a nucleotide sequence as claimed in claim 7;
c) a nucleotide sequence which hybridizes, under high stringency conditions, with a nucleotide sequence as claimed in claim 7;
d) a complementary or RNA nucleotide sequence corresponding to a sequence as defined in a), b) or c);
e) a nucleotide sequence of a representative fragment of a sequence as defined in a), b), c) or d); and
f) a sequence as defined in a), b), c), d) or e) which has been modified.

9. A polypeptide encoded by a nucleotide sequence as claimed in one of claims 6 to 8.

10. The polypeptide as claimed in claim 9, characterized in that it is chosen from the polypeptides encoded by a sequence chosen from SEQ ID No. 12 to SEQ ID No. 689, SEQ ID No. 2053 to SEQ ID No. 2056 and SEQ ID No. 2059 to SEQ ID No. 2601.

11. A polypeptide characterized in that it comprises a polypeptide chosen from:
a) a polypeptide as claimed in either of claims 9 and 10;
b) a polypeptide exhibiting at least 80% identity with a polypeptide as claimed in either of claims 9 and 10;
c) a fragment of at least 5 amino acids of a polypeptide as claimed in either of claims 9 and 10 or as defined in b);
d) a biologically active fragment of a polypeptide as claimed in either of claims 9 and 10 or as defined in b) or c); and
e) a polypeptide as claimed in either of claims 9 and 10 or as defined in b), c) or d) which has been modified.

12. A nucleotide sequence encoding a polypeptide as claimed in claim 11.

13. A nucleotide sequence encoding a polypeptide specific for L. innocua characterized in that it is chosen from SEQ ID No. 12 to SEQ ID No. 689 and SEQ ID No. 2059 to SEQ ID No. 2601.

14. A nucleotide sequence derived from Listeria monocytogenes serotype 4b, characterized in that it corresponds to a sequence chosen from SEQ ID No. 1068 to SEQ ID No. 2041 and SEQ ID No. 2872 to SEQ ID No. 3891.

15. A nucleotide sequence derived from Listeria monocytogenes serotype 4b, characterized in that it is chosen from:
a) a nucleotide sequence comprising at least 75% identity with a sequence chosen from SEQ ID No. 1068 to SEQ ID No. 2041 and SEQ ID No. 2872 to SEQ ID No. 3891;
b) a nucleotide sequence which hybridizes, under high stringency conditions, with a sequence chosen from SEQ ID No. 1068 to SEQ ID No. 2041 and SEQ ID No. 2872 to SEQ ID No. 3891;
c) a nucleotide sequence complementary to a sequence chosen from SEQ ID No. 1068 to SEQ ID No. 2041 and SEQ ID No. 2872 to SEQ ID No. 3891, or complementary to a nucleotide sequence as defined in a) or b), or a nucleotide sequence of the RNA corresponding to either of the sequences a) and b);
d) a nucleotide sequence of a representative fragment of a sequence chosen from SEQ ID No. 1068 to SEQ ID No. 2041 and SEQ ID No. 2872 to SEQ ID No. 3891, or of a representative fragment of a nucleotide sequence as defined in a), b) or c);
e) a nucleotide sequence comprising a sequence as defined in a), b), c) or d); and
f) a nucleotide sequence as defined in a), b), c), d) or e) which has been modified.

16. A nucleotide sequence as claimed in claim 15, characterized in that it is a sequence derived from a sequence chosen from SEQ ID No. 1068 to SEQ ID No. 2041 and SEQ ID No. 2872 to SEQ ID No. 3891, and in that it encodes a polypeptide, said nucleotide sequence preferably being chosen from the sequences SEQ ID No. 690 to SEQ ID No. 1067 and SEQ ID No. 2602 to SEQ ID No. 2871 and SEQ ID No. 2049 to SEQ ID No. 2052.

17. A nucleotide sequence characterized in that it comprises a nucleotide sequence chosen from:
a) a nucleotide sequence as claimed in claim 16;
b) a nucleotide sequence comprising at least 75% identity with a nucleotide sequence as claimed in claim 16;
c) a nucleotide sequence which hybridizes, under high stringency conditions, with a nucleotide sequence as claimed in claim 16;
d) a complementary or RNA nucleotide sequence corresponding to a sequence as defined in a), b) or c);
e) a nucleotide sequence of a representative fragment of a sequence as defined in a), b), c) or d); and
f) a sequence as defined in a), b), c), d) or e) which has been modified.

18. A polypeptide encoded by a nucleotide sequence as claimed in one of claims 15 to 17.

19. The polypeptide as claimed in claim 18, characterized in that it is chosen from the polypeptides encoded by a sequence chosen from SEQ ID No. 690 to SEQ ID No. 1067, SEQ ID No. 2049 to SEQ ID No. 2052 and SEQ ID No. 2602 to SEQ ID No. 2871.

20. A polypeptide characterized in that it comprises a polypeptide chosen from:
a) a polypeptide as claimed in either of claims 18 and 19;
b) a polypeptide exhibiting at least 80% identity with a polypeptide as claimed in either of claims 18 and 19;
c) a fragment of at least 5 amino acids of a polypeptide as claimed in either of claims 18 and 19 or as defined in b);
d) a biologically active fragment of a polypeptide as claimed in either of claims 18 and 19 or as defined in b) or c); and
e) a polypeptide as claimed in either of claims 18 and 19 or as defined in b), c) or d) which has been modified.

21. A nucleotide sequence encoding a polypeptide as claimed in claim 20.

22. A nucleotide sequence encoding a polypeptide specific for L. monocytogenes, characterized in that it is chosen from SEQ ID No. 690 to SEQ ID No. 1067, SEQ ID No. 2602 to SEQ ID No. 2871 and SEQ ID No. 3892 to SEQ ID No. 4025.

23. A nucleotide sequence encoding a polypeptide exhibiting at least 87% identity between L. innocua and L. monocytogenes, characterized in that it is chosen from SEQ ID No. 2049 to SEQ ID No. 2056.

24. The nucleotide sequence as claimed in one of claims 6 to 8, 12, 13, 15 to 17, and 21 to 23, characterized in that it encodes a polypeptide of L. innocua or L. monocytogenes, or one of its fragments:
involved in amino acid biosynthesis or in the biosynthesis of cofactors, prosthetic groups and transporters;
which is a cell envelope polypeptide or a polypeptide located at the surface of L. innocua or L. monocytogenes;
involved in the cellular machinery;
involved in central intermediate metabolism;
involved in energy metabolism;
involved in fatty acid and phospholipid metabolism;
involved in the metabolism of nucleotides, purines, pyrimidines or nucleosides;
involved in regulatory functions;
involved in the replication process;
involved in the transcription process;
involved in the translation process;
involved in the process of protein transport and binding;
involved in adaptation to atypical conditions;
involved in sensitivity to medicinal products and analogs; or
involved in functions relating to transposons.

25. The polypeptide as claimed in one of claims 9 to 11, and 17 to 20, characterized in that it is a polypeptide of L. innocua or L. monocytogenes:
involved in amino acid biosynthesis or in the biosynthesis of cofactors, prosthetic groups and transporters;
which is a cell envelope polypeptide or a polypeptide located at the surface of L. innocua or L. monocytogenes;
involved in the cellular machinery;
involved in central intermediate metabolism;
involved in energy metabolism;
involved in fatty acid and phospholipid metabolism;
involved in the metabolism of nucleotides, purines, pyrimidines or nucleosides;
involved in regulatory functions;
involved in the replication process;
involved in the transcription process;
involved in the translation process;
involved in the process of protein transport and binding;
involved in adaptation to atypical conditions;
involved in sensitivity to medicinal products and analogs; or
involved in functions relating to transposons.

26. A nucleotide sequence which can be used as a primer or as a probe, characterized in that its sequence is chosen from the nucleotide sequences as claimed in one of claims 6 to 8, 12 to 17, and 21 to 23.

27. The nucleotide sequence as claimed in claim 26, characterized in that it is labeled with a radioactive compound or with a nonradioactive compound.

28. The nucleotide sequence as claimed in either of claims 26 and 27, characterized in that it is immobilized on a support, covalently or noncovalently.

29. The nucleotide sequence as claimed in claim 28, characterized in that it is immobilized on a support such as a high density filter or a DNA chip.

30. The nucleotide sequence as claimed in one of claims 27 to 29, for detecting and/or amplifying nucleic acid sequences.

31. A DNA chip or filter, characterized in that it contains at least one nucleotide sequence as claimed in claim 29.

32. The DNA chip or filter as claimed in claim 31, characterized in that it also contains at least one nucleotide sequence of a microorganism other than L. innocua or L. monocytogenes, immobilized on the support of said chip.

33. The DNA chip or filter as claimed in claim 32, characterized in that the microorganism is chosen from a microorganism related to a L. innocua or L. monocytogenes, a bacterium of the genus Listeria and a variant of L. innocua or L. monocytogenes.

34. A kit or set for detecting and/or identifying bacteria belonging to the species L. innocua or L. monocytogenes or to a related microorganism, characterized in that it comprises a DNA chip or a filter as claimed in claim 31.

35. A kit or set for detecting and/or identifying a microorganism, characterized in that it comprises a DNA chip or a filter as claimed in either of claims 32 and 33.

36. A kit or set for detecting and/or identifying the expression of at least one gene of L. innocua or L. monocytogenes, characterized in that it comprises a DNA chip or a filter as claimed in either of claims 32 and 33.

37. A cloning and/or expression vector, characterized in that it contains a nucleotide sequence as claimed in one of claims 5 to 8, 12, 13, 15 to 17, and 21 to 23.

38. A host cell, characterized in that it is transformed with a vector as claimed in claim 37.

39. The host cell as claimed in claim 38, characterized in that it is a bacterium belonging to the genus Listeria.

40. The host cell as claimed in claim 39, characterized in that it is a bacterium belonging to the species L. innocua or L. monocytogenes.

41. A plant or an animal, except a human, comprising a transformed cell as claimed in one of claims 38 to 40.

42. A method for preparing a polypeptide, characterized in that a cell transformed with a vector as claimed in claim 37 is cultured under conditions which allow the expression of said polypeptide, and in that said recombinant polypeptide is recovered.

43. A recombinant polypeptide which can be obtained using a method as claimed in claim 42.

44. A method for preparing a synthetic polypeptide as claimed in one of claims 9 to 11, 18 to 20 and 25, characterized in that chemical synthesis of said polypeptide is carried out.

45. A hybrid polypeptide, characterized in that it comprises at least the sequence of a polypeptide as claimed in one of claims 9 to 11, 18 to 20, 25 and 43, and a sequence of a polypeptide capable of inducing an immune response in humans or animals.

46. A nucleotide sequence encoding a hybrid polypeptide as claimed in claim 45.

47. A vector characterized in that it contains a nucleotide sequence as claimed in claim 46.

48. A monoclonal or polyclonal antibody, its fragments, or a chimeric antibody, characterized in that it is capable of specifically recognizing a polypeptide as claimed in one of claims 9 to 11, 18 to 20, 25, 43 and 45.

49. The antibody as claimed in claim 48, characterized in that it is a labeled antibody.

50. A method for detecting and/or identifying bacteria belonging to the species L. innocua or L. monocytogenes, or to a related microorganism, in a biological sample, characterized in that it comprises the following steps:
a) bringing the biological sample into contact with an antibody as claimed in either of claims 48 and 49;
b) demonstrating the antigen-antibody complex possibly formed.

51. A method for detecting the expression of a gene of L. innocua or L. monocytogenes, characterized in that a strain of L. innocua or L. monocytogenes is brought into contact with an antibody as claimed in claim 74 or 75, and in that the antigen-antibody complex possibly formed is detected.

52. A kit or set for carrying out a method as claimed in claim 50 or 51, characterized in that it comprises the following elements:
a) an antibody as claimed in either of claims 48 and 49;
b) optionally, the reagents for constituting the medium suitable for the immunoreaction;
c) optionally, the reagents for demonstrating the antigen-antibody complexes produced by the immunoreaction.

53. The polypeptide as claimed in one of claims 9 to 11, 18 to 20, 25, 43 and 45, or the antibody as claimed in either of claims 48 and 49, characterized in that it is immobilized on a support, in particular a protein chip.

54. A protein chip, characterized in that it contains at least one polypeptide as claimed in one of claims 9 to 11, 18 to 20, 25, 43 and 45, or at least one antibody as claimed in either of claims 48 and 49, immobilized on the support of said chip.

55. The protein chip as claimed in claim 54, characterized in that it also contains at least one polypeptide of a microorganism other than L. innocua or L. monocytogenes or at least one antibody directed against a compound of a microorganism other than L. innocua or L. monocytogenes, immobilized on the support of said chip.

56. A kit or set for detecting and/or identifying bacteria belonging to the species L. innocua or L. monocytogenes, or to a related microorganism, characterized in that it comprises a protein chip as claimed in either of claims 54 and 55.

57. A kit or set for detecting and/or identifying a microorganism, characterized in that it comprises a protein chip as claimed in claim 56.

58. A method for detecting and/or identifying bacteria belonging to the species L. innocua or L. monocytogenes, or to a related microorganism, in a biological sample, characterized in that it uses a nucleotide sequence as claimed in one of claims 6 to 8, 12, 13, 15 to 17, 21 to 24, 26 to 30 and 46.

59. The method as claimed in claim 58, characterized in that it comprises the following steps:
a) optionally isolating the DNA from the biological sample to be analyzed, or obtaining a cDNA from the RNA of the biological sample;
b) specifically amplifying the DNA of bacteria belonging to the species L. innocua or monocytogenes 4b, or to a related microorganism, using at least one primer as claimed in one of claims 26 to 30;
c) demonstrating the amplification products.

60. The method as claimed in claim 58, characterized in that it comprises the following steps:
a) bringing a nucleotide probe as claimed in one of claims 26 to 30 into contact with a biological sample, the nucleic acid contained in the biological sample having, where appropriate, previously been made accessible to hybridization, under conditions which allow hybridization of the probe to the nucleic acid of a bacterium belonging to the species L. innocua or L. monocytogenes 4b, or to a related microroganism;
b) demonstrating the hybrid possibly formed between the nucleotide probe and the nucleic acid of the biological sample.

61. The method as claimed in claim 58, characterized in that it comprises the following steps:
a) bringing a nucleotide probe immobilized on a support as claimed in claim 28 into contact with a biological sample, the nucleic acid of the sample having, where appropriate, previously been made accessible to hybridization, under conditions which allow hybridization of the probe to the nucleic acid of a bacterium belonging to the species L. innocua or L. monocytogenes, or to a related microorganism;
b) bringing the hybrid formed between the nucleotide probe immobilized on a support and the nucleic acid contained in the biological sample, where appropriate after removing the nucleic acid of the biological sample which has not hybridized with the probe, into contact with a labeled nucleotide probe as claimed in claim 27;
c) demonstrating the new hybrid formed in step b).

62. The method as claimed in claim 61, characterized in that, prior to step a), the DNA of the biological sample or the cDNA optionally obtained by reverse transcription of the RNA of the sample is amplified using at least one primer as claimed in one of claims 26 to 30.

63. A kit or set for detecting and/or identifying bacteria belonging to the species L. innocua or L. monocytogenes, or to a related microorganism, characterized in that it comprises the following elements:
a) a nucleotide probe as claimed in one of claims 26 to 30;
b) optionally, the reagents required to carry out a hybridization reaction;
c) optionally, at least one primer as claimed in one of claims 26 to 30 and also the reagents required for a DNA amplification reaction.

64. A kit or set for detecting and/or identifying bacteria belonging to the species L. innocua or L. monocytogenes, or to a related microorganism, characterized in that it comprises the following elements:
a) a nucleotide probe, termed capture probe, as claimed in claim 28
b) an oligonucleotide probe, termed revealing probe, as claimed in claim 27;
c) optionally, at least one primer as claimed in one of claims 26 to 30 and also the reagents required for a DNA amplification reaction.

65. A kit or set for detecting and/or identifying bacteria belonging to the species L. innocua or L. monocytogenes, or to a related microorganism, characterized in that it comprises the following elements:
a) at least one primer as claimed in one of claims 26 to 30;
b) optionally, the reagents required to carry out a DNA amplification reaction;
c) optionally, a component for verifying the sequence of the amplified fragment, more particularly an oligonucleotide probe as claimed in one of claims 26 to 30.

66. The method as claimed in claims 50, 51 and 58 to 62 or the kit or set as claimed in claims 52, 56, 57 and 63 to 65, for detecting and/or identifying bacteria belonging to the species L. innocua or L. monocytogenes, characterized in that said primer and/or said probe are chosen from the nucleotide sequences as claimed in one of claims 6 to 8, 12, 13, 15 to 17, 21 to 24, 26 to 30 and 46, specific for the species L. innocua or L. monocytogenes, in that said polypeptides are chosen from the polypeptides as claimed in one of claims 9 to 11, 18 to 20, 25, 43 and 45, specific for the species L. innocua or L. monocytogenes, and in that said antibodies are chosen from the antibodies as claimed in either of claims 48 and 49, directed against the polypeptides chosen from the polypeptides as claimed in one of claims 9 to 11, 18 to 20, 25, 43 and 45, specific for the species L. innocua or L. monocytogenes.

67. A strain of L. innocua or L. monocytogenes, characterized in that it contains at least one mutation in at least one nucleotide sequence as claimed in one of claims 6 to 8, 12, 13, 15 to 17, and 21 to 24.

68. The strain of L. innocua or L. monocytogenes as claimed in claim 67, characterized in that the mutation leads to inactivation of the gene.

69. The strain of L. innocua or L. monocytogenes as claimed in claim 67, characterized in that the mutation leads to overexpression of the gene.

70. The use of a nucleotide sequence as claimed in one of claims 6 to 8, 12, 13, 15 to 17, and 21 to 24, of a polypeptide as claimed in one of claims 9 to 11, 18 to 20, 25, 43 and 45, of an antibody as claimed in either of claims 48 and 49, of a cell as claimed in one of claims 38 to 40, and/or of a transformed animal as claimed in claim 41, for selecting an organic or inorganic compound capable of modulating, regulating, inducing or inhibiting gene expression, and/or of modifying cell replication in eukaryotic or prokaryotic cells, or capable of inducing, inhibiting or worsening in an animal or human organism pathological conditions associated with an infection with L. monocytogenes or one of its related microorganisms.

71. A method for selecting a compound capable of binding to a polypeptide as claimed in one of claims 9 to 11, 18 to 20, 25, 43 and 45, capable of binding to a nucleotide sequence as claimed in one of claims 6 to 8, 12, 13, 15 to 17, and 21 to 24, or capable of recognizing an antibody as claimed in either of claims 48 and 49, and/or capable of modulating, regulating, inducing or inhibiting gene expression and/or of modifying cell replication of eukaryotic or prokaryotic cells, or capable of inducing, inhibiting or worsening in an animal or human organism pathological conditions associated with infection with L. monocytogenes, characterized in that it comprises the following steps:
a) bringing said compound into contact with said polypeptide, with said nucleotide sequence or with a transformed cell as claimed in one of claims 38 to 40, and/or administering said compound to a transformed animal as claimed in claim 41;
b) determining the ability of said compound to bind said polypeptide or said nucleotide sequence, or to modulate, regulate, induce or inhibit gene expression, or to modulate cell growth or replication, or to induce, inhibit or worsen in said animal or human organism pathological conditions associated with infection with L. monocytogenes or with a related microorganism.

72. A pharmaceutical composition comprising a compound chosen from the following compounds:
a) a nucleotide sequence as claimed in one of claims 6 to 8, 12, 13, 15 to 17, and 21 to 24;
b) a polypeptide as claimed in one of claims 9 to 11, 18 to 20, 25, 43 and 45;
c) a vector as claimed in claim 37 or 47;
d) an antibody as claimed in claim 48 or 49.

73. The composition as claimed in claim 72, optionally in combination with a pharmaceutically acceptable vehicle.

74. The pharmaceutical composition as claimed in either of claims 72 and 73, for preventing or treating an infection with a bacterium belonging to the species L. monocytogenes, or with a related microorganism.

75. An immunogenic composition, characterized in that it comprises one or more polypeptides as claimed in one of claims 9 to 11, 18 to 20, 25 and 43, and/or one or more polypeptides as claimed in claim 45.

76. The use of a cell as claimed in one or more of claims 38 to 40, or of a vector as claimed in either of claims 37 and 47, for preparing a vaccine composition.

77. A vaccine composition, characterized in that it contains a polynucleotide as claimed in one of claims 6 to 8, 12, 13, 15 to 17, and 21 to 24, a vector as claimed in either of claims 37 and 47, and/or a cell as claimed in one of claims 38 to 40.

78. An immunogenic composition capable of inducing a cellular or humoral immune response, for preventing or treating an infection with a bacterium belonging to the species L. monocytogenes, or with a related microorganism, characterized in that it comprises an immunogenic composition as claimed in either of claims 75 and 77, in combination with a pharmaceutically acceptable vehicle and, optionally, one or more suitable adjuvants of immunity.

79. A genomic library of a bacterium of the genus Listeria.

80. The genomic DNA library of a bacterium of the genus Listeria as claimed in claim 79, characterized in that the DNA library is cloned into a plasmid.

81. The genomic DNA library as claimed in claim 79 or 80, characterized in that said bacterium is L. innocua or L. monocytogenes serotype 4b.

82. The library as claimed in claim 79 or 80, characterized in that it is the Li-shotgun library deposited with the CNCM on Oct. 2, 2000, under the No. I-2565.

83. The library as claimed in claim 79 or 80, characterized in that it is the Lm4b-shotgun library deposited with the CNCM on Oct. 2, 2000, under the No. I-2566.

84. The genomic library as claimed in claim 79, characterized in that the bacterium is L. innocua or L. monocytogenes.

85. The use of the genomic library as claimed in one of claims 79 to 84, for isolating nucleotide sequences specific for L. innocua and L. monocytogenes, characterized in that the nucleotide sequences of L. innocua and L. monocytogenes are aligned and in that the data obtained with this alignment are processed in order to isolate said specific sequences.

86. The pharmaceutical composition as claimed in one of claims 72 to 74, characterized in that it comprises antibodies directed against polypeptides specific for L. innocua or L. monocytogenes.

87. A method for identifying sequences specific for L. innocua or L. monocytogenes, characterized by the alignment of the nucleotide sequences of L. monocytogenes and of those of L. innocua, as claimed in claims 5 to 8, 12 to 17 and 21, and the processing of the data obtained with this alignment in order to isolate said specific sequences.
